(19) United States
(12) Patent Application Publication
(10) Pub. No.: US 2022/0000919 A1
(43) Pub. Date: Jan. 6, 2022

(54) PLACENTAL DERIVED NATURAL KILLER CELLS FOR TREATMENT OF CORONAVIRUS INFECTIONS

(71) Applicant: Celularity Inc., Florham Park, NJ (US)

(72) Inventors: Lin KANG, Basking Ridge, NJ (US); William VAN DER TOUW, Hoboken, NJ (US); Vanesssa VOSKINARIAN-BERSE, Millington, NJ (US); Xuan GUO, Whitehouse Station, NJ (US); Robert J. HARIRI, Bernardsville, NJ (US); Xiaokui ZHANG, Martinsville, NJ (US); Catherine BALINT, Lake Hopatcong, NJ (US); Nassir HABBOUBI, Short Hills, NJ (US); Stacy HERB, Mountaintop, PA (US); Sharmila KOPPISETTI, Parsippany, NJ (US); Tanel MAHLAKOIV, Morristown, NJ (US); Bhavani STOUT, Lebanon, NJ (US); Junhong ZHU, Pine Brook, NJ (US); Corey CASPER, Seattle, WA (US); Shuyang HE, Martinsville, NJ (US)

(73) Assignee: Celularity Inc., Florham Park, NJ (US)

(21) Appl. No.: 17/163,316

(22) Filed: Jan. 29, 2021

Related U.S. Application Data
(60) Provisional application No. 62/967,528, filed on Jan. 29, 2020.
(60) Provisional application No. 62/968,983, filed on Jan. 31, 2020.
(60) Provisional application No. 62/970,591, filed on Feb. 5, 2020.
(60) Provisional application No. 62/971,901, filed on Feb. 7, 2020.
(60) Provisional application No. 62/982,696, filed on Feb. 27, 2020.

(51) Int. Cl. A61K 35/17; C12N 5/0783

(52) U.S. Cl. CPC A61K 35/17; C12N 5/0646

(57) ABSTRACT

Provided herein are methods of using populations of natural killer (NK) cells and/or ILC3 cells derived from a population of hematopoietic stem or progenitor cells in methods for treating a viral infection, e.g., a coronavirus infection.

### 1. FIELD

Provided herein are methods of using populations of natural killer (NK) cells and/or ILC3 cells derived from a population of hematopoietic stem or progenitor cells in methods for treating a viral infection, e.g., a coronavirus infection. Such cells can be derived, e.g., from placental hematopoietic stem cells in media comprising stem cell mobilizing factors, e.g., three-stage methods of producing NK cells and/or ILC3 cells in media comprising stem cell mobilizing factors.

### 2. BACKGROUND

Natural killer (NK) cells are cytotoxic lymphocytes that constitute a major component of the innate immune system.

NK cells are activated in response to interferons or macrophage-derived cytokines. The cytotoxic activity of NK cells is largely regulated by two types of surface receptors, which may be considered “activating receptors” or “inhibitory receptors,” although some receptors, e.g., CD94 and 2B4 (CD244), can work either way depending on ligand interactions.

Among other activities, NK cells play a role in the host rejection of tumors and have been shown capable of killing virus-infected cells. Natural killer cells can become activated by cells lacking, or displaying reduced levels of, major histocompatibility complex (MHC) proteins. Cancer cells with altered or reduced level of self-class IMHC expression result in induction of NK cell sensitivity. Activated and expanded NK cells, and in some cases LAK cells, from peripheral blood have been used in both ex vivo therapy and in vivo treatment of patients having advanced cancer, with some success against bone marrow related diseases, such as leukemia; breast cancer; and certain types of lymphoma.

In spite of the advantageous properties of NK cells in killing tumor cells and virus-infected cells, there remains a need in the art to develop efficient methods to produce and expand natural killer cells that retain tumoricidal functions.

NK cells are innate lymphoid cells (ILCs). Innate lymphoid cells are related through their dependency on transcription factor ID2 for development. One type of ILC, known as the ILC3 cell, is described in the literature as expressing RORγt and producing IL-22, as well as playing a role in the immune response of adults, without manifesting cytotoxic effectors such as perforin, granzymes, and death receptors (Montaldo et al., 2014, Immunity 41:988-1000; Killig et al., 2014, Front. Immunol. 5:142; Withers et al., 2012, J. Immunol. 189(5):2094-2098).

### 3. SUMMARY

The present invention provides methods of treating a viral infection in a subject, comprising administering to the subject an amount of a composition comprising a plurality of placenta derived natural killer cells, effective to treat the viral infection in the subject.

The present invention also provides natural killer cells characterized by expression of one or more markers selected from the group consisting of FGFBP2, GZMH, CCL3L3, GZMM, CXCR4, ZEB2, KLF2, LITAF, RORA, LYAR, CNOT1, IFNG, DUSP2, ATG2A, CD7, PMAIP1, PPP2R5C, NR4A2, ZFP36L2, PIK3R1, KLRF1, SNHG9, MT2A, RGS2, CHD1, DUSP1, EML4, ZFP36, ZC3H12A, DNAJB6, SBDS, IRF1, TSC22D3, TSPYL2, PNRC1, ISCA1, JUNB, WHAMM, RICTOR, TNFAIP3, EPC1, MVD, CLK1, ARL4C, REL, KMT2E, YPEL5, AMD1, BTG2, and IDS which is lower than expression of said markers in peripheral blood natural killer cells and/or expression of one or more markers selected from the group consisting of NDFIP2, LINC00996, MAL, CCL1, MB, SPINK2, C15orf48, CAMK1, KLRC1, TNFSF10, TNFRSF18, IL32, CAPG, AC092580.4, S100A11, TNFRSF4, ENO1, FCER1G, CCND2, KRT81, MRPS6, ANXA2, PTGER2, GLO1, HAVCR2, PYCARD, LAT2, SLC16A3, COTL1, PKM, TALDO1, CD96, NCR3, KRT86, STMN1, LTB, ARPC1B, ARPC5, FKBP1A, TEMPI, GZMK, CD59, PGK1, RGS10, EVL, RAC2, LGALS1, ITGB7, TUBB, PGAM1, PRF1, GZMB, IL2RB, KLRC2, and KLRB1 which is higher than expression of said markers in peripheral blood natural killer cells for use in treating a viral infection. Also provided herein are methods of expanding and differentiating cells, for example, hematopoietic cells, such as hematopoietic stem cells, e.g., CD34⁺ hematopoietic stem cells, to produce natural killer (NK) cells and/or ILC3 cells. In particular, the present invention focuses on novel aromatic compounds (stem cell mobilizing agents/factors) which promote the proliferation/expansion of hematopoietic stem and progenitor cells in order to produce increased populations of differentiated NK and/or ILC3 cells from said hematopoietic progenitor cells.

In one aspect, provided herein are methods of producing NK cell populations and/or ILC3 cell populations that comprise three stages as described herein (and referred to herein as the “three-stage method”). Natural killer cells and/or ILC3 cells produced by the three-stage methods provided herein are referred to herein as “NK cells produced by the three-stage method,” “ILC3 cells produced by the three-stage method,” or “NK cells and/or ILC3 cells produced by the three-stage method.” In certain embodiments, said method comprises one or more further or intermediate steps. In certain embodiments, said method does not comprise any fourth or intermediate step in which the cells are contacted (e.g., cultured).

In one aspect, provided herein is a method of producing NK cells comprising culturing hematopoietic stem cells or progenitor cells, e.g., CD34⁺ stem cells or progenitor cells, in a first medium comprising a stem cell mobilizing agent and thrombopoietin (Tpo) to produce a first population of cells, subsequently culturing said first population of cells in a second medium comprising a stem cell mobilizing agent and interleukin-15 (IL-15), and lacking Tpo, to produce a second population of cells, and subsequently culturing said second population of cells in a third medium comprising IL-2 and IL-15, and lacking a stem cell mobilizing agent and (optionally) low-molecular weight heparin (LMWH), to produce a third population of cells, wherein the third population of cells comprises natural killer cells that are CD56+, CD3−, and wherein at least 70%, for example 80%, of the natural killer cells are viable. In certain embodiments, said first medium and/or said second medium lack leukemia inhibiting factor (LIF) and/or macrophage inflammatory protein-1 alpha (MIP-1α). In certain embodiments, said third medium lacks LIF, MIP-1α, and FMS-like tyrosine kinase-3 ligand (Flt-3L). In specific embodiments, said first medium and said second medium lack LIF and MIP-1α, and said third medium lacks LIF, MIP-1α, and Flt3L. In certain embodiments, none of the first medium, second medium or third medium comprises heparin, e.g., low-molecular weight heparin. In certain embodiments, such natural killer cells comprise natural killer cells that are CD16−. In certain embodiments, such natural killer cells comprise natural killer cells that are CD94+. In certain embodiments, such natural killer cells comprise natural killer cells that are CD94+ or CD16+. In certain embodiments, such natural killer cells comprise natural killer cells that are CD94− or CD16−. In certain embodiments, such natural killer cells comprise natural killer cells that are CD94+ and CD16+. In certain embodiments, such natural killer cells comprise natural killer cells that are CD94− and CD16−. In certain embodiments, at least one, two, or all three of said first medium, second medium, and third medium are not the medium GBGM®. In certain embodiments, the third medium lacks added desulphated glycosaminoglycans. In certain embodiments, the third medium lacks desulphated glycosaminoglycans.

In one aspect, provided herein is a method of producing NK cells comprising (a) culturing hematopoietic stem or progenitor cells in a first medium comprising a stem cell mobilizing agent and thrombopoietin (Tpo) to produce a first population of cells; (b) culturing the first population of cells in a second medium comprising a stem cell mobilizing agent and interleukin-15 (IL-15), and lacking Tpo, to produce a second population of cells; and (c) culturing the second population of cells in a third medium comprising IL-2 and IL-15, and lacking LMWH, to produce a third population of cells; wherein the third population of cells comprises natural killer cells that are CD56+, CD3−, and CD11a+. In certain embodiments, said first medium and/or said second medium lack leukemia inhibiting factor (LIF) and/or macrophage inflammatory protein-1 alpha (MIP-1α). In certain embodiments, said third medium lacks LIF, MIP-1α, and FMS-like tyrosine kinase-3 ligand (Flt-3L). In specific embodiments, said first medium and said second medium lack LIF and MIP-1α, and said third medium lacks LIF, MIP-1α, and Flt3L. In certain embodiments, none of the first medium, second medium or third medium comprises heparin, e.g., low-molecular weight heparin.

In one aspect, provided herein is a method of producing NK cells comprising (a) culturing hematopoietic stem or progenitor cells in a first medium comprising a stem cell mobilizing agent and thrombopoietin (Tpo) to produce a first population of cells; (b) culturing the first population of cells in a second medium comprising a stem cell mobilizing agent and interleukin-15 (IL-15), and lacking Tpo, to produce a second population of cells; and (c) culturing the second population of cells in a third medium comprising IL-2 and IL-15, and lacking each of stem cell factor (SCF) and LMWH, to produce a third population of cells; wherein the third population of cells comprises natural killer cells that are CD56+, CD3−, and CD11a+. In certain embodiments, said first medium and/or said second medium lack leukemia inhibiting factor (LIF) and/or macrophage inflammatory protein-1 alpha (MIP-1α). In certain embodiments, said third medium lacks LIF, MIP-1α, and FMS-like tyrosine kinase-3 ligand (Flt-3L). In specific embodiments, said first medium and said second medium lack LIF and MIP-1α, and said third medium lacks LIF, MIP-1α, and Flt3L. In certain embodiments, none of the first medium, second medium or third medium comprises heparin, e.g., low-molecular weight heparin.

In one aspect, provided herein is a method of producing NK cells comprising (a) culturing hematopoietic stem or progenitor cells in a first medium comprising a stem cell mobilizing agent and thrombopoietin (Tpo) to produce a first population of cells; (b) culturing the first population of cells in a second medium comprising a stem cell mobilizing agent and interleukin-15 (IL-15), and lacking Tpo, to produce a second population of cells; and (c) culturing the second population of cells in a third medium comprising IL-2 and IL-15, and lacking each of SCF, a stem cell mobilizing agent, and LMWH, to produce a third population of cells; wherein the third population of cells comprises natural killer cells that are CD56+, CD3−, and CD11a. In certain embodiments, said first medium and/or said second medium lack leukemia inhibiting factor (LIF) and/or macrophage inflammatory protein-1 alpha (MIP-1α). In certain embodiments, said third medium lacks LIF, MIP-1α, and FMS-like tyrosine kinase-3 ligand (Flt-3L). In specific embodiments, said first medium and said second medium lack LIF and MIP-1α, and said third medium lacks LIF, MIP-1α, and Flt3L. In certain embodiments, none of the first medium, second medium or third medium comprises heparin, e.g., low-molecular weight heparin.

In one aspect, provided herein is a method of producing NK cells comprising (a) culturing hematopoietic stem or progenitor cells in a first medium comprising a stem cell mobilizing agent and thrombopoietin (Tpo) to produce a first population of cells; (b) culturing the first population of cells in a second medium comprising a stem cell mobilizing agent and interleukin-15 (IL-15), and lacking Tpo, to produce a second population of cells; (c) culturing the second population of cells in a third medium comprising IL-2 and IL-15, and lacking each of a stem cell mobilizing agent and LMWH, to produce a third population of cells; and (d) isolating CD11a+ cells from the third population of cells to produce a fourth population of cells; wherein the fourth population of cells comprises natural killer cells that are CD56+, CD3−, and CD11a+. In certain embodiments, said first medium and/or said second medium lack leukemia inhibiting factor (LIF) and/or macrophage inflammatory protein-1 alpha (MIP-1α). In certain embodiments, said third medium lacks LIF, MIP-1α, and FMS-like tyrosine kinase-3 ligand (Flt-3L). In specific embodiments, said first medium and said second medium lack LIF and MIP-1α, and said third medium lacks LIF, MIP-1α, and Flt3L. In certain embodiments, none of the first medium, second medium or third medium comprises heparin, e.g., low-molecular weight heparin.

In certain embodiments, said natural killer cells express perforin and eomesodermin (EOMES). In certain embodiments, said natural killer cells do not express either RAR-related orphan receptor gamma (RORγt) or interleukin-1 receptor 1 (IL1R1).

In one aspect, provided herein is a method of producing ILC3 cells comprising (a) culturing hematopoietic stem or progenitor cells in a first medium comprising a stem cell mobilizing agent and thrombopoietin (Tpo) to produce a first population of cells; (b) culturing the first population of cells in a second medium comprising a stem cell mobilizing agent and interleukin-15 (IL-15), and lacking Tpo, to produce a second population of cells; and (c) culturing the second population of cells in a third medium comprising IL-2 and IL-15, and lacking LMWH, to produce a third population of cells; wherein the third population of cells comprises ILC3 cells that are CD56+, CD3−, and CD11a−. In certain embodiments, said first medium and/or said second medium lack leukemia inhibiting factor (LIF) and/or macrophage inflammatory protein-1 alpha (MIP-1α). In certain embodiments, said third medium lacks LIF, MIP-1α, and FMS-like tyrosine kinase-3 ligand (Flt-3L). In specific embodiments, said first medium and said second medium lack LIF and MIP-1α, and said third medium lacks LIF, MIP-1a, and Flt3L. In certain embodiments, none of the first medium, second medium or third medium comprises heparin, e.g., low-molecular weight heparin.

In one aspect, provided herein is a method of producing ILC3 cells comprising (a) culturing hematopoietic stem or progenitor cells in a first medium comprising a stem cell mobilizing agent and thrombopoietin (Tpo) to produce a first population of cells; (b) culturing the first population of cells in a second medium comprising a stem cell mobilizing agent and interleukin-15 (IL-15), and lacking Tpo, to produce a second population of cells; and (c) culturing the second population of cells in a third medium comprising a stem cell mobilizing agent, IL-2 and IL-15, and lacking LMWH, to produce a third population of cells; wherein the third population of cells comprises ILC3 cells that are CD56+, CD3−, and CD11a−. In certain embodiments, said first medium and/or said second medium lack leukemia inhibiting factor (LIF) and/or macrophage inflammatory protein-1 alpha (MIP-1α). In certain embodiments, said third medium lacks LIF, MIP-1α, and FMS-like tyrosine kinase-3 ligand (Flt-3L). In specific embodiments, said first medium and said second medium lack LIF and MIP-1α, and said third medium lacks LIF, MIP-1α, and Flt3L. In certain embodiments, none of the first medium, second medium or third medium comprises heparin, e.g., low-molecular weight heparin.

In one aspect, provided herein is a method of producing ILC3 cells comprising (a) culturing hematopoietic stem or progenitor cells in a first medium comprising a stem cell mobilizing agent and thrombopoietin (Tpo) to produce a first population of cells; (b) culturing the first population of cells in a second medium comprising a stem cell mobilizing agent and interleukin-15 (IL-15), and lacking Tpo, to produce a second population of cells; and (c) culturing the second population of cells in a third medium comprising SCF, IL-2 and IL-15, and lacking LMWH, to produce a third population of cells; wherein the third population of cells comprises ILC3 cells that are CD56+, CD3−, and CD11a−. In certain embodiments, said first medium and/or said second medium lack leukemia inhibiting factor (LIF) and/or macrophage inflammatory protein-1 alpha (MIP-1α). In certain embodiments, said third medium lacks LIF, MIP-1α, and FMS-like tyrosine kinase-3 ligand (Flt-3L). In specific embodiments, said first medium and said second medium lack LIF and MIP-1α, and said third medium lacks LIF, MIP-1a, and Flt3L. In certain embodiments, none of the first medium, second medium or third medium comprises heparin, e.g., low-molecular weight heparin.

In one aspect, provided herein is a method of producing ILC3 cells comprising (a) culturing hematopoietic stem or progenitor cells in a first medium comprising a stem cell mobilizing agent and thrombopoietin (Tpo) to produce a first population of cells; (b) culturing the first population of cells in a second medium comprising a stem cell mobilizing agent and interleukin-15 (IL-15), and lacking Tpo, to produce a second population of cells; and (c) culturing the second population of cells in a third medium comprising a stem cell mobilizing agent, SCF, IL-2 and IL-15, and lacking LMWH, to produce a third population of cells; wherein the third population of cells comprises ILC3 cells that are CD56+, CD3−, and CD11a−. In certain embodiments, said first medium and/or said second medium lack leukemia inhibiting factor (LIF) and/or macrophage inflammatory protein-1 alpha (MIP-1α). In certain embodiments, said third medium lacks LIF, MIP-1α, and FMS-like tyrosine kinase-3 ligand (Flt-3L). In specific embodiments, said first medium and said second medium lack LIF and MIP-1α, and said third medium lacks LIF, MIP-1α, and Flt3L. In certain embodiments, none of the first medium, second medium or third medium comprises heparin, e.g., low-molecular weight heparin.

In one aspect, provided herein is a method of producing ILC3 cells comprising (a) culturing hematopoietic stem or progenitor cells in a first medium comprising a stem cell mobilizing agent and thrombopoietin (Tpo) to produce a first population of cells; (b) culturing the first population of cells in a second medium comprising a stem cell mobilizing agent and interleukin-15 (IL-15), and lacking Tpo, to produce a second population of cells; (c) culturing the second population of cells in a third medium comprising IL-2 and IL-15, and lacking each of a stem cell mobilizing agent and LMWH, to produce a third population of cells; and (d) isolating CD11a− cells from the third population of cells to produce a fourth population of cells; wherein the fourth population of cells comprises ILC3 cells that are CD56+, CD3−, and CD11a−. In certain embodiments, said first medium and/or said second medium lack leukemia inhibiting factor (LIF) and/or macrophage inflammatory protein-1 alpha (MIP-1α). In certain embodiments, said third medium lacks LIF, MIP-1α, and FMS-like tyrosine kinase-3 ligand (Flt-3L). In specific embodiments, said first medium and said second medium lack LIF and MIP-1α, and said third medium lacks LIF, MIP-1α, and Flt3L. In certain embodiments, none of the first medium, second medium or third medium comprises heparin, e.g., low-molecular weight heparin.

In certain embodiments, said ILC3 cells express RORγt and IL1R1. In certain embodiments, said ILC3 cells do not express either perforin or EOMES. In certain embodiments, said third medium lacks added desulphated glycosaminoglycans. In certain embodiments, said third medium lacks desulphated glycosaminoglycans.

In certain embodiments, said hematopoietic stem or progenitor cells are mammalian cells. In specific embodiments, said hematopoietic stem or progenitor cells are human cells. In specific embodiments, said hematopoietic stem or progenitor cells are primate cells. In specific embodiments, said hematopoietic stem or progenitor cells are canine cells. In specific embodiments, said hematopoietic stem or progenitor cells are rodent cells. In specific embodiments, said hematopoietic stem or progenitor cells are cells from a mammal other than a human, primate, canine or rodent.

In certain aspects, the hematopoietic stem cells or progenitor cells cultured in the first medium are CD34⁺ stem cells or progenitor cells. In certain aspects, the hematopoietic stem cells or progenitor cells are placental hematopoietic stem cells or progenitor cells. In certain aspects, the placental hematopoietic stem cells or progenitor cells are obtained from, or obtainable from placental perfusate (e.g. obtained from or obtainable from isolated nucleated cells from placental perfusate). In certain aspects, said hematopoietic stem or progenitor cells are obtained from, or obtainable from, umbilical cord blood. In certain aspects, said hematopoietic stem or progenitor cells are fetal liver cells. In certain aspects, said hematopoietic stem or progenitor cells are mobilized peripheral blood cells. In certain aspects, said hematopoietic stem or progenitor cells are bone marrow cells.

In certain aspects, said first medium used in the three-stage method comprises a stem cell mobilizing agent and thrombopoietin (Tpo). In certain aspects, the first medium used in the three-stage method comprises, in addition to a stem cell mobilizing agent and Tpo, one or more of Low Molecular Weight Heparin (LMWH), Flt-3 Ligand (Flt-3L), stem cell factor (SCF), IL-6, IL-7, granulocyte colony-stimulating factor (G-CSF), or granulocyte-macrophage-stimulating factor (GM-CSF). In certain aspects, said first medium does not comprise added LMWH. In certain aspects, said first medium does not comprise added desulphated glycosaminoglycans. In certain aspects, said first medium does not comprise LMWH. In certain aspects, said first medium does not comprise desulphated glycosaminoglycans. In certain aspects, the first medium used in the three-stage method comprises, in addition to a stem cell mobilizing agent and Tpo, each of, Flt-3L, SCF, IL-6, IL-7, G-CSF, and GM-CSF. In certain aspects, the first medium used in the three-stage method comprises, in addition to a stem cell mobilizing agent and Tpo, each of Flt-3L, SCF, IL-6, IL-7, G-CSF, and GM-CSF. In certain aspects, said Tpo is present in the first medium at a concentration of from 1 ng/mL to 100 ng/mL, from 1 ng/mL to 50 ng/mL, from 20 ng/mL to 30 ng/mL, or about 25 ng/mL. In certain aspects, when LMWH is present in the first medium, the LMWH is present at a concentration of from 1 U/mL to 10 U/mL; the Flt-3L is present at a concentration of from 1 ng/mL to 50 ng/mL; the SCF is present at a concentration of from 1 ng/mL to 50 ng/mL; the IL-6 is present at a concentration of from 0.01 ng/mL to 0.1 ng/mL; the IL-7 is present at a concentration of from 1 ng/mL to 50 ng/mL; the G-CSF is present at a concentration of from 0.01 ng/mL to 0.50 ng/mL; and the GM-CSF is present at a concentration of from 0.005 ng/mL to 0.1 ng/mL. In certain aspects, in the first medium, the Flt-3L is present at a concentration of from 1 ng/mL to 50 ng/mL; the SCF is present at a concentration of from 1 ng/mL to 50 ng/mL; the IL-6 is present at a concentration of from 0.01 ng/mL to 0.1 ng/mL; the IL-7 is present at a concentration of from 1 ng/mL to 50 ng/mL; the G-CSF is present at a concentration of from 0.01 ng/mL to 0.50 ng/mL; and the GM-CSF is present at a concentration of from 0.005 ng/mL to 0.1 ng/mL. In certain aspects, when LMWH is present in the first medium, the LMWH is present at a concentration of from 4 U/mL to 5 U/mL; the Flt-3L is present at a concentration of from 20 ng/mL to 30 ng/mL; the SCF is present at a concentration of from 20 ng/mL to 30 ng/mL; the IL-6 is present at a concentration of from 0.04 ng/mL to 0.06 ng/mL; the IL-7 is present at a concentration of from 20 ng/mL to 30 ng/mL; the G-CSF is present at a concentration of from 0.20 ng/mL to 0.30 ng/mL; and the GM-CSF is present at a concentration of from 0.005 ng/mL to 0.5 ng/mL. In certain aspects, in the first medium, the Flt-3L is present at a concentration of from 20 ng/mL to 30 ng/mL; the SCF is present at a concentration of from 20 ng/mL to 30 ng/mL; the IL-6 is present at a concentration of from 0.04 ng/mL to 0.06 ng/mL; the IL-7 is present at a concentration of from 20 ng/mL to 30 ng/mL; the G-CSF is present at a concentration of from 0.20 ng/mL to 0.30 ng/mL; and the GM-CSF is present at a concentration of from 0.005 ng/mL to 0.5 ng/mL. In certain aspects, when LMWH is present in the first medium, the LMWH is present at a concentration of about 4.5 U/mL; the Flt-3L is present at a concentration of about 25 ng/mL; the SCF is present at a concentration of about 27 ng/mL; the IL-6 is present at a concentration of about 0.05 ng/mL; the IL-7 is present at a concentration of about 25 ng/mL; the G-CSF is present at a concentration of about 0.25 ng/mL; and the GM-CSF is present at a concentration of about 0.01 ng/mL. In certain aspects, in the first medium, the Flt-3L is present at a concentration of about 25 ng/mL; the SCF is present at a concentration of about 27 ng/mL; the IL-6 is present at a concentration of about 0.05 ng/mL; the IL-7 is present at a concentration of about 25 ng/mL; the G-CSF is present at a concentration of about 0.25 ng/mL; and the GM-CSF is present at a concentration of about 0.01 ng/mL. In certain embodiments, said first medium is not GBGM®.

In certain aspects, said second medium used in the three-stage method comprises a stem cell mobilizing agent and interleukin-15 (IL-15), and lacks Tpo. In certain aspects, the second medium used in the three-stage method comprises, in addition to a stem cell mobilizing agent and IL-15, one or more of LMWH, Flt-3, SCF, IL-6, IL-7, G-CSF, and GM-CSF. In certain aspects, the second medium does not comprise added LMWH. In certain aspects, the second medium does not comprise added desulphated glycosaminoglycans. In certain aspects, the second medium does not comprise LMWH. In certain aspects, the second medium does not comprise desulphated glycosaminoglycans. In certain aspects, the second medium used in the three-stage method comprises, in addition to a stem cell mobilizing agent and IL-15, each of IL-6, IL-7, G-CSF, and GM-CSF. In certain aspects, the second medium used in the three-stage method comprises, in addition to a stem cell mobilizing agent and IL-15, each of Flt-3, SCF, IL-6, IL-7, G-CSF, and GM-CSF. In certain aspects, said IL-15 is present in said second medium at a concentration of from 1 ng/mL to 50 ng/mL, from 10 ng/mL to 30 ng/mL, or about 20 ng/mL. In certain aspects, when LMWH is present in said second medium, the LMWH is present at a concentration of from 1 U/mL to 10 U/mL; the Flt-3L is present at a concentration of from 1 ng/mL to 50 ng/mL; the SCF is present at a concentration of from 1 ng/mL to 50 ng/mL; the IL-6 is present at a concentration of from 0.01 ng/mL to 0.1 ng/mL; the IL-7 is present at a concentration of from 1 ng/mL to 50 ng/mL; the G-CSF is present at a concentration of from 0.01 ng/mL to 0.50 ng/mL; and the GM-CSF is present at a concentration of from 0.005 ng/mL to 0.1 ng/mL. In certain aspects, when LMWH is present in the second medium, the LMWH is present in the second medium at a concentration of from 4 U/mL to 5 U/mL; the Flt-3L is present at a concentration of from 20 ng/mL to 30 ng/mL; the SCF is present at a concentration of from 20 ng/mL to 30 ng/mL; the IL-6 is present at a concentration of from 0.04 ng/mL to 0.06 ng/mL; the IL-7 is present at a concentration of from 20 ng/mL to 30 ng/mL; the G-CSF is present at a concentration of from 0.20 ng/mL to 0.30 ng/mL; and the GM-CSF is present at a concentration of from 0.005 ng/mL to 0.5 ng/mL. In certain aspects, in the second medium, the Flt-3L is present at a concentration of from 20 ng/mL to 30 ng/mL; the SCF is present at a concentration of from 20 ng/mL to 30 ng/mL; the IL-6 is present at a concentration of from 0.04 ng/mL to 0.06 ng/mL; the IL-7 is present at a concentration of from 20 ng/mL to 30 ng/mL; the G-CSF is present at a concentration of from 0.20 ng/mL to 0.30 ng/mL; and the GM-CSF is present at a concentration of from 0.005 ng/mL to 0.5 ng/mL. In certain aspects, when LMWH is present in the second medium, the LMWH is present in the second medium at a concentration of from 4 U/mL to 5 U/mL; the Flt-3L is present at a concentration of from 20 ng/mL to 30 ng/mL; the SCF is present at a concentration of from 20 ng/mL to 30 ng/mL; the IL-6 is present at a concentration of from 0.04 ng/mL to 0.06 ng/mL; the IL-7 is present at a concentration of from 20 ng/mL to 30 ng/mL; the G-CSF is present at a concentration of from 0.20 ng/mL to 0.30 ng/mL; and the GM-CSF is present at a concentration of from 0.005 ng/mL to 0.5 ng/mL. In certain aspects, in the second medium, the Flt-3L is present at a concentration of from 20 ng/mL to 30 ng/mL; the SCF is present at a concentration of from 20 ng/mL to 30 ng/mL; the IL-6 is present at a concentration of from 0.04 ng/mL to 0.06 ng/mL; the IL-7 is present at a concentration of from 20 ng/mL to 30 ng/mL; the G-CSF is present at a concentration of from 0.20 ng/mL to 0.30 ng/mL; and the GM-CSF is present at a concentration of from 0.005 ng/mL to 0.5 ng/mL. In certain aspects, when LMWH is present in the second medium, the LMWH is present in the second medium at a concentration of about 4.5 U/mL; the Flt-3L is present at a concentration of about 25 ng/mL; the SCF is present at a concentration of about 27 ng/mL; the IL-6 is present at a concentration of about 0.05 ng/mL; the IL-7 is present at a concentration of about 25 ng/mL; the G-CSF is present at a concentration of about 0.25 ng/mL; and the GM-CSF is present at a concentration of about 0.01 ng/mL. In certain aspects, in the second medium, the Flt-3L is present at a concentration of about 25 ng/mL; the SCF is present at a concentration of about 27 ng/mL; the IL-6 is present at a concentration of about 0.05 ng/mL; the IL-7 is present at a concentration of about 25 ng/mL; the G-CSF is present at a concentration of about 0.25 ng/mL; and the GM-CSF is present at a concentration of about 0.01 ng/mL. In certain embodiments, said second medium is not GBGM®.

In certain aspects, the stem cell mobilizing factor is a compound having Formula (I), (I-A), (I-B), (I-C), or (I-D), as described below.

In certain aspects, said third medium used in the three-stage method comprises IL-2 and IL-15, and lacks a stem cell mobilizing agent and LMWH. In certain aspects, the third medium used in the three-stage method comprises, in addition to IL-2 and IL-15, one or more of SCF, IL-6, IL-7, G-CSF, or GM-CSF. In certain aspects, the third medium used in the three-stage method comprises, in addition to IL-2 and IL-15, each of SCF, IL-6, IL-7, G-CSF, and GM-CSF. In certain aspects, said IL-2 is present in said third medium at a concentration of from 10 U/mL to 10,000 U/mL and said IL-15 is present in said third medium at a concentration of from 1 ng/mL to 50 ng/mL. In certain aspects, said IL-2 is present in said third medium at a concentration of from 100 U/mL to 10,000 U/mL and said IL-15 is present in said third medium at a concentration of from 1 ng/mL to 50 ng/mL. In certain aspects, said IL-2 is present in said third medium at a concentration of from 300 U/mL to 3,000 U/mL and said IL-15 is present in said third medium at a concentration of from 10 ng/mL to 30 ng/mL. In certain aspects, said IL-2 is present in said third medium at a concentration of about 1,000 U/mL and said IL-15 is present in said third medium at a concentration of about 20 ng/mL. In certain aspects, in said third medium, the SCF is present at a concentration of from 1 ng/mL to 50 ng/mL; the IL-6 is present at a concentration of from 0.01 ng/mL to 0.1 ng/mL; the IL-7 is present at a concentration of from 1 ng/mL to 50 ng/mL; the G-CSF is present at a concentration of from 0.01 ng/mL to 0.50 ng/mL; and the GM-CSF is present at a concentration of from 0.005 ng/mL to 0.1 ng/mL. In certain aspects, in said third medium, the SCF is present at a concentration of from 20 ng/mL to 30 ng/mL; the IL-6 is present at a concentration of from 0.04 ng/mL to 0.06 ng/mL; the IL-7 is present at a concentration of from 20 ng/mL to 30 ng/mL; the G-CSF is present at a concentration of from 0.20 ng/mL to 0.30 ng/mL; and the GM-CSF is present at a concentration of from 0.005 ng/mL to 0.5 ng/mL. In certain aspects, in said third medium, the SCF is present at a concentration of about 22 ng/mL; the IL-6 is present at a concentration of about 0.05 ng/mL; the IL-7 is present at a concentration of about 20 ng/mL; the G-CSF is present at a concentration of about 0.25 ng/mL; and the GM-CSF is present at a concentration of about 0.01 ng/mL. In certain embodiments, said third medium is not GBGM®.

In certain aspects, the third medium comprises 100 ng/mL IL-7, 1000 ng/mL IL-2, 20 ng/mL IL-15, and stem cell mobilizing agent and lacks SCF. In certain aspects, the third medium comprises 20 ng/mL IL-7, 1000 ng/mL IL-2, 20 ng/mL IL-15, and stem cell mobilizing agent and lacks SCF. In certain aspects, the third medium comprises 20 ng/mL IL-7, 20 ng/mL IL-15, and stem cell mobilizing agent stem cell mobilizing agent and lacks SCF. In certain aspects, the third medium comprises 100 ng/mL IL-7, 22 ng/mL SCF, 1000 ng/mL IL-2, and 20 ng/mL IL-15 and lacks stem cell mobilizing agent. In certain aspects, the third medium comprises 22 ng/mL SCF, 1000 ng/mL IL-2, and 20 ng/mL IL-15 and lacks stem cell mobilizing agent. In certain aspects, the third medium comprises 20 ng/mL IL-7, 22 ng/mL SCF, 1000 ng/mL IL-2, and 20 ng/mL IL-15 and lacks stem cell mobilizing agent. In certain aspects, the third medium comprises 20 ng/mL IL-7, 22 ng/mL SCF, and 1000 ng/mL IL-2 and lacks stem cell mobilizing agent.

In certain embodiments, said first medium and/or said second medium lack leukemia inhibiting factor (LIF) and/or macrophage inflammatory protein-1 alpha (MIP-1α). In certain embodiments, said third medium lacks LIF, MIP-1α, and FMS-like tyrosine kinase-3 ligand (Flt-3L). In specific embodiments, said first medium and said second medium lack LIF and MIP-1α, and said third medium lacks LIF, MIP-1α, and Flt3L. In certain embodiments, none of the first medium, second medium or third medium comprises heparin, e.g., low-molecular weight heparin.

Generally, the particularly recited medium components do not refer to possible constituents in an undefined component of said medium, e.g., serum. For example, said Tpo, IL-2, and IL-15 are not comprised within an undefined component of the first medium, second medium or third medium, e.g., said Tpo, IL-2, and IL-15 are not comprised within serum. Further, said LMWH, Flt-3, SCF, IL-6, IL-7, G-CSF, and/or GM-CSF are not comprised within an undefined component of the first medium, second medium or third medium, e.g., said LMWH, Flt-3, SCF, IL-6, IL-7, G-CSF, and/or GM-CSF are not comprised within serum.

In certain aspects, said first medium, second medium or third medium comprises human serum-AB. In certain aspects, any of said first medium, second medium or third medium comprises 1% to 20% human serum-AB, 5% to 15% human serum-AB, or about 2, 5, or 10% human serum-AB.

In certain aspects, any of said first medium, second medium or third medium comprises 2-mercaptoethanol. In certain aspects, any of said first medium, second medium or third medium comprises gentamycin.

In certain embodiments, in the three-stage methods described herein, said hematopoietic stem or progenitor cells are cultured in said first medium for 1, 2, 3, 4, 5, 6, 7, 8, 9, 10, 11, 12, 13, 14, 15, 16, 17, 18, 19, or 20 days before said culturing in said second medium. In certain embodiments, cells are cultured in said second medium for 1, 2, 3, 4, 5, 6, 7, 8, 9, 10, 11, 12, 13, 14, 15, 16, 17, 18, 19, or 20 days before said culturing in said third medium. In certain embodiments, cells are cultured in said third medium for 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days, or for more than 30 days.

In one embodiment, in the three-stage methods described herein, said hematopoietic stem or progenitor cells are cultured in said first medium for 7-13 days to produce a first population of cells; said first population of cells are cultured in said second medium for 2-6 days to produce a second population of cells; and said second population of cells are cultured in said third medium for 10-30 days, i.e., the cells are cultured a total of 19-49 days.

In one embodiment, in the three-stage methods described herein, said hematopoietic stem or progenitor cells are cultured in said first medium for 8-12 days to produce a first population of cells; said first population of cells are cultured in said second medium for 3-5 days to produce a second population of cells; and said second population of cells are cultured in said third medium for 15-25 days, i.e., the cells are cultured a total of 26-42 days.

In a specific embodiment, in the three-stage methods described herein, said hematopoietic stem or progenitor cells are cultured in said first medium for about 10 days to produce a first population of cells; said first population of cells are cultured in said second medium for about 4 days to produce a second population of cells; and said second population of cells are cultured in said third medium for about 21 days, i.e., the cells are cultured a total of about 35 days.

In certain aspects, said culturing in said first medium, second medium and third medium are all performed under static culture conditions, e.g., in a culture dish or culture flask. In certain aspects, said culturing in at least one of said first medium, second medium or third medium are performed in a spinner flask. In certain aspects, said culturing in said first medium and said second medium is performed under static culture conditions, and said culturing in said third medium is performed in a spinner flask.

In certain aspects, said culturing is performed in a spinner flask. In other aspects, said culturing is performed in a G-Rex device. In yet other aspects, said culturing is performed in a WAVE bioreactor.

In certain aspects, said hematopoietic stem or progenitor cells are initially inoculated into said first medium from 1×10⁴ to 1×10⁵ cells/mL. In a specific aspect, said hematopoietic stem or progenitor cells are initially inoculated into said first medium at about 3×10⁴ cells/mL.

In certain aspects, said first population of cells are initially inoculated into said second medium from 5×10⁴ to 5×10⁵ cells/mL. In a specific aspect, said first population of cells is initially inoculated into said second medium at about 1×10⁵ cells/mL.

In certain aspects said second population of cells is initially inoculated into said third medium from 1×10⁵ to 5×10⁶ cells/mL. In certain aspects, said second population of cells is initially inoculated into said third medium from 1×10⁵ to 1×10⁶ cells/mL. In a specific aspect, said second population of cells is initially inoculated into said third medium at about 5×10⁵ cells/mL. In a more specific aspect, said second population of cells is initially inoculated into said third medium at about 5×10⁵ cells/mL in a spinner flask. In a specific aspect, said second population of cells is initially inoculated into said third medium at about 3×10⁵ cells/mL. In a more specific aspect, said second population of cells is initially inoculated into said third medium at about 3×10⁵ cells/mL in a static culture.

In certain aspects, the three-stage method disclosed herein produces at least 5000-fold more natural killer cells as compared to the number of hematopoietic stem cells initially inoculated into said first medium. In certain aspects, said three-stage method produces at least 10,000-fold more natural killer cells as compared to the number of hematopoietic stem cells initially inoculated into said first medium. In certain aspects, said three-stage method produces at least 50,000-fold more natural killer cells as compared to the number of hematopoietic stem cells initially inoculated into said first medium. In certain aspects, said three-stage method produces at least 75,000-fold more natural killer cells as compared to the number of hematopoietic stem cells initially inoculated into said first medium. In certain aspects, the viability of said natural killer cells is determined by 7-aminoactinomycin D (7AAD) staining. In certain aspects, the viability of said natural killer cells is determined by annexin-V staining. In specific aspects, the viability of said natural killer cells is determined by both 7-AAD staining and annexin-V staining. In certain aspects, the viability of said natural killer cells is determined by trypan blue staining.

In certain aspects, the three-stage method disclosed herein produces at least 5000-fold more ILC3 cells as compared to the number of hematopoietic stem cells initially inoculated into said first medium. In certain aspects, said three-stage method produces at least 10,000-fold more ILC3 cells as compared to the number of hematopoietic stem cells initially inoculated into said first medium. In certain aspects, said three-stage method produces at least 50,000-fold more ILC3 cells as compared to the number of hematopoietic stem cells initially inoculated into said first medium. In certain aspects, said three-stage method produces at least 75,000-fold more ILC3 cells as compared to the number of hematopoietic stem cells initially inoculated into said first medium.

In certain aspects, the three-stage method disclosed herein produces natural killer cells that comprise at least 20% CD56+CD3− natural killer cells. In certain aspects, the three-stage method produces natural killer cells that comprise at least 40% CD56+CD3− natural killer cells. In certain aspects, the three-stage method produces natural killer cells that comprise at least 60% CD56+CD3− natural killer cells. In certain aspects, the three-stage method produces natural killer cells that comprise at least 70% CD56+CD3− natural killer cells. In certain aspects, the three-stage method produces natural killer cells that comprise at least 75% CD56+CD3− natural killer cells. In certain aspects, the three-stage method produces natural killer cells that comprise at least 80% CD56+CD3− natural killer cells.

In certain aspects, the three-stage method disclosed herein produces natural killer cells that comprise at least 20% CD56+CD3−CD11a+ natural killer cells. In certain aspects, the three-stage method disclosed herein produces natural killer cells that comprise at least 40% CD56+CD3− CD11a+ natural killer cells. In certain aspects, the three-stage method disclosed herein produces natural killer cells that comprise at least 60% CD56+CD3− CD11a+ natural killer cells. In certain aspects, the three-stage method disclosed herein produces natural killer cells that comprise at least 80% CD56+CD3− CD11a+ natural killer cells.

In certain aspects, the three-stage method disclosed herein produces ILC3 cells that comprise at least 20% CD56+CD3− CD11a− ILC3 cells. In certain aspects, the three-stage method disclosed herein produces ILC3 cells that comprise at least 40% CD56+CD3− CD11a− ILC3 cells. In certain aspects, the three-stage method disclosed herein produces ILC3 cells that comprise at least 60% CD56+CD3− CD11a− ILC3 cells. In certain aspects, the three-stage method disclosed herein produces natural killer cells that comprise at least 80% CD56+CD3− CD11a− ILC3 cells.

In certain aspects, the three-stage method disclosed herein, produces natural killer cells that exhibit at least 20% cytotoxicity against K562 cells when said natural killer cells and said K562 cells are co-cultured in vitro at a ratio of 10:1. In certain aspects, the three-stage method produces natural killer cells that exhibit at least 35% cytotoxicity against the K562 cells when said natural killer cells and said K562 cells are co-cultured in vitro at a ratio of 10:1. In certain aspects, the three-stage method produces natural killer cells that exhibit at least 45% cytotoxicity against the K562 cells when said natural killer cells and said K562 cells are co-cultured in vitro at a ratio of 10:1. In certain aspects, the three-stage method produces natural killer cells that exhibit at least 60% cytotoxicity against the K562 cells when said natural killer cells and said K562 cells are co-cultured in vitro at a ratio of 10:1. In certain aspects, the three-stage method produces natural killer cells that exhibit at least 75% cytotoxicity against the K562 cells when said natural killer cells and said K562 cells are co-cultured in vitro at a ratio of 10:1.

In certain aspects, the three-stage method disclosed herein, produces ILC3 cells that exhibit at least 20% cytotoxicity against K562 cells when said ILC3 cells and said K562 cells are co-cultured in vitro at a ratio of 10:1. In certain aspects, the three-stage method produces ILC3 cells that exhibit at least 35% cytotoxicity against the K562 cells when said ILC3 cells and said K562 cells are co-cultured in vitro at a ratio of 10:1. In certain aspects, the three-stage method produces ILC3 cells that exhibit at least 45% cytotoxicity against the K562 cells when said ILC3 cells and said K562 cells are co-cultured in vitro at a ratio of 10:1. In certain aspects, the three-stage method produces ILC3 cells that exhibit at least 60% cytotoxicity against the K562 cells when said ILC3 cells and said K562 cells are co-cultured in vitro at a ratio of 10:1. In certain aspects, the three-stage method produces ILC3 cells that exhibit at least 75% cytotoxicity against the K562 cells when said ILC3 cells and said K562 cells are co-cultured in vitro at a ratio of 10:1.

In certain aspects, after said third culturing step, said third population of cells, e.g., said population of natural killer cells, is cryopreserved. In certain aspects, after said fourth culturing step, said fourth population of cells, e.g., said population of natural killer cells, is cryopreserved.

In certain aspects, provided herein are populations of cells comprising natural killer cells, i.e., natural killers cells produced by a three-stage method described herein. Accordingly, provided herein is an isolated natural killer cell population produced by a three-stage method described herein. In a specific embodiment, said natural killer cell population comprises at least 20% CD56+CD3− natural killer cells. In a specific embodiment, said natural killer cell population comprises at least 40% CD56+CD3− natural killer cells. In a specific embodiment, said natural killer cell population comprises at least 60% CD56+CD3− natural killer cells. In a specific embodiment, said natural killer cell population comprises at least 80% CD56+CD3− natural killer cells. In specific embodiments, the natural killer cell population is formulated into a pharmaceutical composition suitable for use in vivo, for example, suitable for human use in vivo.

In certain aspects, provided herein are populations of cells comprising ILC3 cells, i.e., natural killer cells produced by a three-stage method described herein. In specific embodiments, the population of cells comprising ILC3 cells is formulated into a pharmaceutical composition suitable for use in vivo, for example, suitable for human use in vivo.

In one embodiment, provided herein is an isolated NK progenitor cell population, wherein said NK progenitor cells are produced according to the three-stage method described herein. In specific embodiments, the NK progenitor cell population is formulated into a pharmaceutical composition suitable for use in vivo, for example, suitable for human use in vivo.

In another embodiment, provided herein is an isolated mature NK cell population, wherein said mature NK cells are produced according to the three-stage method described herein. In specific embodiments, the mature NK cell population is formulated into a pharmaceutical composition suitable for use in vivo, for example, suitable for human use in vivo.

In another embodiment, provided herein is an isolated ILC3 population, wherein said ILC3 cells are produced according to the three-stage method described herein. In specific embodiments, the isolated ILC3 population is formulated into a pharmaceutical composition suitable for use in vivo, for example, suitable for human use in vivo.

In another embodiment, provided herein is a cell population, wherein said cell population is the third population of cells produced by a method described herein. In another embodiment, provided herein is a cell population, wherein said cell population is the fourth population of cells produced by a method described herein.

In another embodiment, provided herein is an isolated NK cell population, wherein said NK cells are activated, wherein said activated NK cells are produced according to the three-stage method described herein. In specific embodiments, the isolated NK population is formulated into a pharmaceutical composition suitable for use in vivo, for example, suitable for human use in vivo.

Accordingly, in another aspect, provided herein is the use of NK cell populations produced using the three-stage methods described herein to suppress tumor cell proliferation, treat viral infection, or treat cancer, e.g., blood cancers and solid tumors. In certain embodiments, the NK cell populations are contacted with, or used in combination with, an immunomodulatory compound, e.g., an immunomodulatory compound described herein, or thalidomide. In certain embodiments, the NK cell populations are treated with, or used in combination with, an immunomodulatory compound, e.g., an immunomodulatory compound described herein, or thalidomide.

In a specific embodiment, said cancer is a solid tumor. In another embodiment, said cancer is a blood cancer. In specific embodiments, the cancer is glioblastoma, primary ductal carcinoma, leukemia, acute T cell leukemia, chronic myeloid lymphoma (CML), acute myelogenous leukemia (AML), chronic myelogenous leukemia (CML), lung carcinoma, colon adenocarcinoma, histiocytic lymphoma, colorectal carcinoma, colorectal adenocarcinoma, prostate cancer, multiple myeloma, or retinoblastoma. In more specific embodiments, the cancer is AML. In more specific embodiments, the cancer is multiple myeloma.

In another specific embodiment, the hematopoietic cells, e.g., hematopoietic stem cells or progenitor cells, from which the NK cell populations are produced, are obtained from placental perfusate, umbilical cord blood or peripheral blood. In one embodiment, the hematopoietic cells, e.g., hematopoietic stem cells or progenitor cells, from which NK cell populations are produced, are obtained from placenta, e.g., from placental perfusate. In one embodiment, the hematopoietic cells, e.g., hematopoietic stem cells or progenitor cells, from which the NK cell populations are produced, are not obtained from umbilical cord blood. In one embodiment, the hematopoietic cells, e.g., hematopoietic stem cells or progenitor cells, from which the NK cell populations are produced, are not obtained from peripheral blood. In another specific embodiment, the hematopoietic cells, e.g., hematopoietic stem cells or progenitor cells, from which the NK cell populations are produced, are combined cells from placental perfusate and cord blood, e.g., cord blood from the same placenta as the perfusate. In another specific embodiment, said umbilical cord blood is isolated from a placenta other than the placenta from which said placental perfusate is obtained. In certain embodiments, the combined cells can be obtained by pooling or combining the cord blood and placental perfusate. In certain embodiments, the cord blood and placental perfusate are combined at a ratio of 100:1, 95:5, 90:10, 85:15, 80:20, 75:25, 70:30, 65:35, 60:40, 55:45: 50:50, 45:55, 40:60, 35:65, 30:70, 25:75, 20:80, 15:85, 10:90, 5:95, 100:1, 95:1, 90:1, 85:1, 80:1, 75:1, 70:1, 65:1, 60:1, 55:1, 50:1, 45:1, 40:1, 35:1, 30:1, 25:1, 20:1, 15:1, 10:1, 5:1, 1:1, 1:5, 1:10, 1:15, 1:20, 1:25, 1:30, 1:35, 1:40, 1:45, 1:50, 1:55, 1:60, 1:65, 1:70, 1:75, 1:80, 1:85, 1:90, 1:95, 1:100, or the like by volume to obtain the combined cells. In a specific embodiment, the cord blood and placental perfusate are combined at a ratio of from 10:1 to 1:10, from 5:1 to 1:5, or from 3:1 to 1:3. In another specific embodiment, the cord blood and placental perfusate are combined at a ratio of 10:1, 5:1, 3:1, 1:1, 1:3, 1:5 or 1:10. In a more specific embodiment, the cord blood and placental perfusate are combined at a ratio of 8.5:1.5 (85%: 15%).

In certain embodiments, the cord blood and placental perfusate are combined at a ratio of 100:1, 95:5, 90:10, 85:15, 80:20, 75:25, 70:30, 65:35, 60:40, 55:45: 50:50, 45:55, 40:60, 35:65, 30:70, 25:75, 20:80, 15:85, 10:90, 5:95, 100:1, 95:1, 90:1, 85:1, 80:1, 75:1, 70:1, 65:1, 60:1, 55:1, 50:1, 45:1, 40:1, 35:1, 30:1, 25:1, 20:1, 15:1, 10:1, 5:1, 1:1, 1:5, 1:10, 1:15, 1:20, 1:25, 1:30, 1:35, 1:40, 1:45, 1:50, 1:55, 1:60, 1:65, 1:70, 1:75, 1:80, 1:85, 1:90, 1:95, 1:100, or the like, as determined by total nucleated cells (TNC) content to obtain the combined cells. In a specific embodiment, the cord blood and placental perfusate are combined at a ratio of from 10:1 to 10:1, from 5:1 to 1:5, or from 3:1 to 1:3. In another specific embodiment, the cord blood and placental perfusate are combined at a ratio of 10:1, 5:1, 3:1, 1:1, 1:3, 1:5 or 1:10.

In one embodiment, therefore, provided herein is a method of treating an individual having cancer or a viral infection, comprising administering to said individual an effective amount of cells from an isolated NK cell population produced using the three-stage methods described herein. In certain embodiments, the cancer is a solid tumor. In certain embodiments, the cancer is a hematological cancer. In a specific embodiment, the hematological cancer is leukemia. In another specific embodiment, the hematological cancer is lymphoma. In another specific embodiment, the hematological cancer is acute myeloid leukemia. In another specific embodiment, the hematological cancer is chronic lymphocytic leukemia. In another specific embodiment, the hematological cancer is chronic myelogenous leukemia. In certain aspects, said natural killer cells have been cryopreserved prior to said contacting or said administering. In other aspects, said natural killer cells have not been cryopreserved prior to said contacting or said administering.

In a specific embodiment, the NK cell populations produced using the three-stage methods described herein have been treated with an immunomodulatory compound, e.g. an immunomodulatory compound described herein, or thalidomide, prior to said administration. In a specific embodiment, the NK cell populations produced using the three-stage methods described herein have been treated with IL2 and IL12 and IL18, IL12 and IL15, IL12 and IL18, IL2 and IL12 and IL15 and IL18, or IL2 and IL15 and IL18 prior to said administration. In another specific embodiment, the isolated NK cell population produced using the three-stage methods described herein has been pretreated with one or more of IL2, IL12, IL18, or IL15 prior to said administration. In another specific embodiment, the method comprises administering to the individual (1) an effective amount of an isolated NK cell population produced using a three-stage method described herein; and (2) an effective amount of an immunomodulatory compound or thalidomide. An “effective amount” in this context means an amount of cells in an NK cell population, and optionally immunomodulatory compound or thalidomide, that results in a detectable improvement in one or more symptoms of said cancer or said infection, compared to an individual having said cancer or said infection who has not been administered said NK cell population and, optionally, an immunomodulatory compound or thalidomide. In a specific embodiment, said immunomodulatory compound is lenalidomide or pomalidomide. In another embodiment, the method additionally comprises administering an anticancer compound to the individual, e.g., one or more of the anticancer compounds described below.

In another embodiment, provided herein is a method of suppressing the proliferation of tumor cells comprising bringing a therapeutically effective amount of an NK cell population into proximity with the tumor cells, e.g., contacting the tumor cells with the cells in an NK cell population. Hereinafter, unless noted otherwise, the term “proximity” refers to sufficient proximity to elicit the desired result; e.g., in certain embodiments, the term proximity refers to contact. In certain embodiments, said contacting takes place in vitro. In certain embodiments, said contacting takes place ex vivo. In other embodiments, said contacting takes place in vivo. A plurality of NK cells can be used in the method of suppressing the proliferation of the tumor cells comprising bringing a therapeutically effective amount of the NK cell population into proximity with the tumor cells, e.g., contacting the tumor cells with the cells in the NK cell population. In certain embodiments, said tumor cells are breast cancer cells, head and neck cancer cells, or sarcoma cells. In certain embodiments, said tumor cells are primary ductal carcinoma cells, leukemia cells, acute T cell leukemia cells, chronic myeloid lymphoma (CML) cells, chronic myelogenous leukemia (CML) cells, lung carcinoma cells, colon adenocarcinoma cells, histiocytic lymphoma cells, colorectal carcinoma cells, colorectal adenocarcinoma cells, or retinoblastoma cells.

In one embodiment, provided herein are a plurality of natural killer cells for use in a method of suppressing the proliferation of tumor cells comprising contacting the tumor cells with the plurality of natural killer cells, wherein the natural killer cells are produced by the methods described herein. In certain embodiments, said contacting takes place in a human individual. In certain embodiments, said method comprises administering said natural killer cells to said individual. In certain embodiments, said tumor cells are multiple myeloma cells. In certain embodiments, said tumor cells are acute myeloid leukemia (AML) cells. In certain embodiments, said individual has relapsed/refractory AML. In certain embodiments, said individual has AML that has failed at least one non-innate lymphoid cell (ILC) therapeutic against AML. In certain embodiments, said individual is 65 years old or greater, and is in first remission. In certain embodiments, said individual has been conditioned with fludarabine, cytarabine, or both prior to administering said NK cells. In certain embodiments, said tumor cells are breast cancer cells, head and neck cancer cells, or sarcoma cells. In certain embodiments, said tumor cells are primary ductal carcinoma cells, leukemia cells, acute T cell leukemia cells, chronic myeloid lymphoma (CML) cells, chronic myelogenous leukemia (CML) cells, lung carcinoma cells, colon adenocarcinoma cells, histiocytic lymphoma cells, colorectal carcinoma cells, colorectal adenocarcinoma cells, or retinoblastoma cells. In certain embodiments, said tumor cells are solid tumor cells, liver tumor cells, lung tumor cells, pancreatic tumor cells, renal tumor cells or glioblastoma multiforme (GBM) cells. In certain embodiments, said natural killer cells are administered with an anti-CD33 antibody, an anti-CD20 antibody, an anti-CD138 antibody or an anti-CD32 antibody. In certain embodiments, said NK cells have or have not been cryopreserved prior to said contacting or said administering.

Administration of an isolated population of NK cells or a pharmaceutical composition thereof may be systemic or local. In specific embodiments, administration is parenteral. In specific embodiments, administration of an isolated population of NK cells or a pharmaceutical composition thereof to a subject is by injection, infusion, intravenous (IV) administration, intrafemoral administration, or intratumor administration. In specific embodiments, administration of an isolated population of NK cells or a pharmaceutical composition thereof to a subject is performed with a device, a matrix, or a scaffold. In specific embodiments, administration an isolated population of NK cells or a pharmaceutical composition thereof to a subject is by injection. In specific embodiments, administration an isolated population of NK cells or a pharmaceutical composition thereof to a subject is via a catheter. In specific embodiments, the injection of NK cells is local injection. In more specific embodiments, the local injection is directly into a solid tumor (e.g., a sarcoma). In specific embodiments, administration of an isolated population of NK cells or a pharmaceutical composition thereof to a subject is by injection by syringe. In specific embodiments, administration of an isolated population of NK cells or a pharmaceutical composition thereof to a subject is via guided delivery. In specific embodiments, administration of an isolated population of NK cells or a pharmaceutical composition thereof to a subject by injection is aided by laparoscopy, endoscopy, ultrasound, computed tomography, magnetic resonance, or radiology.

In a specific embodiment, the isolated NK cell population produced using the three-stage methods described herein has been treated with an immunomodulatory compound, e.g. an immunomodulatory compound described herein, below, or thalidomide, and/or IL2 and IL12 and IL18, IL12 and IL15, IL12 and IL18, IL2 and IL12 and IL15 and IL18, or IL2 and IL15 and IL18, prior to said contacting or bringing into proximity. In another specific embodiment, the isolated NK cell population produced using the three-stage methods described herein has been treated with one or more of IL2, IL12, IL18, or IL15 prior to said contacting or bringing into proximity. In another specific embodiment, an effective amount of an immunomodulatory compound, e.g., an immunomodulatory compound described herein, below, or thalidomide is additionally brought into proximity with the tumor cells e.g., the tumor cells are contacted with the immunomodulatory compound or thalidomide. An “effective amount” in this context means an amount of cells in an NK cell population, and optionally an immunomodulatory compound or thalidomide, that results in a detectable suppression of said tumor cells compared to an equivalent number of tumor cells not contacted or brought into proximity with cells in an NK cell population, and optionally an immunomodulatory compound or thalidomide. In another specific embodiment, the method further comprises bringing an effective amount of an anticancer compound, e.g., an anticancer compound described below, into proximity with the tumor cells, e.g., contacting the tumor cells with the anticancer compound.

In a specific embodiment of this method, the tumor cells are blood cancer cells. In another specific embodiment, the tumor cells are solid tumor cells. In another embodiment, the tumor cells are primary ductal carcinoma cells, leukemia cells, acute T cell leukemia cells, chronic myeloid lymphoma (CML) cells, acute myelogenous leukemia cells (AML), chronic myelogenous leukemia (CML) cells, glioblastoma cells, lung carcinoma cells, colon adenocarcinoma cells, histiocytic lymphoma cells, multiple myeloma cells, retinoblastoma cell, colorectal carcinoma cells, prostate cancer cells, or colorectal adenocarcinoma cells. In more specific embodiments, the tumor cells are AML cells. In more specific embodiments, the tumor cells are multiple myeloma cells. In another specific embodiment, said contacting or bringing into proximity takes place in vitro. In another specific embodiment, said contacting or bringing into proximity takes place ex vivo. In another specific embodiment, said contacting or bringing into proximity takes place in vivo. In a more specific embodiment, said in vivo contacting or bringing into proximity takes place in a human. In a specific embodiment, said tumor cells are solid tumor cells. In a specific embodiment, said tumor cells are liver tumor cells. In a specific embodiment, said tumor cells are lung tumor cells. In a specific embodiment, said tumor cells are pancreatic tumor cells. In a specific embodiment, said tumor cells are renal tumor cells. In a specific embodiment, said tumor cells are glioblastoma multiforme (GBM) cells. In a specific embodiment, said natural killer cells are administered with an antibody. In a specific embodiment, said natural killer cells are administered with an anti-CD33 antibody. In a specific embodiment, said natural killer cells are administered with an anti-CD20 antibody. In a specific embodiment, said natural killer cells are administered with an anti-CD138 antibody. In a specific embodiment, said natural killer cells are administered with an anti-CD32 antibody.

In another aspect, provided herein is a method of treating an individual having multiple myeloma, comprising administering to the individual (1) lenalidomide; (2) melphalan; and (3) NK cells, wherein said NK cells are effective to treat multiple myeloma in said individual. In a specific embodiment, said NK cells are cord blood NK cells, or NK cells produced from cord blood hematopoietic cells, e.g., hematopoietic stem cells. In another embodiment, said NK cells have been produced by any of the methods described herein for producing NK cells, e.g., for producing NK cell populations using a three-stage method. In another embodiment, said NK cells have been expanded prior to said administering. In another embodiment, said lenalidomide, melphalan, and/or NK cells are administered separately from each other. In certain specific embodiments of the method of treating an individual with multiple myeloma, said NK cell populations are produced by a three-stage method, as described herein.

In another aspect, provided herein is a method of treating an individual having acute myelogenous leukemia (AML), comprising administering to the individual NK cells (optionally activated by pretreatment with IL2 and IL12 and IL18, IL12 and IL15, IL12 and IL18, IL2 and IL12 and IL15 and IL18, or IL2 and IL15 and IL18), wherein said NK cells are effective to treat AML in said individual. In a specific embodiment, the isolated NK cell population produced using the three-stage methods described herein has been pretreated with one or more of IL2, IL12, IL18, or IL15 prior to said administering. In a specific embodiment, said NK cells are cord blood NK cells, or NK cells produced from cord blood hematopoietic cells, e.g., hematopoietic stem cells. In another embodiment, said NK cells have been produced by any of the methods described herein for producing NK cells, e.g., for producing NK cell populations using a three-stage method as set forth herein. In certain specific embodiments of the method of treating an individual with AML, said NK cell populations are produced by a three-stage method, as described herein. In a particular embodiment, the AML to be treated by the foregoing methods comprises refractory AML, poor-prognosis AML, or childhood AML. In certain embodiments, said individual has AML that has failed at least one non-natural killer or non-innate lymphoid cell therapeutic against AML. In specific embodiments, said individual is 65 years old or greater, and is in first remission. In specific embodiments, said individual has been conditioned with fludarabine, cytarabine, or both prior to administering said natural killer cells.

In another aspect, provided herein is a method of treating an individual having chronic lymphocytic leukemia (CLL), comprising administering to the individual a therapeutically effective dose of (1) lenalidomide; (2) melphalan; (3) fludarabine; and (4) NK cells, e.g., a NK cell population produced using a three-stage method described herein, wherein said NK cells are effective to treat said CLL in said individual. In a specific embodiment, said NK cells are cord blood NK cells, or NK cells produced from cord blood hematopoietic cells, e.g., hematopoietic stem cells. In another embodiment, said NK cells have been produced by any of the methods described herein for producing NK cells, e.g., for producing NK cell populations using a three-stage method described herein. In a specific embodiment of any of the above methods, said lenalidomide, melphalan, fludarabine, and expanded NK cells are administered to said individual separately. In certain specific embodiments of the method of treating an individual with CLL, said NK cell populations are produced by a three-stage method, as described herein.

In another embodiment, provided herein is a method of suppressing the proliferation of tumor cells comprising bringing a therapeutically effective amount of an ILC3 cell population into proximity with the tumor cells, e.g., contacting the tumor cells with the cells in an ILC3 cell population. Hereinafter, unless noted otherwise, the term “proximity” refers to sufficient proximity to elicit the desired result; e.g., in certain embodiments, the term proximity refers to contact. In certain embodiments, said contacting takes place in vitro. In certain embodiments, said contacting takes place ex vivo. In other embodiments, said contacting takes place in vivo. A plurality of ILC3 cells can be used in the method of suppressing the proliferation of the tumor cells comprising bringing a therapeutically effective amount of the ILC3 cell population into proximity with the tumor cells, e.g., contacting the tumor cells with the cells in the ILC3 cell population. In certain embodiments, said tumor cells are breast cancer cells, head and neck cancer cells, or sarcoma cells. In certain embodiments, said tumor cells are primary ductal carcinoma cells, leukemia cells, acute T cell leukemia cells, chronic myeloid lymphoma (CML) cells, chronic myelogenous leukemia (CML) cells, lung carcinoma cells, colon adenocarcinoma cells, histiocytic lymphoma cells, colorectal carcinoma cells, colorectal adenocarcinoma cells, or retinoblastoma cells.

In one embodiment, provided herein are a plurality of ILC3 cells for use in a method of suppressing the proliferation of tumor cells comprising contacting the tumor cells with the plurality of ILC3 cells, wherein the ILC3 cells are produced by the methods described herein. In certain embodiments, said contacting takes place in a human individual. In certain embodiments, said method comprises administering said ILC3 cells to said individual. In certain embodiments, said tumor cells are multiple myeloma cells. In certain embodiments, said tumor cells are acute myeloid leukemia (AML) cells. In certain embodiments, said individual has relapsed/refractory AML. In certain embodiments, said individual has AML that has failed at least one non-innate lymphoid cell (ILC) therapeutic against AML. In certain embodiments, said individual is 65 years old or greater, and is in first remission. In certain embodiments, said individual has been conditioned with fludarabine, cytarabine, or both prior to administering said ILC3 cells. In certain embodiments, said tumor cells are breast cancer cells, head and neck cancer cells, or sarcoma cells. In certain embodiments, said tumor cells are primary ductal carcinoma cells, leukemia cells, acute T cell leukemia cells, chronic myeloid lymphoma (CML) cells, chronic myelogenous leukemia (CML) cells, lung carcinoma cells, colon adenocarcinoma cells, histiocytic lymphoma cells, colorectal carcinoma cells, colorectal adenocarcinoma cells, or retinoblastoma cells. In certain embodiments, said tumor cells are solid tumor cells, liver tumor cells, lung tumor cells, pancreatic tumor cells, renal tumor cells or glioblastoma multiforme (GBM) cells. In certain embodiments, said ILC3 cells are administered with an anti-CD33 antibody, an anti-CD20 antibody, an anti-CD138 antibody or an anti-CD32 antibody. In certain embodiments, said ILC3 cells have or have not been cryopreserved prior to said contacting or said administering.

Administration of an isolated population of ILC3 cells or a pharmaceutical composition thereof may be systemic or local. In specific embodiments, administration is parenteral. In specific embodiments, administration of an isolated population of ILC3 cells or a pharmaceutical composition thereof to a subject is by injection, infusion, intravenous (IV) administration, intrafemoral administration, or intratumor administration. In specific embodiments, administration of an isolated population of ILC3 cells or a pharmaceutical composition thereof to a subject is performed with a device, a matrix, or a scaffold. In specific embodiments, administration an isolated population of ILC3 cells or a pharmaceutical composition thereof to a subject is by injection. In specific embodiments, administration an isolated population of ILC3 cells or a pharmaceutical composition thereof to a subject is via a catheter. In specific embodiments, the injection of ILC3 cells is local injection. In more specific embodiments, the local injection is directly into a solid tumor (e.g., a sarcoma). In specific embodiments, administration of an isolated population of ILC3 cells or a pharmaceutical composition thereof to a subject is by injection by syringe. In specific embodiments, administration of an isolated population of ILC3 cells or a pharmaceutical composition thereof to a subject is via guided delivery. In specific embodiments, administration of an isolated population of ILC3 cells or a pharmaceutical composition thereof to a subject by injection is aided by laparoscopy, endoscopy, ultrasound, computed tomography, magnetic resonance, or radiology.

In a specific embodiment, the isolated ILC3 cell population produced using the three-stage methods described herein has been treated with an immunomodulatory compound, e.g. an immunomodulatory compound described herein, below, or thalidomide, and/or IL2 and IL12 and IL18, IL12 and IL15, IL12 and IL18, IL2 and IL12 and IL15 and IL18, or IL2 and IL15 and IL18, prior to said contacting or bringing into proximity. In a specific embodiment, the isolated NK cell population produced using the three-stage methods described herein has been treated with one or more of IL2, IL12, IL18, or IL15 prior to said contacting or bringing into proximity. In another specific embodiment, an effective amount of an immunomodulatory compound, e.g., an immunomodulatory compound described herein, below, or thalidomide is additionally brought into proximity with the tumor cells e.g., the tumor cells are contacted with the immunomodulatory compound or thalidomide. An “effective amount” in this context means an amount of cells in an ILC3 cell population, and optionally an immunomodulatory compound or thalidomide, that results in a detectable suppression of said tumor cells compared to an equivalent number of tumor cells not contacted or brought into proximity with cells in an ILC3 cell population, and optionally an immunomodulatory compound or thalidomide. In another specific embodiment, the method further comprises bringing an effective amount of an anticancer compound, e.g., an anticancer compound described below, into proximity with the tumor cells, e.g., contacting the tumor cells with the anticancer compound.

In a specific embodiment of this method, the tumor cells are blood cancer cells. In another specific embodiment, the tumor cells are solid tumor cells. In another embodiment, the tumor cells are primary ductal carcinoma cells, leukemia cells, acute T cell leukemia cells, chronic myeloid lymphoma (CML) cells, acute myelogenous leukemia cells (AML), chronic myelogenous leukemia (CML) cells, glioblastoma cells, lung carcinoma cells, colon adenocarcinoma cells, histiocytic lymphoma cells, multiple myeloma cells, retinoblastoma cell, colorectal carcinoma cells, prostate cancer cells, or colorectal adenocarcinoma cells. In another specific embodiment, said contacting or bringing into proximity takes place in vitro. In another specific embodiment, said contacting or bringing into proximity takes place ex vivo. In another specific embodiment, said contacting or bringing into proximity takes place in vivo. In a more specific embodiment, said in vivo contacting or bringing into proximity takes place in a human. In a specific embodiment, said tumor cells are solid tumor cells. In a specific embodiment, said tumor cells are liver tumor cells. In a specific embodiment, said tumor cells are lung tumor cells. In a specific embodiment, said tumor cells are pancreatic tumor cells. In a specific embodiment, said tumor cells are renal tumor cells. In a specific embodiment, said tumor cells are glioblastoma multiforme (GBM) cells. In a specific embodiment, said ILC3 cells are administered with an antibody. In a specific embodiment, said ILC3 cells are administered with an anti-CD33 antibody. In a specific embodiment, said ILC3 cells are administered with an anti-CD20 antibody. In a specific embodiment, said ILC3 cells are administered with an anti-CD138 antibody. In a specific embodiment, said ILC3 cells are administered with an anti-CD32 antibody.

In another aspect, provided herein is a method of treating an individual having multiple myeloma, comprising administering to the individual (1) lenalidomide; (2) melphalan; and (3) ILC3 cells, wherein said ILC3 cells are effective to treat multiple myeloma in said individual. In a specific embodiment, said ILC3 cells are cord blood ILC3 cells, or ILC3 cells produced from cord blood hematopoietic cells, e.g., hematopoietic stem cells. In another embodiment, said ILC3 cells have been produced by any of the methods described herein for producing ILC3 cells, e.g., for producing ILC3 cell populations using a three-stage method. In another embodiment, said ILC3 cells have been expanded prior to said administering. In another embodiment, said lenalidomide, melphalan, and/or ILC3 cells are administered separately from each other. In certain specific embodiments of the method of treating an individual with multiple myeloma, said ILC3 cell populations are produced by a three-stage method, as described herein.

In another aspect, provided herein is a method of treating an individual having acute myelogenous leukemia (AML), comprising administering to the individual ILC3 cells (optionally activated by pretreatment with IL2 and IL12 and IL18, IL12 and IL15, IL12 and IL18, IL2 and IL12 and IL15 and IL18, or IL2 and IL15 and IL18), wherein said ILC3 cells are effective to treat AML in said individual. In a specific embodiment, the isolated NK cell population produced using the three-stage methods described herein has been pretreated with one or more of IL2, IL12, IL18, or IL15 prior to said administering. In a specific embodiment, said ILC3 cells are cord blood ILC3 cells, or ILC3 cells produced from cord blood hematopoietic cells, e.g., hematopoietic stem cells. In another embodiment, said ILC3 cells have been produced by any of the methods described herein for producing ILC3 cells, e.g., for producing ILC3 cell populations using a three-stage method as set forth herein. In certain specific embodiments of the method of treating an individual with AML, said ILC3 cell populations are produced by a three-stage method, as described herein. In a particular embodiment, the AML to be treated by the foregoing methods comprises refractory AML, poor-prognosis AML, or childhood AML. In certain embodiments, said individual has AML that has failed at least one non-ILC3 or non-innate lymphoid cell therapeutic against AML. In specific embodiments, said individual is 65 years old or greater, and is in first remission. In specific embodiments, said individual has been conditioned with fludarabine, cytarabine, or both prior to administering said ILC3 cells. In another aspect, provided herein is a method of treating an individual having chronic lymphocytic leukemia (CLL), comprising administering to the individual a therapeutically effective dose of (1) lenalidomide; (2) melphalan; (3) fludarabine; and (4) ILC3 cells, e.g., a ILC3 cell population produced using a three-stage method described herein, wherein said ILC3 cells are effective to treat said CLL in said individual. In a specific embodiment, said ILC3 cells are cord blood ILC3 cells, or ILC3 cells produced from cord blood hematopoietic cells, e.g., hematopoietic stem cells. In another embodiment, said ILC3 cells have been produced by any of the methods described herein for producing ILC3 cells, e.g., for producing ILC3 cell populations using a three-stage method described herein. In a specific embodiment of any of the above methods, said lenalidomide, melphalan, fludarabine, and expanded ILC3 cells are administered to said individual separately. In certain specific embodiments of the method of treating an individual with CLL, said ILC3 cell populations are produced by a three-stage method, as described herein.

In certain embodiments, the NK cell populations produced using a three-stage method described herein are cryopreserved, e.g., cryopreserved using a method described herein. In a certain embodiments, the NK cell populations produced using a three-stage method described herein are cryopreserved in a cryopreservation medium, e.g., a cryopreservation medium described herein. In a specific embodiment, cryopreservation of the NK progenitor cell populations and/or NK cell populations produced using a three-stage method described herein comprises (1) preparing a cell suspension solution comprising an NK progenitor cell population and/or an NK cell population produced using a three-stage method described herein; (2) adding cryopreservation medium to the cell suspension solution from step (1) to obtain a cryopreserved cell suspension; (3) cooling the cryopreserved cell suspension from step (3) to obtain a cryopreserved sample; and (4) storing the cryopreserved sample below −80° C.

In certain embodiments of the methods of treatment or tumor suppression above, NK cell populations produced by a three-stage method described herein are combined with other natural killer cells, e.g., natural killer cells isolated from placental perfusate, umbilical cord blood or peripheral blood, or produced from hematopoietic cells by a different method. In specific embodiments, the natural killer cell populations are combined with natural killer cells from another source, or made by a different method, in a ratio of about 100:1, 95:5, 90:10, 85:15, 80:20, 75:25, 70:30, 65:35, 60:40, 55:45: 50:50, 45:55, 40:60, 35:65, 30:70, 25:75, 20:80, 15:85, 10:90, 5:95, 100:1, 95:1, 90:1, 85:1, 80:1, 75:1, 70:1, 65:1, 60:1, 55:1, 50:1, 45:1, 40:1, 35:1, 30:1, 25:1, 20:1, 15:1, 10:1, 5:1, 1:1, 1:5, 1:10, 1:15, 1:20, 1:25, 1:30, 1:35, 1:40, 1:45, 1:50, 1:55, 1:60, 1:65, 1:70, 1:75, 1:80, 1:85, 1:90, 1:95, 1:100, or the like.

In certain embodiments, the ILC3 cell populations produced using a three-stage method described herein are cryopreserved, e.g., cryopreserved using a method described herein. In a certain embodiments, the ILC3 cell populations produced using a three-stage method described herein are cryopreserved in a cryopreservation medium, e.g., a cryopreservation medium described herein. In a specific embodiment, cryopreservation of the ILC3 progenitor cell populations and/or ILC3 cell populations produced using a three-stage method described herein comprises (1) preparing a cell suspension solution comprising an ILC3 progenitor cell population and/or an ILC3 cell population produced using a three-stage method described herein; (2) adding cryopreservation medium to the cell suspension solution from step (1) to obtain a cryopreserved cell suspension; (3) cooling the cryopreserved cell suspension from step (3) to obtain a cryopreserved sample; and (4) storing the cryopreserved sample below −80° C.

In certain embodiments of the methods of treatment or tumor suppression above, ILC3 cell populations produced by a three-stage method described herein are combined with other ILC3 cells, e.g., ILC3 cells isolated from placental perfusate, umbilical cord blood or peripheral blood, or produced from hematopoietic cells by a different method. In specific embodiments, the ILC3 cell populations are combined with ILC3 cells from another source, or made by a different method, in a ratio of about 100:1, 95:5, 90:10, 85:15, 80:20, 75:25, 70:30, 65:35, 60:40, 55:45: 50:50, 45:55, 40:60, 35:65, 30:70, 25:75, 20:80, 15:85, 10:90, 5:95, 100:1, 95:1, 90:1, 85:1, 80:1, 75:1, 70:1, 65:1, 60:1, 55:1, 50:1, 45:1, 40:1, 35:1, 30:1, 25:1, 20:1, 15:1, 10:1, 5:1, 1:1, 1:5, 1:10, 1:15, 1:20, 1:25, 1:30, 1:35, 1:40, 1:45, 1:50, 1:55, 1:60, 1:65, 1:70, 1:75, 1:80, 1:85, 1:90, 1:95, 1:100, or the like.

In another aspect, provided herein is a method of repairing the gastrointestinal tract after chemotherapy comprising administering to an individual a plurality of ILC3 cells, wherein the ILC3 cells are produced by a three-stage method described herein. A plurality of ILC3 cells can be used in the method of repairing the gastrointestinal tract after chemotherapy comprising administering to an individual a plurality of the ILC3 cells, wherein the ILC3 cells are produced by a three-stage method described herein.

In another aspect, provided herein is a method of protecting an individual against radiation comprising administering to an individual a plurality of ILC3 cells, wherein the ILC3 cells are produced by a three-stage method described herein. A plurality of ILC3 cells can be used in the method of protecting an individual against radiation comprising administering to an individual a plurality of the ILC3 cells, wherein the ILC3 cells are produced by a three-stage method described herein. In certain aspects of the method, said ILC3 cells are used as an adjunct to bone marrow transplantation.

In another aspect, provided herein is a method of reconstituting the thymus of an individual comprising administering to an individual a plurality of ILC3 cells, wherein the ILC3 cells are produced by a three-stage method described herein. A plurality of ILC3 cells can be used in the method of reconstituting the thymus of an individual comprising administering to an individual a plurality of the ILC3 cells, wherein the ILC3 cells are produced by a three-stage method described herein.

In another aspect, provided herein is a composition comprising isolated NK cells produced by a three-stage method described herein. In a specific embodiment, said NK cells are produced from hematopoietic cells, e.g., hematopoietic stem or progenitor cells isolated from placental perfusate, umbilical cord blood, and/or peripheral blood. In another specific embodiment, said NK cells comprise at least 70% of cells in the composition. In another specific embodiment, said NK cells comprise at least 80%, 85%, 90%, 95%, 98% or 99% of cells in the composition. In certain embodiments, at least 80%, 82%, 84%, 86%, 88% or 90% of NK cells in said composition are CD3⁻ and CD56⁺. In certain embodiments, at least 65%, 70%, 75%, 80%, 82%, 84%, 86%, 88% or 90% of NK cells in said composition are CD16−. In certain embodiments, at least 5%, 10%, 15%, 20%, 25%, 30%, 35%, 40%, 45%, 50%, 55% or 60% of NK cells in said composition are CD94+.

In certain aspects, provided herein is a population of natural killer cells that is CD56+CD3− CD117+CD11a+, wherein said natural killer cells express perforin and/or EOMES, and do not express one or more of RORγt, aryl hydrocarbon receptor, and IL1R1. In certain aspects, said natural killer cells express perforin and EOMES, and do not express any of RORγt, aryl hydrocarbon receptor, or IL1R1. In certain aspects, said natural killer cells additionally express T-bet, GZMB, NKp46, NKp30, and NKG2D. In certain aspects, said natural killer cells express CD94. In certain aspects, said natural killer cells do not express CD94.

In certain aspects, provided herein is a population of ILC3 cells that is CD56+CD3− CD117+CD11a−, wherein said ILC3 cells express one or more of RORγt, aryl hydrocarbon receptor, and IL1R1, and do not express one or more of CD94, perforin, and EOMES. In certain aspects, said ILC3 cells express RORγt, aryl hydrocarbon receptor, and IL1R1, and do not express any of CD94, perforin, or EOMES. In certain aspects, said ILC3 cells additionally express CD226 and/or 2B4. In certain aspects, said ILC3 cells additionally express one or more of IL-22, TNFα, and DNAM-1. In certain aspects, said ILC3 cells express CD226, 2B4, IL-22, TNFα, and DNAM-1.

In certain aspects, provided herein is a method of producing a cell population comprising natural killer cells and ILC3 cells, comprising (a) culturing hematopoietic stem or progenitor cells in a first medium comprising a stem cell mobilizing agent and thrombopoietin (Tpo) to produce a first population of cells; (b) culturing the first population of cells in a second medium comprising a stem cell mobilizing agent and interleukin-15 (IL-15), and lacking Tpo, to produce a second population of cells; (c) culturing the second population of cells in a third medium comprising IL-2 and IL-15, and lacking each of a stem cell mobilizing agent and LMWH, to produce a third population of cells; and (d) separating CD11a+ cells and CD11a− cells from the third population of cells; and (e) combining the CD11a+ cells with the CD11a− cells in a ratio of 50:1, 40:1, 30:1, 20:1, 10:1, 5:1, 4:1, 3:1, 2:1, 1:1, 1:2, 1:3, 1:4, 1:5, 1:10, 1:20, 1:30, 1:40, or 1:50 to produce a fourth population of cells. In certain embodiments, said first medium and/or said second medium lack leukemia inhibiting factor (LIF) and/or macrophage inflammatory protein-1 alpha (MIP-1α). In certain embodiments, said third medium lacks LIF, MIP-1α, and FMS-like tyrosine kinase-3 ligand (Flt-3L). In specific embodiments, said first medium and said second medium lack LIF and MIP-1α, and said third medium lacks LIF, MIP-1a, and Flt3L. In certain embodiments, none of the first medium, second medium or third medium comprises heparin, e.g., low-molecular weight heparin. In certain aspects, in the fourth population of cells, the CD11a+ cells and CD11a− cells are combined in a ratio of 50:1. In certain aspects, in the fourth population of cells, the CD11a+ cells and CD11a− cells are combined in a ratio of 20:1. In certain aspects, in the fourth population of cells, the CD11a+ cells and CD11a− cells are combined in a ratio of 10:1. In certain aspects, in the fourth population of cells, the CD11a+ cells and CD11a− cells are combined in a ratio of 5:1. In certain aspects, in the fourth population of cells, the CD11a+ cells and CD11a− cells are combined in a ratio of 1:1. In certain aspects, in the fourth population of cells, the CD11a+ cells and CD11a− cells are combined in a ratio of 1:5. In certain aspects, in the fourth population of cells, the CD11a+ cells and CD11a− cells are combined in a ratio of 1:10. In certain aspects, in the fourth population of cells, the CD11a+ cells and CD11a− cells are combined in a ratio of 1:20. In certain aspects, in the fourth population of cells, the CD11a+ cells and CD11a− cells are combined in a ratio of 1:50.

In certain aspects, a plurality of the NK cells in said population expresses one or more of the microRNAS dme-miR-7, hsa-let-7a, hsa-let-7c, hsa-let-7e, hsa-let-7g, hsa-miR-103, hsa-miR-106a, hsa-miR-10b, hsa-miR-1183, hsa-miR-124, hsa-miR-1247, hsa-miR-1248, hsa-miR-1255A, hsa-miR-126, hsa-miR-140-3p, hsa-miR-144, hsa-miR-151-3p, hsa-miR-155, hsa-miR-15a, hsa-miR-16, hsa-miR-17, hsa-miR-181a, hsa-miR-182, hsa-miR-192, hsa-miR-199a-3p, hsa-miR-200a, hsa-miR-20a, hsa-miR-214, hsa-miR-221, hsa-miR-29a, hsa-miR-29b, hsa-miR-30b, hsa-miR-30c, hsa-miR-31, hsa-miR-335, hsa-miR-374b, hsa-miR-454, hsa-miR-484, hsa-miR-513C, hsa-miR-516-3p, hsa-miR-520h, hsa-miR-548K, hsa-miR-548P, hsa-miR-600, hsa-miR-641, hsa-miR-643, hsa-miR-874, hsa-miR-875-5p, and hsa-miR-92a-2 at a detectably higher level as peripheral blood natural killer cells. In certain aspects, a plurality of the NK cells in said population expresses one or more of the microRNAS miR188-5p, miR-339-5p, miR-19a, miR-34c, miR-18a, miR-500, miR-22, miR-222, miR-7a, miR-532-3p, miR-223, miR-26b, miR-26a, miR-191, miR-181d, miR-322, and miR342-3p at a detectably lower level than peripheral blood natural killer cells. In certain aspects, a plurality of the NK cells in said population expresses one or more of the microRNAS miR-181a, miR-30b, and miR30c at an equivalent level to peripheral blood natural killer cells.

In a specific embodiment, said NK cells are from a single individual, that is, said hemtopoietic stem and progenitor cells are from a single individual. In a more specific embodiment, said NK cells comprise natural killer cells from at least two different individuals, that is, said hemtopoietic stem and progenitor cells are from at least two different individuals. In another specific embodiment, said NK cells are from a different individual than the individual for whom treatment with the NK cells is intended, that is, said hemtopoietic stem and progenitor cells are from a different individual than the individual for whom treatment with the NK cells is intended. In another specific embodiment, said NK cells have been contacted or brought into proximity with an immunomodulatory compound or thalidomide in an amount and for a time sufficient for said NK cells to express detectably more granzyme B or perforin than an equivalent number of natural killer cells, i.e. NK cells, not contacted or brought into proximity with said immunomodulatory compound or thalidomide. In another specific embodiment, a composition comprising said NK cells additionally comprises an immunomodulatory compound or thalidomide. In certain embodiments, the immunomodulatory compound is a compound described below, e.g., an amino-substituted isoindoline compound. In certain embodiments, the immunomodulatory compound is lenalidomide. In certain embodiments, the immunomodulatory compound is pomalidomide.

In another specific embodiment, a composition comprising said NK cells additionally comprises one or more anticancer compounds, e.g., one or more of the anticancer compounds described below.

In a more specific embodiment, the composition comprises NK cells produced by a three-stage method described herein and natural killer cells from another source or made by another method. In a specific embodiment, said other source is placental blood and/or umbilical cord blood. In another specific embodiment, said other source is peripheral blood. In more specific embodiments, the NK cells are combined with natural killer cells from another source, or made by another method in a ratio of about 100:1, 95:5, 90:10, 85:15, 80:20, 75:25, 70:30, 65:35, 60:40, 55:45: 50:50, 45:55, 40:60, 35:65, 30:70, 25:75, 20:80, 15:85, 10:90, 5:95, 100:1, 95:1, 90:1, 85:1, 80:1, 75:1, 70:1, 65:1, 60:1, 55:1, 50:1, 45:1, 40:1, 35:1, 30:1, 25:1, 20:1, 15:1, 10:1, 5:1, 1:1, 1:5, 1:10, 1:15, 1:20, 1:25, 1:30, 1:35, 1:40, 1:45, 1:50, 1:55, 1:60, 1:65, 1:70, 1:75, 1:80, 1:85, 1:90, 1:95, 1:100, or the like.

In another specific embodiment, the composition comprises NK cells produced using a three-stage method described herein and either isolated placental perfusate or isolated placental perfusate cells. In a more specific embodiment, said placental perfusate is from the same individual as said NK cells. In another more specific embodiment, said placental perfusate comprises placental perfusate from a different individual than said NK cells. In another specific embodiment, all, or substantially all (e.g., greater than 90%, 95%, 98% or 99%) of cells in said placental perfusate are fetal cells. In another specific embodiment, the placental perfusate or placental perfusate cells, comprise fetal and maternal cells. In a more specific embodiment, the fetal cells in said placental perfusate comprise less than about 90%, 80%, 70%, 60% or 50% of the cells in said perfusate. In another specific embodiment, said perfusate is obtained by passage of a 0.9% NaCl solution through the placental vasculature. In another specific embodiment, said perfusate comprises a culture medium. In another specific embodiment, said perfusate has been treated to remove erythrocytes. In another specific embodiment, said composition comprises an immunomodulatory compound, e.g., an immunomodulatory compound described below, e.g., an amino-substituted isoindoline compound. In another specific embodiment, the composition additionally comprises one or more anticancer compounds, e.g., one or more of the anticancer compounds described below.

In another specific embodiment, the composition comprises NK cells produced using a three-stage method described herein and placental perfusate cells. In a more specific embodiment, said placental perfusate cells are from the same individual as said NK cells. In another more specific embodiment, said placental perfusate cells are from a different individual than said NK cells. In another specific embodiment, the composition comprises isolated placental perfusate and isolated placental perfusate cells, wherein said isolated perfusate and said isolated placental perfusate cells are from different individuals. In another more specific embodiment of any of the above embodiments comprising placental perfusate, said placental perfusate comprises placental perfusate from at least two individuals. In another more specific embodiment of any of the above embodiments comprising placental perfusate cells, said isolated placental perfusate cells are from at least two individuals. In another specific embodiment, said composition comprises an immunomodulatory compound. In another specific embodiment, the composition additionally comprises one or more anticancer compounds, e.g., one or more of the anticancer compounds described below.

In another aspect, provided herein is a composition, e.g., a pharmaceutical composition, comprising an isolated NK cell population, e.g., produced by any embodiment of the three-stage method described herein. In a specific embodiment, said isolated NK cell population is produced from hematopoietic cells, e.g., hematopoietic stem or progenitor cells isolated from placenta, e.g., from placental perfusate, umbilical cord blood, and/or peripheral blood. In another specific embodiment, said isolated NK cell population comprises at least 70% of cells in the composition. In another specific embodiment, said isolated NK cell population comprises at least 80%, 85%, 90%, 95%, 98% or 99% of cells in the composition. In another specific embodiment, said NK cells comprise at least 70% of cells in the composition. In certain embodiments, at least 80%, 82%, 84%, 86%, 88% or 90% of NK cells in said composition are CD3⁻ and CD56⁺. In certain embodiments, at least 65%, 70%, 75%, 80%, 82%, 84%, 86%, 88% or 90% of NK cells in said composition are CD16−. In certain embodiments, at least 5%, 10%, 15%, 20%, 25%, 30%, 35%, 40%, 45%, 50%, 55% or 60% of NK cells in said composition are CD94+.

In another specific embodiment, said isolated NK cells in said composition are from a single individual, that is, said hemtopoietic stem and progenitor cells are from a single individual. In a more specific embodiment, said isolated NK cells comprise NK cells from at least two different individuals, that is, said hemtopoietic stem and progenitor cells are from at least two different individuals. In another specific embodiment, said isolated NK cells in said composition are from a different individual than the individual for whom treatment with the NK cells is intended, that is, said hemtopoietic stem and progenitor cells are from a different individual than the individual for whom treatment with the NK cells is intended. In another specific embodiment, said NK cells have been contacted or brought into proximity with an immunomodulatory compound or thalidomide in an amount and for a time sufficient for said NK cells to express detectably more granzyme B or perforin than an equivalent number of natural killer cells, i.e. NK cells not contacted or brought into proximity with said immunomodulatory compound or thalidomide. In another specific embodiment, said composition additionally comprises an immunomodulatory compound or thalidomide. In certain embodiments, the immunomodulatory compound is a compound described below.

In another specific embodiment, the composition additionally comprises one or more anticancer compounds, e.g., one or more of the anticancer compounds described below.

In a more specific embodiment, the composition comprises NK cells from another source, or made by another method. In a specific embodiment, said other source is placental blood and/or umbilical cord blood. In another specific embodiment, said other source is peripheral blood. In more specific embodiments, the NK cell population in said composition is combined with NK cells from another source, or made by another method in a ratio of about 100:1, 95:5, 90:10, 85:15, 80:20, 75:25, 70:30, 65:35, 60:40, 55:45: 50:50, 45:55, 40:60, 35:65, 30:70, 25:75, 20:80, 15:85, 10:90, 5:95, 100:1, 95:1, 90:1, 85:1, 80:1, 75:1, 70:1, 65:1, 60:1, 55:1, 50:1, 45:1, 40:1, 35:1, 30:1, 25:1, 20:1, 15:1, 10:1, 5:1, 1:1, 1:5, 1:10, 1:15, 1:20, 1:25, 1:30, 1:35, 1:40, 1:45, 1:50, 1:55, 1:60, 1:65, 1:70, 1:75, 1:80, 1:85, 1:90, 1:95, 1:100, or the like.

In another specific embodiment, the composition comprises an NK cell population and either isolated placental perfusate or isolated placental perfusate cells. In a more specific embodiment, said placental perfusate is from the same individual as said NK cell population. In another more specific embodiment, said placental perfusate comprises placental perfusate from a different individual than said NK cell population. In another specific embodiment, all, or substantially all (e.g., greater than 90%, 95%, 98% or 99%), of cells in said placental perfusate are fetal cells. In another specific embodiment, the placental perfusate or placental perfusate cells, comprise fetal and maternal cells. In a more specific embodiment, the fetal cells comprise less than about 90%, 80%, 70%, 60% or 50% of the cells in said placental perfusate. In another specific embodiment, said perfusate is obtained by passage of a 0.9% NaCl solution through the placental vasculature. In another specific embodiment, said perfusate comprises a culture medium. In another specific embodiment, said perfusate has been treated to remove erythrocytes. In another specific embodiment, said composition comprises an immunomodulatory compound, e.g., an immunomodulatory compound described below, e.g., an amino-substituted isoindoline compound. In another specific embodiment, the composition additionally comprises one or more anticancer compounds, e.g., one or more of the anticancer compounds described below.

In another specific embodiment, the composition comprises an NK cell population and placental perfusate cells. In a more specific embodiment, said placental perfusate cells are from the same individual as said NK cell population. In another more specific embodiment, said placental perfusate cells are from a different individual than said NK cell population. In another specific embodiment, the composition comprises isolated placental perfusate and isolated placental perfusate cells, wherein said isolated perfusate and said isolated placental perfusate cells are from different individuals. In another more specific embodiment of any of the above embodiments comprising placental perfusate, said placental perfusate comprises placental perfusate from at least two individuals. In another more specific embodiment of any of the above embodiments comprising placental perfusate cells, said isolated placental perfusate cells are from at least two individuals. In another specific embodiment, said composition comprises an immunomodulatory compound. In another specific embodiment, the composition additionally comprises one or more anticancer compounds, e.g., one or more of the anticancer compounds described below.

### 3.1. Terminology

As used herein, the terms “immunomodulatory compound” and “IMiD™” do not encompass thalidomide.

As used herein, “lenalidomide” means 3-(4′aminoisoindoline-1′-one)-1-piperidine-2,6-dione (Chemical Abstracts Service name) or 2,6-Piperidinedione,3-(4-amino-1,3-dihydro-1-oxo-2H-isoindol-2-yl)-(International Union of Pure and Applied Chemistry (IUPAC) name). As used herein, “pomalidomide” means 4-amino-2-(2,6-dioxopiperidin-3-yl)isoindole-1,3-dione.

As used herein, “multipotent,” when referring to a cell, means that the cell has the capacity to differentiate into a cell of another cell type. In certain embodiments, “a multipotent cell” is a cell that has the capacity to grow into a subset of the mammalian body's approximately 260 cell types. Unlike a pluripotent cell, a multipotent cell does not have the capacity to form all of the cell types.

As used herein, “feeder cells” refers to cells of one type that are co-cultured with cells of a second type, to provide an environment in which the cells of the second type can be maintained, and perhaps proliferate. Without being bound by any theory, feeder cells can provide, for example, peptides, polypeptides, electrical signals, organic molecules (e.g., steroids), nucleic acid molecules, growth factors (e.g., bFGF), other factors (e.g., cytokines), and metabolic nutrients to target cells. In certain embodiments, feeder cells grow in a monolayer.

As used herein, the “natural killer cells” or “NK cells” produced using the methods described herein, without further modification, include natural killer cells from any tissue source.

As used herein, the “ILC3 cells” produced using the methods described herein, without further modification, include ILC3 cells from any tissue source.

As used herein, “placental perfusate” means perfusion solution that has been passed through at least part of a placenta, e.g., a human placenta, e.g., through the placental vasculature, and includes a plurality of cells collected by the perfusion solution during passage through the placenta.

As used herein, “placental perfusate cells” means nucleated cells, e.g., total nucleated cells, isolated from, or isolatable from, placental perfusate.

As used herein, “tumor cell suppression,” “suppression of tumor cell proliferation,” and the like, includes slowing the growth of a population of tumor cells, e.g., by killing one or more of the tumor cells in said population of tumor cells, for example, by contacting or bringing, e.g., NK cells or an NK cell population produced using a three-stage method described herein into proximity with the population of tumor cells, e.g., contacting the population of tumor cells with NK cells or an NK cell population produced using a three-stage method described herein. In certain embodiments, said contacting takes place in vitro or ex vivo. In other embodiments, said contacting takes place in vivo.

As used herein, the term “hematopoietic cells” includes hematopoietic stem cells and hematopoietic progenitor cells.

As used herein, the “undefined component” is a term of art in the culture medium field that refers to components whose constituents are not generally provided or quantified. Examples of an “undefined component” include, without limitation, serum, for example, human serum (e.g., human serum AB) and fetal serum (e.g., fetal bovine serum or fetal calf serum).

As used herein, “+”, when used to indicate the presence of a particular cellular marker, means that the cellular marker is detectably present in fluorescence activated cell sorting over an isotype control; or is detectable above background in quantitative or semi-quantitative RT-PCR.

As used herein, “−” when used to indicate the presence of a particular cellular marker, means that the cellular marker is not detectably present in fluorescence activated cell sorting over an isotype control; or is not detectable above background in quantitative or semi-quantitative RT-PCR.

### 4. BRIEF DESCRIPTION OF THE FIGURES

FIG. 1 shows expansion of NK cells for compounds CRL1-CRL11.

FIG. 2 shows expansion of NK cells for compounds CRL12-CRL22.

FIG. 3 shows expansion of NK cells relative to SRI positive control.

FIG. 4 shows expansion of CD34+ cells from which the NK cells were derived.

FIG. 5 shows cytotoxicity of the expanded NK cultures.

FIGS. 6A-6C show that PNK cells highly express genes encoding the cytotoxic machinery. FIG. 6A CYNK cells were combined with peripheral blood derived NK cells (PB-NK) at 1:1 ratio and gene expression analyzed on single cell level using 10× Genomics Chromium platform and Illumina sequencing. Bioinformatics analysis utilized 10× Genomics Cell Ranger analysis pipeline. Transcript analysis was restricted to Granzyme B (GZMB) expressing cells. FIG. 6B A representative tSNE plot depicting PNK and PB-NK cells as distinct populations. FIG. 6C tSNE plots of selected NK cell-associated genes. The data is representative of two donors.

FIG. 7 shows that PNK and PB-NK cells differentially express genes encoding NK cell receptors. The expression of selected NK cell receptor genes analyzed by real-time quantitative PCR in peripheral blood NK cells (PB-NK) and CD11a+-bead-purified PNK cells. An alternative name indicated above the histogram for selected markers. The data represents mean±SD of three donors for CYNK and PBNK cells (n=3). * p<0.05, ** p<0.005, *** p<0.001.

FIG. 8 shows the gating strategy for PB-NK and CYNK cells. CYNK and PBMC cells were thawed and stained with fluorophore-coupled antibodies targeting NK cell receptors. The figure demonstrates representative dot plots and the gating strategy for the identification of CYNK and PB-NK cells. See FIG. 9 for further characterization of the populations.

FIG. 9 shows differential expression of surface proteins on CYNK and PB-NK cells. CYNK and PB-NK cells were pre-gated as indicated in FIG. 8.

FIG. 10 shows that CYNK cells form a distinct cell population from PB-NK cells based on surface protein expression. tSNE plots demonstrating differential clustering of CYNK and PB-NK cells based on their surface markers. tSNE plots were generated of flow cytometry data using FlowJo software.

FIG. 11 shows direct and indirect antiviral mechanisms of NK cell action.

FIGS. 12A-12B show expression of NK cell activating receptors on CYNK-001 cells. FIG. 12A shows representative dot plots demonstrating the gating strategy for the analysis of CYNK-001 cells. Thawed CYNK-001 cells were stained with fluorophore-conjugated antibodies recognizing indicated NK cell markers and analyzed by flow cytometry. CYNK-001 are defined as live CD3− CD14−CD19−CD56+ cells. FIG. 12B shows representative histograms of the expression of indicated NK activating cell receptors on CYNK-001 cells. FMO—fluorescence minus one control.

FIG. 13 shows expression of selected genes in CYNK-001 cells. scRNAseq data representing median-normalized average counts per cell of each indicated gene on PNK-007 cells. KLRK1 encodes NKG2D, CD226 encodes DNAM-1, NCR1 encodes NKp46, NCR2 encodes NKp44, NCR3 encodes NKp30. Data compiled of 2 donors. Mean+/−SD

FIG. 14 shows Inflammation Marker Analysis (ferritin, D-Dimer, C-reactive protein, and IL-6) of First Three Patients Enrolled in the clinical study.

FIG. 15 shows in vitro antiviral cytolytic activity of CYNK-001.

FIG. 16 shows in vitro antiviral cytolytic activity of CYNK-001.

FIG. 17 shows in vitro antiviral cytolytic activity of CYNK-001.

FIG. 18 shows in vitro antiviral cytolytic activity of CYNK-001.

FIG. 19 shows antiviral activity of CYNK-001 against IAV-induced severe infection in mice.

FIG. 20 shows antiviral activity of CYNK-001 against IAV-induced severe infection in mice.

FIG. 21 shows that CYNK-001 reduced proinflammatory cytofines and chemokines in BALF.

FIG. 22 shows that CYNK-001 altered immune cell profiles in BALF as measured by FACS.

FIG. 23 shows that CYNK-001 altered immune cell profiles in BALF as measured by FACS.

FIG. 24 shows that CYNK-001 altered immune cell profiles in lung by immunohistochemistry.

FIG. 25 shows that CYNK-001 altered immune cell profiles in lung by immunohistochemistry.

FIG. 26 shows expression of selected genes in CYNK-001 cells. scRNAseq data representing median-normalized average counts per cell of each indicated gene on CYNK-001 cells. KLRK1 encodes NKG2D, CD226 encodes DNAM-1, NCR1 encodes NKp46, NCR2 encodes NKp44 and NCR3 encodes NKp30. Data compiled of 2 donors. Mean+/−SD.

FIG. 27 shows that CYNK-001 cell degranulation upon contact with influenza virus-infected A549 cells. A549 cells were infected with Influenza A virus strain A/PR8/8/34 (PR8) using the indicated MOI and CYNK-001 were added 24 h post infection. After 5 h incubation, CYNK-001 cells were collected and CD107a expression was analyzed by flow cytometry. CD107a was analyzed on live single cells negative for lineage markers (CD3, CD14, CD19) and expressing CD56. Paired t test was used to analyze the CD107a expression on CYNK-001 cells upon contact co-culture with infected cells (MOI 0.1 or 1) comparing to non-infected cells (MOI 0). * P<0.05, ** P<0.01. CYNK-001 cells from 4 donors analyzed.

FIGS. 28A-28B show cytolysis of CYNK-001 cells against virus-infected A549 cells. A549 cells were infected with Influenza A virus strain A/PR8/8/34 (PR8) using the indicated MOI and CYNK-001 were added 24 h post infection. Cytolysis of virus-infected A549 cell was monitored using a real-time impedance-based assay over 24 h. FIG. 28A A representative cytolysis curve over 24 h of one CYNK-001 donor. Mean+/−SD of 3 technical replicates. FIG. 28B Specific cytolysis was calculated by subtracting CYNK-001 cell cytolysis on non-infected cells (MOI 0) from cytolysis values on infected cells. Paired t test was used to analyze cytolysis of infected cells (MOI 0.1, 1 and 3) comparing to non-infected cells (MOI 0). * P<0.05, ** P<0.01. CYNK-001 cells from 4 donors analyzed.

FIG. 29 shows CYNK-001 cell production of pro-inflammatory cytokines upon contact with influenza virus-infected A549 cells. A549 cells were infected with Influenza A virus strain A/PR8/8/34 (PR8) using the indicated MOI and CYNK-001 were added 24 h post infection. After 5 h incubation, CYNK-001 cells were collected for intracellular cytokine staining and analysis using flow cytometry. IFN-γ and TNF-α expression was analyzed on live single cells negative for lineage markers (CD3, CD14, CD19) and expressing CD56. Paired t test was used to analyze cytokine expression on CYNK-001 cells upon contact co-culture with infected cells (MOI 0.1 or 1) comparing to non-infected cells (MOI 0). * P<0.05. CYNK-001 cells from 3-4 donors analyzed.

FIG. 30 shows CYNK-001 cell secretion of pro-inflammatory cytokines upon contact with influenza virus-infected A549 cells. A549 cells were infected with Influenza A virus strain A/PR8/8/34 (PR8) using the indicated MOI and CYNK-001 were added 24 h post infection. Cell culture supernatant was collected 24 h after addition of CYNK-001 cells. Cytokines in the supernatant were analyzed using Multiplex assay. * P<0.05, ** P<0.01. Technical triplicates were analyzed. Data represent mean+/−standard deviation.

FIG. 31 shows SARS-CoV-2 virus infection induces NK cell activating ligand expression in Calu-3 cells. Calu-3 were infected with SARS-CoV-2 at a multiplicity of infection (MOI) of 1 or 5 and cells were collected for antibody staining and analysis using flow cytometry 48 h post infection. First gated on single live cells. NK cell ligands were stained using recombinant Fc-coupled NK cell receptors (NKp46-Fc, NKp44-Fc and NKG2D-Fc) or antibodies recognizing NKG2D ligands (MICA/B, ULBP-1, ULBP-3 and ULBP2/5/6). Valproic acid (VPA) at 5 mM was used as a positive control for NKG2D ligand induction. FMO—fluorescence minus one. Mean+/−standard deviation.

### 5. DETAILED DESCRIPTION

The present invention provides methods of treating a viral infection in a subject, comprising administering to the subject an amount of a composition comprising a plurality of placenta derived natural killer cells, effective to treat the viral infection in the subject.

In some embodiments, said administration is intravenous. In other embodiments, said administration is by bronchiolar lavage or whole lung lavage.

In some embodiments, said natural killer cells have been cryopreserved prior to said administering.

In some embodiments, said subject is administered about 1×10⁴, 3×10⁴, 1×10⁵, 3×10⁵, 1×10⁶, 3×10⁶, 1×10⁷, 3×10⁷, 1×10⁸, or 3×10⁸ natural killer cells per kilogram of the subject.

In some embodiments, the treatment comprises administration of more than one dose of the cell population comprising human placenta-derived natural killer cells. In some embodiments, the treatment comprises administration of two, three, four, or more doses of the cell population comprising human placenta-derived natural killer cells.

In some embodiments, the subject is a mammal. In preferred embodiments, the subject is a human.

In some embodiments, the treating further comprises administering to the subject an effective amount of an additional anti-viral treatment.

In some embodiments, said composition comprises a population of cells that comprise at least 20% CD56+CD3− natural killer cells. In preferred embodiments, said composition comprises a population of cells that comprise at least 40% CD56+CD3− natural killer cells. In more preferred embodiments, said composition comprises a population of cells that comprise at least 60% CD56+CD3− natural killer cells. In yet more pe=referred embodiments, said composition comprises a population of cells that comprise at least 80% CD56+CD3− natural killer cells.

In some embodiments, said placenta derived natural killer cells are human placenta derived natural killer cells. In some embodiments, said placenta derived natural killer cells are hematopoietic stem cell-derived natural killer cells. In preferred embodiments, said placenta derived natural killer cells are CD34+ hematopoietic stem cell-derived natural killer cells.

In some embodiments, said placenta derived natural killer cells are characterized by expression of one or more markers selected from the group consisting of FGFBP2, GZMH, CCL3L3, GZMM, CXCR4, ZEB2, KLF2, LITAF, RORA, LYAR, CNOT1, IFNG, DUSP2, ATG2A, CD7, PMAIP1, PPP2R5C, NR4A2, ZFP36L2, PIK3R1, KLRF1, SNHG9, MT2A, RGS2, CHD1, DUSP1, EML4, ZFP36, ZC3H12A, DNAJB6, SBDS, IRF1, TSC22D3, TSPYL2, PNRC1, ISCA1, JUNB, WHAMM, RICTOR, TNFAIP3, EPC1, MVD, CLK1, ARL4C, REL, KMT2E, YPEL5, AMD1, BTG2, and IDS which is lower than expression of said markers in peripheral blood natural killer cells and/or expression of one or more markers selected from the group consisting of NDFIP2, LINC00996, MAL, CCL1, MB, SPINK2, C15orf48, CAMK1, KLRC1, TNFSF10, TNFRSF18, IL32, CAPG, AC092580.4, S100A11, TNFRSF4, ENO1, FCER1G, CCND2, KRT81, MRPS6, ANXA2, PTGER2, GLO1, HAVCR2, PYCARD, LAT2, SLC16A3, COTL1, PKM, TALDO1, CD96, NCR3, KRT86, STMN1, LTB, ARPC1B, ARPC5, FKBP1A, TIMP1, GZMK, CD59, PGK1, RGS10, EVL, RAC2, LGALS1, ITGB7, TUBB, PGAM1, PRF1, GZMB, IL2RB, KLRC2, and KLRB1 which is higher than expression of said markers in peripheral blood natural killer cells.

In some embodiments, said placenta derived natural killer cells are characterized by expression of one or more markers selected from the group consisting of FGFBP2, GZMH, CCL3L3, GZMM, CXCR4, ZEB2, KLF2, LITAF, RORA, LYAR, CNOT1, IFNG, DUSP2, ATG2A, CD7, PMAIP1, PPP2R5C, NR4A2, ZFP36L2, PIK3R1, KLRF1, SNHG9, MT2A, RGS2, CHD1, DUSP1, EML4, ZFP36, ZC3H12A, DNAJB6, SBDS, IRF1, TSC22D3, TSPYL2, PNRC1, ISCA1, JUNB, WHAMM, RICTOR, TNFAIP3, EPC1, MVD, CLK1, ARL4C, REL, KMT2E, YPEL5, AMD1, BTG2, and IDS which is lower than expression of said markers in peripheral blood natural killer cells. In preferred embodiments, said placenta derived natural killer cells are characterized by expression of 2, 3, 4, 5, 6, 7, 8, 9, 10, or more markers selected from the group consisting of FGFBP2, GZMH, CCL3L3, GZMM, CXCR4, ZEB2, KLF2, LITAF, RORA, LYAR, CNOT1, IFNG, DUSP2, ATG2A, CD7, PMAIP1, PPP2R5C, NR4A2, ZFP36L2, PIK3R1, KLRF1, SNHG9, MT2A, RGS2, CHD1, DUSP1, EML4, ZFP36, ZC3H12A, DNAJB6, SBDS, IRF1, TSC22D3, TSPYL2, PNRC1, ISCA1, JUNB, WHAMM, RICTOR, TNFAIP3, EPC1, MVD, CLK1, ARL4C, REL, KMT2E, YPEL5, AMD1, BTG2, and IDS is lower than expression of said markers in peripheral blood natural killer cells.

In some embodiments, said placenta derived natural killer cells are characterized by expression of one or more markers selected from the group consisting of NDFIP2, LINC00996, MAL, CCL1, MB, SPINK2, C15orf48, CAMK1, KLRC1, TNFSF10, TNFRSF18, IL32, CAPG, AC092580.4, S100A11, TNFRSF4, ENO1, FCER1G, CCND2, KRT81, MRPS6, ANXA2, PTGER2, GLO1, HAVCR2, PYCARD, LAT2, SLC16A3, COTL1, PKM, TALDO1, CD96, NCR3, KRT86, STMN1, LTB, ARPC1B, ARPC5, FKBP1A, TIMP1, GZMK, CD59, PGK1, RGS10, EVL, RAC2, LGALS1, ITGB7, TUBB, PGAM1, PRF1, GZMB, IL2RB, KLRC2, and KLRB1 which is higher than expression of said markers in peripheral blood natural killer cells. In preferred embodiments, said placenta derived natural killer cells are characterized by expression of 2, 3, 4, 5, 6, 7, 8, 9, 10, or more markers selected from the group consisting of NDFIP2, LINC00996, MAL, CCL1, MB, SPINK2, C15orf48, CAMK1, KLRC1, TNFSF10, TNFRSF18, IL32, CAPG, AC092580.4, S100A11, TNFRSF4, ENO1, FCER1G, CCND2, KRT81, MRPS6, ANXA2, PTGER2, GLO1, HAVCR2, PYCARD, LAT2, SLC16A3, COTL1, PKM, TALDO1, CD96, NCR3, KRT86, STMN1, LTB, ARPC1B, ARPC5, FKBP1A, TIMP1, GZMK, CD59, PGK1, RGS10, EVL, RAC2, LGALS1, ITGB7, TUBB, PGAM1, PRF1, GZMB, IL2RB, KLRC2, and KLRB1 is higher than expression of said markers in peripheral blood natural killer cells.

In some embodiments, said human placenta derived natural killer cells are CYNK cells.

In some embodiments, said viral infection is a coronavirus infection. In preferred embodiments, said coronavirus infection is selected from the group consisting of human coronavirus 229E (HCoV-229E), human coronavirus OC43 (HCoV-OC43), SARS-CoV, human coronavirus NL63 (HCoV-NL63, New Haven coronavirus), human coronavirus HKU1, middle east respiratory syndrome coronavirus (MERS-CoV, novel coronavirus 2012, HCoV-EMC), and novel coronavirus 2019-nCoV (Wuhan pneumonia, Wuhan coronavirus). In more preferred embodiments, said coronavirus infection is novel coronavirus 2019-nCoV (Wuhan pneumonia, Wuhan coronavirus, SARS-CoV-2).

The present invention also provides natural killer cells characterized by expression of one or more markers selected from the group consisting of FGFBP2, GZMH, CCL3L3, GZMM, CXCR4, ZEB2, KLF2, LITAF, RORA, LYAR, CNOT1, IFNG, DUSP2, ATG2A, CD7, PMAIP1, PPP2R5C, NR4A2, ZFP36L2, PIK3R1, KLRF1, SNHG9, MT2A, RGS2, CHD1, DUSP1, EML4, ZFP36, ZC3H12A, DNAJB6, SBDS, IRF1, TSC22D3, TSPYL2, PNRC1, ISCA1, JUNB, WHAMM, RICTOR, TNFAIP3, EPC1, MVD, CLK1, ARL4C, REL, KMT2E, YPEL5, AMD1, BTG2, and IDS which is lower than expression of said markers in peripheral blood natural killer cells and/or expression of one or more markers selected from the group consisting of NDFIP2, LINC00996, MAL, CCL1, MB, SPINK2, C15orf48, CAMK1, KLRC1, TNFSF10, TNFRSF18, IL32, CAPG, AC092580.4, S100A11, TNFRSF4, ENO1, FCER1G, CCND2, KRT81, MRPS6, ANXA2, PTGER2, GLO1, HAVCR2, PYCARD, LAT2, SLC16A3, COTL1, PKM, TALDO1, CD96, NCR3, KRT86, STMN1, LTB, ARPC1B, ARPC5, FKBP1A, TEMPI, GZMK, CD59, PGK1, RGS10, EVL, RAC2, LGALS1, ITGB7, TUBB, PGAM1, PRF1, GZMB, IL2RB, KLRC2, and KLRB1 which is higher than expression of said markers in peripheral blood natural killer cells for use in treating a viral infection.

In some embodiments, the natural killer cells are characterized by expression of one or more markers selected from the group consisting of FGFBP2, GZMH, CCL3L3, GZMM, CXCR4, ZEB2, KLF2, LITAF, RORA, LYAR, CNOT1, IFNG, DUSP2, ATG2A, CD7, PMAIP1, PPP2R5C, NR4A2, ZFP36L2, PIK3R1, KLRF1, SNHG9, MT2A, RGS2, CHD1, DUSP1, EML4, ZFP36, ZC3H12A, DNAJB6, SBDS, IRF1, TSC22D3, TSPYL2, PNRC1, ISCA1, JUNB, WHAMM, RICTOR, TNFAIP3, EPC1, MVD, CLK1, ARL4C, REL, KMT2E, YPEL5, AMD1, BTG2, and IDS which is lower than expression of said markers in peripheral blood natural killer cells.

In some embodiments, the natural killer cells are characterized by expression of 2, 3, 4, 5, 6, 7, 8, 9, 10, or more markers selected from the group consisting of FGFBP2, GZMH, CCL3L3, GZMM, CXCR4, ZEB2, KLF2, LITAF, RORA, LYAR, CNOT1, IFNG, DUSP2, ATG2A, CD7, PMAIP1, PPP2R5C, NR4A2, ZFP36L2, PIK3R1, KLRF1, SNHG9, MT2A, RGS2, CHD1, DUSP1, EML4, ZFP36, ZC3H12A, DNAJB6, SBDS, IRF1, TSC22D3, TSPYL2, PNRC1, ISCA1, JUNB, WHAMM, RICTOR, TNFAIP3, EPC1, MVD, CLK1, ARL4C, REL, KMT2E, YPEL5, AMD1, BTG2, and IDS which is lower than expression of said markers in peripheral blood natural killer cells.

In some embodiments, the natural killer cells are characterized by expression of one or more markers selected from the group consisting of NDFIP2, LINC00996, MAL, CCL1, MB, SPINK2, C15orf48, CAMK1, KLRC1, TNFSF10, TNFRSF18, IL32, CAPG, AC092580.4, S100A11, TNFRSF4, ENO1, FCER1G, CCND2, KRT81, MRPS6, ANXA2, PTGER2, GLO1, HAVCR2, PYCARD, LAT2, SLC16A3, COTL1, PKM, TALDO1, CD96, NCR3, KRT86, STMN1, LTB, ARPC1B, ARPC5, FKBP1A, TIMP1, GZMK, CD59, PGK1, RGS10, EVL, RAC2, LGALS1, ITGB7, TUBB, PGAM1, PRF1, GZMB, IL2RB, KLRC2, and KLRB1 which is higher than expression of said markers in peripheral blood natural killer cells.

In some embodiments, the natural killer cells are characterized by expression of 2, 3, 4, 5, 6, 7, 8, 9, 10, or more markers selected from the group consisting of NDFIP2, LINC00996, MAL, CCL1, MB, SPINK2, C15orf48, CAMK1, KLRC1, TNFSF10, TNFRSF18, IL32, CAPG, AC092580.4, S100A11, TNFRSF4, ENO1, FCER1G, CCND2, KRT81, MRPS6, ANXA2, PTGER2, GLO1, HAVCR2, PYCARD, LAT2, SLC16A3, COTL1, PKM, TALDO1, CD96, NCR3, KRT86, STMN1, LTB, ARPC1B, ARPC5, FKBP1A, TIMP1, GZMK, CD59, PGK1, RGS10, EVL, RAC2, LGALS1, ITGB7, TUBB, PGAM1, PRF1, GZMB, IL2RB, KLRC2, and KLRB1 which is higher than expression of said markers in peripheral blood natural killer cells.

In some embodiments, said viral infection is a coronavirus infection. In preferred embodiments, said coronavirus infection is selected from the group consisting of human coronavirus 229E (HCoV-229E), human coronavirus OC43 (HCoV-OC43), SARS-CoV, human coronavirus NL63 (HCoV-NL63, New Haven coronavirus), human coronavirus HKU1, middle east respiratory syndrome coronavirus (MERS-CoV, novel coronavirus 2012, HCoV-EMC), and novel coronavirus 2019-nCoV (Wuhan pneumonia, Wuhan coronavirus). In more preferred embodiments, said coronavirus infection is novel coronavirus 2019-nCoV (Wuhan pneumonia, Wuhan coronavirus, SARS-CoV-2).

In some embodiments, the treatment comprises an improvement in score as measured by the Ordinal Scale for Clinical Improvement (OSCI). In some embodiments, the treatment comprises a reduction in the time to improvement in score as measured by the Ordinal Scale for Clinical Improvement (OSCI). In some embodiments, the treatment comprises an improvement in stratus by OSCI. In some embodiments, the treatment comprises an improvement in time to and/or rate of clinical improvement by NEWS2 Score. In some embodiments, the treatment comprises medical discharge or a reduced time to medical discharge. In some embodiments, the treatment comprises reduced hospital utilization. In some embodiments, the treatment comprises reduced mortality.

In some embodiments, the treatment comprises clearance of the virus or reduced time to clearance of the virus. In some embodiments, the treatment comprises improved time to and/or rate of pulmonary clearance. In some embodiments, the treatment comprises reduced duration of hospitalization. In some embodiments, the treatment comprises an increase in supplemental oxygen-free days, a reduced need for supplemental oxygen, or a reduced time to cessation of supplemental oxygen. In some embodiments, the treatment comprises a reduction in the requirement for ventilation. In some embodiments, the treatment comprises an improvement in SOFA score. In some embodiments, the treatment comprises an improvement in radiologic evaluation score. In some embodiments, the treatment comprises an improvement in cytokine and/or chemokine assessment, preferably wherein the improvement in cytokine and/or chemokine assessment comprises a reduction in one or more inflammatory markers. In some embodiments, the treatment comprises reduced or eliminated viral detection by RT-PCR.

In some embodiments, the treatment comprises two or more doses of natural killer cells. In some embodiments, the treatment comprises a first dose and one or more subsequent doses of natural killer cells. In some embodiments, the treatment comprises a first dose of between about 50×10⁶ natural killer cells to about and about 600×10⁶ natural killer cells. In some embodiments, the treatment comprises a first dose and one or more subsequent doses of natural killer cells, wherein the one or more subsequent doses comprise about 150×10⁶ natural killer cells to about and about 2400×10⁶ natural killer cells. In some embodiments, the treatment comprises a first dose and one or more subsequent doses of natural killer cells, wherein the one or more subsequent doses of natural killer cells are administered from about one to about five days after the previous doses, preferably wherein the one or more subsequent doses of natural killer cells are administered about three days after the previous dose.

In some embodiments, the treatment comprises an initial dose of 150×10⁶ cells on Day 1 followed by 600×10⁶ cells IV Days 4 and 7 or an initial dose of 150×10⁶ cells on Day 1 followed by 600×10⁶ cells IV Day 7.

Also provided herein are novel methods of producing and expanding NK cells and/or ILC3 cells from hematopoietic cells, e.g., hematopoietic stem cells or progenitor cells. Also provided herein are methods, e.g., three-stage methods, of producing NK cell populations and/or ILC3 cell populations from hematopoietic cells, e.g., hematopoietic stem cells or progenitor cells. The hematopoietic cells (e.g., CD34+ hematopoietic stem cells) used to produce the NK cells and/or ILC3 cells, and NK cell populations and/or ILC3 cell populations, may be obtained from any source, for example, without limitation, placenta, umbilical cord blood, placental blood, peripheral blood, spleen or liver. In certain embodiments, the NK cells and/or ILC3 cells or NK cell populations and/or ILC3 cell populations are produced from expanded hematopoietic cells, e.g., hematopoietic stem cells and/or hematopoietic progenitor cells. In one embodiment, hematopoietic cells are collected from a source of such cells, e.g., placenta, for example from placental perfusate, umbilical cord blood, placental blood, peripheral blood, spleen, liver (e.g., fetal liver) and/or bone marrow.

The hematopoietic cells used to produce the NK cells and/or ILC3 cells, and NK cell populations and/or ILC3 cell populations, may be obtained from any animal species. In certain embodiments, the hematopoietic stem or progenitor cells are mammalian cells. In specific embodiments, said hematopoietic stem or progenitor cells are human cells. In specific embodiments, said hematopoietic stem or progenitor cells are primate cells. In specific embodiments, said hematopoietic stem or progenitor cells are canine cells. In specific embodiments, said hematopoietic stem or progenitor cells are rodent cells.

5.1. Hematopoietic Cells

Hematopoietic cells useful in the methods disclosed herein can be any hematopoietic cells able to differentiate into NK cells and/or ILC3 cells, e.g., precursor cells, hematopoietic progenitor cells, hematopoietic stem cells, or the like. Hematopoietic cells can be obtained from tissue sources such as, e.g., bone marrow, cord blood, placental blood, peripheral blood, liver or the like, or combinations thereof. Hematopoietic cells can be obtained from placenta. In a specific embodiment, the hematopoietic cells are obtained from placental perfusate. In one embodiment, the hematopoietic cells are not obtained from umbilical cord blood. In one embodiment, the hematopoietic cells are not obtained from peripheral blood. Hematopoietic cells from placental perfusate can comprise a mixture of fetal and maternal hematopoietic cells, e.g., a mixture in which maternal cells comprise greater than 5% of the total number of hematopoietic cells. In certain embodiments, hematopoietic cells from placental perfusate comprise at least about 90%, 95%, 98%, 99% or 99.5% fetal cells.

In another specific embodiment, the hematopoietic cells, e.g., hematopoietic stem cells or progenitor cells, from which the NK cell populations and/or ILC3 cell populations produced using a three-stage method described herein are produced, are obtained from placental perfusate, umbilical cord blood, fetal liver, mobilized peripheral blood, or bone marrow. In another specific embodiment, the hematopoietic cells, e.g., hematopoietic stem cells or progenitor cells, from which the NK cell populations and/or ILC3 cell populations produced using a three-stage method described herein are produced, are combined cells from placental perfusate and cord blood, e.g., cord blood from the same placenta as the perfusate. In another specific embodiment, said umbilical cord blood is isolated from a placenta other than the placenta from which said placental perfusate is obtained. In certain embodiments, the combined cells can be obtained by pooling or combining the cord blood and placental perfusate. In certain embodiments, the cord blood and placental perfusate are combined at a ratio of 100:1, 95:5, 90:10, 85:15, 80:20, 75:25, 70:30, 65:35, 60:40, 55:45: 50:50, 45:55, 40:60, 35:65, 30:70, 25:75, 20:80, 15:85, 10:90, 5:95, 100:1, 95:1, 90:1, 85:1, 80:1, 75:1, 70:1, 65:1, 60:1, 55:1, 50:1, 45:1, 40:1, 35:1, 30:1, 25:1, 20:1, 15:1, 10:1, 5:1, 1:1, 1:5, 1:10, 1:15, 1:20, 1:25, 1:30, 1:35, 1:40, 1:45, 1:50, 1:55, 1:60, 1:65, 1:70, 1:75, 1:80, 1:85, 1:90, 1:95, 1:100, or the like by volume to obtain the combined cells. In a specific embodiment, the cord blood and placental perfusate are combined at a ratio of from 10:1 to 1:10, from 5:1 to 1:5, or from 3:1 to 1:3. In another specific embodiment, the cord blood and placental perfusate are combined at a ratio of 10:1, 5:1, 3:1, 1:1, 1:3, 1:5 or 1:10. In a more specific embodiment, the cord blood and placental perfusate are combined at a ratio of 8.5:1.5 (85%: 15%).

In certain embodiments, the cord blood and placental perfusate are combined at a ratio of 100:1, 95:5, 90:10, 85:15, 80:20, 75:25, 70:30, 65:35, 60:40, 55:45: 50:50, 45:55, 40:60, 35:65, 30:70, 25:75, 20:80, 15:85, 10:90, 5:95, 100:1, 95:1, 90:1, 85:1, 80:1, 75:1, 70:1, 65:1, 60:1, 55:1, 50:1, 45:1, 40:1, 35:1, 30:1, 25:1, 20:1, 15:1, 10:1, 5:1, 1:1, 1:5, 1:10, 1:15, 1:20, 1:25, 1:30, 1:35, 1:40, 1:45, 1:50, 1:55, 1:60, 1:65, 1:70, 1:75, 1:80, 1:85, 1:90, 1:95, 1:100, or the like by total nucleated cells (TNC) content to obtain the combined cells. In a specific embodiment, the cord blood and placental perfusate are combined at a ratio of from 10:1 to 10:1, from 5:1 to 1:5, or from 3:1 to 1:3. In another specific embodiment, the cord blood and placental perfusate are combined at a ratio of 10:1, 5:1, 3:1, 1:1, 1:3, 1:5 or 1:10.

In another specific embodiment, the hematopoietic cells, e.g., hematopoietic stem cells or progenitor cells from which said NK cell populations and/or ILC3 cell populations produced using a three-stage method described herein are produced, are from both umbilical cord blood and placental perfusate, but wherein said umbilical cord blood is isolated from a placenta other than the placenta from which said placental perfusate is obtained.

In certain embodiments, the hematopoietic cells are CD34⁺ cells. In specific embodiments, the hematopoietic cells useful in the methods disclosed herein are CD34⁺CD38⁺ or CD34⁺CD38⁻. In a more specific embodiment, the hematopoietic cells are CD34⁺CD38⁻ Lin⁻. In another specific embodiment, the hematopoietic cells are one or more of CD2⁻, CD3⁻, CD11b⁻, CD11c⁻, CD14⁻, CD16⁻, CD19⁻, CD24⁻, CD56⁻, CD66b⁻ and/or glycophorin A⁻. In another specific embodiment, the hematopoietic cells are CD2⁻, CD3⁻, CD11b⁻, CD11c⁻, CD14⁻, CD16⁻, CD19⁻, CD24⁻, CD56⁻, CD66b⁻ and glycophorin A⁻. In another more specific embodiment, the hematopoietic cells are CD34⁺CD38⁻CD33⁻CD117⁻. In another more specific embodiment, the hematopoietic cells are CD34⁺CD38⁻CD33⁻CD117⁻CD235⁻CD36⁻.

In another embodiment, the hematopoietic cells are CD45⁺. In another specific embodiment, the hematopoietic cells are CD34⁺CD45⁺. In another embodiment, the hematopoietic cell is Thy-1⁺. In a specific embodiment, the hematopoietic cell is CD34⁺ Thy-1⁺. In another embodiment, the hematopoietic cells are CD133⁺. In specific embodiments, the hematopoietic cells are CD34⁺CD133⁺ or CD133⁺ Thy-1⁺. In another specific embodiment, the CD34⁺ hematopoietic cells are CXCR4⁺. In another specific embodiment, the CD34⁺ hematopoietic cells are CXCR4⁻. In another embodiment, the hematopoietic cells are positive for KDR (vascular growth factor receptor 2). In specific embodiments, the hematopoietic cells are CD34⁺KDR⁺, CD133⁺KDR⁺ or Thy-1⁺KDR⁺. In certain other embodiments, the hematopoietic cells are positive for aldehyde dehydrogenase (ALDH⁺), e.g., the cells are CD34⁺ALDH⁺.

In certain other embodiments, the CD34⁺ cells are CD45⁻. In specific embodiments, the CD34⁺ cells, e.g., CD34⁺, CD45⁻ cells express one or more, or all, of the miRNAs hsa-miR-380, hsa-miR-512, hsa-miR-517, hsa-miR-518c, hsa-miR-519b, hsa-miR-520a, hsa-miR-337, hsa-miR-422a, hsa-miR-549, and/or hsa-miR-618.

In certain embodiments, the hematopoietic cells are CD34⁻.

The hematopoietic cells can also lack certain markers that indicate lineage commitment, or a lack of developmental naiveté. For example, in another embodiment, the hematopoietic cells are HLA-DR⁻. In specific embodiments, the hematopoietic cells are CD34⁺HLA-DR⁻, CD133⁺HLA-DR⁻, Thy-1⁺HLA-DR⁻ or ALDH⁺HLA-DR⁻ In another embodiment, the hematopoietic cells are negative for one or more, or all, of lineage markers CD2, CD3, CD11b, CD11c, CD14, CD16, CD19, CD24, CD56, CD66b and glycophorin A.

Thus, hematopoietic cells can be selected for use in the methods disclosed herein on the basis of the presence of markers that indicate an undifferentiated state, or on the basis of the absence of lineage markers indicating that at least some lineage differentiation has taken place. Methods of isolating cells, including hematopoietic cells, on the basis of the presence or absence of specific markers is discussed in detail below.

Hematopoietic cells used in the methods provided herein can be a substantially homogeneous population, e.g., a population comprising at least about 95%, at least about 98% or at least about 99% hematopoietic cells from a single tissue source, or a population comprising hematopoietic cells exhibiting the same hematopoietic cell-associated cellular markers. For example, in various embodiments, the hematopoietic cells can comprise at least about 95%, 98% or 99% hematopoietic cells from bone marrow, cord blood, placental blood, peripheral blood, or placenta, e.g., placenta perfusate.

Hematopoietic cells used in the methods provided herein can be obtained from a single individual, e.g., from a single placenta, or from a plurality of individuals, e.g., can be pooled. Where the hematopoietic cells are obtained from a plurality of individuals and pooled, the hematopoietic cells may be obtained from the same tissue source. Thus, in various embodiments, the pooled hematopoietic cells are all from placenta, e.g., placental perfusate, all from placental blood, all from umbilical cord blood, all from peripheral blood, and the like.

Hematopoietic cells used in the methods disclosed herein can, in certain embodiments, comprise hematopoietic cells from two or more tissue sources. For example, in certain embodiments, when hematopoietic cells from two or more sources are combined for use in the methods herein, a plurality of the hematopoietic cells used to produce natural killer cells using a three-stage method described herein comprise hematopoietic cells from placenta, e.g., placenta perfusate. In various embodiments, the hematopoietic cells used to produce NK cell populations and/or ILC3 cell populations produced using a three-stage method described herein, comprise hematopoietic cells from placenta and from cord blood; from placenta and peripheral blood; from placenta and placental blood, or placenta and bone marrow. In one embodiment, the hematopoietic cells comprise hematopoietic cells from placental perfusate in combination with hematopoietic cells from cord blood, wherein the cord blood and placenta are from the same individual, i.e., wherein the perfusate and cord blood are matched. In embodiments in which the hematopoietic cells comprise hematopoietic cells from two tissue sources, the hematopoietic cells from the sources can be combined in a ratio of, for example, 1:10, 2:9, 3:8, 4:7:, 5:6, 6:5, 7:4, 8:3, 9:2, 1:10, 1:9, 1:8, 1:7, 1:6, 1:5, 1:4, 1:3, 1:2, 1:1, 2:1, 3:1, 4:1, 5:1, 6:1, 7:1, 8:1 or 9:1.

5.1.1. Placental Hematopoietic Stem Cells

In certain embodiments, the hematopoietic cells used in the methods provided herein are placental hematopoietic cells. In one embodiment, placental hematopoietic cells are CD34⁺. In a specific embodiment, the placental hematopoietic cells are predominantly (e.g., at least about 50%, 55%, 60%, 65%, 70%, 75%, 80%, 85%, 90%, 95% or 98%) CD34⁺CD38⁻ cells. In another specific embodiment, the placental hematopoietic cells are predominantly (e.g., at least about 50%, 55%, 60%, 65%, 70%, 75%, 80%, 85%, 90%, 95% or 98%) CD34⁺CD38⁺ cells. Placental hematopoietic cells can be obtained from a post-partum mammalian (e.g., human) placenta by any means known to those of skill in the art, e.g., by perfusion.

In another embodiment, the placental hematopoietic cell is CD45⁻. In a specific embodiment, the hematopoietic cell is CD34⁺CD45⁻. In another specific embodiment, the placental hematopoietic cells are CD34⁺CD45⁺.

5.2. Production of Natural Killer and/or ILC3 Cells and Natural Killer Cell and/or ILC3 Cell Populations

Production of NK cells and/or ILC3 cells and NK cell and/or ILC3 cell populations by the present methods comprises expanding a population of hematopoietic cells. During cell expansion, a plurality of hematopoietic cells within the hematopoietic cell population differentiate into NK cells and/or ILC3 cells. In one aspect, provided herein is a method of producing NK cells comprising culturing hematopoietic stem cells or progenitor cells, e.g., CD34⁺ stem cells or progenitor cells, in a first medium comprising a stem cell mobilizing agent and thrombopoietin (Tpo) to produce a first population of cells, subsequently culturing said first population of cells in a second medium comprising a stem cell mobilizing agent and interleukin-15 (IL-15), and lacking Tpo, to produce a second population of cells, and subsequently culturing said second population of cells in a third medium comprising IL-2 and IL-15, and lacking a stem cell mobilizing agent and LMWH, to produce a third population of cells, wherein the third population of cells comprises natural killer cells that are CD56+, CD3−, and wherein at least 70%, for example at least 80%, of the natural killer cells are viable. In certain embodiments, such natural killer cells comprise natural killer cells that are CD16−. In certain embodiments, such natural killer cells comprise natural killer cells that are CD94+. In certain embodiments, such natural killer cells comprise natural killer cells that are CD94+ or CD16+. In certain embodiments, such natural killer cells comprise natural killer cells that are CD94− or CD16−. In certain embodiments, such natural killer cells comprise natural killer cells that are CD94+ and CD16+. In certain embodiments, such natural killer cells comprise natural killer cells that are CD94− and CD16−. In certain embodiments, said first medium and/or said second medium lack leukemia inhibiting factor (LIF) and/or macrophage inflammatory protein-1 alpha (MIP-1α). In certain embodiments, said third medium lacks LIF, MIP-1α, and FMS-like tyrosine kinase-3 ligand (Flt-3L). In specific embodiments, said first medium and said second medium lack LIF and MIP-1α, and said third medium lacks LIF, MIP-1α, and Flt3L. In certain embodiments, none of the first medium, second medium or third medium comprises heparin, e.g., low-molecular weight heparin.

In one aspect, provided herein is a method of producing NK cells comprising (a) culturing hematopoietic stem or progenitor cells in a first medium comprising a stem cell mobilizing agent and thrombopoietin (Tpo) to produce a first population of cells; (b) culturing the first population of cells in a second medium comprising a stem cell mobilizing agent and interleukin-15 (IL-15), and lacking Tpo, to produce a second population of cells; and (c) culturing the second population of cells in a third medium comprising IL-2 and IL-15, and lacking LMWH, to produce a third population of cells; wherein the third population of cells comprises natural killer cells that are CD56+, CD3−, and CD11a+. In certain embodiments, said first medium and/or said second medium lack leukemia inhibiting factor (LIF) and/or macrophage inflammatory protein-1 alpha (MIP-1α). In certain embodiments, said third medium lacks LIF, MIP-1α, and FMS-like tyrosine kinase-3 ligand (Flt-3L). In specific embodiments, said first medium and said second medium lack LIF and MIP-1α, and said third medium lacks LIF, MIP-1α, and Flt3L. In certain embodiments, none of the first medium, second medium or third medium comprises heparin, e.g., low-molecular weight heparin.

In one aspect, provided herein is a method of producing NK cells comprising (a) culturing hematopoietic stem or progenitor cells in a first medium comprising a stem cell mobilizing agent and thrombopoietin (Tpo) to produce a first population of cells; (b) culturing the first population of cells in a second medium comprising a stem cell mobilizing agent and interleukin-15 (IL-15), and lacking Tpo, to produce a second population of cells; and (c) culturing the second population of cells in a third medium comprising IL-2 and IL-15, and lacking each of stem cell factor (SCF) and LMWH, to produce a third population of cells; wherein the third population of cells comprises natural killer cells that are CD56+, CD3−, and CD11a+. In certain embodiments, said first medium and/or said second medium lack leukemia inhibiting factor (LIF) and/or macrophage inflammatory protein-1 alpha (MIP-1α). In certain embodiments, said third medium lacks LIF, MIP-1α, and FMS-like tyrosine kinase-3 ligand (Flt-3L). In specific embodiments, said first medium and said second medium lack LIF and MIP-1α, and said third medium lacks LIF, MIP-1α, and Flt3L. In certain embodiments, none of the first medium, second medium or third medium comprises heparin, e.g., low-molecular weight heparin.

In one aspect, provided herein is a method of producing NK cells comprising (a) culturing hematopoietic stem or progenitor cells in a first medium comprising a stem cell mobilizing agent and thrombopoietin (Tpo) to produce a first population of cells; (b) culturing the first population of cells in a second medium comprising a stem cell mobilizing agent and interleukin-15 (IL-15), and lacking Tpo, to produce a second population of cells; and (c) culturing the second population of cells in a third medium comprising IL-2 and IL-15, and lacking each of SCF, a stem cell mobilizing agent, and LMWH, to produce a third population of cells; wherein the third population of cells comprises natural killer cells that are CD56+, CD3−, and CD11a+. In certain embodiments, said first medium and/or said second medium lack leukemia inhibiting factor (LIF) and/or macrophage inflammatory protein-1 alpha (MIP-1α). In certain embodiments, said third medium lacks LIF, MIP-1α, and FMS-like tyrosine kinase-3 ligand (Flt-3L). In specific embodiments, said first medium and said second medium lack LIF and MIP-1α, and said third medium lacks LIF, MIP-1α, and Flt3L. In certain embodiments, none of the first medium, second medium or third medium comprises heparin, e.g., low-molecular weight heparin.

In one aspect, provided herein is a method of producing NK cells comprising (a) culturing hematopoietic stem or progenitor cells in a first medium comprising a stem cell mobilizing agent and thrombopoietin (Tpo) to produce a first population of cells; (b) culturing the first population of cells in a second medium comprising a stem cell mobilizing agent and interleukin-15 (IL-15), and lacking Tpo, to produce a second population of cells; (c) culturing the second population of cells in a third medium comprising IL-2 and IL-15, and lacking each of a stem cell mobilizing agent and LMWH, to produce a third population of cells; and (d) isolating CD11a+ cells from the third population of cells to produce a fourth population of cells; wherein the fourth population of cells comprises natural killer cells that are CD56+, CD3−, and CD11a+. In certain embodiments, said first medium and/or said second medium lack leukemia inhibiting factor (LIF) and/or macrophage inflammatory protein-1 alpha (MIP-1α). In certain embodiments, said third medium lacks LIF, MIP-1α, and FMS-like tyrosine kinase-3 ligand (Flt-3L). In specific embodiments, said first medium and said second medium lack LIF and MIP-1α, and said third medium lacks LIF, MIP-1α, and Flt3L. In certain embodiments, none of the first medium, second medium or third medium comprises heparin, e.g., low-molecular weight heparin.

In certain embodiments, of any of the above embodiments, said natural killer cells express perforin and EOMES. In certain embodiments, said natural killer cells do not express either RORγt or IL1R1.

In one aspect, provided herein is a method of producing ILC3 cells comprising (a) culturing hematopoietic stem or progenitor cells in a first medium comprising a stem cell mobilizing agent and thrombopoietin (Tpo) to produce a first population of cells; (b) culturing the first population of cells in a second medium comprising a stem cell mobilizing agent and interleukin-15 (IL-15), and lacking Tpo, to produce a second population of cells; and (c) culturing the second population of cells in a third medium comprising IL-2 and IL-15, and lacking LMWH, to produce a third population of cells; wherein the third population of cells comprises ILC3 cells that are CD56+, CD3−, and CD11a−. In certain embodiments, said first medium and/or said second medium lack leukemia inhibiting factor (LIF) and/or macrophage inflammatory protein-1 alpha (MIP-1α). In certain embodiments, said third medium lacks LIF, MIP-1α, and FMS-like tyrosine kinase-3 ligand (Flt-3L). In specific embodiments, said first medium and said second medium lack LIF and MIP-1α, and said third medium lacks LIF, MIP-1α, and Flt3L. In certain embodiments, none of the first medium, second medium or third medium comprises heparin, e.g., low-molecular weight heparin.

In one aspect, provided herein is a method of producing ILC3 cells comprising (a) culturing hematopoietic stem or progenitor cells in a first medium comprising a stem cell mobilizing agent and thrombopoietin (Tpo) to produce a first population of cells; (b) culturing the first population of cells in a second medium comprising a stem cell mobilizing agent and interleukin-15 (IL-15), and lacking Tpo, to produce a second population of cells; and (c) culturing the second population of cells in a third medium comprising a stem cell mobilizing agent, IL-2 and IL-15, and lacking LMWH, to produce a third population of cells; wherein the third population of cells comprises ILC3 cells that are CD56+, CD3−, and CD11a−. In certain embodiments, said first medium and/or said second medium lack leukemia inhibiting factor (LIF) and/or macrophage inflammatory protein-1 alpha (MIP-1α). In certain embodiments, said third medium lacks LIF, MIP-1α, and FMS-like tyrosine kinase-3 ligand (Flt-3L). In specific embodiments, said first medium and said second medium lack LIF and MIP-1α, and said third medium lacks LIF, MIP-1α, and Flt3L. In certain embodiments, none of the first medium, second medium or third medium comprises heparin, e.g., low-molecular weight heparin.

In one aspect, provided herein is a method of producing ILC3 cells comprising (a) culturing hematopoietic stem or progenitor cells in a first medium comprising a stem cell mobilizing agent and thrombopoietin (Tpo) to produce a first population of cells; (b) culturing the first population of cells in a second medium comprising a stem cell mobilizing agent and interleukin-15 (IL-15), and lacking Tpo, to produce a second population of cells; and (c) culturing the second population of cells in a third medium comprising SCF, IL-2 and IL-15, and lacking LMWH, to produce a third population of cells; wherein the third population of cells comprises ILC3 cells that are CD56+, CD3−, and CD11a−. In certain embodiments, said first medium and/or said second medium lack leukemia inhibiting factor (LIF) and/or macrophage inflammatory protein-1 alpha (MIP-1α). In certain embodiments, said third medium lacks LIF, MIP-1α, and FMS-like tyrosine kinase-3 ligand (Flt-3L). In specific embodiments, said first medium and said second medium lack LIF and MIP-1α, and said third medium lacks LIF, MIP-1α, and Flt3L. In certain embodiments, none of the first medium, second medium or third medium comprises heparin, e.g., low-molecular weight heparin.

In one aspect, provided herein is a method of producing ILC3 cells comprising (a) culturing hematopoietic stem or progenitor cells in a first medium comprising a stem cell mobilizing agent and thrombopoietin (Tpo) to produce a first population of cells; (b) culturing the first population of cells in a second medium comprising a stem cell mobilizing agent and interleukin-15 (IL-15), and lacking Tpo, to produce a second population of cells; and (c) culturing the second population of cells in a third medium comprising a stem cell mobilizing agent, SCF, IL-2 and IL-15, and lacking LMWH, to produce a third population of cells; wherein the third population of cells comprises ILC3 cells that are CD56+, CD3−, and CD11a−. In certain embodiments, said first medium and/or said second medium lack leukemia inhibiting factor (LIF) and/or macrophage inflammatory protein-1 alpha (MIP-1α). In certain embodiments, said third medium lacks LIF, MIP-1α, and FMS-like tyrosine kinase-3 ligand (Flt-3L). In specific embodiments, said first medium and said second medium lack LIF and MIP-1α, and said third medium lacks LIF, MIP-1α, and Flt3L. In certain embodiments, none of the first medium, second medium or third medium comprises heparin, e.g., low-molecular weight heparin.

In one aspect, provided herein is a method of producing ILC3 cells comprising (a) culturing hematopoietic stem or progenitor cells in a first medium comprising a stem cell mobilizing agent and thrombopoietin (Tpo) to produce a first population of cells; (b) culturing the first population of cells in a second medium comprising a stem cell mobilizing agent and interleukin-15 (IL-15), and lacking Tpo, to produce a second population of cells; (c) culturing the second population of cells in a third medium comprising IL-2 and IL-15, and lacking each of a stem cell mobilizing agent and LMWH, to produce a third population of cells; and (d) isolating CD11a− cells, or removing CD11a+ cells, from the third population of cells to produce a fourth population of cells; wherein the fourth population of cells comprises ILC3 cells that are CD56+, CD3−, and CD11a−. In certain embodiments, said first medium and/or said second medium lack leukemia inhibiting factor (LIF) and/or macrophage inflammatory protein-1 alpha (MIP-1α). In certain embodiments, said third medium lacks LIF, MIP-1α, and FMS-like tyrosine kinase-3 ligand (Flt-3L). In specific embodiments, said first medium and said second medium lack LIF and MIP-1α, and said third medium lacks LIF, MIP-1α, and Flt3L. In certain embodiments, none of the first medium, second medium or third medium comprises heparin, e.g., low-molecular weight heparin.

In certain embodiments, said ILC3 cells express RORγt and IL1R1. In certain embodiments, said ILC3 cells do not express either perforin or EOMES.

### 5.2.1. Production of NK Cell and/or ILC3 Cell Populations Using a Three-Stage Method

In one embodiment, provided herein is a three-stage method of producing NK cell and/or ILC3 cell populations. In certain embodiments, the method of expansion and differentiation of the hematopoietic cells, as described herein, to produce NK cell and/or ILC3 cell populations according to a three-stage method described herein comprises maintaining the cell population comprising said hematopoietic cells at between about 2×10⁴ and about 6×10⁶ cells per milliliter. In certain aspects, said hematopoietic stem or progenitor cells are initially inoculated into said first medium from 1×10⁴ to 1×10⁵ cells/mL. In a specific aspect, said hematopoietic stem or progenitor cells are initially inoculated into said first medium at about 3×10⁴ cells/mL.

In certain aspects, said first population of cells are initially inoculated into said second medium from 5×10⁴ to 5×10⁵ cells/mL. In a specific aspect, said first population of cells is initially inoculated into said second medium at about 1×10⁵ cells/mL.

In certain aspects said second population of cells is initially inoculated into said third medium from 1×10⁵ to 5×10⁶ cells/mL. In certain aspects, said second population of cells is initially inoculated into said third medium from 1×10⁵ to 1×10⁶ cells/mL. In a specific aspect, said second population of cells is initially inoculated into said third medium at about 5×10⁵ cells/mL. In a more specific aspect, said second population of cells is initially inoculated into said third medium at about 5×10⁵ cells/mL in a spinner flask. In a specific aspect, said second population of cells is initially inoculated into said third medium at about 3×10⁵ cells/mL. In a more specific aspect, said second population of cells is initially inoculated into said third medium at about 3×10⁵ cells/mL in a static culture.

In a certain embodiment, the three-stage method comprises a first stage (“stage 1”) comprising culturing hematopoietic stem cells or progenitor cells, e.g., CD34⁺ stem cells or progenitor cells, in a first medium for a specified time period, e.g., as described herein, to produce a first population of cells. In certain embodiments, the first medium comprises a stem cell mobilizing agent and thrombopoietin (Tpo). In certain embodiments, the first medium comprises in addition to a stem cell mobilizing agent and Tpo, one or more of LMWH, Flt-3L, SCF, IL-6, IL-7, G-CSF, and GM-CSF. In a specific embodiment, the first medium comprises in addition to a stem cell mobilizing agent and Tpo, each of LMWH, Flt-3L, SCF, IL-6, IL-7, G-CSF, and GM-CSF. In a specific embodiment, the first medium lacks added LMWH. In a specific embodiment, the first medium lacks added desulphated glycosaminoglycans. In a specific embodiment, the first medium lacks LMWH. In a specific embodiment, the first medium lacks desulphated glycosaminoglycans. In a specific embodiment, in addition to a stem cell mobilizing agent and Tpo, each of Flt-3L, SCF, IL-6, IL-7, G-CSF, and GM-CSF. In specific embodiments, the first medium lacks leukemia inhibiting factor (LIF), macrophage inhibitory protein-1 alpha (MIP-1α) or both.

In certain embodiments, subsequently, in “stage 2” said cells are cultured in a second medium for a specified time period, e.g., as described herein, to produce a second population of cells. In certain embodiments, the second medium comprises a stem cell mobilizing agent and interleukin-15 (IL-15) and lacks Tpo. In certain embodiments, the second medium comprises, in addition to a stem cell mobilizing agent and IL-15, one or more of LMWH, Flt-3, SCF, IL-6, IL-7, G-CSF, and GM-CSF. In certain embodiments, the second medium comprises, in addition to a stem cell mobilizing agent and IL-15, each of LMWH, Flt-3, SCF, IL-6, IL-7, G-CSF, and GM-CSF. In a specific embodiment, the second medium lacks added LMWH. In a specific embodiment, the second medium lacks added desulphated glycosaminoglycans. In a specific embodiment, the second medium lacks heparin, e.g., LMWH. In a specific embodiment, the second medium lacks desulphated glycosaminoglycans. In certain embodiments, the second medium comprises, in addition to a stem cell mobilizing agent and IL-15, each of Flt-3, SCF, IL-6, IL-7, G-CSF, and GM-CSF. In specific embodiments, the second medium lacks leukemia inhibiting factor (LIF), macrophage inhibitory protein-1 alpha (MIP-1α) or both.

In certain embodiments, subsequently, in “stage 3” said cells are cultured in a third medium for a specified time period, e.g., as described herein, to produce a third population of cell, e.g., natural killer cells. In certain embodiments, the third medium comprises IL-2 and IL-15, and lacks a stem cell mobilizing agent and LMWH. In certain embodiments, the third medium comprises in addition to IL-2 and IL-15, one or more of SCF, IL-6, IL-7, G-CSF, and GM-CSF. In certain embodiments, the third medium comprises, in addition to IL-2 and IL-15, each of SCF, IL-6, IL-7, G-CSF, and GM-CSF. In specific embodiments, the first medium lacks one, two, or all three of LIF, MIP-1α, and Flt3L. In specific embodiments, the third medium lacks added desulphated glycosaminoglycans. In specific embodiments, the third medium lacks desulphated glycosaminoglycans. In specific embodiments, the third medium lacks heparin, e.g., LMWH.

In a specific embodiment, the three-stage method is used to produce NK cell and/or ILC3 cell populations. In certain embodiments, the three-stage method is conducted in the absence of stromal feeder cell support. In certain embodiments, the three-stage method is conducted in the absence of exogenously added steroids (e.g., cortisone, hydrocortisone, or derivatives thereof).

In certain aspects, said first medium used in the three-stage method comprises a stem cell mobilizing agent and thrombopoietin (Tpo). In certain aspects, the first medium used in the three-stage method comprises, in addition to a stem cell mobilizing agent and Tpo, one or more of Low Molecular Weight Heparin (LMWH), Flt-3 Ligand (Flt-3L), stem cell factor (SCF), IL-6, IL-7, granulocyte colony-stimulating factor (G-CSF), or granulocyte-macrophage-stimulating factor (GM-CSF). In certain aspects, the first medium used in the three-stage method comprises, in addition to a stem cell mobilizing agent and Tpo, each of LMWH, Flt-3L, SCF, IL-6, IL-7, G-CSF, and GM-CSF. In certain aspects, the first medium used in the three-stage method comprises, in addition to a stem cell mobilizing agent and Tpo, each of Flt-3L, SCF, IL-6, IL-7, G-CSF, and GM-CSF. In a specific aspect, the first medium lacks added LMWH. In a specific aspect, the first medium lacks added desulphated glycosaminoglycans. In a specific aspect, the first medium lacks LMWH. In a specific aspect, the first medium lacks desulphated glycosaminoglycans. In certain aspects, said Tpo is present in the first medium at a concentration of from 1 ng/mL to 100 ng/mL, from 1 ng/mL to 50 ng/mL, from 20 ng/mL to 30 ng/mL, or about 25 ng/mL. In other aspects, said Tpo is present in the first medium at a concentration of from 100 ng/mL to 500 ng/mL, from 200 ng/mL to 300 ng/mL, or about 250 ng/mL. In certain aspects, when LMWH is present in the first medium, the LMWH is present at a concentration of from 1 U/mL to 10 U/mL; the Flt-3L is present at a concentration of from 1 ng/mL to 50 ng/mL; the SCF is present at a concentration of from 1 ng/mL to 50 ng/mL; the IL-6 is present at a concentration of from 0.01 ng/mL to 0.1 ng/mL; the IL-7 is present at a concentration of from 1 ng/mL to 50 ng/mL; the G-CSF is present at a concentration of from 0.01 ng/mL to 0.50 ng/mL; and the GM-CSF is present at a concentration of from 0.005 ng/mL to 0.1 ng/mL. In certain aspects, in the first medium, the Flt-3L is present at a concentration of from 1 ng/mL to 50 ng/mL; the SCF is present at a concentration of from 1 ng/mL to 50 ng/mL; the IL-6 is present at a concentration of from 0.01 ng/mL to 0.1 ng/mL; the IL-7 is present at a concentration of from 1 ng/mL to 50 ng/mL; the G-CSF is present at a concentration of from 0.01 ng/mL to 0.50 ng/mL; and the GM-CSF is present at a concentration of from 0.005 ng/mL to 0.1 ng/mL. In certain aspects, when LMWH is present in the first medium, the LMWH is present at a concentration of from 4 U/mL to 5 U/mL; the Flt-3L is present at a concentration of from 20 ng/mL to 30 ng/mL; the SCF is present at a concentration of from 20 ng/mL to 30 ng/mL; the IL-6 is present at a concentration of from 0.04 ng/mL to 0.06 ng/mL; the IL-7 is present at a concentration of from 20 ng/mL to 30 ng/mL; the G-CSF is present at a concentration of from 0.20 ng/mL to 0.30 ng/mL; and the GM-CSF is present at a concentration of from 0.005 ng/mL to 0.5 ng/mL. In certain aspects, in the first medium, the Flt-3L is present at a concentration of from 20 ng/mL to 30 ng/mL; the SCF is present at a concentration of from 20 ng/mL to 30 ng/mL; the IL-6 is present at a concentration of from 0.04 ng/mL to 0.06 ng/mL; the IL-7 is present at a concentration of from 20 ng/mL to 30 ng/mL; the G-CSF is present at a concentration of from 0.20 ng/mL to 0.30 ng/mL; and the GM-CSF is present at a concentration of from 0.005 ng/mL to 0.5 ng/mL. In certain aspects, when LMWH is present in the first medium, the LMWH is present at a concentration of about 4.5 U/mL; the Flt-3L is present at a concentration of about 25 ng/mL; the SCF is present at a concentration of about 27 ng/mL; the IL-6 is present at a concentration of about 0.05 ng/mL; the IL-7 is present at a concentration of about 25 ng/mL; the G-CSF is present at a concentration of about 0.25 ng/mL; and the GM-CSF is present at a concentration of about 0.01 ng/mL. In certain aspects, in the first medium, the Flt-3L is present at a concentration of about 25 ng/mL; the SCF is present at a concentration of about 27 ng/mL; the IL-6 is present at a concentration of about 0.05 ng/mL; the IL-7 is present at a concentration of about 25 ng/mL; the G-CSF is present at a concentration of about 0.25 ng/mL; and the GM-CSF is present at a concentration of about 0.01 ng/mL. In certain embodiments, said first medium additionally comprises one or more of the following: antibiotics such as gentamycin; antioxidants such as transferrin, insulin, and/or beta-mercaptoethanol; sodium selenite; ascorbic acid; ethanolamine; and glutathione. In certain embodiments, the medium that provides the base for the first medium is a cell/tissue culture medium known to those of skill in the art, e.g., a commercially available cell/tissue culture medium such as SCGM™, STEMMACS™, GBGM®, AIM-V®, X-VIVO™ 10, X-VIVO™ 15, OPTMIZER, STEMSPAN® H3000, CELLGRO COMPLETE™, DMEM:Ham's F12 (“F12”) (e.g., 2:1 ratio, or high glucose or low glucose DMEM), Advanced DMEM (Gibco), EL08-1D2, Myelocult™ H5100, IMDM, and/or RPMI-1640; or is a medium that comprises components generally included in known cell/tissue culture media, such as the components included in GBGM®, AIM-V®, X-VIVO™ 10, X-VIVO™ 15, OPTMIZER, STEMSPAN® H3000, CELLGRO COMPLETE™, DMEM:Ham's F12 (“F12”) (e.g., 2:1 ratio, or high glucose or low glucose DMEM), Advanced DMEM (Gibco), EL08-1D2, Myelocult™ H5100, IMDM, and/or RPMI-1640. In certain embodiments, said first medium is not GBGM®. In specific embodiments of any of the above embodiments, the first medium lacks LIF, MIP-1α, or both.

In certain aspects, said second medium used in the three-stage method comprises a stem cell mobilizing agent and interleukin-15 (IL-15), and lacks Tpo. In certain aspects, the second medium used in the three-stage method comprises, in addition to a stem cell mobilizing agent and IL-15, one or more of LMWH, Flt-3, SCF, IL-6, IL-7, G-CSF, and GM-CSF. In certain aspects, the second medium used in the three-stage method comprises, in addition to a stem cell mobilizing agent and IL-15, each of LMWH, Flt-3, SCF, IL-6, IL-7, G-CSF, and GM-CSF. In certain aspects, the second medium used in the three-stage method comprises, in addition to a stem cell mobilizing agent and IL-15, each of Flt-3, SCF, IL-6, IL-7, G-CSF, and GM-CSF. In a specific aspect, the second medium lacks added LMWH. In a specific aspect, the second medium lacks added desulphated glycosaminoglycans. In a specific aspect, the second medium lacks LMWH. In a specific aspect, the second medium lacks desulphated glycosaminoglycans. In certain aspects, said IL-15 is present in said second medium at a concentration of from 1 ng/mL to 50 ng/mL, from 10 ng/mL to 30 ng/mL, or about 20 ng/mL. In certain aspects, when LMWH is present in said second medium, the LMWH is present at a concentration of from 1 U/mL to 10 U/mL; the Flt-3L is present at a concentration of from 1 ng/mL to 50 ng/mL; the SCF is present at a concentration of from 1 ng/mL to 50 ng/mL; the IL-6 is present at a concentration of from 0.01 ng/mL to 0.1 ng/mL; the IL-7 is present at a concentration of from 1 ng/mL to 50 ng/mL; the G-CSF is present at a concentration of from 0.01 ng/mL to 0.50 ng/mL; and the GM-CSF is present at a concentration of from 0.005 ng/mL to 0.1 ng/mL. In certain aspects, in said second medium, the Flt-3L is present at a concentration of from 1 ng/mL to 50 ng/mL; the SCF is present at a concentration of from 1 ng/mL to 50 ng/mL; the IL-6 is present at a concentration of from 0.01 ng/mL to 0.1 ng/mL; the IL-7 is present at a concentration of from 1 ng/mL to 50 ng/mL; the G-CSF is present at a concentration of from 0.01 ng/mL to 0.50 ng/mL; and the GM-CSF is present at a concentration of from 0.005 ng/mL to 0.1 ng/mL. In certain aspects, when LMWH is present in the second medium, the LMWH is present in the second medium at a concentration of from 4 U/mL to 5 U/mL; the Flt-3L is present at a concentration of from 20 ng/mL to 30 ng/mL; the SCF is present at a concentration of from 20 ng/mL to 30 ng/mL; the IL-6 is present at a concentration of from 0.04 ng/mL to 0.06 ng/mL; the IL-7 is present at a concentration of from 20 ng/mL to 30 ng/mL; the G-CSF is present at a concentration of from 0.20 ng/mL to 0.30 ng/mL; and the GM-CSF is present at a concentration of from 0.005 ng/mL to 0.5 ng/mL. In certain aspects, in the second medium, the Flt-3L is present at a concentration of from 20 ng/mL to 30 ng/mL; the SCF is present at a concentration of from 20 ng/mL to 30 ng/mL; the IL-6 is present at a concentration of from 0.04 ng/mL to 0.06 ng/mL; the IL-7 is present at a concentration of from 20 ng/mL to 30 ng/mL; the G-CSF is present at a concentration of from 0.20 ng/mL to 0.30 ng/mL; and the GM-CSF is present at a concentration of from 0.005 ng/mL to 0.5 ng/mL. In certain aspects, when LMWH is present in the second medium, the LMWH is present in the second medium at a concentration of from 4 U/mL to 5 U/mL; the Flt-3L is present at a concentration of from 20 ng/mL to 30 ng/mL; the SCF is present at a concentration of from 20 ng/mL to 30 ng/mL; the IL-6 is present at a concentration of from 0.04 ng/mL to 0.06 ng/mL; the IL-7 is present at a concentration of from 20 ng/mL to 30 ng/mL; the G-CSF is present at a concentration of from 0.20 ng/mL to 0.30 ng/mL; and the GM-CSF is present at a concentration of from 0.005 ng/mL to 0.5 ng/mL. In certain aspects, in the second medium, the Flt-3L is present at a concentration of from 20 ng/mL to 30 ng/mL; the SCF is present at a concentration of from 20 ng/mL to 30 ng/mL; the IL-6 is present at a concentration of from 0.04 ng/mL to 0.06 ng/mL; the IL-7 is present at a concentration of from 20 ng/mL to 30 ng/mL; the G-CSF is present at a concentration of from 0.20 ng/mL to 0.30 ng/mL; and the GM-CSF is present at a concentration of from 0.005 ng/mL to 0.5 ng/mL. In certain aspects, when LMWH is present in the second medium, the LMWH is present in the second medium at a concentration of about 4.5 U/mL; the Flt-3L is present at a concentration of about 25 ng/mL; the SCF is present at a concentration of about 27 ng/mL; the IL-6 is present at a concentration of about 0.05 ng/mL; the IL-7 is present at a concentration of about 25 ng/mL; the G-CSF is present at a concentration of about 0.25 ng/mL; and the GM-CSF is present at a concentration of about 0.01 ng/mL. In certain aspects, in the second medium, the Flt-3L is present at a concentration of about 25 ng/mL; the SCF is present at a concentration of about 27 ng/mL; the IL-6 is present at a concentration of about 0.05 ng/mL; the IL-7 is present at a concentration of about 25 ng/mL; the G-CSF is present at a concentration of about 0.25 ng/mL; and the GM-CSF is present at a concentration of about 0.01 ng/mL. In certain embodiments, said second medium additionally comprises one or more of the following: antibiotics such as gentamycin; antioxidants such as transferrin, insulin, and/or beta-mercaptoethanol; sodium selenite; ascorbic acid; ethanolamine; and glutathione. In certain embodiments, the medium that provides the base for the second medium is a cell/tissue culture medium known to those of skill in the art, e.g., a commercially available cell/tissue culture medium such as SCGM™, STEMMACS™, GBGM®, AIM-V®, X-VIVO™ 10, X-VIVO™ 15, OPTMIZER, STEMSPAN® H3000, CELLGRO COMPLETE™, DMEM:Ham's F12 (“F12”) (e.g., 2:1 ratio, or high glucose or low glucose DMEM), Advanced DMEM (Gibco), EL08-1D2, Myelocult™H5100, IMDM, and/or RPMI-1640; or is a medium that comprises components generally included in known cell/tissue culture media, such as the components included in GBGM®, AIM-V®, X-VIVO™ 10, X-VIVO™ 15, OPTMIZER, STEMSPAN® H3000, CELLGRO COMPLETE™, DMEM:Ham's F12 (“F12”) (e.g., 2:1 ratio, or high glucose or low glucose DMEM), Advanced DMEM (Gibco), EL08-1D2, Myelocult™ H5100, IMDM, and/or RPMI-1640. In certain embodiments, said second medium is not GBGM®. In specific embodiments of any of the above embodiments, the first medium lacks LIF, MIP-1α, or both.

In certain aspects, said third medium used in the three-stage method comprises IL-2 and IL-15, and lacks a stem cell mobilizing agent and LMWH. In certain aspects, said third medium used in the three-stage method comprises IL-2 and IL-15, and lacks LMWH. In certain aspects, said third medium used in the three-stage method comprises IL-2 and IL-15, and lacks SCF and LMWH. In certain aspects, said third medium used in the three-stage method comprises IL-2 and IL-15, and lacks SCF, a stem cell mobilizing agent and LMWH. In certain aspects, said third medium used in the three-stage method comprises a stem cell mobilizing agent, IL-2 and IL-15, and lacks LMWH. In certain aspects, said third medium used in the three-stage method comprises SCF, IL-2 and IL-15, and lacks LMWH. In certain aspects, said third medium used in the three-stage method comprises a stem cell mobilizing agent, SCF, IL-2 and IL-15, and lacks LMWH. In certain aspects, said third medium used in the three-stage method comprises IL-2 and IL-15, and lacks a stem cell mobilizing agent and LMWH. In certain aspects, the third medium used in the three-stage method comprises, in addition to IL-2 and IL-15, one or more of SCF, IL-6, IL-7, G-CSF, or GM-CSF. In certain aspects, the third medium used in the three-stage method comprises, in addition to IL-2 and IL-15, each of SCF, IL-6, IL-7, G-CSF, and GM-CSF. In certain aspects, said IL-2 is present in said third medium at a concentration of from 10 U/mL to 10,000 U/mL and said IL-15 is present in said third medium at a concentration of from 1 ng/mL to 50 ng/mL. In certain aspects, said IL-2 is present in said third medium at a concentration of from 100 U/mL to 10,000 U/mL and said IL-15 is present in said third medium at a concentration of from 1 ng/mL to 50 ng/mL. In certain aspects, said IL-2 is present in said third medium at a concentration of from 300 U/mL to 3,000 U/mL and said IL-15 is present in said third medium at a concentration of from 10 ng/mL to 30 ng/mL. In certain aspects, said IL-2 is present in said third medium at a concentration of about 1,000 U/mL and said IL-15 is present in said third medium at a concentration of about 20 ng/mL. In certain aspects, in said third medium, the SCF is present at a concentration of from 1 ng/mL to 50 ng/mL; the IL-6 is present at a concentration of from 0.01 ng/mL to 0.1 ng/mL; the IL-7 is present at a concentration of from 1 ng/mL to 50 ng/mL; the G-CSF is present at a concentration of from 0.01 ng/mL to 0.50 ng/mL; and the GM-CSF is present at a concentration of from 0.005 ng/mL to 0.1 ng/mL. In certain aspects, in said third medium, the SCF is present at a concentration of from 20 ng/mL to 30 ng/mL; the IL-6 is present at a concentration of from 0.04 ng/mL to 0.06 ng/mL; the IL-7 is present at a concentration of from 20 ng/mL to 30 ng/mL; the G-CSF is present at a concentration of from 0.20 ng/mL to 0.30 ng/mL; and the GM-CSF is present at a concentration of from 0.005 ng/mL to 0.5 ng/mL. In certain aspects, in said third medium, the SCF is present at a concentration of about 22 ng/mL; the IL-6 is present at a concentration of about 0.05 ng/mL; the IL-7 is present at a concentration of about 20 ng/mL; the G-CSF is present at a concentration of about 0.25 ng/mL; and the GM-CSF is present at a concentration of about 0.01 ng/mL. In certain aspects, the third medium comprises 100 ng/mL IL-7, 1000 ng/mL IL-2, 20 ng/mL IL-15, and 10 stem cell mobilizing agent and lacks SCF. In certain aspects, the third medium comprises 20 ng/mL IL-7, 1000 ng/mL IL-2, 20 ng/mL IL-15, and stem cell mobilizing agent and lacks SCF. In certain aspects, the third medium comprises 20 ng/mL IL-7, 20 ng/mL IL-15, and stem cell mobilizing agent and lacks SCF. In certain aspects, the third medium comprises 100 ng/mL IL-7, 22 ng/mL SCF, 1000 ng/mL IL-2, and 20 ng/mL IL-15 and lacks stem cell mobilizing agent. In certain aspects, the third medium comprises 22 ng/mL SCF, 1000 ng/mL IL-2, and 20 ng/mL IL-15 and lacks stem cell mobilizing agent. In certain aspects, the third medium comprises 20 ng/mL IL-7, 22 ng/mL SCF, 1000 ng/mL IL-2, and 20 ng/mL IL-15 and lacks stem cell mobilizing agent. In certain aspects, the third medium comprises 20 ng/mL IL-7, 22 ng/mL SCF, and 1000 ng/mL IL-2 and lacks stem cell mobilizing agent. In specific embodiments of any of the above embodiments, the first medium lacks one, two, or all three of LIF, MIP-1α, Flt-3L.

In certain embodiments, said third medium additionally comprises one or more of the following: antibiotics such as gentamycin; antioxidants such as transferrin, insulin, and/or beta-mercaptoethanol; sodium selenite; ascorbic acid; ethanolamine; and glutathione. In certain embodiments, the medium that provides the base for the third medium is a cell/tissue culture medium known to those of skill in the art, e.g., a commercially available cell/tissue culture medium such as SCGM™, STEMMACS™, GBGM®, AIM-V®, X-VIVO™ 10, X-VIVO™ 15, OPTMIZER, STEMSPAN® H3000, CELLGRO COMPLETE™, DMEM:Ham's F12 (“F12”) (e.g., 2:1 ratio, or high glucose or low glucose DMEM), Advanced DMEM (Gibco), EL08-1D2, Myelocult™ H5100, IMDM, and/or RPMI-1640; or is a medium that comprises components generally included in known cell/tissue culture media, such as the components included in GBGM®, AIM-V®, X-VIVO™ 10, X-VIVO™ 15, OPTMIZER, STEMSPAN® H3000, CELLGRO COMPLETE™, DMEM:Ham's F12 (“F12”) (e.g., 2:1 ratio, or high glucose or low glucose DMEM), Advanced DMEM (Gibco), EL08-1D2, Myelocult™ H5100, IMDM, and/or RPMI-1640. In certain embodiments, said third medium is not GBGM®.

Generally, the particularly recited medium components do not refer to possible constituents in an undefined component of said medium. For example, said Tpo, IL-2, and IL-15 are not comprised within an undefined component of the first medium, second medium or third medium, e.g., said Tpo, IL-2, and IL-15 are not comprised within serum. Further, said LMWH, Flt-3, SCF, IL-6, IL-7, G-CSF, and/or GM-CSF are not comprised within an undefined component of the first medium, second medium or third medium, e.g., said LMWH, Flt-3, SCF, IL-6, IL-7, G-CSF, and/or GM-CSF are not comprised within serum.

In certain aspects, said first medium, second medium or third medium comprises human serum-AB. In certain aspects, any of said first medium, second medium or third medium comprises 1% to 20% human serum-AB, 5% to 15% human serum-AB, or about 2, 5, or 10% human serum-AB.

In certain embodiments, in the three-stage methods described herein, said hematopoietic stem or progenitor cells are cultured in said first medium for 1, 2, 3, 4, 5, 6, 7, 8, 9, 10, 11, 12, 13, 14, 15, 16, 17, 18, 19, or 20 days. In certain embodiments, in the three-stage methods described herein, cells are cultured in said second medium for 1, 2, 3, 4, 5, 6, 7, 8, 9, 10, 11, 12, 13, 14, 15, 16, 17, 18, 19, or 20 days. In certain embodiments, in the three-stage methods described herein, cells are cultured in said third medium for 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days, or for more than 30 days.

In a specific embodiment, in the three-stage methods described herein, said hematopoietic stem or progenitor cells are cultured in said first medium for 7-13 days to produce a first population of cells, before said culturing in said second medium; said first population of cells are cultured in said second medium for 2-6 days to produce a second population of cells before said culturing in said third medium; and said second population of cells are cultured in said third medium for 10-30 days, i.e., the cells are cultured a total of 19-49 days.

In a specific embodiment, in the three-stage methods described herein, in the three-stage methods described herein, said hematopoietic stem or progenitor cells are cultured in said first medium for 8-12 days to produce a first population of cells, before said culturing in said second medium; said first population of cells are cultured in said second medium for 3-5 days to produce a second population of cells before said culturing in said third medium; and said second population of cells are cultured in said third medium for 15-25 days, i.e., the cells are cultured a total of 26-42 days.

In a specific embodiment, in the three-stage methods described herein, said hematopoietic stem or progenitor cells are cultured in said first medium for about 10 days to produce a first population of cells, before said culturing in said second medium; said first population of cells are cultured in said second medium for about 4 days to produce a second population of cells before said culturing in said third medium; and said second population of cells are cultured in said third medium for about 21 days, i.e., the cells are cultured a total of about 35 days.

In certain aspects, the three-stage method disclosed herein produces at least 5000-fold more natural killer cells as compared to the number of hematopoietic stem cells initially inoculated into said first medium. In certain aspects, said three-stage method produces at least 10,000-fold more natural killer cells as compared to the number of hematopoietic stem cells initially inoculated into said first medium. In certain aspects, said three-stage method produces at least 50,000-fold more natural killer cells as compared to the number of hematopoietic stem cells initially inoculated into said first medium. In certain aspects, said three-stage method produces at least 75,000-fold more natural killer cells as compared to the number of hematopoietic stem cells initially inoculated into said first medium. In certain aspects, the viability of said natural killer cells is determined by 7-aminoactinomycin D (7AAD) staining. In certain aspects, the viability of said natural killer cells is determined by annexin-V staining. In specific aspects, the viability of said natural killer cells is determined by both 7-AAD staining and annexin-V staining. In certain aspects, the viability of said natural killer cells is determined by trypan blue staining.

In certain aspects, the three-stage method disclosed herein produces at least 5000-fold more ILC3 cells as compared to the number of hematopoietic stem cells initially inoculated into said first medium. In certain aspects, said three-stage method produces at least 10,000-fold more ILC3 cells as compared to the number of hematopoietic stem cells initially inoculated into said first medium. In certain aspects, said three-stage method produces at least 50,000-fold more ILC3 cells as compared to the number of hematopoietic stem cells initially inoculated into said first medium. In certain aspects, said three-stage method produces at least 75,000-fold more ILC3 cells as compared to the number of hematopoietic stem cells initially inoculated into said first medium.

In certain aspects, the three-stage method produces natural killer cells that comprise at least 20% CD56+CD3− natural killer cells. In certain aspects, the three-stage method produces natural killer cells that comprise at least 40% CD56+CD3− natural killer cells. In certain aspects, the three-stage method produces natural killer cells that comprise at least 60% CD56+CD3− natural killer cells. In certain aspects, the three-stage method produces natural killer cells that comprise at least 70% CD56+CD3− natural killer cells. In certain aspects, the three-stage method produces natural killer cells that comprise at least 80% CD56+CD3− natural killer cells.

In certain aspects, the three-stage method disclosed herein produces natural killer cells that comprise at least 20% CD56+CD3−CD11a+ natural killer cells. In certain aspects, the three-stage method disclosed herein produces natural killer cells that comprise at least 40% CD56+CD3− CD11a+ natural killer cells. In certain aspects, the three-stage method disclosed herein produces natural killer cells that comprise at least 60% CD56+CD3− CD11a+ natural killer cells. In certain aspects, the three-stage method disclosed herein produces natural killer cells that comprise at least 80% CD56+CD3− CD11a+ natural killer cells.

In certain aspects, the three-stage method disclosed herein produces ILC3 cells that comprise at least 20% CD56+CD3− CD11a− ILC3 cells. In certain aspects, the three-stage method disclosed herein produces ILC3 cells that comprise at least 40% CD56+CD3− CD11a− ILC3 cells. In certain aspects, the three-stage method disclosed herein produces ILC3 cells that comprise at least 60% CD56+CD3− CD11a− ILC3 cells. In certain aspects, the three-stage method disclosed herein produces natural killer cells that comprise at least 80% CD56+CD3− CD11a− ILC3 cells.

In certain aspects, the three-stage method produces natural killer cells that exhibit at least 20% cytotoxicity against K562 cells when said natural killer cells and said K562 cells are co-cultured in vitro or ex vivo at a ratio of 10:1. In certain aspects, the three-stage method produces natural killer cells that exhibit at least 35% cytotoxicity against the K562 cells when said natural killer cells and said K562 cells are co-cultured in vitro or ex vivo at a ratio of 10:1. In certain aspects, the three-stage method produces natural killer cells that exhibit at least 45% cytotoxicity against the K562 cells when said natural killer cells and said K562 cells are co-cultured in vitro or ex vivo at a ratio of 10:1. In certain aspects, the three-stage method produces natural killer cells that exhibit at least 60% cytotoxicity against the K562 cells when said natural killer cells and said K562 cells are co-cultured in vitro or ex vivo at a ratio of 10:1. In certain aspects, the three-stage method produces natural killer cells that exhibit at least 75% cytotoxicity against the K562 cells when said natural killer cells and said K562 cells are co-cultured in vitro or ex vivo at a ratio of 10:1.

In certain aspects, the three-stage method produces ILC3 cells that exhibit at least 20% cytotoxicity against K562 cells when said ILC3 cells and said K562 cells are co-cultured in vitro or ex vivo at a ratio of 10:1. In certain aspects, the three-stage method produces ILC3 cells that exhibit at least 35% cytotoxicity against the K562 cells when said ILC3 cells and said K562 cells are co-cultured in vitro or ex vivo at a ratio of 10:1. In certain aspects, the three-stage method produces ILC3 cells that exhibit at least 45% cytotoxicity against the K562 cells when said ILC3 cells and said K562 cells are co-cultured in vitro or ex vivo at a ratio of 10:1. In certain aspects, the three-stage method produces ILC3 cells that exhibit at least 60% cytotoxicity against the K562 cells when said ILC3 cells and said K562 cells are co-cultured in vitro or ex vivo at a ratio of 10:1. In certain aspects, the three-stage method produces ILC3 cells that exhibit at least 75% cytotoxicity against the K562 cells when said ILC3 cells and said K562 cells are co-cultured in vitro or ex vivo at a ratio of 10:1.

In certain aspects, after said third culturing step, said third population of cells, e.g., said population of natural killer cells and/or ILC3 cells, is cryopreserved. In certain aspects, after said fourth step, said fourth population of cells, e.g., said population of natural killer cells and/or ILC3 cells, is cryopreserved.

In certain aspects, provided herein are populations of cells comprising natural killer cells, i.e., natural killer cells produced by a three-stage method described herein. Accordingly, provided herein is an isolated natural killer cell population produced by a three-stage method described herein. In a specific embodiment, said natural killer cell population comprises at least 20% CD56+CD3− natural killer cells. In a specific embodiment, said natural killer cell population comprises at least 40% CD56+CD3− natural killer cells. In a specific embodiment, said natural killer cell population comprises at least 60% CD56+CD3− natural killer cells. In a specific embodiment, said natural killer cell population comprises at least 80% CD56+CD3− natural killer cells. In a specific embodiment, said natural killer cell population comprises at least 60% CD16− cells. In a specific embodiment, said natural killer cell population comprises at least 80% CD16− cells. In a specific embodiment, said natural killer cell population comprises at least 20% CD94+ cells. In a specific embodiment, said natural killer cell population comprises at least 40% CD94+ cells.

In certain aspects, provided herein is a population of natural killer cells that is CD56+CD3− CD117+CD11a+, wherein said natural killer cells express perforin and/or EOMES, and do not express one or more of RORγt, aryl hydrocarbon receptor (AHR), and IL1R1. In certain aspects, said natural killer cells express perforin and EOMES, and do not express any of RORγt, aryl hydrocarbon receptor, or IL1R1. In certain aspects, said natural killer cells additionally express T-bet, GZMB, NKp46, NKp30, and NKG2D. In certain aspects, said natural killer cells express CD94. In certain aspects, said natural killer cells do not express CD94.

In certain aspects, provided herein is a population of ILC3 cells that is CD56+CD3− CD117+CD11a−, wherein said ILC3 cells express one or more of RORγt, aryl hydrocarbon receptor, and IL1R1, and do not express one or more of CD94, perforin, and EOMES. In certain aspects, said ILC3 cells express RORγt, aryl hydrocarbon receptor, and IL1R1, and do not express any of CD94, perforin, or EOMES. In certain aspects, said ILC3 cells additionally express CD226 and/or 2B4. In certain aspects, said ILC3 cells additionally express one or more of IL-22, TNFα, and DNAM-1. In certain aspects, said ILC3 cells express CD226, 2B4, IL-22, TNFα, and DNAM-1.

In certain aspects, provided herein is a method of producing a cell population comprising natural killer cells and ILC3 cells, comprising (a) culturing hematopoietic stem or progenitor cells in a first medium comprising a stem cell mobilizing agent and thrombopoietin (Tpo) to produce a first population of cells; (b) culturing the first population of cells in a second medium comprising a stem cell mobilizing agent and interleukin-15 (IL-15), and lacking Tpo, to produce a second population of cells; (c) culturing the second population of cells in a third medium comprising IL-2 and IL-15, and lacking each of a stem cell mobilizing agent and LMWH, to produce a third population of cells; and (d) separating CD11a+ cells and CD11a− cells from the third population of cells; and (e) combining the CD11a+ cells with the CD11a− cells in a ratio of 50:1, 40:1, 30:1, 20:1, 10:1, 5:1, 4:1, 3:1, 2:1, 1:1, 1:2, 1:3, 1:4, 1:5, 1:10, 1:20, 1:30, 1:40, or 1:50 to produce a fourth population of cells. In certain embodiments, said first medium and/or said second medium lack leukemia inhibiting factor (LIF) and/or macrophage inflammatory protein-1 alpha (MIP-1α). In certain embodiments, said third medium lacks LIF, MIP-1α, and FMS-like tyrosine kinase-3 ligand (Flt-3L). In specific embodiments, said first medium and said second medium lack LIF and MIP-1α, and said third medium lacks LIF, MIP-1α, and Flt3L. In certain embodiments, none of the first medium, second medium or third medium comprises heparin, e.g., low-molecular weight heparin. In certain aspects, in the fourth population of cells, the CD11a+ cells and CD11a− cells are combined in a ratio of 50:1. In certain aspects, in the fourth population of cells, the CD11a+ cells and CD11a− cells are combined in a ratio of 20:1. In certain aspects, in the fourth population of cells, the CD11a+ cells and CD11a− cells are combined in a ratio of 10:1. In certain aspects, in the fourth population of cells, the CD11a+ cells and CD11a− cells are combined in a ratio of 5:1. In certain aspects, in the fourth population of cells, the CD11a+ cells and CD11a− cells are combined in a ratio of 1:1. In certain aspects, in the fourth population of cells, the CD11a+ cells and CD11a− cells are combined in a ratio of 1:5. In certain aspects, in the fourth population of cells, the CD11a+ cells and CD11a− cells are combined in a ratio of 1:10. In certain aspects, in the fourth population of cells, the CD11a+ cells and CD11a− cells are combined in a ratio of 1:20. In certain aspects, in the fourth population of cells, the CD11a+ cells and CD11a− cells are combined in a ratio of 1:50.

5.3. Stem Cell Mobilizing Factors

5.3.1. Chemistry Definitions

To facilitate understanding of the disclosure of stem cell mobilizing factors set forth herein, a number of terms are defined below.

Generally, the nomenclature used herein and the laboratory procedures in biology, cellular biology, biochemistry, organic chemistry, medicinal chemistry, and pharmacology described herein are those well known and commonly employed in the art. Unless defined otherwise, all technical and scientific terms used herein generally have the same meaning as commonly understood by one of ordinary skill in the art to which this disclosure belongs.

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

As used herein, any “R” group(s) such as, without limitation, R^{a}, R^{b}, R^{c}, R^{d}, R^{e}, R^{f}, R^{g}, R^{h}, R^{m}, R^{G}, R^{J}, R^{K}, R^{U}, R^{V}, R^{Y}, and R^{Z} represent substituents that can be attached to the indicated atom. An R group may be substituted or unsubstituted. If two “R” groups are described as being “taken together” the R groups and the atoms they are attached to can form a cycloalkyl, cycloalkenyl, aryl, heteroaryl or heterocycle. For example, without limitation, if R^{a} and R^{b} of an NR^{a}R^{b} group are indicated to be “taken together,” it means that they are covalently bonded to one another to form a ring:

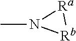

In addition, if two “R” groups are described as being “taken together” with the atom(s) to which they are attached to form a ring as an alternative, the R groups are not limited to the variables or substituents defined previously.

Whenever a group is described as being “optionally substituted” that group may be unsubstituted or substituted with one or more of the indicated substituents. Likewise, when a group is described as being “unsubstituted or substituted” if substituted, the substituent(s) may be selected from one or more the indicated substituents. If no substituents are indicated, it is meant that the indicated “optionally substituted” or “substituted” group may be substituted with one or more group(s) individually and independently selected from alkyl, alkenyl, alkynyl, cycloalkyl, cycloalkenyl, acylalkyl, hydroxy, alkoxy, alkoxyalkyl, aminoalkyl, amino acid, aryl, heteroaryl, heterocyclyl, aryl(alkyl), heteroaryl(alkyl), heterocyclyl(alkyl), hydroxyalkyl, acyl, cyano, halogen, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, isocyanato, thiocyanato, isothiocyanato, azido, nitro, silyl, sulfenyl, sulfinyl, sulfonyl, haloalkyl, haloalkoxy, trihalomethanesulfonyl, trihalomethanesulfonamido, an amino, a mono-substituted amino group and a di-substituted amino group.

As used herein, “Cₐ to C_{b}” in which “a” and “b” are integers refer to the number of carbon atoms in an alkyl, alkenyl or alkynyl group, or the number of carbon atoms in the ring of a cycloalkyl, cycloalkenyl, aryl, heteroaryl or heteroalicyclyl group. That is, the alkyl, alkenyl, alkynyl, ring(s) of the cycloalkyl, ring(s) of the cycloalkenyl, ring(s) of the aryl, ring(s) of the heteroaryl or ring(s) of the heteroalicyclyl can contain from “a” to “b”, inclusive, carbon atoms. Thus, for example, a “C₁ to C₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—. If no “a” and “b” are designated with regard to an alkyl, alkenyl, alkynyl, cycloalkyl cycloalkenyl, aryl, heteroaryl or heteroalicyclyl group, the broadest range described in these definitions is to be assumed.

As used herein, “alkyl” refers to a straight or branched hydrocarbon chain that comprises a fully saturated (no double or triple bonds) hydrocarbon group. The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 10 carbon atoms. The alkyl group could also be a lower alkyl having 1 to 6 carbon atoms. The alkyl group of the compounds may be designated as “C₁-C₄ alkyl” or similar designations. By way of example only, “C₁-C₄ alkyl” indicates that there are one to four carbon atoms in the alkyl chain, i.e., the alkyl chain is selected from methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl and hexyl. The alkyl group may be substituted or unsubstituted.

As used herein, “alkenyl” refers to an alkyl group that contains in the straight or branched hydrocarbon chain one or more double bonds. Examples of alkenyl groups include allenyl, vinylmethyl and ethenyl. An alkenyl group may be unsubstituted or substituted.

As used herein, “alkynyl” refers to an alkyl group that contains in the straight or branched hydrocarbon chain one or more triple bonds. Examples of alkynyls include ethynyl and propynyl. An alkynyl group may be unsubstituted or substituted.

As used herein, “cycloalkyl” refers to a completely saturated (no double or triple bonds) mono- or multi-cyclic hydrocarbon ring system. When composed of two or more rings, the rings may be joined together in a fused fashion. Cycloalkyl groups can contain 3 to 10 atoms in the ring(s) or 3 to 8 atoms in the ring(s). A cycloalkyl group may be unsubstituted or substituted. Typical cycloalkyl groups include, but are in no way limited to, cyclopropyl, cyclobutyl, cyclopentyl, cyclohexyl, cycloheptyl and cyclooctyl.

As used herein, “cycloalkenyl” refers to a mono- or multi-cyclic hydrocarbon ring system that contains one or more double bonds in at least one ring; although, if there is more than one, the double bonds cannot form a fully delocalized pi-electron system throughout all the rings (otherwise the group would be “aryl,” as defined herein). Cycloalkenyl groups can contain 3 to 10 atoms in the ring(s) or 3 to 8 atoms in the ring(s). When composed of two or more rings, the rings may be connected together in a fused fashion. A cycloalkenyl group may be unsubstituted or substituted.

As used herein, “aryl” refers to a carbocyclic (all carbon) monocyclic or multicyclic aromatic ring system (including fused ring systems where two carbocyclic rings share a chemical bond) that has a fully delocalized pi-electron system throughout all the rings. The number of carbon atoms in an aryl group can vary. For example, the aryl group can be a C₆-C₁₄ aryl group, a C₆-C₁₀ aryl group, or a C₆ aryl group. Examples of aryl groups include, but are not limited to, benzene, naphthalene and azulene. An aryl group may be substituted or unsubstituted.

As used herein, “heteroaryl” refers to a monocyclic or multicyclic aromatic ring system (a ring system with fully delocalized pi-electron system) that contain(s) one, two, three or more heteroatoms, that is, an element other than carbon, including but not limited to, nitrogen, oxygen and sulfur. The number of atoms in the ring(s) of a heteroaryl group can vary. For example, the heteroaryl group can contain 4 to 14 atoms in the ring(s), 5 to 10 atoms in the ring(s) or 5 to 6 atoms in the ring(s). Furthermore, the term “heteroaryl” includes fused ring systems where two rings, such as at least one aryl ring and at least one heteroaryl ring, or at least two heteroaryl rings, share at least one chemical bond. Examples of heteroaryl rings include, but are not limited to, those described herein and the following: furan, furazan, thiophene, benzothiophene, phthalazine, pyrrole, oxazole, benzoxazole, 1,2,3-oxadiazole, 1,2,4-oxadiazole, thiazole, 1,2,3-thiadiazole, 1,2,4-thiadiazole, benzothiazole, imidazole, benzimidazole, indole, indazole, pyrazole, benzopyrazole, isoxazole, benzoisoxazole, isothiazole, triazole, benzotriazole, thiadiazole, tetrazole, pyridine, pyridazine, pyrimidine, pyrazine, purine, pteridine, quinoline, isoquinoline, quinazoline, quinoxaline, cinnoline and triazine. A heteroaryl group may be substituted or unsubstituted.

As used herein, “heterocyclyl” or “heteroalicyclyl” refers to three-, four-, five-, six-, seven-, eight-, nine-, ten-, up to 18-membered monocyclic, bicyclic, and tricyclic ring system wherein carbon atoms together with from 1 to 5 heteroatoms constitute said ring system. A heterocycle may optionally contain one or more unsaturated bonds situated in such a way, however, that a fully delocalized pi-electron system does not occur throughout all the rings. The heteroatom(s) is an element other than carbon including, but not limited to, oxygen, sulfur, and nitrogen. A heterocycle may further contain one or more carbonyl or thiocarbonyl functionalities, so as to make the definition include oxo-systems and thio-systems such as lactams, lactones, cyclic imides, cyclic thioimides and cyclic carbamates. When composed of two or more rings, the rings may be joined together in a fused fashion. Additionally, any nitrogens in a heterocyclyl may be quaternized. Heterocyclyl or heteroalicyclic groups may be unsubstituted or substituted. Examples of such “heterocyclyl” or “heteroalicyclyl” groups include, but are not limited to, those described herein and the following: 1,3-dioxin, 1,3-dioxane, 1,4-dioxane, 1,2-dioxolane, 1,3-dioxolane, 1,4-dioxolane, 1,3-oxathiane, 1,4-oxathiin, 1,3-oxathiolane, 1,3-dithiole, 1,3-dithiolane, 1,4-oxathiane, tetrahydro-1,4-thiazine, 1,3-thiazinane, 2H-1,2-oxazine, maleimide, succinimide, barbituric acid, thiobarbituric acid, dioxopiperazine, hydantoin, dihydrouracil, trioxane, hexahydro-1,3,5-triazine, imidazoline, imidazolidine, isoxazoline, isoxazolidine, oxazoline, oxazolidine, oxazolidinone, thiazoline, thiazolidine, morpholine, oxirane, piperidine N-Oxide, piperidine, piperazine, pyrrolidine, pyrrolidone, pyrrolidione, 4-piperidone, pyrazoline, pyrazolidine, 2-oxopyrrolidine, tetrahydropyran, 4H-pyran, tetrahydrothiopyran, thiamorpholine, thiamorpholine sulfoxide, thiamorpholine sulfone, and their benzo-fused analogs (e.g., benzimidazolidinone, tetrahydroquinoline, and 3,4-methylenedioxyphenyl).

As used herein, “aralkyl” and “aryl(alkyl)” refer to an aryl group connected, as a substituent, via a lower alkylene group. The lower alkylene and aryl group of an aralkyl may be substituted or unsubstituted. Examples include but are not limited to benzyl, 2-phenylalkyl, 3-phenylalkyl and naphthylalkyl.

As used herein, “heteroaralkyl” and “heteroaryl(alkyl)” refer to a heteroaryl group connected, as a substituent, via a lower alkylene group. The lower alkylene and heteroaryl group of heteroaralkyl may be substituted or unsubstituted. Examples include but are not limited to 2-thienylalkyl, 3-thienylalkyl, furylalkyl, thienylalkyl, pyrrolylalkyl, pyridylalkyl, isoxazolylalkyl, imidazolylalkyl and their benzo-fused analogs.

A “heteroalicyclyl(alkyl)” and “heterocyclyl(alkyl)” refer to a heterocyclic or a heteroalicyclylic group connected, as a substituent, via a lower alkylene group. The lower alkylene and heterocyclyl of a heteroalicyclyl(alkyl) may be substituted or unsubstituted. Examples include but are not limited tetrahydro-2H-pyran-4-yl(methyl), piperidin-4-yl(ethyl), piperidin-4-yl(propyl), tetrahydro-2H-thiopyran-4-yl(methyl), and 1,3-thiazinan-4-yl(methyl).

“Lower alkylene groups” are straight-chained —CH₂— tethering groups, forming bonds to connect molecular fragments via their terminal carbon atoms. Examples include but are not limited to methylene (—CH₂—), ethylene (—CH₂CH₂—), propylene (—CH₂CH₂CH₂—), and butylene (—CH₂CH₂CH₂CH₂—). A lower alkylene group can be substituted by replacing one or more hydrogen of the lower alkylene group with a substituent(s) listed under the definition of “substituted.”

As used herein, “alkoxy” refers to the formula —OR wherein R is an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl) is defined herein. A non-limiting list of alkoxys are methoxy, ethoxy, n-propoxy, 1-methylethoxy (isopropoxy), n-butoxy, iso-butoxy, sec-butoxy, tert-butoxy, phenoxy and benzoxy. An alkoxy may be substituted or unsubstituted.

As used herein, “acyl” refers to a hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl) connected, as substituents, via a carbonyl group. Examples include formyl, acetyl, propanoyl, benzoyl and acryl. An acyl may be substituted or unsubstituted.

As used herein, “acylalkyl” refers to an acyl connected, as a substituent, via a lower alkylene group. Examples include aryl-C(═O)—(CH₂)ₙ— and heteroaryl-C(═O)—(CH₂)ₙ—, where n is an integer in the range of 1 to 6.

As used herein, “alkoxyalkyl” refers to an alkoxy group connected, as a substituent, via a lower alkylene group. Examples include C₁₋₄ alkyl-O—(CH₂)ₙ—, wherein n is an integer in the range of 1 to 6.

As used herein, “aminoalkyl” refers to an optionally substituted amino group connected, as a substituent, via a lower alkylene group. Examples include H₂N(CH₂)ₙ—, wherein n is an integer in the range of 1 to 6.

As used herein, “hydroxyalkyl” refers to an alkyl group in which one or more of the hydrogen atoms are replaced by a hydroxy group. Exemplary hydroxyalkyl groups include but are not limited to, 2-hydroxy ethyl, 3-hydroxypropyl, 2-hydroxypropyl, and 2,2-dihydroxy ethyl. A hydroxyalkyl may be substituted or unsubstituted.

As used herein, “haloalkyl” refers to an alkyl group in which one or more of the hydrogen atoms are replaced by a halogen (e.g., mono-haloalkyl, di-haloalkyl and tri-haloalkyl). Such groups include but are not limited to, chloromethyl, fluoromethyl, difluoromethyl, trifluoromethyl, chloro-fluoroalkyl, chloro-difluoroalkyl and 2-fluoroisobutyl. A haloalkyl may be substituted or unsubstituted.

As used herein, “haloalkoxy” refers to an alkoxy group in which one or more of the hydrogen atoms are replaced by a halogen (e.g., mono-haloalkoxy, di-haloalkoxy and tri-haloalkoxy). Such groups include but are not limited to, chloromethoxy, fluoromethoxy, difluoromethoxy, trifluoromethoxy, chloro-fluoroalkyl, chloro-difluoroalkoxy and 2-fluoroisobutoxy. A haloalkoxy may be substituted or unsubstituted.

A “sulfenyl” group refers to an “—SR” group in which R can be hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). A sulfenyl may be substituted or unsubstituted.

A “sulfinyl” group refers to an “—S(═O)—R” group in which R can be the same as defined with respect to sulfenyl. A sulfinyl may be substituted or unsubstituted.

A “sulfonyl” group refers to an “SO₂R” group in which R can be the same as defined with respect to sulfenyl. A sulfonyl may be substituted or unsubstituted.

An “O-carboxy” group refers to a “RC(═O)O—” group in which R can be hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl), as defined herein. An O-carboxy may be substituted or unsubstituted.

The terms “ester” and “C-carboxy” refer to a “—C(═O)OR” group in which R can be the same as defined with respect to O-carboxy. An ester and C-carboxy may be substituted or unsubstituted.

A “thiocarbonyl” group refers to a “—C(═S)R” group in which R can be the same as defined with respect to O-carboxy. A thiocarbonyl may be substituted or unsubstituted.

A “trihalomethanesulfonyl” group refers to an “X₃CSO₂—” group wherein each X is a halogen.

A “trihalomethanesulfonamido” group refers to an “X₃CS(O)₂N(R_{A})—” group wherein each X is a halogen, and R_{A} hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl).

The term “amino” as used herein refers to a —NH₂ group.

As used herein, the term “hydroxy” refers to a —OH group.

A “cyano” group refers to a “—CN” group.

The term “azido” as used herein refers to a —N₃ group.

An “isocyanato” group refers to a “—NCO” group.

A “thiocyanato” group refers to a “—CNS” group.

An “isothiocyanato” group refers to an “—NCS” group.

A “carbonyl” group refers to a C═O group.

An “S-sulfonamido” group refers to a “—SO₂N(R_{A}R_{B})” group in which R_{A} and R_{B} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An S-sulfonamido may be substituted or unsubstituted.

An “N-sulfonamido” group refers to a “RSO₂N(R_{A})—” group in which R and R_{A} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An N-sulfonamido may be substituted or unsubstituted.

An “O-carbamyl” group refers to a “—OC(═O)N(R_{A}R_{B})” group in which R_{A} and R_{B} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An O-carbamyl may be substituted or unsubstituted.

An “N-carbamyl” group refers to an “ROC(═O)N(R_{A})—” group in which R and R_{A} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An N-carbamyl may be substituted or unsubstituted.

An “O-thiocarbamyl” group refers to a “—OC(═S)—N(R_{A}R_{B})” group in which R_{A} and R_{B} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An O-thiocarbamyl may be substituted or unsubstituted.

An “N-thiocarbamyl” group refers to an “ROC(═S)N(R_{A})—” group in which R and R_{A} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An N-thiocarbamyl may be substituted or unsubstituted.

A “C-amido” group refers to a “—C(═O)N(R_{A}R_{B})” group in which R_{A} and R_{B} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). A C-amido may be substituted or unsubstituted.

An “N-amido” group refers to a “RC(═O)N(R_{A})—” group in which R and R_{A} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). An N-amido may be substituted or unsubstituted.

A “urea” group refers to “N(R)—C(═O)—NR_{A}R_{B} group in which R can be hydrogen or an alkyl, and R_{A} and R_{B} can be independently hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl). A urea may be substituted or unsubstituted.

The term “halogen atom” or “halogen” as used herein, means any one of the radio-stable atoms of column 7 of the Periodic Table of the Elements, such as, fluorine, chlorine, bromine and iodine.

As used herein, “ [symbol] ” indicates a single or double bond, unless stated otherwise.

Where the numbers of substituents is not specified (e.g. haloalkyl), there may be one or more substituents present. For example “haloalkyl” may include one or more of the same or different halogens. As another example, “C₁-C₃ alkoxyphenyl” may include one or more of the same or different alkoxy groups containing one, two or three atoms.

As used herein, the abbreviations for any protective groups, amino acids and other compounds, are, unless indicated otherwise, in accord with their common usage, recognized abbreviations, or the IUPAC-IUB Commission on Biochemical Nomenclature (See, Biochem. 11:942-944 (1972)).

In certain embodiments, “optically active” and “enantiomerically active” refer to a collection of molecules, which has an enantiomeric excess of no less than about 50%, no less than about 70%, no less than about 80%, no less than about 90%, no less than about 91%, no less than about 92%, no less than about 93%, no less than about 94%, no less than about 95%, no less than about 96%, no less than about 97%, no less than about 98%, no less than about 99%, no less than about 99.5%, or no less than about 99.8%. In certain embodiments, the compound comprises about 95% or more of the desired enantiomer and about 5% or less of the less preferred enantiomer based on the total weight of the two enantiomers in question.

In describing an optically active compound, the prefixes R and S are used to denote the absolute configuration of the optically active compound about its chiral center(s). The (+) and (−) are used to denote the optical rotation of an optically active compound, that is, the direction in which a plane of polarized light is rotated by the optically active compound. The (−) prefix indicates that an optically active compound is levorotatory, that is, the compound rotates the plane of polarized light to the left or counterclockwise. The (+) prefix indicates that an optically active compound is dextrorotatory, that is, the compound rotates the plane of polarized light to the right or clockwise. However, the sign of optical rotation, (+) and (−), is not related to the absolute configuration of a compound, R and S.

The term “isotopic variant” refers to a compound that contains an unnatural proportion of an isotope at one or more of the atoms that constitute such a compound. In certain embodiments, an “isotopic variant” of a compound contains unnatural proportions of one or more isotopes, including, but not limited to, hydrogen (¹H), deuterium (²H), tritium (³H), carbon-11 (¹¹C), carbon-12 (¹²C), carbon-13 (¹³C), carbon-14 (¹⁴C), nitrogen-13 (¹³N), nitrogen-14 (¹⁴N), nitrogen-15 (¹⁵N), oxygen-14 (¹⁴O), oxygen-15 (¹⁵O), oxygen-16 (¹⁶O), oxygen-17 (¹⁷O), oxygen-18 (¹⁸O), fluorine-17 (¹⁷F), fluorine-18 (¹⁸F), phosphorus-31 (³¹P), phosphorus-32 (³²P), phosphorus-33 (³³P), sulfur-32 (³²S), sulfur-33 (³³S), sulfur-34 (³⁴S), sulfur-35 (³⁵S), sulfur-36 (³⁶S), chlorine-35 (³⁵Cl), chlorine-36 (³⁶Cl), chlorine-37 (³⁷Cl), bromine-79 (⁷⁹Br), bromine-81 (⁸¹Br), iodine-123 (¹²³I), iodine-125 (¹²⁵I), iodine-127 (¹²⁷I), iodine-129 (¹²⁹I), and iodine-131 (¹³¹I). In certain embodiments, an “isotopic variant” of a compound is in a stable form, that is, non-radioactive. In certain embodiments, an “isotopic variant” of a compound contains unnatural proportions of one or more isotopes, including, but not limited to, hydrogen (¹H), deuterium (²H), carbon-12 (¹²C), carbon-13 (¹³C), nitrogen-14 (¹⁴N), nitrogen-15 (¹⁵N), oxygen-16 (¹⁶O), oxygen-17 (¹⁷O), oxygen-18 (¹⁸O), fluorine-17 (¹⁷F), phosphorus-31 (³¹P), sulfur-32 (³²S), sulfur-33 (³³S), sulfur-34 (³⁴S), sulfur-36 (³⁶S), chlorine-35 (³⁵Cl), chlorine-37 (³⁷Cl), bromine-79 (⁷⁹Br), bromine-81 (⁸¹Br), and iodine-127 (¹²⁷I). In certain embodiments, an “isotopic variant” of a compound is in an unstable form, that is, radioactive. In certain embodiments, an “isotopic variant” of a compound contains unnatural proportions of one or more isotopes, including, but not limited to, tritium (³H), carbon-11 (¹¹C), carbon-14 (¹⁴C), nitrogen-13 (¹³N), oxygen-14 (¹⁴O), oxygen-15 (¹⁵O), fluorine-18 (¹⁸F), phosphorus-32 (³²P), phosphorus-33 (³³P), sulfur-35 (³⁵S), chlorine-36 (³⁶Cl), iodine-123 (¹²³I), iodine-125 (¹²⁵I), iodine-129 (¹²⁹I), and iodine-131 (¹³¹I). It will be understood that, in a compound as provided herein, any hydrogen can be ²H, for example, or any carbon can be ¹³C, for example, or any nitrogen can be ¹⁵N, for example, or any oxygen can be ¹⁸O, for example, where feasible according to the judgment of one of skill. In certain embodiments, an “isotopic variant” of a compound contains unnatural proportions of deuterium (D).

The term “solvate” refers to a complex or aggregate formed by one or more molecules of a solute, e.g., a compound provided herein, and one or more molecules of a solvent, which present in a stoichiometric or non-stoichiometric amount. Suitable solvents include, but are not limited to, water, methanol, ethanol, n-propanol, isopropanol, and acetic acid. In certain embodiments, the solvent is pharmaceutically acceptable. In one embodiment, the complex or aggregate is in a crystalline form. In another embodiment, the complex or aggregate is in a noncrystalline form. Where the solvent is water, the solvate is a hydrate. Examples of hydrates include, but are not limited to, a hemihydrate, monohydrate, dihydrate, trihydrate, tetrahydrate, and pentahydrate.

The phrase “an enantiomer, a mixture of enantiomers, a mixture of two or more diastereomers, or an isotopic variant thereof; or a pharmaceutically acceptable salt, solvate, hydrate, or prodrug thereof” has the same meaning as the phrase “(i) an enantiomer, a mixture of enantiomers, a mixture of two or more diastereomers, or an isotopic variant of the compound referenced therein; (ii) a pharmaceutically acceptable salt, solvate, hydrate, or prodrug of the compound referenced therein; or (iii) a pharmaceutically acceptable salt, solvate, hydrate, or prodrug of an enantiomer, a mixture of enantiomers, a mixture of two or more diastereomers, or an isotopic variant of the compound referenced therein.”

5.3.2. Stem Cell Mobilizing Compounds

In certain aspects, the stem cell mobilizing factor is a compound having Formula (I), (I-A), (I-B), (I-C), or (I-D), as described below.

### Formula (I)

Some embodiments disclosed herein relate to a compound of Formula (I), or a pharmaceutically acceptable salt thereof, having the structure:

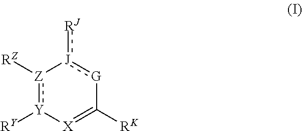

wherein: each [symbol] can independently represent a single bond or a double bond; R^{J} can be selected from the group consisting of —NR^{a}R^{b}, —OR^{b}, and ═O; wherein if R^{J} is ═O, then [symbol] joining G and J represents a single bond and G is N and the N is substituted with R^{G}; otherwise [symbol] joining G and J represents a double bond and G is N; R^{a} can be hydrogen or C₁-C₄ alkyl; R^{b} can be R^{c} or —(C₁-C₄ alkyl)-R^{c}; R^{c} can be selected from the group consisting of: —OH, —O(C₁-C₄ alkyl), —O(C₁-C₄ haloalkyl); —C(═O)NH₂; unsubstituted C₆₋₁₀ aryl; substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{c} moiety indicated as substituted can be substituted with one or more substituents E, wherein each E can be independently selected from the group consisting of: —OH, C₁-C₄ alkyl, C₁-C₄ haloalkyl, —O(C₁-C₄ alkyl), and —O(C₁-C₄ haloalkyl); R^{K} can be selected from the group consisting of: hydrogen, unsubstituted C₁₋₆ alkyl; substituted C₁₋₆ alkyl; —NH(C₁₋₄ alkyl); —N(C₁₋₄ alkyl)₂, unsubstituted C₆₋₁₀ aryl; substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{K} moiety indicated as substituted can be substituted with one or more substituents Q, wherein each Q is independently selected from the group consisting of: —OH, C₁₋₄ alkyl, C₁₋₄ haloalkyl, halo, cyano, —O—(C₁₋₄ alkyl), and —O—(C₁₋₄ haloalkyl); R^{G} can be selected from the group consisting of hydrogen, C₁₋₄ alkyl, and —(C₁₋₄ alkyl)-C(═O)NH₂; R^{Y} and R^{Z} can each independently be absent or be selected from the group consisting of: hydrogen, halo, C₁₋₆ alkyl, —OH, —O—(C₁₋₄ alkyl), —NH(C₁₋₄ alkyl), and —N(C₁₋₄ alkyl)₂; or R^{Y} and R^{Z} taken together with the atoms to which they are attached can joined together to form a ring selected from:

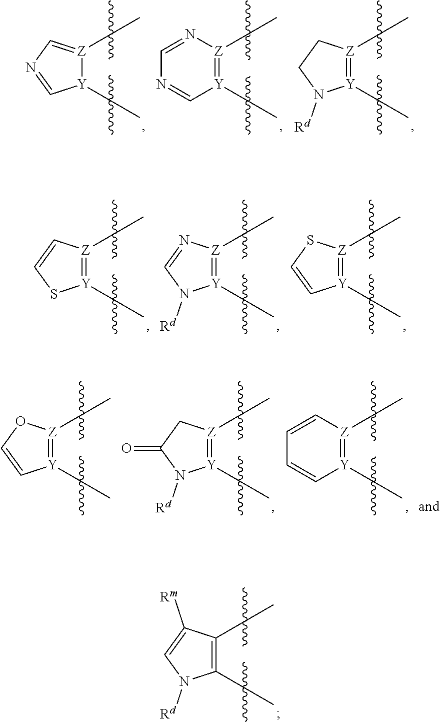

wherein said ring can be optionally substituted with one, two, or three groups independently selected from C₁₋₄ alkyl, C₁₋₄ haloalkyl, halo, cyano, —OH, —O—(C₁₋₄ alkyl), —N(C₁₋₄ alkyl)₂, unsubstituted C₆-C₁₀ aryl, C₆-C₁₀ aryl substituted with 1-5 halo atoms, and —O—(C₁₋₄ haloalkyl); and wherein if R^{Y} and R^{Z} taken together forms

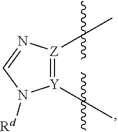

then R^{J} can be —OR^{b} or ═O; R^{d} can be hydrogen or C₁-C₄ alkyl; R^{m} can be selected from the group consisting of C₁₋₄ alkyl, halo, and cyano; J can be C; and X, Y, and Z can each be independently N or C, wherein the valency of any carbon atom is filled as needed with hydrogen atoms.

In some embodiments, [symbol] can represent a single bond. In other embodiments, [symbol] can represent a double bond. In some embodiments, [symbol] joining Y and Z can represent a single bond. In other embodiments, [symbol] joining Y and Z can represent a double bond. In some embodiments, when [symbol] joining G and J representes a single bond, G can be N and the N is substituted with R^{G}. In other embodiments, when [symbol] joining G and J represents a double bond, G can be N. In some embodiments, when [symbol] joining G and J representes a double bond, then [symbol] joining J and R^{J} can be a single bond. In some embodiments, when [symbol] joining G and J representes a double bond, then [symbol] joining J and R^{J} can not be a double bond. In some embodiments, when [symbol] joining J and R^{J} representes a double bond, then [symbol] joining G and J can be a single bond. In some embodiments, when [symbol] joining J and R^{J} representes a double bond, then [symbol] joining G and J can not be a double bond.

In some embodiments, R^{J} can be —NR^{a}R^{b}. In other embodiments, R^{J} can be —OR^{b}. In still other embodiments, R^{J} can be ═O. In some embodiments, when R^{J} is ═O, then [symbol] joining G and J represents a single bond and G is N and the N is substituted with R^{G}. In some embodiments, R^{G} is —CH₂CH₂—C(═O)NH₂.

In some embodiments, R^{a} can be hydrogen. In some embodiments, R^{a} can be C₁-C₄ alkyl. For example, R^{a} can be methyl, ethyl, n-propyl, iso-propyl, n-butyl, iso-butyl or tert-butyl.

In some embodiments, R^{b} can be R^{c}. In some embodiments, R^{b} can be —(C₁-C₄ alkyl)-R^{c}. For example, R^{b} can be —CH₂—R^{C}, —CH₂CH₂—R^{C}, —CH₂CH₂CH₂—R^{c}, or —CH₂CH₂CH₂CH₂—R^{c}. In some embodiments, when R^{b} is —CH₂CH₂—R^{c}, R^{C} can be —O(C₁-C₄ alkyl). In other embodiments, when R^{b} is —CH₂CH₂—R^{c}, R^{C} can be —O(C₁-C₄ haloalkyl). In still other embodiments, when R^{b} is —CH₂CH₂—R^{c}, R^{c} can be —C(═O)NH₂.

In some embodiments, R^{c} can be —OH. In some embodiments, R^{c} can be —O(C₁-C₄ alkyl). In some embodiments, R^{c} can be —O(C₁-C₄ haloalkyl). In some embodiments, R^{c} can be —C(═O)NH₂. In some embodiments, R^{c} can be unsubstituted C₆₋₁₀ aryl. In some embodiments, R^{c} can be substituted C₆₋₁₀ aryl. In some embodiments, R^{c} can be unsubstituted five- to ten-membered heteroaryl having 1-4 atoms selected from the group consisting of O, N, and S. In some embodiments, R^{c} can be substituted five- to ten-membered heteroaryl having 1-4 atoms selected from the group consisting of O, N, and S. In some embodiments, when a R^{c} moiety is indicated as substituted, the moiety can be substituted with one or more, for example, one, two, three, or four substituents E. In some embodiments, E can be —OH. In some embodiments, E can be C₁-C₄ alkyl. In some embodiments, E can be C₁-C₄ haloalkyl. In some embodiments, E can be —O(C₁-C₄ alkyl). In some embodiments, E can be —O(C₁-C₄ haloalkyl).

In some embodiments, when R^{b} is —CH₂CH₂—R^{C}, R^{c} can be unsubstituted C₆₋₁₀ aryl. In other embodiments, when R^{b} is —CH₂CH₂—R^{C}, R^{c} can be substituted C₆₋₁₀ aryl. In still other embodiments, when R^{b} is —CH₂CH₂—R^{C}, R^{c} can be unsubstituted five- to ten-membered heteroaryl having 1-4 atoms selected from the group consisting of O, N, and S. In yet still other embodiments, R^{b} can be —(C₁-C₄ alkyl)-R^{c} and R^{c} can be substituted five- to ten-membered heteroaryl having 1-4 atoms selected from the group consisting of O, N, and S. When a R^{c} moiety is indicated as substituted, the moiety can be substituted with one or more, for example, one, two, three, or four substituents E. In some embodiments, E can be —OH. In other embodiments, E can be C₁-C₄ alkyl. In still other embodiments, E can be C₁-C₄ haloalkyl. In still other embodiments, E can be —O(C₁-C₄ alkyl). In still other embodiments, E can be —O(C₁-C₄ haloalkyl).

In some embodiments, when R^{b} is —CH₂CH₂—R^{C}, R^{c} can be phenyl. In other embodiments, when R^{b} is —CH₂CH₂—R^{C}, R^{c} can be naphthyl. In still other embodiments, when R^{b} is —CH₂CH₂—R^{c}, R^{C} can be hydroxyphenyl. In still other embodiments, when R^{b} is —CH₂CH₂—R^{c}, R^{c} can be indolyl.

In some embodiments, R^{K} can be hydrogen. In other embodiments, R^{K} can be unsubstituted C₁₋₆ alkyl. For example, in some embodiments, R^{K} can be methyl, ethyl, n-propyl, iso-propyl, n-butyl, iso-butyl, tert-butyl, pentyl (branched and straight-chained), or hexyl (branched and straight-chained). In other embodiments, R^{K} can be substituted C₁₋₆ alkyl. In other embodiments, R^{K} can be —NH(C₁₋₄ alkyl). For example, in some embodiments, R^{K} can be —NH(CH₃), —NH(CH₂CH₃), —NH(isopropyl), or —NH(sec-butyl). In other embodiments, R^{K} can be —N(C₁₋₄ alkyl)₂.

In some embodiments, R^{K} can be unsubstituted C₆₋₁₀ aryl. In other embodiments, R^{K} can be substituted C₆₋₁₀ aryl. In other embodiments, R^{K} can be unsubstituted five- to ten-membered heteroaryl having 1-4 atoms selected from the group consisting of O, N, and S. In other embodiments, R^{K} can be substituted five- to ten-membered heteroaryl having 1-4 atoms selected from the group consisting of O, N, and S. When a R^{K} moiety is indicated as substituted, the moiety can be substituted with one or more, for example, one, two, three, or four substituents substituents Q. In some embodiments, Q can be —OH. In other embodiments, Q can be C₁₋₄ alkyl. In still other embodiments, Q can be C₁₋₄ haloalkyl. In still other embodiments, Q can be halo. In still other embodiments, Q can be cyano. In still other embodiments, Q can be —O—(C₁₋₄ alkyl). In still other embodiments, Q can be —O—(C₁₋₄ haloalkyl).

In some embodiments, R^{K} can be phenyl or naphthyl. In other embodiments, R^{K} can be benzothiophenyl. In other embodiments, R^{K} can be benzothiophenyl. In other embodiments, R^{K} can be benzothiophenyl. In still other embodiments, R^{K} can be pyridinyl. In yet still other embodiments, R^{K} can be pyridinyl substituted with one or more substituents Q. For example, R^{K} can be methylpyridinyl, ethylpyridinyl cyanopyridinyl, chloropyridinyl, fluoropyridinyl, or bromopyridinyl.

In some embodiments, R^{G} can be hydrogen. In some embodiments, R^{G} can be C₁₋₄ alkyl. In some embodiments, R^{G} can be —(C₁₋₄ alkyl)-C(═O)NH₂.

In some embodiments, R^{Y} and R^{Z} can independently be absent. In other embodiments, R^{Y} and R^{Z} can independently be hydrogen. In other embodiments, R^{Y} and R^{Z} can independently be halo. In other embodiments, R^{Y} and R^{Z} can independently be C₁₋₆ alkyl. In other embodiments, R^{Y} and R^{Z} can independently be —OH. In still other embodiments, R^{Y} and R^{Z} can independently be —O—(C₁₋₄ alkyl). In other embodiments, R^{Y} and R^{Z} can independently be —NH(C₁₋₄ alkyl). For example, R^{Y} and R^{Z} can independently be —NH(CH₃), —NH(CH₂CH₃), —NH(isopropyl), or —NH(sec-butyl). In other embodiments, R^{Y} and R^{Z} can independently be —N(C₁₋₄ alkyl)₂.

In some embodiments, R^{Y} and R^{Z} taken together with the atoms to which they are attached can be joined together to form a ring. In some embodiments, R^{Y} and R^{Z} taken together with the atoms to which they are attached can be joined together to form

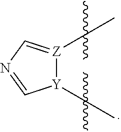

In other embodiments, R^{Y} and R^{Z} taken together with the atoms to which they are attached can be joined together to form

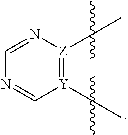

In other embodiments, R^{Y} and R^{Z} taken together with the atoms to which they are attached can be joined together to form

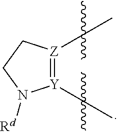

In still other embodiments, R^{Y} and R^{Z} taken together with the atoms to which they are attached can be joined together to form

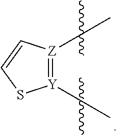

In yet still other embodiments, R^{Y} and R^{Z} taken together with the atoms to which they are attached can be joined together to form

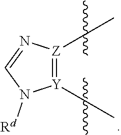

In other embodiments, R^{Y} and R^{Z} taken together with the atoms to which they are attached can be joined together to form

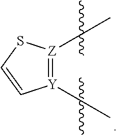

In yet other embodiments, R^{Y} and R^{Z} taken together with the atoms to which they are attached can be joined together to form

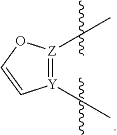

In yet still other embodiments, R^{Y} and R^{Z} taken together with the atoms to which they are attached can be joined together to form

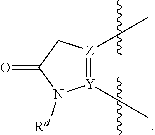

In other embodiments, R^{Y} and R^{Z} taken together with the atoms to which they are attached can be joined together to form

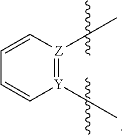

In still other embodiments, R^{Y} and R^{Z} taken together with the atoms to which they are attached can be joined together to form and

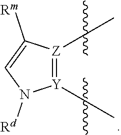

In some embodiments, when R^{Y} and R^{Z} taken together with the atoms to which they are attached can be joined together to form a ring, the ring can be substituted with one, two, or three groups independently selected from C₁-C₄ alkyl, —N(C₁-C₄ alkyl)₂, cyano, unsubstituted phenyl, and phenyl substituted with 1-5 halo atoms.

In some embodiments, when R^{Y} and R^{Z} taken together forms

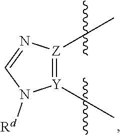

then R^{J} can be —OR^{b} or ═O.

In some embodiments, R^{Y} and R^{Z} taken together with the atoms to which they are attached can be joined together to form

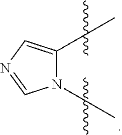 [SMILES: *c1cncn1*]

In other embodiments, R^{Y} and R^{Z} taken together with the atoms to which they are attached can be joined together to form

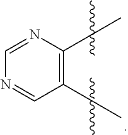 [SMILES: *c1cncnc1*]

In other embodiments, R^{Y} and R^{Z} taken together with the atoms to which they are attached can be joined together to form

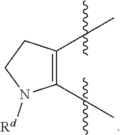

In other embodiments, R^{Y} and R^{Z} taken together with the atoms to which they are attached can be joined together to form

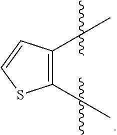 [SMILES: *c1ccsc1*]

In other embodiments, R^{Y} and R^{Z} taken together with the atoms to which they are attached can be joined together to form

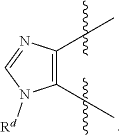

In other embodiments, R^{Y} and R^{Z} taken together with the atoms to which they are attached can be joined together to form

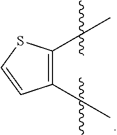 [SMILES: *c1ccsc1*]

In other embodiments, R^{Y} and R^{Z} taken together with the atoms to which they are attached can be joined together to form

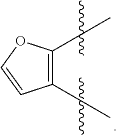 [SMILES: *c1ccoc1*]

In other embodiments, R^{Y} and R^{Z} taken together with the atoms to which they are attached can be joined together to form

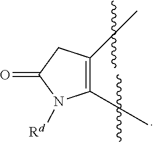

In other embodiments, R^{Y} and R^{Z} taken together with the atoms to which they are attached can be joined together to form

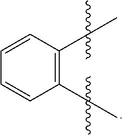 [SMILES: *c1ccccc1*]

In other embodiments, R^{Y} and R^{Z} taken together with the atoms to which they are attached can be joined together to form

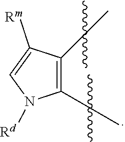

In some embodiments, when R^{Y} and R^{Z} taken together with the atoms to which they are attached can be joined together to form a ring, the ring can be substituted with one, two, or three groups independently selected from C₁-C₄ alkyl, —N(C₁-C₄ alkyl)₂, cyano, unsubstituted phenyl, and phenyl substituted with 1-5 halo atoms. In some embodiments, R^{Y} and R^{Z} taken together with the atoms to which they are attached can be

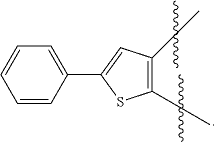 [SMILES: *c1cc(-c2ccccc2)sc1*]

In other embodiments, R^{Y} and R^{Z} taken together with the atoms to which they are attached can be

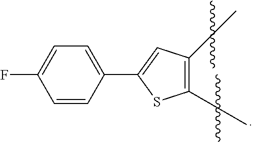 [SMILES: *c1cc(-c2ccc(F)cc2)sc1*]

In still other embodiments, R^{Y} and R^{Z} taken together with the atoms to which they are attached can be

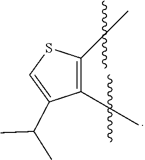 [SMILES: *c1scc(C(C)C)c1*]

In yet still other embodiments, R^{Y} and R^{Z} taken together with the atoms to which they are attached can be

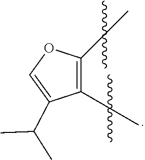 [SMILES: *c1occ(C(C)C)c1*]

In other embodiments, R^{Y} and R^{Z} taken together with the atoms to which they are attached can be

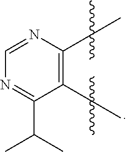 [SMILES: *c1ncnc(C(C)C)c1*]

In some embodiments, R^{d} can be hydrogen. In other embodiments, R^{d} can be C₁-C₄ alkyl. For example R^{d} can be methyl, ethyl, n-propyl, iso-propyl, n-butyl, iso-butyl or tert-butyl. In still other embodiments, R^{d} can be halo. In other embodiments, R^{d} can be cyano.

In some embodiments, R^{m} can be hydrogen. In other embodiments, R^{m} can be C₁-C₄ alkyl. For example R^{m} can be methyl, ethyl, n-propyl, iso-propyl, n-butyl, iso-butyl or tert-butyl. In still other embodiments, R^{m} can be halo. For example, R^{m} can be fluoro, chloro, bromo, or iodo. In other embodiments, R^{m} can be cyano.

In some embodiments, X, Y, and Z can each be independently N or C, wherein the valency of any carbon atom is filled as needed with hydrogen atoms. In some embodiments, X can be N, Y can be N, and Z can be N. In other embodiments, X can be N, Y can be N, and Z can be CH. In some embodiments, X can be N, Y can be CH, and Z can be N. In still other embodiments, X can be CH, Y can be N, and Z can be N. In yet still other embodiments, X can be CH, Y can be CH, and Z can be N. In other embodiments, X can be CH, Y can be N, and Z can be CH. In yet other embodiments, X can be N, Y can be CH, and Z can be CH. In other embodiments, X can be CH, Y can be CH, and Z can be CH.

In some embodiments, R^{a} can be hydrogen; R^{b} can be —(C₁-C₄ alkyl)-R^{c}; R^{c} can be selected from the group consisting of: —C(═O)NH₂; unsubstituted C₆₋₁₀ aryl; substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{c} moiety indicated as substituted is substituted with one or more substituents E, wherein each E can be independently selected from the group consisting of: —OH, C₁-C₄ alkyl, C₁-C₄ haloalkyl, —O(C₁-C₄ alkyl), and —O(C₁-C₄ haloalkyl); R^{K} can be selected from the group consisting of: hydrogen, unsubstituted C₁₋₆ alkyl; —NH(C₁₋₄ alkyl); —N(C₁₋₄ alkyl)₂, unsubstituted C₆₋₁₀ aryl; substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{K} moiety indicated as substituted is substituted with one or more substituents Q, wherein each Q can be independently selected from the group consisting of: —OH, C₁₋₄ alkyl, C₁₋₄ haloalkyl, halo, cyano, —O—(C₁₋₄ alkyl), and —O—(C₁₋₄ haloalkyl); R^{G} can be —(C₁₋₄ alkyl)-C(═O)NH₂; R^{Y} and R^{Z} can each be independently absent or be selected from the group consisting of: hydrogen, C₁₋₆ alkyl, and —NH(C₁₋₄ alkyl); or R^{Y} and R^{Z} taken together with the atoms to which they are attached can be joined together to form a ring selected from:

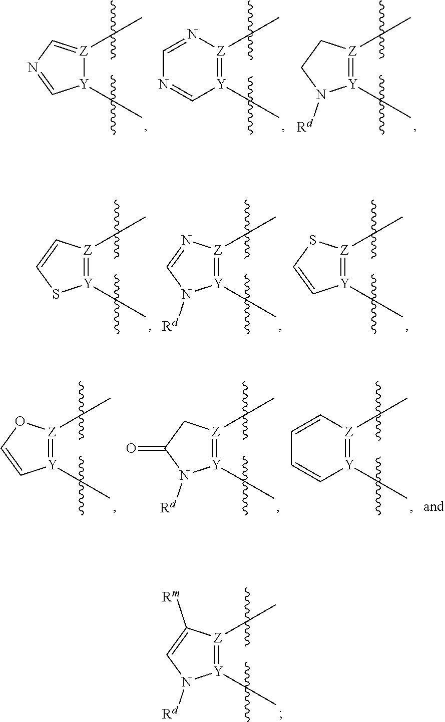

wherein said ring can be optionally substituted with one, two, or three groups independently selected from C₁₋₄ alkyl, C₁₋₄ haloalkyl, halo, cyano, —OH, —O—(C₁₋₄ alkyl), —N(C₁₋₄ alkyl)₂, unsubstituted C₆-C₁₀ aryl, C₆-C₁₀ aryl substituted with 1-5 halo atoms, and —O—(C₁₋₄ haloalkyl); R^{d} can be C₁-C₄ alkyl; R^{m} can be cyano; and X, Y, and Z can each be independently N or C, wherein the valency of any carbon atom is filled as needed with hydrogen atoms.

In some embodiments, R^{a} can be hydrogen; R^{b} can be —CH₂CH₂—R^{c}; R^{c} can be selected from the group consisting of: unsubstituted phenyl, substituted phenyl, indolyl, and —C(═O)NH₂; R^{K} can be selected from the group consisting of: hydrogen, methyl, substituted pyridinyl, unsubstituted benzothiophenyl, and —NH(C₁-C₄ alkyl); R^{G} can be —CH₂CH₂—C(═O)NH₂; R^{Y} can be —NH(C₁-C₄ alkyl); R^{Z} can be absent or hydrogen; or R^{Y} and R^{Z} taken together with the atoms to which they are attached can be joined together to form a ring selected from:

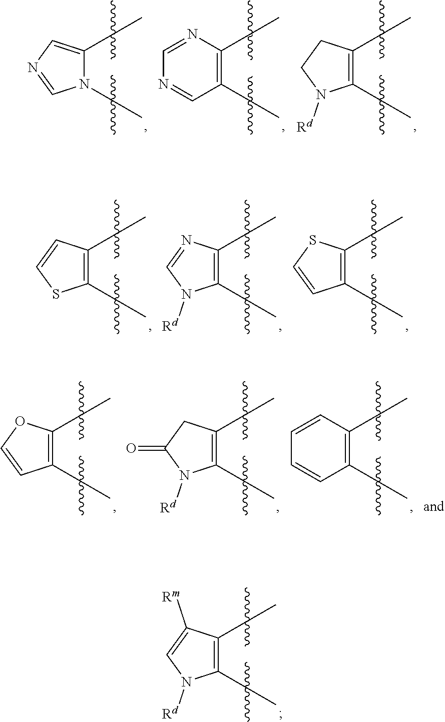

wherein said ring can be optionally substituted with one, two, or three groups independently selected from C₁-C₄ alkyl, —N(C₁-C₄ alkyl)₂, cyano, unsubstituted phenyl, and phenyl substituted with 1-5 halo atoms; R^{d} can be C₁-C₄ alkyl; R^{m} can be cyano; and X can be N or CH.

In some embodiments, when R^{J} is —NR^{a}R^{b}; G can be N: [symbol] joining G and J can be a double bond; R^{a} can hydrogen; R^{b} can be —CH₂CH₂—R^{c}; R^{c} can be unsubstituted five- to ten-membered heteroaryl having 1-4 atoms selected from the group consisting of O, N, and S; or R^{c} can be substituted C₆₋₁₀ aryl, substituted with one or more E, wherein E is —OH, R^{K} can be unsubstituted five- to ten-membered heteroaryl having 1-4 atoms selected from the group consisting of O, N, and S; or R^{K} can be substituted five- to ten-membered heteroaryl having 1-4 atoms selected from the group consisting of O, N, and S; substituted with one or more Q, wherein Q can be selected from cyano, halo, or C₁-C₄ alkyl; R^{Y} and R^{Z} taken together can be

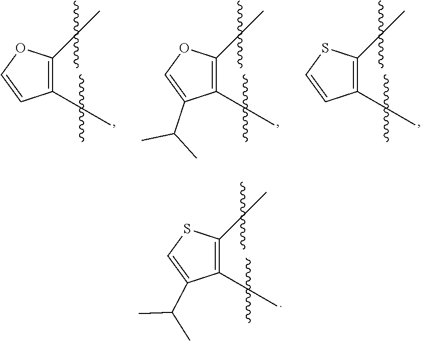

In some embodiments, when R^{J} is —NR^{a}R^{b}; G can be N; [symbol] joining G and J can be a double bond; R^{a} can hydrogen; R^{b} can be —CH₂CH₂—R^{c}; R^{c} can be unsubstituted five- to ten-membered heteroaryl having 1-4 atoms selected from the group consisting of O, N, and S; or R^{c} can be substituted C₆₋₁₀ aryl, substituted with one or more E, wherein E is —OH, R^{K} can be hydrogen, C₁₋₄ alkyl, or unsubstituted five- to ten-membered heteroaryl having 1-4 atoms selected from the group consisting of O, N, and S; and R^{Y} and R^{Z} taken together can be

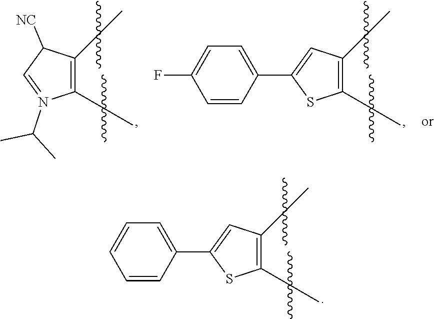

In some embodiments, when R^{J} is —NR^{a}R^{b}; G can be N; [symbol] joining G and J can be a double bond; R^{a} can hydrogen; R^{b} can be —CH₂CH₂—R^{c}; R^{c} can be unsubstituted five- to ten-membered heteroaryl having 1-4 atoms selected from the group consisting of O, N, and S; or R^{c} can be substituted C₆₋₁₀ aryl, substituted with one or more E, wherein E is —OH, R^{K} can be hydrogen, C₁₋₄ alkyl, or unsubstituted five- to ten-membered heteroaryl having 1-4 atoms selected from the group consisting of O, N, and S; and R^{Y} and R^{Z} taken together can be

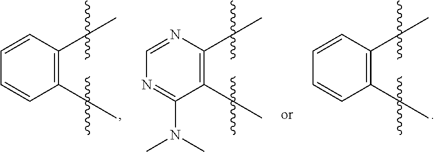 [SMILES: *c1ccccc1* ; *c1ncnc(N(C)C)c1* ; *c1ccccc1*]

In some embodiments, when R^{J} is —NR^{a}R^{b}; G can be N; [symbol] joining G and J can be a double bond, R^{a} can be hydrogen; R^{b} can be —CH₂CH₂—R^{c}; R^{c} can be substituted C₆₋₁₀ aryl; substituted with one or more E, wherein E can be —OH; R^{K} can be unsubstituted five- to ten-membered heteroaryl having 1-4 atoms selected from the group consisting of O, N, and S; R^{Y} can be —NH(C₁₋₄ alkyl); R^{Z} can be hydrogen; J can be C; X can be N; Y can be C; Z can be C; and [symbol] joining Y and Z can be a double bond. In some embodiments, the compound of Formula (I) can be 4-(2-((2-(benzo[b]thiophen-3-yl)-6-(isopropylamino)pyrimidin-4-yl)amino)ethyl)phenol.

In some embodiments, when R^{J} is —NR^{a}R^{b}; G can be N; [symbol] joining G and J can be a double bond; R^{a} can be hydrogen; R^{b} can be —CH₂CH₂—R^{C}, R^{c} can be substituted C₆₋₁₀ aryl, substituted with one or more E, wherein E can be —OH; R^{K} can be unsubstituted five- to ten-membered heteroaryl having 1-4 atoms selected from the group consisting of O, N, and S; R^{Y} and R^{Z} taken together is

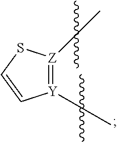

wherein the ring is substituted with C₁-C₄ alkyl; J can be C; X can be N; Y can be C; and Z can be C. In some embodiments, the compound of Formula (I) can be 4-(2-((2-(benzo[b]thiophen-3-yl)-7-isopropylthieno[3,2-d]pyrimidin-4-yl)amino)ethyl)phenol.

In some embodiments, when R^{J} is —NR^{a}R^{b}; G can be N; [symbol] joining G and J can be a double bond; R^{a} can be hydrogen; R^{b} can be —CH₂CH₂—R^{C}, R^{c} can be substituted C₆₋₁₀ aryl, substituted with one or more E, wherein E can be —OH; R^{K} can be unsubstituted five- to ten-membered heteroaryl having 1-4 atoms selected from the group consisting of O, N, and S; R^{Y} and R^{Z} taken together is

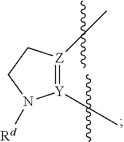

R^{d} can be C₁-C₄ alkyl; J can be C; X can be N; Y can be C; and Z can be C. In some embodiments, the compound of Formula (I) can be 4-(2-((2-(benzo[b]thiophen-3-yl)-7-isopropyl-6,7-dihydro-5H-pyrrolo[2,3-d]pyrimidin-4-yl)amino)ethyl)phenol.

In some embodiments, when R^{J} is —NR^{a}R^{b}; G can be N; [symbol] joining G and J can be a double bond; R^{a} can be hydrogen; R^{b} can be —CH₂CH₂—R^{C}, R^{c} can be substituted C₆₋₁₀ aryl, substituted with one or more E, wherein E can be —OH; R^{K} can be unsubstituted five- to ten-membered heteroaryl having 1-4 atoms selected from the group consisting of O, N, and S; R^{Y} and R^{Z} taken together is

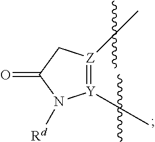

R^{d} can be C₁-C₄ alkyl; J can be C; X can be N; Y can be C; and Z can be C. In some embodiments, the compound of Formula (I) can be 2-(benzo[b]thiophen-3-yl)-4-((4-hydroxyphenethyl)amino)-7-isopropyl-5,7-dihydro-6H-pyrrolo[2,3-d]pyrimidin-6-one.

In some embodiments, when R^{J} is —OR^{b}; G can be N; [symbol] joining G and J can be a double bond; R^{b} can be —CH₂CH₂—R^{c}; R^{c} can be —C(═O)NH₂; R^{K} can unsubstituted five- to ten-membered heteroaryl having 1-4 atoms selected from the group consisting of O, N, and S; R^{Y} and R^{Z} taken together can be

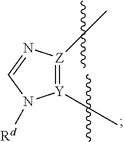

R^{d} can be C₁-C₄ alkyl; J can be C; X can be N; Y can be C; and Z is C. In some embodiments, the compound of Formula (I) can be 3-((2-(benzo[b]thiophen-3-yl)-9-isopropyl-9H-purin-6-yl)oxy)propanamide.

In some embodiments, when R^{J} is is —NR^{a}R^{b}; G can be N; [symbol] joining G and J can be a double bond; R^{b} can be —CH₂CH₂—R^{c}; R^{c} can be substituted C₆₋₁₀ aryl, substituted with one or more E, wherein E is —OH; R^{K} is unsubstituted five- to ten-membered heteroaryl having 1-4 atoms selected from the group consisting of O, N, and S; R^{Y} and R^{Z} taken together can be

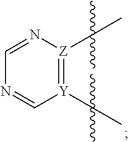

wherein said ring is substituted with —N(C₁₋₄ alkyl)₂; J can be C; X can be N; Y can be C; and Z is C. In some embodiments, the compound of Formula (I) can be 4-(2-((2-(benzo[b]thiophen-3-yl)-8-(dimethylamino)pyrimido[5,4-d]pyrimidin-4-yl)amino)ethyl)phenol.

In some embodiments, when R^{J} is is —NR^{a}R^{b}; G can be N; [symbol] joining G and J can be a double bond; R^{a} can be hydrogen; R^{b} can be —CH₂CH₂—R^{c}; R^{c} can be unsubstituted five- to ten-membered heteroaryl having 1-4 atoms selected from the group consisting of O, N, and S; R^{K} can be substituted five- to ten-membered heteroaryl having 1-4 atoms selected from the group consisting of O, N, and S; wherein a R^{K} moiety indicated as substituted is substituted with one or more Q, wherein Q is cyano; R^{Y} can be —NH(C₁₋₄ alkyl); R^{Z} can be absent; J can be C; X can be C; Y can be C; Z can be N; and [symbol] joining Y and Z can be a double bond. In some embodiments, the compound of Formula (I) can be 5-(2-((2-(1H-indol-3-yl)ethyl)amino)-6-(sec-butylamino)pyrimidin-4-yl)nicotinonitrile.

In some embodiments, when R^{J} is —NR^{a}R^{b}; G can be N; [symbol] joining G and J can be a double bond; R^{a} can be hydrogen; R^{b} can be —CH₂CH₂—R^{c}; R^{c} can be unsubstituted five- to ten-membered heteroaryl having 1-4 atoms selected from the group consisting of O, N, and S; R^{K} can be unsubstituted C₁₋₆ alkyl; R^{Y} and R^{Z} taken together can

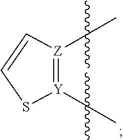

wherein the ring is substituted with unsubstituted C₆-C₁₀ aryl; J can be C; X can be N; Y can be C; Z can be C. In some embodiments, the compound of Formula (I) can be N-(2-(1H-indol-3-yl)ethyl)-2-methyl-6-phenylthieno[2,3-d]pyrimidin-4-amine

In some embodiments, when R^{J} can be —NR^{a}R^{b}; G can be N; [symbol] joining G and J can be a double bond; R^{a} can be hydrogen; R^{b} can be —CH₂CH₂—R^{c}; R^{c} can be unsubstituted five- to ten-membered heteroaryl having 1-4 atoms selected from the group consisting of O, N, and S; R^{K} can be hydrogen; R^{Y} and R^{Z} taken together can be

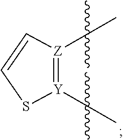

wherein the ring is substituted with substituted C₆-C₁₀ aryl; J can be C; X can be N; Y can be C; and Z can be C. In some embodiments, the compound of Formula (I) can be N-(2-(1H-indol-3-yl)ethyl)-6-(4-fluorophenyl)thieno[2,3-d]pyrimidin-4-amine

In some embodiments, when R^{J} is ═O; G can be N substituted with R^{G}; [symbol] joining G and J can be a single bond; R^{G} can be —(C₁₋₄ alkyl)-C(═O)NH₂; R^{K} can be unsubstituted five- to ten-membered heteroaryl having 1-4 atoms selected from the group consisting of O, N, and S; R^{Y} and R^{Z} taken together can be

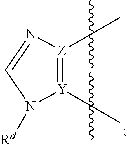

R^{d} can be C₁-C₄ alkyl; J can be C; X can be N; Y can be C; and Z can be C. In some embodiments, the compound of Formula (I) can be 3-(2-(benzo[b]thiophen-3-yl)-9-isopropyl-6-oxo-6,9-dihydro-1H-purin-1-yl)propanamide.

In some embodiments, when R^{J} is —NR^{a}R^{b}; G can be N; [symbol] joining G and J can be a double bond R^{a} can be hydrogen R^{b} can be —CH₂CH₂—R^{c}; R^{c} can be unsubstituted five- to ten-membered heteroaryl having 1-4 atoms selected from the group consisting of O, N, and S; R^{K} can be substituted five- to ten-membered heteroaryl having 1-4 atoms selected from the group consisting of O, N, and S; wherein a R^{K} moiety indicated as substituted is substituted with one or more Q, wherein Q can be halo; R^{Y} and R^{Z} taken together can be

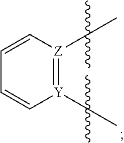

J can be C; X can be N; Y can be C; and Z can be C. In some embodiments, the compound of Formula (I) can be N-(2-(1H-indol-3-yl)ethyl)-2-(5-fluoropyridin-3-yl)quinazolin-4-amine.

In some embodiments, when R^{J} is —NR^{a}R^{b}; G is N; [symbol] joining G and J can be a double bond; R^{a} can be hydrogen R^{b} can be —CH₂CH₂—R^{c}; R^{c} can be unsubstituted five- to ten-membered heteroaryl having 1-4 atoms selected from the group consisting of O, N, and S; R^{K} can be substituted five- to ten-membered heteroaryl having 1-4 atoms selected from the group consisting of O, N, and S; wherein a R^{K} moiety indicated as substituted is substituted with one or more Q, wherein Q can be cyano; R^{Y} and R^{Z} taken together is

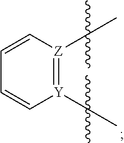

J can be C; X can be N; Y can be C; and Z can be C. In some embodiments, the compound of Formula (I) can be 5-(4-((2-(1H-indol-3-yl)ethyl)amino)quinazolin-2-yl)nicotinonitrile.

In some embodiments, when R^{J} is —NR^{a}R^{b}; G can be N; [symbol] joining G and J can be a double bond; R^{a} can be hydrogen R^{b} can be —CH₂CH₂—R^{c}; R^{c} can be unsubstituted five- to ten-membered heteroaryl having 1-4 atoms selected from the group consisting of O, N, and S; R^{K} can be —NH(C₁₋₄ alkyl); R^{Y} and R^{Z} taken together can be

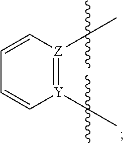

J can be C; X can be N; Y can be C; and Z can be C. In some embodiments, the compound of Formula (I) can be N-(2-(1H-indol-3-yl)ethyl)-N²-(sec-butyl)quinazoline-2,4-diamine.

In some embodiments, when R^{J} is —NR^{a}R^{b}; G can be N; [symbol] joining G and J can be a double bond; R^{a} can be hydrogen; R^{b} can be —CH₂CH₂—R^{c}; R^{c} can be substituted C₆₋₁₀ aryl, substituted with one or more E, wherein E is —OH; R^{K} can be unsubstituted five- to ten-membered heteroaryl having 1-4 atoms selected from the group consisting of O, N, and S; R^{Y} and R^{Z} taken together can be

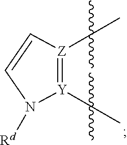

wherein the ring is substituted with cyano; R^{d} can be C₁-C₄ alkyl; J can be C; X can be N; Y can be C; and Z can be C. In some embodiments, the compound of Formula (I) can be 2-(benzo[b]thiophen-3-yl)-4-((4-hydroxyphenethyl)amino)-7-isopropyl-7H-pyrrolo[2,3-d]pyrimidine-5-carbonitrile.

In some embodiments, when R^{J} is —NR^{a}R^{b}; G can be N; [symbol] joining G and J can be a double bond; R^{a} can be hydrogen; R^{b} can be —CH₂CH₂—R^{c}; R^{c} can be unsubstituted five- to ten-membered heteroaryl having 1-4 atoms selected from the group consisting of O, N, and S; R^{K} can be unsubstituted five- to ten-membered heteroaryl having 1-4 atoms selected from the group consisting of O, N, and S; R^{Y} and R^{Z} taken together can be

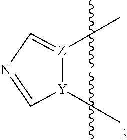

wherein the ring is substituted with C₁₋₄ alkyl; J can be C; X can be C; Y can be N; and Z can be C; wherein the valency of any carbon atom is filled as needed with hydrogen atoms. In some embodiments, the compound of Formula (I) can be N-(2-(1H-indol-3-yl)ethyl)-6-(benzo[b]thiophen-3-yl)-3-isopropylimidazo[1,5-a]pyrazin-8-amine.

In some embodiments, when R^{J} is —NR^{a}R^{b}; G can be N; [symbol] joining G and J can be a double bond; R^{a} can be hydrogen; R^{b} can be —CH₂CH₂—R^{c}; R^{c} can be substituted C₆₋₁₀ aryl, substituted with one or more E, wherein E is —OH; R^{K} can be unsubstituted five- to ten-membered heteroaryl having 1-4 atoms selected from the group consisting of O, N, and S; R^{Y} and R^{Z} taken together can be

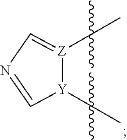

wherein the ring can be substituted with C₁₋₄ alkyl; J can be C; X can be C; Y can be N; and Z can be C; wherein the valency of any carbon atom is filled as needed with hydrogen atoms. In some embodiments, the compound of Formula (I) can be 4-(2-((6-(benzo[b]thiophen-3-yl)-3-isopropylimidazo[1,5-a]pyrazin-8-yl)amino)ethyl)phenol.

In some embodiments, when R^{J} is —NR^{a}R^{b}; G can be N; [symbol] joining G and J represents a double bond; R^{a} can be hydrogen R^{b} can be —CH₂CH₂—R^{c}; R^{c} can be unsubstituted five- to ten-membered heteroaryl having 1-4 atoms selected from the group consisting of O, N, and S; R^{K} can be substituted five- to ten-membered heteroaryl having 1-4 atoms selected from the group consisting of O, N, and S; wherein a R^{K} moiety indicated as substituted is substituted with one or more Q, wherein Q is cyano; R^{Y} and R^{Z} taken together is

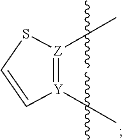

wherein the ring is substituted with C₁-C₄ alkyl; J can be C; X can be N; Y can be C; and Z can be C. In some embodiments, the compound of Formula (I) can be 5-(4-((2-(1H-indol-3-yl)ethyl)amino)-7-isopropylthieno[3,2-d]pyrimidin-2-yl)nicotinonitrile.

In some embodiments, when R^{J} is —NR^{a}R^{b}; G can be N; [symbol] joining G and J represents a double bond; R^{a} can be hydrogen; R^{b} can be —CH₂CH₂—R^{c}; R^{c} can be unsubstituted five- to ten-membered heteroaryl having 1-4 atoms selected from the group consisting of O, N, and S; R^{K} can be substituted five- to ten-membered heteroaryl having 1-4 atoms selected from the group consisting of O, N, and S; wherein a R^{K} moiety indicated as substituted is substituted with one or more Q, wherein Q is halo; R^{Y} and R^{Z} taken together can be

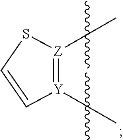

wherein the ring is substituted with C₁-C₄ alkyl; J can be C; X can be N; Y can be C; and Z can be C. In some embodiments, the compound of Formula (I) can be N-(2-(1H-indol-3-yl)ethyl)-2-(5-fluoropyridin-3-yl)-7-isopropylthieno[3,2d]pyrimidin-4-amine.

In some embodiments, when R^{J} is —NR^{a}R^{b}; G can be N; [symbol] joining G and J can be a double bond; R^{a} can be hydrogen; R^{b} can be —CH₂CH₂—R^{c}; R^{c} can be unsubstituted five- to ten-membered heteroaryl having 1-4 atoms selected from the group consisting of O, N, and S; R^{K} can be substituted five- to ten-membered heteroaryl having 1-4 atoms selected from the group consisting of O, N, and S; wherein a R^{K} moiety indicated as substituted is substituted with one or more Q, wherein Q is halo; R^{Y} and R^{Z} taken together can be

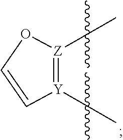

J can be C; X can be N; Y can be C; and Z can be C. In some embodiments, the compound of Formula (I) can be N-(2-(1H-indol-3-yl)ethyl)-2-(5-fluoropyridin-3-yl)furo[3,2-d]pyrimidin-4-amine.

In some embodiments, when R^{J} is —NR^{a}R^{b}; G can be N; [symbol] joining G and J can be a double bond; R^{a} can be hydrogen; R^{b} can be —CH₂CH₂—R^{c}; R^{c} can be unsubstituted five- to ten-membered heteroaryl having 1-4 atoms selected from the group consisting of O, N, and S; R^{K} can be substituted five- to ten-membered heteroaryl having 1-4 atoms selected from the group consisting of O, N, and S; wherein a R^{K} moiety indicated as substituted is substituted with one or more Q, wherein Q is C₁-C₄ alkyl; R^{Y} and R^{Z} taken together can be

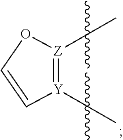

J can be C; X can be N; Y can be C; and Z can be C. In some embodiments, the compound of Formula (I) can be N-(2-(1H-indol-3-yl)ethyl)-2-(5-methylpyridin-3-yl)furo[3,2-d]pyrimidin-4-amine.

In some embodiments, when R^{J} is —NR^{a}R^{b}; G can be N; [symbol] joining G and J can be a double bond; R^{a} can be hydrogen; R^{b} can be —CH₂CH₂—R^{c}; R^{c} can be unsubstituted five- to ten-membered heteroaryl having 1-4 atoms selected from the group consisting of O, N, and S; R^{K} can be substituted five- to ten-membered heteroaryl having 1-4 atoms selected from the group consisting of O, N, and S; wherein a R^{K} moiety indicated as substituted is substituted with one or more Q, wherein Q is C₁-C₄ alkyl; R^{Y} and R^{Z} taken together can be

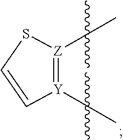

wherein the ring is substituted with C₁-C₄ alkyl J can be C; X can be N; Y can be C; and Z can be C. In some embodiments, the compound of Formula (I) can be N-(2-(1H-indol-3-yl)ethyl)-7-isopropyl-2-(5-methylpyridin-3-yl)thieno[3,2-d]pyrimidin-4-amine.

In some embodiments, when R^{J} is —NR^{a}R^{b}; G is N; [symbol] joining G and J can be a double bond; R^{a} can be hydrogen; R^{b} can be —CH₂CH₂—R^{c}; R^{c} can be unsubstituted five- to ten-membered heteroaryl having 1-4 atoms selected from the group consisting of O, N, and S; R^{K} can be substituted five- to ten-membered heteroaryl having 1-4 atoms selected from the group consisting of O, N, and S; wherein a R^{K} moiety indicated as substituted is substituted with one or more Q, wherein Q is cyano; R^{Y} and R^{Z} taken together can be

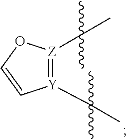

J can be C; X can be N; Y can be C; and Z can be C. In some embodiments, the compound of Formula (I) can be 5-(4-((2-(1H-indol-3-yl)ethyl)amino)furo[3,2-d]pyrimidin-2-yl)nicotinonitrile.

In some embodiments, provided herein is compound of Formula (I), wherein the compound can be selected from:
4-(2-((2-(benzo[b]thiophen-3-yl)-6-(isopropylamino)pyrimidin-4-yl)amino)ethyl)phenol;
4-(2-((2-(benzo[b]thiophen-3-yl)-7-isopropylthieno[3,2-d]pyrimidin-4-yl)amino)ethyl)phenol;
4-(2-((2-(benzo[b]thiophen-3-yl)-7-isopropyl-6,7-dihydro-5H-pyrrolo[2,3-d]pyrimidin-4-yl)amino)ethyl)phenol;
2-(benzo[b]thiophen-3-yl)-4-((4-hydroxyphenethyl)amino)-7-isopropyl-5,7-dihydro-6H-pyrrolo[2,3-d]pyrimidin-6-one;
3-((2-(benzo[b]thiophen-3-yl)-9-isopropyl-9H-purin-6-yl)oxy)propanamide;
4-(2-((2-(benzo[b]thiophen-3-yl)-8-(dimethylamino)pyrimido[5,4-d]pyrimidin-4-yl)amino)ethyl)phenol;
5-(2-((2-(1H-indol-3-yl)ethyl)amino)-6-(sec-butylamino)pyrimidin-4-yl)nicotinonitrile;
N-(2-(1H-indol-3-yl)ethyl)-2-methyl-6-phenylthieno[2,3-d]pyrimidin-4-amine;
N-(2-(1H-indol-3-yl)ethyl)-6-(4-fluorophenyl)thieno[2,3-d]pyrimidin-4-amine;
3-(2-(benzo[b]thiophen-3-yl)-9-isopropyl-6-oxo-6,9-dihydro-1H-purin-1-yl)propanamide;
N-(2-(1H-indol-3-yl)ethyl)-2-(5-fluoropyridin-3-yl)quinazolin-4-amine;
5-(4-((2-(1H-indol-3-yl)ethyl)amino)quinazolin-2-yl)nicotinonitrile;
N⁴-(2-(1H-indol-3-yl)ethyl)-N²-(sec-butyl)quinazoline-2,4-diamine;
2-(benzo[b]thiophen-3-yl)-4-((4-hydroxyphenethyl)amino)-7-isopropyl-7H-pyrrolo[2,3-d]pyrimidine-5-carbonitrile;
N-(2-(1H-indol-3-yl)ethyl)-6-(benzo[b]thiophen-3-yl)-3-isopropylimidazo[1,5-a]pyrazin-8-amine;
4-(2-((6-(benzo[b]thiophen-3-yl)-3-isopropylimidazo[1,5-a]pyrazin-8-yl)amino)ethyl)phenol;
5-(4-((2-(1H-indol-3-yl)ethyl)amino)-7-isopropylthieno[3,2-d]pyrimidin-2-yl)nicotinonitrile;
N-(2-(1H-indol-3-yl)ethyl)-2-(5-fluoropyridin-3-yl)-7-isopropylthieno[3,2-d]pyrimidin-4-amine;
N-(2-(1H-indol-3-yl)ethyl)-2-(5-fluoropyridin-3-yl)furo[3,2-d]pyrimidin-4-amine;
N-(2-(1H-indol-3-yl)ethyl)-2-(5-methylpyridin-3-yl)furo[3,2-d]pyrimidin-4-amine;
N-(2-(1H-indol-3-yl)ethyl)-7-isopropyl-2-(5-methylpyridin-3-yl)thieno[3,2-d]pyrimidin-4-amine;
5-(4-((2-(1H-indol-3-yl)ethyl)amino)furo[3,2-d]pyrimidin-2-yl)nicotinonitrile; and pharmaceutically acceptable salts thereof.

### Formula (I-A)

In some embodiments provided herein, the compound of Formula (I) can have the structure of Formula (I-A):

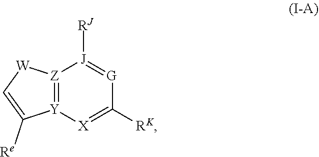

including pharmaceutically acceptable salts thereof, wherein: R^{J} can be —NR^{a}R^{b}; R^{a} can be hydrogen or C₁-C₄ alkyl; R^{b} can be R^{c} or —(C₁-C₄ alkyl)-R^{c}; R^{c} can be selected from the group consisting of: unsubstituted C₆₋₁₀ aryl; substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{c} moiety indicated as substituted is substituted with one or more substituents E, wherein each E can be independently selected from the group consisting of: —OH, C₁-C₄ alkyl, C₁-C₄ haloalkyl, —O(C₁-C₄ alkyl), and —O(C₁-C₄ haloalkyl); R^{K} can be selected from the group consisting of: hydrogen, unsubstituted C₁₋₆ alkyl;
—NH(C₁₋₄ alkyl); —N(C₁₋₄ alkyl)₂, unsubstituted C₆₋₁₀ aryl; substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{K} moiety indicated as substituted is substituted with one or more substituents Q, wherein each Q can be independently selected from the group consisting of: —OH, C₁₋₄ alkyl, C₁₋₄ haloalkyl, halo, cyano, —O—(C₁₋₄ alkyl), and —O—(C₁₋₄ haloalkyl); Y and Z can each be C; X can be N or CH; W can be O or S; and R^{e} can be hydrogen or C₁-C₄ alkyl.

In some embodiments, R^{a} can be hydrogen. In other embodiments, R^{a} can be C₁-C₄ alkyl.

In some embodiments, R^{b} can be —(C₁-C₄ alkyl)-R^{c}. For example, R^{b} can be —CH₂—R^{C}, —CH₂CH₂—R^{c}, —CH₂CH₂CH₂—R^{c}, or —CH₂CH₂CH₂CH₂—R^{c}.

In some embodiments, R^{c} can be —OH. In some embodiments, R^{c} can be —O(C₁-C₄ alkyl). In some embodiments, R^{c} can be —O(C₁-C₄ haloalkyl). In some embodiments, R^{c} can be —C(═O)NH₂. In some embodiments, R^{c} can be unsubstituted C₆₋₁₀ aryl. In some embodiments, R^{c} can be substituted C₆₋₁₀ aryl. In some embodiments, R^{c} can be unsubstituted five- to ten-membered heteroaryl having 1-4 atoms selected from the group consisting of O, N, and S. In some embodiments, R^{c} can be substituted five- to ten-membered heteroaryl having 1-4 atoms selected from the group consisting of O, N, and S. In some embodiments, when a R^{c} moiety is indicated as substituted, the moiety can be substituted with one or more, for example, one, two, three, or four substituents E. In some embodiments, E can be —OH. In some embodiments, E can be C₁-C₄ alkyl. In some embodiments, E can be C₁-C₄ haloalkyl. In some embodiments, E can be —O(C₁-C₄ alkyl). In some embodiments, E can be —O(C₁-C₄ haloalkyl). In some embodiments R^{c} can be phenyl. In other embodiments, R^{c} can be hydroxyphenyl. In still other embodiments, R^{c} can be indolyl.

In some embodiments, R^{K} can be unsubstituted five- to ten-membered heteroaryl having 1-4 atoms selected from the group consisting of O, N, and S. In some embodiments, R^{K} can be substituted five- to ten-membered heteroaryl having 1-4 atoms selected from the group consisting of O, N, and S; wherein the substituted heteroaryl can substituted with one or more substituents Q, wherein each Q can independently selected from the group consisting of: —OH, C₁₋₄ alkyl, C₁₋₄ haloalkyl, halo, cyano, —O—(C₁₋₄ alkyl), and —O—(C₁₋₄ haloalkyl). In some embodiments, R^{K} can be pyridinyl. In other embodiments, R^{K} can be pyridinyl substituted with one or more substituents Q. For example, R^{K} can be methylpyridinyl, ethylpyridinyl cyanopyridinyl, chloropyridinyl, fluoropyridinyl, or bromopyridinyl.

In some embodiments, R^{e} can be hydrogen. In some embodiments, R^{e} can be C₁-C₄ alkyl. For example, R^{e} can be methyl, ethyl, n-propyl, iso-propyl, n-butyl, iso-butyl or tert-butyl.

In some embodiments, R^{a} can be hydrogen; R^{b} can be —(C₁-C₄ alkyl)-R^{c}; R^{c} can be selected from the group consisting of: unsubstituted C₆₋₁₀ aryl; substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{c} moiety indicated as substituted is substituted with one or more substituents E, wherein each E can be independently selected from the group consisting of: —OH, C₁-C₄ alkyl, C₁-C₄ haloalkyl, —O(C₁-C₄ alkyl), and —O(C₁-C₄ haloalkyl); R^{K} can be selected from the group consisting of: unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein the substituted heteroaryl is substituted with one or more substituents Q, wherein each Q can be independently selected from the group consisting of: —OH, C₁₋₄ alkyl, C₁₋₄ haloalkyl, halo, cyano, —O—(C₁₋₄ alkyl), and —O—(C₁₋₄ haloalkyl); and R^{e} can be C₁-C₄ alkyl.

In some embodiments, R^{a} can be hydrogen; R^{b} can be —(CH₂—CH₂)—R^{c}; R^{c} can be selected from the group consisting of: substituted phenyl and unsubstituted indolyl; wherein the substituted phenyl is substituted with one substituent E, wherein E can be —OH; R^{K} can be selected from the group consisting of: unsubstituted benzothiophenyl and substituted pyridinyl; wherein the substituted pyridinyl is substituted with one substituent Q, wherein Q can be selected from the group consisting of: C₁₋₄ alkyl, halo, and cyano; and R^{e} can be isopropyl.

In some embodiments, when W is O, R^{J} can be —NR^{a}R^{b}; R^{a} can be hydrogen; R^{b} can be —CH₂CH₂—R^{c}; R^{c} can be selected from the group consisting of: unsubstituted C₆₋₁₀ aryl; substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{c} moiety indicated as substituted is substituted with one or more substituents E, wherein each E can be independently selected from the group consisting of: —OH, C₁-C₄ alkyl, and —O(C₁-C₄ alkyl); R^{K} can be selected from the group consisting of unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{K} moiety indicated as substituted is substituted with one or more substituents Q, wherein each Q can be independently selected from the group consisting of: —C₁₋₄ alkyl, halo, cyano, and —O—(C₁₋₄ alkyl); Y and Z can each be C; X can be N or CH; and R^{e} can be hydrogen or C₁-C₄ alkyl.

In some embodiments, when W is S, R^{J} can be —NR^{a}R^{b}; R^{a} can be hydrogen; R^{b} can be —CH₂CH₂—R^{c}; R^{c} can be selected from the group consisting of: unsubstituted C₆₋₁₀ aryl; substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{c} moiety indicated as substituted is substituted with one or more substituents E, wherein each E can be independently selected from the group consisting of: —OH, C₁-C₄ alkyl, and —O(C₁-C₄ alkyl); R^{K} can be selected from the group consisting of unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{K} moiety indicated as substituted is substituted with one or more substituents Q, wherein each Q can be independently selected from the group consisting of: —C₁₋₄ alkyl, halo, cyano, and —O—(C₁₋₄ alkyl); Y and Z can each be C; X can be N or CH; and R^{e} can be hydrogen or C₁-C₄ alkyl.

In some embodiments, when R^{J} is —NR^{a}R^{b}; G can be N; R^{a} can be hydrogen; R^{b} can be —CH₂CH₂—R^{c}; R^{c} can be unsubstituted five- to ten-membered heteroaryl having 1-4 atoms selected from the group consisting of O, N, and S; R^{K} can be substituted five- to ten-membered heteroaryl having 1-4 atoms selected from the group consisting of O, N, and S; wherein a R^{K} moiety indicated as substituted is substituted with one or more Q, wherein Q is C₁-C₄ alkyl; W can be S; R^{e} can be C₁-C₄ alkyl; J can be C; X can be N; Y can be C; and Z can be C. In some embodiments, the compound of Formula (I-A) can be N-(2-(1H-indol-3-yl)ethyl)-7-isopropyl-2-(5-methylpyridin-3-yl)thieno[3,2-d]pyrimidin-4-amine.

In some embodiments, when R^{J} is —NR^{a}R^{b}; G can be N; R^{a} can be hydrogen R^{b} can be —CH₂CH₂—R^{c}; R^{c} can be unsubstituted five- to ten-membered heteroaryl having 1-4 atoms selected from the group consisting of O, N, and S; R^{K} can be substituted five- to ten-membered heteroaryl having 1-4 atoms selected from the group consisting of O, N, and S; wherein a R^{K} moiety indicated as substituted is substituted with one or more Q, wherein Q is cyano; W can be S; R^{e} can be C₁-C₄ alkyl; J can be C; X can be N; Y can be C; and Z can be C. In some embodiments, the compound of Formula (I-A) can be 5-(4-((2-(1H-indol-3-yl)ethyl)amino)-7-isopropylthieno[3,2-d]pyrimidin-2-yl)nicotinonitrile.

In some embodiments, when R^{J} is —NR^{a}R^{b}; G can be N; R^{a} can be hydrogen; R^{b} can be —CH₂CH₂—R^{c}; R^{c} can be unsubstituted five- to ten-membered heteroaryl having 1-4 atoms selected from the group consisting of O, N, and S; R^{K} can be substituted five- to ten-membered heteroaryl having 1-4 atoms selected from the group consisting of O, N, and S; wherein a R^{K} moiety indicated as substituted is substituted with one or more Q, wherein Q is halo; W can be S; R^{e} can be C₁-C₄ alkyl; J can be C; X can be N; Y can be C; and Z can be C. In some embodiments, the compound of Formula (I-A) can be N-(2-(1H-indol-3-yl)ethyl)-2-(5-fluoropyridin-3-yl)-7-isopropylthieno[3,2d]pyrimidin-4-amine.

In some embodiments, when R^{J} is —NR^{a}R^{b}; G can be N; R^{a} can be hydrogen; R^{b} can be —CH₂CH₂—R^{C}, R^{c} can be substituted C₆₋₁₀ aryl, substituted with one or more E, wherein E can be —OH; R^{K} can be unsubstituted five- to ten-membered heteroaryl having 1-4 atoms selected from the group consisting of O, N, and S; W can be S; R^{e} can be C₁-C₄ alkyl; J can be C; X can be N; Y can be C; and Z can be C. In some embodiments, the compound of Formula (I-A) can be 4-(2-((2-(benzo[b]thiophen-3-yl)-7-isopropylthieno[3,2-d]pyrimidin-4-yl)amino)ethyl)phenol.

In some embodiments, when R^{J} is —NR^{a}R^{b}; G can be N; R^{a} can be hydrogen; R^{b} can be —CH₂CH₂—R^{c}; R^{c} can be unsubstituted five- to ten-membered heteroaryl having 1-4 atoms selected from the group consisting of O, N, and S; R^{K} can be substituted five- to ten-membered heteroaryl having 1-4 atoms selected from the group consisting of O, N, and S; wherein a R^{K} moiety indicated as substituted is substituted with one or more Q, wherein Q is halo; W can be O; R^{e} can be hydrogen; J can be C; X can be N; Y can be C; and Z can be C. In some embodiments, the compound of Formula (I-A) can be N-(2-(1H-indol-3-yl)ethyl)-2-(5-fluoropyridin-3-yl)furo[3,2-d]pyrimidin-4-amine.

In some embodiments, when R^{J} is —NR^{a}R^{b}; G can be N; [symbol] joining G and J can be a double bond; R^{a} can be hydrogen; R^{b} can be —CH₂CH₂—R^{c}; R^{c} can be unsubstituted five- to ten-membered heteroaryl having 1-4 atoms selected from the group consisting of O, N, and S; R^{K} can be substituted five- to ten-membered heteroaryl having 1-4 atoms selected from the group consisting of O, N, and S; wherein a R^{K} moiety indicated as substituted is substituted with one or more Q, wherein Q is C₁-C₄ alkyl; W can be O; R^{e} can be hydrogen; J can be C; X can be N; Y can be C; and Z can be C. In some embodiments, the compound of Formula (I-A) can be N-(2-(1H-indol-3-yl)ethyl)-2-(5-methylpyridin-3-yl)furo[3,2-d]pyrimidin-4-amine.

In some embodiments, when R^{J} is —NR^{a}R^{b}; G is NR^{a} can be hydrogen; R^{b} can be —CH₂CH₂—R^{c}; R^{c} can be unsubstituted five- to ten-membered heteroaryl having 1-4 atoms selected from the group consisting of O, N, and S; R^{K} can be substituted five- to ten-membered heteroaryl having 1-4 atoms selected from the group consisting of O, N, and S; wherein a R^{K} moiety indicated as substituted is substituted with one or more Q, wherein Q is cyano; W can be O; R^{e} can be hydrogen; J can be C; X can be N; Y can be C; and Z can be C. In some embodiments, the compound of Formula (I-A) can be 5-(4-((2-(1H-indol-3-yl)ethyl)amino)furo[3,2-d]pyrimidin-2-yl)nicotinonitrile.

In some embodiments, the compound of Formula (I-A), or a pharmaceutically acceptable salt thereof, can selected from the group consisting of:
N-(2-(1H-indol-3-yl)ethyl)-7-isopropyl-2-(5-methylpyridin-3-yl)thieno[3,2-d]pyrimidin-4-amine;
5-(4-((2-(1H-indol-3-yl)ethyl)amino)-7-isopropylthieno[3,2d]pyrimidin-2-yl)nicotinonitrile;
N-(2-(1H-indol-3-yl)ethyl)-2-(5-fluoropyridin-3-yl)-7-isopropylthieno[3,2-d]pyrimidin-4-amine;
4-(2-((2-(benzo[b]thiophen-3-yl)-7-isopropylthieno[3,2-d]pyrimidin-4-yl)amino)ethyl)phenol;
N-(2-(1H-indol-3-yl)ethyl)-2-(5-fluoropyridin-3-yl)furo[3,2-d]pyrimidin-4-amine;
N-(2-(1H-indol-3-yl)ethyl)-2-(5-methylpyridin-3-yl)furo[3,2-d]pyrimidin-4-amine; and
5-(4-((2-(1H-indol-3-yl)ethyl)amino)furo[3,2d]pyrimidin-2-yl)nicotinonitrile.

### Formula (I-B)

In other embodiments provided herein, the compound of Formula (I) can have the structure of Formula (I-B):

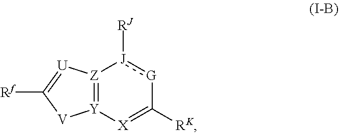

including pharmaceutically acceptable salts thereof, wherein: R^{a} can be hydrogen or C₁-C₄ alkyl; R^{b} can be R^{c} or —(C₁₋₄ alkyl)-R^{c}; R^{c} can be selected from the group consisting of: —OH, —O(C₁-C₄ alkyl), —O(C₁-C₄ haloalkyl); —C(═O)NH₂; unsubstituted C₆₋₁₀ aryl; substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{c} moiety indicated as substituted is substituted with one or more substituents E, wherein each E can be independently selected from the group consisting of: —OH, C₁-C₄ alkyl, C₁-C₄ haloalkyl, —O(C₁-C₄ alkyl), and —O(C₁-C₄ haloalkyl); R^{K} can be selected from the group consisting of: hydrogen, unsubstituted C₁₋₆ alkyl; substituted C₁₋₆ alkyl; —NH(C₁₋₄ alkyl); —N(C₁₋₄ alkyl)₂, unsubstituted C₆₋₁₀ aryl; substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{K} moiety indicated as substituted is substituted with one or more substituents Q, wherein each Q can be independently selected from the group consisting of: —OH, C₁₋₄ alkyl, C₁₋₄ haloalkyl, halo, cyano, —O—(C₁₋₄ alkyl), and —O—(C₁₋₄ haloalkyl); R^{G} can be selected from the group consisting of hydrogen, C₁₋₄ alkyl, and —(C₁₋₄ alkyl)-C(═O)NH₂; R^{f} can be selected from the group consisting of hydrogen, C₁₋₄ alkyl, unsubstituted C₆-C₁₀ aryl, and C₆-C₁₀ aryl substituted with 1-5 halo atoms; U can be N or CR^{U}; V can be S or NR^{V}; R^{U} can be selected from the group consisting of hydrogen, C₁₋₄ alkyl, halo, and cyano; R^{V} can be hydrogen or C₁-C₄ alkyl; wherein when U is CR^{U} and V is NR^{V}, R^{U} is selected from the group consisting of C₁₋₄ alkyl, halo, and cyano; Y and Z can each be C; and X can be N or CH.

In some embodiments, R^{a} can be hydrogen. In other embodiments, R^{a} can be C₁-C₄ alkyl.

In some embodiments, R^{b} can be —(C₁-C₄ alkyl)-R^{c}. For example, R^{b} can be —CH₂—R^{c}, —CH₂CH₂—R^{c}, —CH₂CH₂CH₂—R^{c}, or —CH₂CH₂CH₂CH₂—R^{C}. In certain embodiments, R^{b} can be —(CH₂CH₂)—R^{C}. In certain embodiments, R^{b} can be —(CH₂CH₂)—C(═O)NH₂. In certain embodiments, R^{b} can be —(CH₂CH₂)-(indolyl). In certain embodiments, R^{b} can be —(CH₂CH₂)-(hydroxyphenyl).

In some embodiments, R^{c} can be —OH. In some embodiments, R^{c} can be —O(C₁-C₄ alkyl). In some embodiments, R^{c} can be —O(C₁-C₄ haloalkyl). In some embodiments, R^{c} can be —C(═O)NH₂. In some embodiments, R^{c} can be unsubstituted C₆₋₁₀ aryl. In some embodiments, R^{c} can be substituted C₆₋₁₀ aryl. In some embodiments, R^{c} can be unsubstituted five- to ten-membered heteroaryl having 1-4 atoms selected from the group consisting of O, N, and S. In some embodiments, R^{c} can be substituted five- to ten-membered heteroaryl having 1-4 atoms selected from the group consisting of O, N, and S. In some embodiments, when a R^{c} moiety is indicated as substituted, the moiety can be substituted with one or more, for example, one, two, three, or four substituents E. In some embodiments, E can be —OH. In some embodiments, E can be C₁-C₄ alkyl. In some embodiments, E can be C₁-C₄ haloalkyl. In some embodiments, E can be —O(C₁-C₄ alkyl). In some embodiments, E can be —O(C₁-C₄ haloalkyl).

In some embodiments, R^{K} can be hydrogen. In other embodiments, R^{K} can be C₁-C₄ alkyl. For example, R^{K} can be methyl, ethyl, n-propyl, iso-propyl, n-butyl, iso-butyl or tert-butyl. In some embodiments, R^{K} can be selected from the group consisting of: unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein the substituted heteroaryl can substituted with one or more substituents Q, wherein each Q can independently selected from the group consisting of: —OH, C₁₋₄ alkyl, C₁₋₄ haloalkyl, halo, cyano, —O—(C₁₋₄ alkyl), and —O—(C₁₋₄ haloalkyl). In certain embodiments, R^{K} can be benzothiophenyl. In other embodiments, R^{K} can be pyridinyl substituted with one or more substituents Q. For example, R^{K} can be methylpyridinyl, ethylpyridinyl cyanopyridinyl, chloropyridinyl, fluoropyridinyl, or bromopyridinyl.

In some embodiments, R^{G} can be selected from the group consisting of hydrogen, C₁₋₄ alkyl, and —(C₁₋₄ alkyl)-C(═O)NH₂. In certain embodiments, R^{G} can be —(CH₂CH₂)—C(═O)NH₂.

In some embodiments, R^{f} can be hydrogen. In other embodiments, R^{f} can be CM alkyl. For example, R^{f} can be methyl, ethyl, n-propyl, iso-propyl, n-butyl, iso-butyl or tert-butyl. In some embodiments, R^{f} can be unsubstituted C₆-C₁₀ aryl. In other embodiments, R^{f} can be C₆-C₁₀ aryl substituted with 1-5 halo atoms. In certain embodiments, R^{f} can be phenyl substituted with 1-5 halo atoms. In certain embodiments, R^{f} can be fluorophenyl.

In some embodiments, U can be N. In other embodiments, U can be CR^{U}.

In some embodiments, V can be S. In other embodiments, V can be NR^{V}.

In some embodiments, R^{U} can be hydrogen. In some embodiments, R^{U} can be C₁₋₄ alkyl. In other embodiments R^{U} can be halo. For example, R^{U} can be fluoro, chloro, bromo, or iodo. In still other embodiments, R^{U} can be cyano.

In some embodiments, R^{V} can be hydrogen. In other embodiments, R^{V} can be C₁₋₄ alkyl. For example, R^{V} can be methyl, ethyl, n-propyl, iso-propyl, n-butyl, iso-butyl or tert-butyl. In some embodiments, Y and Z can each be C and X can be N. In other embodiments, Y and Z can each be C and X can be CH.

In some embodiments, R^{a} can be hydrogen; R^{b} can be —(C₁₋₄ alkyl)-R^{c}; R^{c} can be selected from the group consisting of: —C(═O)NH₂, unsubstituted C₆₋₁₀ aryl; substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{c} moiety indicated as substituted can be substituted with one or more substituents E, wherein each E can be independently selected from the group consisting of: —OH, C₁-C₄ alkyl, C₁-C₄ haloalkyl, —O(C₁-C₄ alkyl), and —O(C₁-C₄ haloalkyl); R^{K} can be selected from the group consisting of: unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein the substituted heteroaryl is substituted with one or more substituents Q, wherein each Q can be independently selected from the group consisting of: —OH, C₁₋₄ alkyl, C₁₋₄ haloalkyl, halo, cyano, —O—(C₁₋₄ alkyl), and —O—(C₁₋₄ haloalkyl); R^{G} is C₁₋₄ alkyl or —(C₁₋₄ alkyl)-C(═O)NH₂; R^{f} can be selected from the group consisting of hydrogen, unsubstituted phenyl, and phenyl substituted with 1-5 halo atoms; Y and Z each can be C; and X can be CH.

In some embodiments, R^{a} can be hydrogen; R^{b} can be —(CH₂—CH₂)—R^{c}; R^{c} can be selected from the group consisting of: —C(═O)NH₂, substituted phenyl and unsubstituted indolyl; wherein the substituted phenyl is substituted with one substituent E, wherein E can be —OH; R^{K} can be selected from the group consisting of: unsubstituted benzothiohenyl and substituted pyridinyl; wherein the substituted pyridinyl is substituted with one substituent Q, wherein Q can be selected from the group consisting of: C₁₋₄ alkyl, halo, and cyano; R^{G} can be —(CH₂CH₂)—C(═O)NH₂; R^{f} can be selected from the group consisting of hydrogen, phenyl, and fluorophenyl; Y and Z each can be C; and X can be CH.

In some embodiments, when V is S, R^{a} can be hydrogen or C₁-C₄ alkyl; R^{b} can be R^{c} or —(CH₂—CH₂)—R^{c}; R^{c} can be selected from the group consisting of: —C(═O)NH₂; unsubstituted C₆₋₁₀ aryl; substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{c} moiety indicated as substituted is substituted with one or more substituents E, wherein each E can be independently selected from the group consisting of: —OH, C₁-C₄ alkyl, and —O(C₁-C₄ alkyl); R^{K} can be selected from the group consisting of: hydrogen, unsubstituted C₁₋₆ alkyl; substituted C₁₋₆ alkyl; —NH(C₁₋₄ alkyl); and —N(C₁₋₄ alkyl)₂; wherein a R^{K} moiety indicated as substituted is substituted with one or more substituents Q, wherein each Q can be independently selected from the group consisting of: —OH, C₁₋₄ alkyl, halo, cyano, and —O—(C₁₋₄ alkyl; R^{G} can be selected from the group consisting of hydrogen, C₁₋₄ alkyl, and —(C₁₋₄ alkyl)-C(═O)NH₂; R^{f} can be selected from the group consisting of hydrogen, C₁₋₄ alkyl, unsubstituted C₆-C₁₀ aryl, and C₆-C₁₀ aryl substituted with 1-5 halo atoms; U can be CR^{U}; R^{U} can be selected from the group consisting of hydrogen, C₁₋₄ alkyl, halo, and cyano; Y and Z can each be C; and X can be N.

In some embodiments, when V is NR^{V}, R^{a} can be hydrogen or C₁-C₄ alkyl; R^{b} can be R^{c} or —(CH₂—CH₂)—R^{c}; R^{c} can be selected from the group consisting of: —C(═O)NH₂; unsubstituted C₆₋₁₀ aryl; substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{c} moiety indicated as substituted is substituted with one or more substituents E, wherein each E can be independently selected from the group consisting of: —OH, C₁-C₄ alkyl, C₁-C₄, and —O(C₁-C₄ alkyl); R^{K} can be selected from the group consisting of: unsubstituted C₆₋₁₀ aryl; substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{K} moiety indicated as substituted is substituted with one or more substituents Q, wherein each Q can be independently selected from the group consisting of: —OH, C₁₋₄ alkyl, halo, cyano, and —O—(C₁₋₄ alkyl); R^{G} can be selected from the group consisting of hydrogen, C₁₋₄ alkyl, and —(C₁₋₄ alkyl)-C(═O)NH₂; R^{f} can be hydrogen; U can be N or CR^{U}; R^{U} can be selected from the group consisting of C₁₋₄ alkyl, halo, and cyano; R^{V} can be hydrogen or C₁-C₄ alkyl; Y and Z can each be C; and X can be N or CH.

In some embodiments, when R^{J} is —OR^{b}; G can be N; [symbol] joining G and J can be a double bond; R^{b} can be —CH₂CH₂—R^{c}; R^{c} can be

—C(═O)NH₂; R^{K} can unsubstituted five- to ten-membered heteroaryl having 1-4 atoms selected from the group consisting of O, N, and S; U can N; V can be NR^{V}; R^{V} can be C₁-C₄ alkyl; R^{f} can be hydrogen; J can be C; X can be N; Y can be C; and Z can be C. In some embodiments, the compound of Formula (I-B) can be 3-((2-(benzo[b]thiophen-3-yl)-9-isopropyl-9H-purin-6-yl)oxy)propanamide.

In some embodiments, when R^{J} is ═O; G can be N substituted with R^{G}; [symbol] joining G and J can be a single bond; R^{G} can be —(C₁₋₄ alkyl)-C(═O)NH₂; R^{K} can be unsubstituted five- to ten-membered heteroaryl having 1-4 atoms selected from the group consisting of O, N, and S; U can N; V can be NR^{V}; R^{V} can be C₁-C₄ alkyl; R^{f} can be hydrogen; J can be C; X can be N; Y can be C; and Z can be C. In some embodiments, the compound of Formula (I-B) can be 3-(2-(benzo[b]thiophen-3-yl)-9-isopropyl-6-oxo-6,9-dihydro-1H-purin-1-yl)propanamide.

In some embodiments, when R^{J} is —NR^{a}R^{b}; G can be N; [symbol] joining G and J can be a double bond; R^{a} can be hydrogen; R^{b} can be —CH₂CH₂—R^{c}; R^{c} can be substituted C₆₋₁₀ aryl, substituted with one or more E, wherein E is —OH; R^{K} can be unsubstituted five- to ten-membered heteroaryl having 1-4 atoms selected from the group consisting of O, N, and S; U can be CR^{U}; R^{U} can be cyano; V can be NR^{V}; R^{V} can be C₁-C₄ alkyl; R^{f} can be hydrogen; J can be C; X can be N; Y can be C; and Z can be C. In some embodiments, the compound of Formula (I-B) can be 2-(benzo[b]thiophen-3-yl)-4-((4-hydroxyphenethyl)amino)-7-isopropyl-7H-pyrrolo[2,3-d]pyrimidine-5-carbonitrile.

In some embodiments, when R^{J} is —NR^{a}R^{b}; G can be N; [symbol] joining G and J can be a double bond; R^{a} can be hydrogen; R^{b} can be —CH₂CH₂—R^{c}; R^{c} can be unsubstituted five- to ten-membered heteroaryl having 1-4 atoms selected from the group consisting of O, N, and S; R^{K} can be unsubstituted C₁₋₆ alkyl; U can be CR^{U}; R^{U} can be hydrogen; V can be S; R^{f} can be phenyl; J can be C; X can be N; Y can be C; Z can be C. In some embodiments, the compound of Formula (I-B) can be N-(2-(1H-indol-3-yl)ethyl)-2-methyl-6-phenylthieno[2,3-d]pyrimidin-4-amine.

In some embodiments, when R^{J} can be —NR^{a}R^{b}; G can be N; [symbol] joining G and J can be a double bond; R^{a} can be hydrogen; R^{b} can be —CH₂CH₂—R^{c}; R^{C} can be unsubstituted five- to ten-membered heteroaryl having 1-4 atoms selected from the group consisting of O, N, and S; R^{K} can be hydrogen; U can be CR^{U}; R^{U} can be hydrogen; V can be S; R^{f} can be fluorophenyl; J can be C; X can be N; Y can be C; and Z can be C. In some embodiments, the compound of Formula (I-B) can be N-(2-(1H-indol-3-yl)ethyl)-6-(4-fluorophenyl)thieno[2,3-d]pyrimidin-4-amine.

In some embodiments, the compound of Formula (I-B), or a pharmaceutically acceptable salt thereof, can selected from the group consisting of:
3-((2-(benzo[b]thiophen-3-yl)-9-isopropyl-9H-purin-6-yl)oxy)propanamide;
3-(2-(benzo[b]thiophen-3-yl)-9-isopropyl-6-oxo-6,9-dihydro-1H-purin-1-yl)propanamide;
2-(benzo[b]thiophen-3-yl)-4-((4-hydroxyphenethyl)amino)-7-isopropyl-7H-pyrrolo[2,3-d]pyrimidine-5-carbonitrile;
N-(2-(1H-indol-3-yl)ethyl)-2-methyl-6-phenylthieno[2,3-d]pyrimidin-4-amine; and
N-(2-(1H-indol-3-yl)ethyl)-6-(4-fluorophenyl)thieno[2,3-d]pyrimidin-4-amine.

### Formula (I-C)

In still other embodiments provided herein, the compound of Formula (I) can have the structure of Formula (I-C):

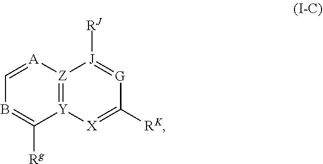

including pharmaceutically acceptable salts thereof, wherein: R^{J} can be —NR^{a}R^{b}; R^{a} can be hydrogen or C₁-C₄ alkyl; R^{b} can be R^{c} or —(C₁-C₄ alkyl)-R^{c}; R^{c} can be selected from the group consisting of: unsubstituted C₆₋₁₀ aryl; substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{c} moiety indicated as substituted is substituted with one or more substituents E, wherein each E can be independently selected from the group consisting of: —OH, C₁-C₄ alkyl, C₁-C₄ haloalkyl, —O(C₁-C₄ alkyl), and —O(C₁-C₄ haloalkyl); R^{K} can be selected from the group consisting of: hydrogen, unsubstituted C₁₋₆ alkyl; —NH(C₁₋₄ alkyl); —N(C₁₋₄ alkyl)₂, unsubstituted C₆₋₁₀ aryl; substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{K} moiety indicated as substituted is substituted with one or more substituents Q, wherein each Q can be independently selected from the group consisting of: —OH, C₁₋₄ alkyl, C₁₋₄ haloalkyl, halo, cyano, —O—(C₁₋₄ alkyl), and —O—(C₁₋₄ haloalkyl); A can be N or CH; B can be N or CH; R^{g} can be selected from the group consisting of hydrogen, C₁₋₄ alkyl, and —N(C₁₋₄ alkyl)₂; Y and Z can each be C; and X can be N or CH.

In some embodiments, R^{K} can be —NH(C₁₋₄ alkyl). For example, in some embodiments, R^{K} can be —NH(CH₃), —NH(CH₂CH₃), —NH(isopropyl), or —NH(sec-butyl). In some embodiments, R^{K} can be unsubstituted benzothiophenyl. In other embodiments, R^{K} can be substituted pyridinyl. For example, R^{K} can be methylpyridinyl, ethylpyridinyl, cyanopyridinyl, chloropyridinyl, fluoropyridinyl, or bromopyridinyl.

In some embodiments, A can be N and B can be N. In other embodiments, A can be N and B can be CH. In still other embodiments, A can be CH and B can be N. In yet still other embodiments, A can be CH and B can be CH.

In some embodiments, R^{g} can be hydrogen. In other embodiments, R^{g} can be —N(C₁₋₄ alkyl)₂. In certain embodiments, R^{g} can be

—N(CH₃)₂.

In some embodiments, R^{a} can be hydrogen; R^{b} can be —(C₁-C₄ alkyl)-R^{c}; R^{c} can be selected from the group consisting of: unsubstituted C₆₋₁₀ aryl; substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{c} moiety indicated as substituted is substituted with one or more substituents E, wherein each E can be independently selected from the group consisting of: —OH, C₁-C₄ alkyl, C₁-C₄ haloalkyl, —O(C₁-C₄ alkyl), and —O(C₁-C₄ haloalkyl); R^{K} can be selected from the group consisting of: —NH(C₁₋₄ alkyl); unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein the substituted heteroaryl is substituted with one or more substituents Q, wherein each Q can be independently selected from the group consisting of: —OH, C₁₋₄ alkyl, C₁₋₄ haloalkyl, halo, cyano, —O—(C₁₋₄ alkyl), and —O—(C₁₋₄ haloalkyl); and R^{g} can be hydrogen or —N(C₁₋₄ alkyl)₂.

In some embodiments, R^{a} can be hydrogen; R^{b} can be —(C₁-C₄ alkyl)-R^{c}; R^{c} can be selected from the group consisting of: substituted phenyl and unsubstituted indolyl; wherein the substituted phenyl is substituted with one or more substituents E, wherein each E can be independently selected from the group consisting of: —OH, C₁-C₄ alkyl, C₁-C₄ haloalkyl, —O(C₁-C₄ alkyl), and —O(C₁-C₄ haloalkyl); R^{K} can be selected from the group consisting of: —NH(C₁₋₄ alkyl); unsubstituted benzothiophenyl; and substituted pyridinyl; wherein the substituted pyridinyl is substituted with one or more substituents Q, wherein each Q can be independently selected from the group consisting of: —OH, C₁₋₄ alkyl, C₁₋₄ haloalkyl, halo, cyano, —O—(C₁₋₄ alkyl), and —O—(C₁₋₄ haloalkyl); and R^{g} can be hydrogen or —N(C₁₋₄ alkyl)₂.

In some embodiments, R^{a} can be hydrogen; R^{b} can be —(CH₂CH₂)—R^{c}; R^{c} can be selected from the group consisting of: substituted phenyl and unsubstituted indolyl; wherein the substituted phenyl is substituted with one substituent E, wherein E can be —OH; R^{K} can be selected from the group consisting of: —NH(sec-butyl); unsubstituted benzothiohenyl, and substituted pyridinyl; wherein the substituted pyridinyl is substituted with one or more substituents Q, wherein each Q can be independently selected from the group consisting of: C₁₋₄ alkyl, halo, and cyano; and R^{g} can be hydrogen or —N(CH₃)₂.

In some embodiments, when A is C and B is C, R^{J} can be —NR^{a}R^{b}; G can be N; R^{a} can be hydrogen; R^{b} can be —CH₂CH₂—R^{c}; R^{c} can be substituted C₆₋₁₀ aryl, substituted with one or more E, wherein E is —OH; or unsubstituted five- to ten-membered heteroaryl having 1-4 atoms selected from the group consisting of O, N, and S; R^{K} can be unsubstituted five- to ten-membered heteroaryl having 1-4 atoms selected from the group consisting of O, N, and S; R^{g} can be hydrogen; J can be C; X can be N; Y can be C; and Z is C.

In some embodiments, when R^{J} is —NR^{a}R^{b}; G can be N; R^{a} can be hydrogen; R^{b} can be —CH₂CH₂—R^{c}; R^{c} can be substituted C₆₋₁₀ aryl, substituted with one or more E, wherein E is —OH; R^{K} is unsubstituted five- to ten-membered heteroaryl having 1-4 atoms selected from the group consisting of O, N, and S; A can be N; B can be N; R^{g} can be —N(C₁₋₄ alkyl)₂; J can be C; X can be N; Y can be C; and Z is C. In some embodiments, the compound of Formula (I-C) can be 4-(2-((2-(benzo[b]thiophen-3-yl)-8-(dimethylamino)pyrimido[5,4-d]pyrimidin-4-yl)amino)ethyl)phenol.

In some embodiments, when R^{J} is —NR^{a}R^{b}; G can be N; R^{a} can be hydrogen R^{b} can be —CH₂CH₂—R^{c}; R^{c} can be unsubstituted five- to ten-membered heteroaryl having 1-4 atoms selected from the group consisting of O, N, and S; R^{K} can be substituted five- to ten-membered heteroaryl having 1-4 atoms selected from the group consisting of O, N, and S; wherein a R^{K} moiety indicated as substituted is substituted with one or more Q, wherein Q can be halo; A can be CH; B can be CH; R^{g} can be hydrogen; J can be C; X can be N; Y can be C; and Z can be C. In some embodiments, the compound of Formula (I-C) can be N-(2-(1H-indol-3-yl)ethyl)-2-(5-fluoropyridin-3-yl)quinazolin-4-amine.

In some embodiments, when R^{J} is —NR^{a}R^{b}; G is N; [symbol] joining G and J can be a double bond; R^{a} can be hydrogen R^{b} can be —CH₂CH₂—R^{c}; R^{c} can be unsubstituted five- to ten-membered heteroaryl having 1-4 atoms selected from the group consisting of O, N, and S; R^{K} can be substituted five- to ten-membered heteroaryl having 1-4 atoms selected from the group consisting of O, N, and S; wherein a R^{K} moiety indicated as substituted is substituted with one or more Q, wherein Q can be cyano; A can be CH; B can be CH; R^{g} can be hydrogen; J can be C; X can be N; Y can be C; and Z can be C. In some embodiments, the compound of Formula (I-C) can be 5-(4-((2-(1H-indol-3-yl)ethyl)amino)quinazolin-2-yl)nicotinonitrile.

In some embodiments, when R^{J} is —NR^{a}R^{b}; G can be N; [symbol] joining G and J can be a double bond; R^{a} can be hydrogen R^{b} can be —CH₂CH₂—R^{c}; R^{c} can be unsubstituted five- to ten-membered heteroaryl having 1-4 atoms selected from the group consisting of O, N, and S; R^{K} can be —NH(C₁₋₄ alkyl); A can be CH; B can be CH; R^{g} can be hydrogen; J can be C; X can be N; Y can be C; and Z can be C. In some embodiments, the compound of Formula (I-C) can be N⁴-(2-(1H-indol-3-yl)ethyl)-N²-(sec-butyl)quinazoline-2,4-diamine.

In some embodiments, the compound of Formula (I-C), or a pharmaceutically acceptable salt thereof, can selected from the group consisting of:
4-(2-((2-(benzo[b]thiophen-3-yl)-8-(dimethylamino)pyrimido[5,4-d]pyrimidin-4-yl)amino)ethyl)phenol;
N-(2-(1H-indol-3-yl)ethyl)-2-(5-fluoropyridin-3-yl)quinazolin-4-amine;
5-(4-((2-(1H-indol-3-yl)ethyl)amino)quinazolin-2-yl)nicotinonitrile; and
N⁴-(2-(1H-indol-3-yl)ethyl)-N²-(sec-butyl)quinazoline-2,4-diamine.

### Formula (I-D)

In yet still other embodiments provided herein, the compound of Formula (I) can have the structure of Formula (I-D):

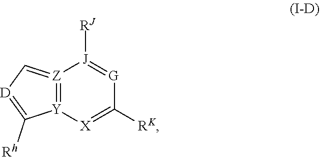

including pharmaceutically acceptable salts thereof, wherein: R^{J} can be —NR^{a}R^{b}; R^{a} can be hydrogen or C₁-C₄ alkyl; R^{b} can be R^{c} or —(C₁₋₄ alkyl)-R^{c}; R^{c} can be selected from the group consisting of: unsubstituted C₆₋₁₀ aryl; substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{c} moiety indicated as substituted is substituted with one or more substituents E, wherein each E can be independently selected from the group consisting of: —OH, C₁-C₄ alkyl, C₁-C₄ haloalkyl, —O(C₁-C₄ alkyl), and —O(C₁-C₄ haloalkyl); R^{K} can be selected from the group consisting of: unsubstituted C₆₋₁₀ aryl; substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{K} moiety indicated as substituted is substituted with one or more substituents Q, wherein each Q can be independently selected from the group consisting of: —OH, C₁₋₄ alkyl, C₁₋₄ haloalkyl, halo, cyano, —O—(C₁₋₄ alkyl), and —O—(C₁₋₄ haloalkyl); R^{h} can be hydrogen or C₁₋₄ alkyl; D can be N or CH; Y can be N; Z can be C; and X can be N or CH.

In some embodiments, R^{h} can be hydrogen. In other embodiments, R^{h} can be C₁₋₄ alkyl. For example, R^{h} can be methyl, ethyl, n-propyl, iso-propyl, n-butyl, iso-butyl or tert-butyl.

In some embodiments, D can be N. In other embodiments, D can be CH.

In some embodiments, when D is N, Y can be N, Z can be C, and X can be N. In other embodiments, when D is N, Y can be N, Z can be C, and X can be CH. In some embodiments, when D is CH, Y can be N, Z can be C, and X can be N. In other embodiments, when D is CH, Y can be N, Z can be C, and X can be CH.

In some embodiments, R^{a} can be hydrogen; R^{b} can be —(C₁₋₄ alkyl)-R^{c}; R^{c} can be selected from the group consisting of: unsubstituted C₆₋₁₀ aryl; substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{c} moiety indicated as substituted is substituted with one or more substituents E, wherein each E can be independently selected from the group consisting of: —OH, C₁-C₄ alkyl, C₁-C₄ haloalkyl, —O(C₁-C₄ alkyl), and —O(C₁-C₄ haloalkyl); R^{K} can be selected from the group consisting of: unsubstituted C₆₋₁₀ aryl; substituted C₆₋₁₀ aryl; unsubstituted five- to ten-membered heteroaryl having 1-4 atoms selected from the group consisting of O, N, and S; and substituted five- to ten-membered heteroaryl having 1-4 atoms selected from the group consisting of O, N, and S; wherein a R^{K} moiety indicated as substituted is substituted with one or more substituents Q, wherein each Q can be independently selected from the group consisting of: —OH, C₁₋₄ alkyl, C₁₋₄ haloalkyl, halo, cyano, —O—(C₁₋₄ alkyl), and —O—(C₁₋₄ haloalkyl); and R^{h} can be hydrogen or C₁₋₄ alkyl.

In some embodiments, R^{a} can be hydrogen; R^{b} can be —(C₁-C₄ alkyl)-R^{c}; R^{c} can be selected from the group consisting of: substituted phenyl and unsubstituted indolyl; wherein the substituted phenyl is substituted with one or more substituents E, wherein each E can be independently selected from the group consisting of: —OH, C₁-C₄ alkyl, C₁-C₄ haloalkyl, —O(C₁-C₄ alkyl), and —O(C₁-C₄ haloalkyl); R^{K} can be unsubstituted benzothiophenyl; and R^{h} can be hydrogen or C₁₋₄ alkyl.

In some embodiments, R^{a} can be hydrogen; R^{b} can be —(CH₂—CH₂)—R^{c}; R^{c} can be selected from the group consisting of: substituted phenyl and unsubstituted indolyl; wherein the substituted phenyl is substituted with one substituent E, wherein E can be —OH; R^{K} can be unsubstituted benzothiophenyl; and R^{h} can be hydrogen or C₁₋₄ alkyl.

In some embodiments, when D is N; R^{J} is —NR^{a}R^{b}; G can be N; R^{a} can be hydrogen; R^{b} can be —CH₂CH₂—R^{c}; R^{c} can be unsubstituted five- to ten-membered heteroaryl having 1-4 atoms selected from the group consisting of O, N, and S; or substituted C₆₋₁₀ aryl, substituted with one or more E, wherein E is —OH; R^{K} can be unsubstituted five- to ten-membered heteroaryl having 1-4 atoms selected from the group consisting of O, N, and S; R^{h} can be C₁₋₄ alkyl; J can be C; X can be C; Y can be N; and Z can be C; wherein the valency of any carbon atom is filled as needed with hydrogen atoms.

In some embodiments, when R^{J} is —NR^{a}R^{b}; G can be N; R^{a} can be hydrogen; R^{b} can be —CH₂CH₂—R^{c}; R^{c} can be unsubstituted five- to ten-membered heteroaryl having 1-4 atoms selected from the group consisting of O, N, and S or substituted C₆₋₁₀ aryl, substituted with one or more E, wherein E is —OH; R^{K} can be unsubstituted five- to ten-membered heteroaryl having 1-4 atoms selected from the group consisting of O, N, and S; D can be N; R^{h} can be C₁₋₄ alkyl; J can be C; X can be C; Y can be N; and Z can be C; wherein the valency of any carbon atom is filled as needed with hydrogen atoms. In some embodiments, the compound of Formula (I-D) can be N-(2-(1H-indol-3-yl)ethyl)-6-(benzo[b]thiophen-3-yl)-3-isopropylimidazo[1,5-a]pyrazin-8-amine.

In some embodiments, when R^{J} is —NR^{a}R^{b}; G can be N; [symbol] joining G and J can be a double bond; R^{a} can be hydrogen; R^{b} can be —CH₂CH₂—R^{c}; R^{c} can be substituted C₆₋₁₀ aryl, substituted with one or more E, wherein E is —OH; R^{K} can be unsubstituted five- to ten-membered heteroaryl having 1-4 atoms selected from the group consisting of O, N, and S; D can be N; R^{h} can be C₁₋₄ alkyl; J can be C; X can be C; Y can be N; and Z can be C; wherein the valency of any carbon atom is filled as needed with hydrogen atoms. In some embodiments, the compound of Formula (I-D) can be 4-(2-((6-(benzo[b]thiophen-3-yl)-3-isopropylimidazo[1,5-a]pyrazin-8-yl)amino)ethyl)phenol.

In some embodiments, the compound of Formula (I-D), or a pharmaceutically acceptable salt thereof, can selected from the group consisting of: N-(2-(1H-indol-3-yl)ethyl)-6-(benzo[b]thiophen-3-yl)-3-isopropylimidazo[1,5-a]pyrazin-8-amine; and 4-(2-((6-(benzo[b]thiophen-3-yl)-3-isopropylimidazo[1,5-a]pyrazin-8-yl)amino)ethyl)phenol.

The compounds provided herein may be enantiomerically pure, such as a single enantiomer or a single diastereomer, or be stereoisomeric mixtures, such as a mixture of enantiomers, e.g., a racemic mixture of two enantiomers; or a mixture of two or more diastereomers. As such, one of skill in the art will recognize that administration of a compound in its (R) form is equivalent, for compounds that undergo epimerization in vivo, to administration of the compound in its (S) form. Conventional techniques for the preparation/isolation of individual enantiomers include synthesis from a suitable optically pure precursor, asymmetric synthesis from achiral starting materials, or resolution of an enantiomeric mixture, for example, chiral chromatography, recrystallization, resolution, diastereomeric salt formation, or derivatization into diastereomeric adducts followed by separation.

5.4. Isolation of NK Cells

Methods of isolating natural killer cells are known in the art and can be used to isolate the natural killer cells, e.g., NK cells produced using the three-stage method, described herein. For example, NK cells can be isolated or enriched, for example, by staining cells, in one embodiment, with antibodies to CD56 and CD3, and selecting for CD56⁺CD3⁻ cells. In certain embodiments, the NK cells are enriched for CD56⁺CD3⁻ cells in comparison with total cells produced using the three-stage method, described herein. NK cells, e.g., cells produced using the three-stage method, described herein, can be isolated using a commercially available kit, for example, the NK Cell Isolation Kit (Miltenyi Biotec). NK cells, e.g., cells produced using the three-stage method, described herein, can also be isolated or enriched by removal of cells other than NK cells in a population of cells that comprise the NK cells, e.g., cells produced using the three-stage method, described herein. For example, NK cells, e.g., cells produced using the three-stage method, described herein, may be isolated or enriched by depletion of cells displaying non-NK cell markers using, e.g., antibodies to one or more of CD3, CD4, CD14, CD19, CD20, CD36, CD66b, CD123, HLA DR and/or CD235a (glycophorin A). Negative isolation can be carried out using a commercially available kit, e.g., the NK Cell Negative Isolation Kit (Dynal Biotech). Cells isolated by these methods may be additionally sorted, e.g., to separate CD11a+ and CD11a− cells, and/or CD117+ and CD117− cells, and/or CD16⁺ and CD16⁻ cells, and/or CD94⁺ and CD94⁻. In certain embodiments, cells, e.g., cells produced by the three-step methods described herein, are sorted to separate CD11a+ and CD11a− cells. In specific embodiments, CD11a+ cells are isolated. In certain embodiments, the cells are enriched for CD11a⁺ cells in comparison with total cells produced using the three-stage method, described herein. In specific embodiments, CD11a− cells are isolated. In certain embodiments, the cells are enriched for CD11a− cells in comparison with total cells produced using the three-stage method, described herein. In certain embodiments, cells are sorted to separate CD117+ and CD117− cells. In specific embodiments, CD117+ cells are isolated. In certain embodiments, the cells are enriched for CD117⁺ cells in comparison with total cells produced using the three-stage method, described herein. In specific embodiments, CD117− cells are isolated. In certain embodiments, the cells are enriched for CD117− cells in comparison with total cells produced using the three-stage method, described herein. In certain embodiments, cells are sorted to separate CD16⁺ and CD16⁻ cells. In specific embodiments, CD16⁺ cells are isolated. In certain embodiments, the cells are enriched for CD16⁺ cells in comparison with total cells produced using the three-stage method, described herein. In specific embodiments, CD16⁻ cells are isolated. In certain embodiments, the cells are enriched for CD16− cells in comparison with total cells produced using the three-stage method, described herein. In certain embodiments, cells are sorted to separate CD94⁺ and CD94⁻ cells. In specific embodiments, CD94⁺ cells are isolated. In certain embodiments, the cells are enriched for CD94⁺ cells in comparison with total cells produced using the three-stage method, described herein. In specific embodiments, CD94⁻ cells are isolated. In certain embodiments, the cells are enriched for CD94− cells in comparison with total cells produced using the three-stage method, described herein. In certain embodiments, isolation is performed using magnetic separation. In certain embodiments, isolation is performed using flow cytometry.

Methods of isolating ILC3 cells are known in the art and can be used to isolate the ILC3 cells, e.g., ILC3 cells produced using the three-stage method, described herein. For example, ILC3 cells can be isolated or enriched, for example, by staining cells, in one embodiment, with antibodies to CD56, CD3, and CD11a, and selecting for CD56⁺CD3⁻CD11a⁻ cells. ILC3 cells, e.g., cells produced using the three-stage method, described herein, can also be isolated or enriched by removal of cells other than ILC3 cells in a population of cells that comprise the ILC3 cells, e.g., cells produced using the three-stage method, described herein. For example, ILC3 cells, e.g., cells produced using the three-stage method, described herein, may be isolated or enriched by depletion of cells displaying non-ILC3 cell markers using, e.g., antibodies to one or more of CD3, CD4, CD11a, CD14, CD19, CD20, CD36, CD66b, CD94, CD123, HLA DR and/or CD235a (glycophorin A). Cells isolated by these methods may be additionally sorted, e.g., to separate CD117⁺ and CD117⁻ cells. NK cells can be isolated or enriched, for example, by staining cells, in one embodiment, with antibodies to CD56, CD3, CD94, and CD11a, and selecting for CD56⁺CD3⁻CD94⁺CD11a⁺ cells. NK cells, e.g., cells produced using the three-stage method, described herein, can also be isolated or enriched by removal of cells other than NK cells in a population of cells that comprise the NK cells, e.g., cells produced using the three-stage method, described herein. In certain embodiments, the NK cells are enriched for CD56⁺CD3⁻CD94⁺CD11a⁺ cells in comparison with total cells produced using the three-stage method, described herein.

In one embodiment, ILC3 cells are isolated or enriched by selecting for CD56⁺CD3⁻CD11a⁻ cells. In certain embodiments, the ILC3 cells are enriched for CD56⁺CD3⁻CD11a⁻ cells in comparison with total cells produced using the three-stage method, described herein. In one embodiment, ILC3 cells are isolated or enriched by selecting for CD56⁺CD3⁻CD11a⁻CD117+ cells. In certain embodiments, the ILC3 cells are enriched for CD56⁺CD3⁻CD11a⁻CD117+ cells in comparison with total cells produced using the three-stage method, described herein. In one embodiment, ILC3 cells are isolated or enriched by selecting for CD56⁺CD3⁻CD11a⁻CD117⁺CDIL1R1⁺ cells. In certain embodiments, the ILC3 cells are enriched for CD56⁺CD3⁻CD11a⁻CD117⁺CDIL1R1⁺ cells in comparison with total cells produced using the three-stage method, described herein.

In one embodiment, NK cells are isolated or enriched by selecting for CD56⁺CD3⁻CD94⁺CD11a⁺ cells. In certain embodiments, the NK cells are enriched for CD56⁺CD3⁻CD94⁺CD11a⁺ cells in comparison with total cells produced using the three-stage method, described herein. In one embodiment, NK cells are isolated or enriched by selecting for CD56⁺CD3⁻CD94⁺CD11a⁺CD117⁻ cells. In certain embodiments, the NK cells are enriched for CD56⁺CD3⁻CD94⁺CD11a⁺CD117⁻ cells in comparison with total cells produced using the three-stage method, described herein.

Cell separation can be accomplished by, e.g., flow cytometry, fluorescence-activated cell sorting (FACS), or, in one embodiment, magnetic cell sorting using microbeads conjugated with specific antibodies. The cells may be isolated, e.g., using a magnetic activated cell sorting (MACS) technique, a method for separating particles based on their ability to bind magnetic beads (e.g., about 0.5-100 μm diameter) that comprise one or more specific antibodies, e.g., anti-CD56 antibodies. Magnetic cell separation can be performed and automated using, e.g., an AUTOMACS™ Separator (Miltenyi). A variety of useful modifications can be performed on the magnetic microspheres, including covalent addition of antibody that specifically recognizes a particular cell surface molecule or hapten. The beads are then mixed with the cells to allow binding. Cells are then passed through a magnetic field to separate out cells having the specific cell surface marker. In one embodiment, these cells can then isolated and re-mixed with magnetic beads coupled to an antibody against additional cell surface markers. The cells are again passed through a magnetic field, isolating cells that bound both the antibodies. Such cells can then be diluted into separate dishes, such as microtiter dishes for clonal isolation.

5.5. Placental Perfusate

NK cells and/or ILC3 cells, e.g., NK cell and/or ILC3 cell populations produced according to the three-stage method described herein may be produced from hematopoietic cells, e.g., hematopoietic stem or progenitors from any source, e.g., placental tissue, placental perfusate, umbilical cord blood, placental blood, peripheral blood, spleen, liver, or the like. In certain embodiments, the hematopoietic stem cells are combined hematopoietic stem cells from placental perfusate and from cord blood from the same placenta used to generate the placental perfusate. Placental perfusate comprising placental perfusate cells that can be obtained, for example, by the methods disclosed in U.S. Pat. Nos. 7,045,148 and 7,468,276 and U.S. Patent Application Publication No. 2009/0104164, the disclosures of which are hereby incorporated in their entireties.

5.5.1. Cell Collection Composition

The placental perfusate and perfusate cells, from which hematopoietic stem or progenitors may be isolated, or useful in tumor suppression or the treatment of an individual having tumor cells, cancer or a viral infection, e.g., in combination with the NK cells and/or ILC3 cells, e.g., NK cell and/or ILC3 cell populations produced according to the three-stage method provided herein, can be collected by perfusion of a mammalian, e.g., human post-partum placenta using a placental cell collection composition. Perfusate can be collected from the placenta by perfusion of the placenta with any physiologically-acceptable solution, e.g., a saline solution, culture medium, or a more complex cell collection composition. A cell collection composition suitable for perfusing a placenta, and for the collection and preservation of perfusate cells is described in detail in related U.S. Application Publication No. 2007/0190042, which is incorporated herein by reference in its entirety.

The cell collection composition can comprise any physiologically-acceptable solution suitable for the collection and/or culture of stem cells, for example, a saline solution (e.g., phosphate-buffered saline, Kreb's solution, modified Kreb's solution, Eagle's solution, 0.9% NaCl. etc), a culture medium (e.g., DMEM, H.DMEM, etc), and the like.

The cell collection composition can comprise one or more components that tend to preserve placental cells, that is, prevent the placental cells from dying, or delay the death of the placental cells, reduce the number of placental cells in a population of cells that die, or the like, from the time of collection to the time of culturing. Such components can be, e.g., an apoptosis inhibitor (e.g., a caspase inhibitor or INK inhibitor); a vasodilator (e.g., magnesium sulfate, an antihypertensive drug, atrial natriuretic peptide (ANP), adrenocorticotropin, corticotropin-releasing hormone, sodium nitroprusside, hydralazine, adenosine triphosphate, adenosine, indomethacin or magnesium sulfate, a phosphodiesterase inhibitor, etc); a necrosis inhibitor (e.g., 2-(1H-Indol-3-yl)-3-pentylamino-maleimide, pyrrolidine dithiocarbamate, or clonazepam); a TNF-α inhibitor; and/or an oxygen-carrying perfluorocarbon (e.g., perfluorooctyl bromide, perfluorodecyl bromide, etc).

The cell collection composition can comprise one or more tissue-degrading enzymes, e.g., a metalloprotease, a serine protease, a neutral protease, a hyaluronidase, an RNase, or a DNase, or the like. Such enzymes include, but are not limited to, collagenases (e.g., collagenase I, II, III or IV, a collagenase from Clostridium histolyticum, etc); dispase, thermolysin, elastase, trypsin, LIB ERASE, hyaluronidase, and the like.

The cell collection composition can comprise a bacteriocidally or bacteriostatically effective amount of an antibiotic. In certain non-limiting embodiments, the antibiotic is a macrolide (e.g., tobramycin), a cephalosporin (e.g., cephalexin, cephradine, cefuroxime, cefprozil, cefaclor, cefixime or cefadroxil), a clarithromycin, an erythromycin, a penicillin (e.g., penicillin V) or a quinolone (e.g., ofloxacin, ciprofloxacin or norfloxacin), a tetracycline, a streptomycin, etc. In a particular embodiment, the antibiotic is active against Gram(+) and/or Gram(−) bacteria, e.g., Pseudomonas aeruginosa, Staphylococcus aureus, and the like.

The cell collection composition can also comprise one or more of the following compounds: adenosine (about 1 mM to about 50 mM); D-glucose (about 20 mM to about 100 mM); magnesium ions (about 1 mM to about 50 mM); a macromolecule of molecular weight greater than 20,000 daltons, in one embodiment, present in an amount sufficient to maintain endothelial integrity and cellular viability (e.g., a synthetic or naturally occurring colloid, a polysaccharide such as dextran or a polyethylene glycol present at about 25 g/l to about 100 g/1, or about 40 g/l to about 60 g/1); an antioxidant (e.g., butylated hydroxyanisole, butylated hydroxytoluene, glutathione, vitamin C or vitamin E present at about 25 μM to about 100 μM); a reducing agent (e.g., N-acetylcysteine present at about 0.1 mM to about 5 mM); an agent that prevents calcium entry into cells (e.g., verapamil present at about 2 μM to about 25 μM); nitroglycerin (e.g., about 0.05 g/L to about 0.2 g/L); an anticoagulant, in one embodiment, present in an amount sufficient to help prevent clotting of residual blood (e.g., heparin or hirudin present at a concentration of about 1000 units/1 to about 100,000 units/1); or an amiloride containing compound (e.g., amiloride, ethyl isopropyl amiloride, hexamethylene amiloride, dimethyl amiloride or isobutyl amiloride present at about 1.0 μM to about 5 μM).

5.5.2. Collection and Handling of Placenta

Generally, a human placenta is recovered shortly after its expulsion after birth. In one embodiment, the placenta is recovered from a patient after informed consent and after a complete medical history of the patient is taken and is associated with the placenta. In one embodiment, the medical history continues after delivery.

Prior to recovery of perfusate, the umbilical cord blood and placental blood are removed. In certain embodiments, after delivery, the cord blood in the placenta is recovered. The placenta can be subjected to a conventional cord blood recovery process. Typically a needle or cannula is used, with the aid of gravity, to exsanguinate the placenta (see, e.g., Anderson, U.S. Pat. No. 5,372,581; Hessel et al., U.S. Pat. No. 5,415,665). The needle or cannula is usually placed in the umbilical vein and the placenta can be gently massaged to aid in draining cord blood from the placenta. Such cord blood recovery may be performed commercially, e.g., LifeBank Inc., Cedar Knolls, N.J., ViaCord, Cord Blood Registry and CryoCell. In one embodiment, the placenta is gravity drained without further manipulation so as to minimize tissue disruption during cord blood recovery.

Typically, a placenta is transported from the delivery or birthing room to another location, e.g., a laboratory, for recovery of cord blood and collection of perfusate. The placenta can be transported in a sterile, thermally insulated transport device (maintaining the temperature of the placenta between 20-28° C.), for example, by placing the placenta, with clamped proximal umbilical cord, in a sterile zip-lock plastic bag, which is then placed in an insulated container. In another embodiment, the placenta is transported in a cord blood collection kit substantially as described in U.S. Pat. No. 7,147,626. In one embodiment, the placenta is delivered to the laboratory four to twenty-four hours following delivery. In certain embodiments, the proximal umbilical cord is clamped, for example within 4-5 cm (centimeter) of the insertion into the placental disc prior to cord blood recovery. In other embodiments, the proximal umbilical cord is clamped after cord blood recovery but prior to further processing of the placenta.

The placenta, prior to collection of the perfusate, can be stored under sterile conditions and at either room temperature or at a temperature of 5 to 25° C. (centigrade). The placenta may be stored for a period of longer than forty eight hours, or for a period of four to twenty-four hours prior to perfusing the placenta to remove any residual cord blood. The placenta can be stored in an anticoagulant solution at a temperature of 5° C. to 25° C. (centigrade). Suitable anticoagulant solutions are well known in the art. For example, a solution of heparin or warfarin sodium can be used. In one embodiment, the anticoagulant solution comprises a solution of heparin (e.g., 1% w/w in 1:1000 solution). In some embodiments, the exsanguinated placenta is stored for no more than 36 hours before placental perfusate is collected.

5.5.3. Placental Perfusion

Methods of perfusing mammalian placentae and obtaining placental perfusate are disclosed, e.g., in Hariri, U.S. Pat. Nos. 7,045,148 and 7,255,879, and in U.S. Application Publication Nos. 2009/0104164, 2007/0190042 and 20070275362, issued as U.S. Pat. No. 8,057,788, the disclosures of which are hereby incorporated by reference herein in their entireties.

Perfusate can be obtained by passage of perfusion solution, e.g., saline solution, culture medium or cell collection compositions described above, through the placental vasculature. In one embodiment, a mammalian placenta is perfused by passage of perfusion solution through either or both of the umbilical artery and umbilical vein. The flow of perfusion solution through the placenta may be accomplished using, e.g., gravity flow into the placenta. For example, the perfusion solution is forced through the placenta using a pump, e.g., a peristaltic pump. The umbilical vein can be, e.g., cannulated with a cannula, e.g., a TEFLON® or plastic cannula, that is connected to a sterile connection apparatus, such as sterile tubing. The sterile connection apparatus is connected to a perfusion manifold.

In preparation for perfusion, the placenta can be oriented in such a manner that the umbilical artery and umbilical vein are located at the highest point of the placenta. The placenta can be perfused by passage of a perfusion solution through the placental vasculature, or through the placental vasculature and surrounding tissue. In one embodiment, the umbilical artery and the umbilical vein are connected simultaneously to a pipette that is connected via a flexible connector to a reservoir of the perfusion solution. The perfusion solution is passed into the umbilical vein and artery. The perfusion solution exudes from and/or passes through the walls of the blood vessels into the surrounding tissues of the placenta, and is collected in a suitable open vessel from the surface of the placenta that was attached to the uterus of the mother during gestation. The perfusion solution may also be introduced through the umbilical cord opening and allowed to flow or percolate out of openings in the wall of the placenta which interfaced with the maternal uterine wall. In another embodiment, the perfusion solution is passed through the umbilical veins and collected from the umbilical artery, or is passed through the umbilical artery and collected from the umbilical veins, that is, is passed through only the placental vasculature (fetal tissue).

In one embodiment, for example, the umbilical artery and the umbilical vein are connected simultaneously, e.g., to a pipette that is connected via a flexible connector to a reservoir of the perfusion solution. The perfusion solution is passed into the umbilical vein and artery. The perfusion solution exudes from and/or passes through the walls of the blood vessels into the surrounding tissues of the placenta, and is collected in a suitable open vessel from the surface of the placenta that was attached to the uterus of the mother during gestation. The perfusion solution may also be introduced through the umbilical cord opening and allowed to flow or percolate out of openings in the wall of the placenta which interfaced with the maternal uterine wall. Placental cells that are collected by this method, which can be referred to as a “pan” method, are typically a mixture of fetal and maternal cells.

In another embodiment, the perfusion solution is passed through the umbilical veins and collected from the umbilical artery, or is passed through the umbilical artery and collected from the umbilical veins. Placental cells collected by this method, which can be referred to as a “closed circuit” method, are typically almost exclusively fetal.

The closed circuit perfusion method can, in one embodiment, be performed as follows. A post-partum placenta is obtained within about 48 hours after birth. The umbilical cord is clamped and cut above the clamp. The umbilical cord can be discarded, or can processed to recover, e.g., umbilical cord stem cells, and/or to process the umbilical cord membrane for the production of a biomaterial. The amniotic membrane can be retained during perfusion, or can be separated from the chorion, e.g., using blunt dissection with the fingers. If the amniotic membrane is separated from the chorion prior to perfusion, it can be, e.g., discarded, or processed, e.g., to obtain stem cells by enzymatic digestion, or to produce, e.g., an amniotic membrane biomaterial, e.g., the biomaterial described in U.S. Application Publication No. 2004/0048796. After cleaning the placenta of all visible blood clots and residual blood, e.g., using sterile gauze, the umbilical cord vessels are exposed, e.g., by partially cutting the umbilical cord membrane to expose a cross-section of the cord. The vessels are identified, and opened, e.g., by advancing a closed alligator clamp through the cut end of each vessel. The apparatus, e.g., plastic tubing connected to a perfusion device or peristaltic pump, is then inserted into each of the placental arteries. The pump can be any pump suitable for the purpose, e.g., a peristaltic pump. Plastic tubing, connected to a sterile collection reservoir, e.g., a blood bag such as a 250 mL collection bag, is then inserted into the placental vein. Alternatively, the tubing connected to the pump is inserted into the placental vein, and tubes to a collection reservoir(s) are inserted into one or both of the placental arteries. The placenta is then perfused with a volume of perfusion solution, e.g., about 750 ml of perfusion solution. Cells in the perfusate are then collected, e.g., by centrifugation.

In one embodiment, the proximal umbilical cord is clamped during perfusion, and, more specifically, can be clamped within 4-5 cm (centimeter) of the cord's insertion into the placental disc.

The first collection of perfusion fluid from a mammalian placenta during the exsanguination process is generally colored with residual red blood cells of the cord blood and/or placental blood. The perfusion fluid becomes more colorless as perfusion proceeds and the residual cord blood cells are washed out of the placenta. Generally from 30 to 100 mL of perfusion fluid is adequate to initially flush blood from the placenta, but more or less perfusion fluid may be used depending on the observed results.

In certain embodiments, cord blood is removed from the placenta prior to perfusion (e.g., by gravity drainage), but the placenta is not flushed (e.g., perfused) with solution to remove residual blood. In certain embodiments, cord blood is removed from the placenta prior to perfusion (e.g., by gravity drainage), and the placenta is flushed (e.g., perfused) with solution to remove residual blood.

The volume of perfusion liquid used to perfuse the placenta may vary depending upon the number of placental cells to be collected, the size of the placenta, the number of collections to be made from a single placenta, etc. In various embodiments, the volume of perfusion liquid may be from 50 mL to 5000 mL, 50 mL to 4000 mL, 50 mL to 3000 mL, 100 mL to 2000 mL, 250 mL to 2000 mL, 500 mL to 2000 mL, or 750 mL to 2000 mL. Typically, the placenta is perfused with 700-800 mL of perfusion liquid following exsanguination.

The placenta can be perfused a plurality of times over the course of several hours or several days. Where the placenta is to be perfused a plurality of times, it may be maintained or cultured under aseptic conditions in a container or other suitable vessel, and perfused with a cell collection composition, or a standard perfusion solution (e.g., a normal saline solution such as phosphate buffered saline (“PBS”) with or without an anticoagulant (e.g., heparin, warfarin sodium, coumarin, bishydroxycoumarin), and/or with or without an antimicrobial agent (e.g., β-mercaptoethanol (0.1 mM); antibiotics such as streptomycin (e.g., at 40-100 μg/ml), penicillin (e.g., at 40 U/ml), amphotericin B (e.g., at 0.5 μg/ml). In one embodiment, an isolated placenta is maintained or cultured for a period of time without collecting the perfusate, such that the placenta is maintained or cultured for 1, 2, 3, 4, 5, 6, 7, 8, 9, 10, 11, 12, 13, 14, 15, 16, 17, 18, 19, 20, 21, 22, 23, or 24 hours, or 2 or 3 or more days before perfusion and collection of perfusate. The perfused placenta can be maintained for one or more additional time(s), e.g., 1, 2, 3, 4, 5, 6, 7, 8, 9, 10, 11, 12, 13, 14, 15, 16, 17, 18, 19, 20, 21, 22, 23, 24 or more hours, and perfused a second time with, e.g., 700-800 mL perfusion fluid. The placenta can be perfused 1, 2, 3, 4, 5 or more times, for example, once every 1, 2, 3, 4, 5 or 6 hours. In one embodiment, perfusion of the placenta and collection of perfusion solution, e.g., placental cell collection composition, is repeated until the number of recovered nucleated cells falls below 100 cells/ml. The perfusates at different time points can be further processed individually to recover time-dependent populations of cells, e.g., total nucleated cells. Perfusates from different time points can also be pooled.

5.5.4. Placental Perfusate and Placental Perfusate Cells

Typically, placental perfusate from a single placental perfusion comprises about 100 million to about 500 million nucleated cells, including hematopoietic cells from which NK cells and/or ILC3 cells, e.g., NK cells and/or ILC3 cells produced according to the three-stage method described herein, may be produced by the method disclosed herein. In certain embodiments, the placental perfusate or perfusate cells comprise CD34⁺ cells, e.g., hematopoietic stem or progenitor cells. Such cells can, in a more specific embodiment, comprise CD34⁺CD45⁻ stem or progenitor cells, CD34⁺CD45⁺ stem or progenitor cells, or the like. In certain embodiments, the perfusate or perfusate cells are cryopreserved prior to isolation of hematopoietic cells therefrom. In certain other embodiments, the placental perfusate comprises, or the perfusate cells comprise, only fetal cells, or a combination of fetal cells and maternal cells.

5.6. NK Cells

5.6.1. NK Cells Produced by Three-Stage Method

In another embodiment, provided herein is an isolated NK cell population, wherein said NK cells are produced according to the three-stage method described above.

In one embodiment, provided herein is an isolated NK cell population produced by a three-stage method described herein, wherein said NK cell population comprises a greater percentage of CD3−CD56+ cells than an NK progenitor cell population produced by a three-stage method described herein, e.g., an NK progenitor cell population produced by the same three-stage method with the exception that the third culture step used to produce the NK progenitor cell population was of shorter duration than the third culture step used to produce the NK cell population. In a specific embodiment, said NK cell population comprises about 70% or more, in some embodiments, 75%, 80%, 85%, 90%, 95%, 98%, or 99% CD3−CD56+ cells. In another specific embodiment, said NK cell population comprises no less than 80%, 85%, 90%, 95%, 98%, or 99% CD3−CD56+ cells. In another specific embodiment, said NK cell population comprises between 70%-75%, 75%-80%, 80%-85%, 85%-90%, 90%-95%, or 95%-99% CD3−CD56+ cells.

In certain embodiments, said CD3 CD56⁺ cells in said NK cell population comprises CD3⁻CD56⁺ cells that are additionally NKp46⁺. In certain embodiments, said CD3⁻CD56⁺ cells in said NK cell population comprises CD3⁻CD56⁺ cells that are additionally CD16−. In certain embodiments, said CD3⁻CD56⁺ cells in said NK cell population comprises CD3⁻CD56⁺ cells that are additionally CD16+. In certain embodiments, said CD3⁻CD56⁺ cells in said NK cell population comprises CD3⁻CD56⁺ cells that are additionally CD94−. In certain embodiments, said CD3⁻CD56⁺ cells in said NK cell population comprises CD3⁻CD56⁺ cells that are additionally CD94+. In certain embodiments, said CD3⁻CD56⁺ cells in said NK cell population comprises CD3⁻CD56⁺ cells that are additionally CD11a⁺. In certain embodiments, said CD3⁻CD56⁺ cells in said NK cell population comprises CD3⁻CD56⁺ cells that are additionally NKp30⁺. In certain embodiments, said CD3⁻CD56⁺ cells in said NK cell population comprises CD3⁻CD56⁺ cells that are additionally CD161⁺. In certain embodiments, said CD3⁻CD56⁺ cells in said NK cell population comprises CD3⁻CD56⁺ cells that are additionally DNAM-1⁺. In certain embodiments, said CD3⁻CD56⁺ cells in said NK cell population comprises CD3⁻CD56⁺ cells that are additionally T-bet⁺.

In one embodiment, an NK cell population produced by a three-stage method described herein comprises cells which are CD117+. In one embodiment, an NK cell population produced by a three-stage method described herein comprises cells which are NKG2D+. In one embodiment, an NK cell population produced by a three-stage method described herein comprises cells which are NKp44+. In one embodiment, an NK cell population produced by a three-stage method described herein comprises cells which are CD244+. In one embodiment, an NK cell population produced by a three-stage method described herein comprises cells which express perforin. In one embodiment, an NK cell population produced by a three-stage method described herein comprises cells which express EOMES. In one embodiment, an NK cell population produced by a three-stage method described herein comprises cells which express granzyme B. In one embodiment, an NK cell population produced by a three-stage method described herein comprises cells which secrete IFNγ, GM-CSF and/or TNFα.

5.7. ILC3 Cells

5.7.1. ILC3 Cells Produced by Three-Stage Method

In another embodiment, provided herein is an isolated ILC3 cell population, wherein said ILC3 cells are produced according to the three-stage method described above.

In one embodiment, provided herein is an isolated ILC3 cell population produced by a three-stage method described herein, wherein said ILC3 cell population comprises a greater percentage of CD3−CD56+ cells than an ILC3 progenitor cell population produced by a three-stage method described herein, e.g., an ILC3 progenitor cell population produced by the same three-stage method with the exception that the third culture step used to produce the ILC3 progenitor cell population was of shorter duration than the third culture step used to produce the ILC3 cell population. In a specific embodiment, said ILC3 cell population comprises about 70% or more, in some embodiments, 75%, 80%, 85%, 90%, 95%, 98%, or 99% CD3−CD56+ cells. In another specific embodiment, said ILC3 cell population comprises no less than 80%, 85%, 90%, 95%, 98%, or 99% CD3−CD56+ cells. In another specific embodiment, said ILC3 cell population comprises between 70%-75%, 75%-80%, 80%-85%, 85%-90%, 90%-95%, or 95%-99% CD3−CD56+ cells.

In certain embodiments, said CD3 CD56⁺ cells in said ILC3 cell population comprises CD3⁻CD56⁺ cells that are additionally NKp46⁻. In certain embodiments, said CD3⁻CD56⁺ cells in said ILC3 cell population comprises CD3⁻CD56⁺ cells that are additionally CD16−. In certain embodiments, said CD3⁻CD56⁺ cells in said ILC3 cell population comprises CD3⁻CD56⁺ cells that are additionally IL1R1+. In certain embodiments, said CD3⁻CD56⁺ cells in said ILC3 cell population comprises CD3⁻CD56⁺ cells that are additionally CD94−. In certain embodiments, said CD3⁻CD56⁺ cells in said ILC3 cell population comprises CD3⁻CD56⁺ cells that are additionally RORγt+. In certain embodiments, said CD3⁻CD56⁺ cells in said ILC3 cell population comprises CD3⁻CD56⁺ cells that are additionally CD11a⁻. In certain embodiments, said CD3⁻CD56⁺ cells in said ILC3 cell population comprises CD3⁻CD56⁺ cells that are additionally T-bet+.

In one embodiment, an ILC3 cell population produced by a three-stage method described herein comprises cells which are CD117+. In one embodiment, an ILC3 cell population produced by a three-stage method described herein comprises cells which are NKG2D⁻. In one embodiment, an ILC3 cell population produced by a three-stage method described herein comprises cells which are NKp30⁻. In one embodiment, an ILC3 cell population produced by a three-stage method described herein comprises cells which are CD244+. In one embodiment, an ILC3 cell population produced by a three-stage method described herein comprises cells which are DNAM-1+. In one embodiment, an ILC3 cell population produced by a three-stage method described herein comprises cells which express AHR. In one embodiment, an ILC3 cell population produced by a three-stage method described herein comprises cells which do not express perforin. In one embodiment, an ILC3 cell population produced by a three-stage method described herein comprises cells which do not express EOMES. In one embodiment, an ILC3 cell population produced by a three-stage method described herein comprises cells which do not express granzyme B. In one embodiment, an ILC3 cell population produced by a three-stage method described herein comprises cells which secrete IL-22 and/or IL-8.

In certain aspects, cell populations produced by the three-stage method described herein comprise CD11a+ cells and CD11a− cells in a ratio of 50:1, 40:1, 30:1, 20:1, 10:1, 5:1, 4:1, 3:1, 2:1, 1:1, 1:2, 1:3, 1:4, 1:5, 1:10, 1:20, 1:30, 1:40, or 1:50. In certain aspects, a population of cells described herein comprises CD11a+ cells and CD11a− cells in a ratio of 50:1. In certain aspects, a population of cells described herein comprises CD11a+ cells and CD11a− cells in a ratio of 20:1. In certain aspects, a population of cells described herein comprises CD11a+ cells and CD11a− cells in a ratio of 10:1. In certain aspects, a population of cells described herein comprises CD11a+ cells and CD11a− cells in a ratio of 5:1. In certain aspects, a population of cells described herein comprises CD11a+ cells and CD11a− cells in a ratio of 1:1. In certain aspects, a population of cells described herein comprises CD11a+ cells and CD11a− cells in a ratio of 1:5. In certain aspects, a population of cells described herein comprises CD11a+ cells and CD11a− cells in a ratio of 1:10. In certain aspects, a population of cells described herein comprises CD11a+ cells and CD11a− cells in a ratio of 1:20. In certain aspects, a population of cells described herein comprises CD11a+ cells and CD11a− cells in a ratio of 1:50.

In certain aspects, cell populations described herein are produced by combining the CD11a+ cells with the CD11a− cells in a ratio of 50:1, 40:1, 30:1, 20:1, 10:1, 5:1, 4:1, 3:1, 2:1, 1:1, 1:2, 1:3, 1:4, 1:5, 1:10, 1:20, 1:30, 1:40, or 1:50 to produce a combined population of cells. In certain aspects, a combined population of cells described herein comprises CD11a+ cells and CD11a− cells combined in a ratio of 50:1. In certain aspects, a combined population of cells described herein comprises CD11a+ cells and CD11a− cells combined in a ratio of 20:1. In certain aspects, a combined population of cells described herein comprises CD11a+ cells and CD11a− cells combined in a ratio of 10:1. In certain aspects, a combined population of cells described herein comprises CD11a+ cells and CD11a− cells combined in a ratio of 5:1. In certain aspects, a combined population of cells described herein comprises CD11a+ cells and CD11a− cells combined in a ratio of 1:1. In certain aspects, a combined population of cells described herein comprises CD11a+ cells and CD11a− cells combined in a ratio of 1:5. In certain aspects, a combined population of cells described herein comprises CD11a+ cells and CD11a− cells combined in a ratio of 1:10. In certain aspects, a combined population of cells described herein comprises CD11a+ cells and CD11a− cells combined in a ratio of 1:20. In certain aspects, a combined population of cells described herein comprises CD11a+ cells and CD11a− cells combined in a ratio of 1:50.

In certain aspects, cell populations produced by the three-stage method described herein comprise NK cells and ILC3 cells in a ratio of 50:1, 40:1, 30:1, 20:1, 10:1, 5:1, 4:1, 3:1, 2:1, 1:1, 1:2, 1:3, 1:4, 1:5, 1:10, 1:20, 1:30, 1:40, or 1:50. In certain aspects, a population of cells described herein comprises NK cells and ILC3 cells in a ratio of 50:1. In certain aspects, a population of cells described herein comprises NK cells and ILC3 cells in a ratio of 20:1. In certain aspects, a population of cells described herein comprises NK cells and ILC3 cells in a ratio of 10:1. In certain aspects, a population of cells described herein comprises NK cells and ILC3 cells in a ratio of 5:1. In certain aspects, a population of cells described herein comprises NK cells and ILC3 cells in a ratio of 1:1. In certain aspects, a population of cells described herein comprises NK cells and ILC3 cells in a ratio of 1:5. In certain aspects, a population of cells described herein comprises NK cells and ILC3 cells in a ratio of 1:10. In certain aspects, a population of cells described herein comprises NK cells and ILC3 cells in a ratio of 1:20. In certain aspects, a population of cells described herein comprises NK cells and ILC3 cells in a ratio of 1:50.

In certain aspects, cell populations described herein are produced by combining the NK cells with the ILC3 cells in a ratio of 50:1, 40:1, 30:1, 20:1, 10:1, 5:1, 4:1, 3:1, 2:1, 1:1, 1:2, 1:3, 1:4, 1:5, 1:10, 1:20, 1:30, 1:40, or 1:50 to produce a combined population of cells. In certain aspects, a combined population of cells described herein comprises NK cells and ILC3 cells combined in a ratio of 50:1. In certain aspects, a combined population of cells described herein comprises NK cells and ILC3 cells combined in a ratio of 20:1. In certain aspects, a combined population of cells described herein comprises NK cells and ILC3 cells combined in a ratio of 10:1. In certain aspects, a combined population of cells described herein comprises NK cells and ILC3 cells combined in a ratio of 5:1. In certain aspects, a combined population of cells described herein comprises NK cells and ILC3 cells combined in a ratio of 1:1. In certain aspects, a combined population of cells described herein comprises NK cells and ILC3 cells combined in a ratio of 1:5. In certain aspects, a combined population of cells described herein comprises NK cells and ILC3 cells combined in a ratio of 1:10. In certain aspects, a combined population of cells described herein comprises NK cells and ILC3 cells combined in a ratio of 1:20. In certain aspects, a combined population of cells described herein comprises NK cells and ILC3 cells combined in a ratio of 1:50.

5.8. NK Cells and/or ILC3 Cells in Combination with Placental Perfusate

Further provided herein are compositions comprising NK cells and/or ILC3 cells according to the three-stage method described herein, in combination with placental perfusate, placental perfusate cells and/or adherent placental cells, e.g., for use in suppressing the proliferation of a tumor cell or plurality of tumor cells.

5.8.1. Combinations of NK Cells and/or ILC3 Cells and Perfusate or Perfusate Cells

Further provided herein are compositions comprising combinations of NK cell and/or ILC3 cell populations produced according to the three-stage method described herein, and placental perfusate and/or placental perfusate cells. In one embodiment, for example, provided herein is a volume of placental perfusate supplemented with NK cells and/or ILC3 cells produced using the methods described herein. In specific embodiments of a volume of placental perfusate supplemented with NK cells and ILC3 cells, the NK cells and ILC3 cells are present in ratios as described herein. In specific embodiments, for example, each milliliter of placental perfusate is supplemented with about 1×10⁴, 5×10⁴, 1×10⁵, 5×10⁵, 1×10⁶, 5×10⁶, 1×10⁷, 5×10⁷, 1×10⁸, 5×10⁸ or more NK cells and/or ILC3 cells produced using the methods described herein. In another embodiment, placental perfusate cells are supplemented with NK cells and/or ILC3 cells produced using the methods described herein. In certain other embodiments, when placental perfusate cells are combined with NK cells and/or ILC3 cells produced using the methods described herein, the placental perfusate cells generally comprise about, greater than about, or fewer than about, 50%, 45%, 40%, 35%, 30%, 25%, 20%, 15%, 10%, 8%, 6%, 4%, 2% or 1% of the total number of cells. In certain other embodiments, when NK cells and/or ILC3 cells produced using the methods described herein are combined with a plurality of placental perfusate cells and/or combined natural killer cells, the NK cells and/or ILC3 cells or NK cell populations generally comprise about, greater than about, or fewer than about, 50%, 45%, 40%, 35%, 30%, 25%, 20%, 15%, 10%, 8%, 6%, 4%, 2% or 1% of the total number of cells. In certain other embodiments, when NK cells and/or ILC3 cells produced using the methods described herein are used to supplement placental perfusate, the volume of solution (e.g., saline solution, culture medium or the like) in which the cells are suspended comprises about, greater than about, or less than about, 50%, 45%, 40%, 35%, 30%, 25%, 20%, 15%, 10%, 8%, 6%, 4%, 2% or 1% of the total volume of perfusate plus cells, where the NK cells and/or ILC3 cells are suspended to about 1×10⁴, 5×10⁴, 1×10⁵, 5×10⁵, 1×10⁶, 5×10⁶, 1×10⁷, 5×10⁷, 1×10⁸, 5×10⁸ or more cells per milliliter prior to supplementation.

In other embodiments, any of the above combinations of cells is, in turn, combined with umbilical cord blood or nucleated cells from umbilical cord blood.

Further provided herein is pooled placental perfusate that is obtained from two or more sources, e.g., two or more placentas, and combined, e.g., pooled. Such pooled perfusate can comprise approximately equal volumes of perfusate from each source, or can comprise different volumes from each source. The relative volumes from each source can be randomly selected, or can be based upon, e.g., a concentration or amount of one or more cellular factors, e.g., cytokines, growth factors, hormones, or the like; the number of placental cells in perfusate from each source; or other characteristics of the perfusate from each source. Perfusate from multiple perfusions of the same placenta can similarly be pooled.

Similarly, provided herein are placental perfusate cells, and placenta-derived intermediate natural killer cells, that are obtained from two or more sources, e.g., two or more placentas, and pooled. Such pooled cells can comprise approximately equal numbers of cells from the two or more sources, or different numbers of cells from one or more of the pooled sources. The relative numbers of cells from each source can be selected based on, e.g., the number of one or more specific cell types in the cells to be pooled, e.g., the number of CD34⁺ cells, etc.

Further provided herein are NK cells and/or ILC3 cells produced using the methods described herein, and combinations of such cells with placental perfusate and/or placental perfusate cells, that have been assayed to determine the degree or amount of tumor suppression (that is, the potency) to be expected from, e.g., a given number of NK cells and/or ILC3 cells or NK cell and/or ILC3 cell populations or a given volume of perfusate. For example, an aliquot or sample number of cells is contacted or brought into proximity with a known number of tumor cells under conditions in which the tumor cells would otherwise proliferate, and the rate of proliferation of the tumor cells in the presence of placental perfusate, perfusate cells, placental natural killer cells, or combinations thereof, overtime (e.g., 1, 2, 3, 4, 5, 6, 7, 8, 9, or 10 weeks, or longer) is compared to the proliferation of an equivalent number of the tumor cells in the absence of perfusate, perfusate cells, placental natural killer cells, or combinations thereof. The potency of the cells can be expressed, e.g., as the number of cells or volume of solution required to suppress tumor cell growth, e.g., by about 10%, 15%, 20%, 25%, 30%, 35%, 40%, 45%, 50%, or the like.

In certain embodiments, NK cells and/or ILC3 cells produced using the methods described herein, are provided as pharmaceutical grade administrable units. Such units can be provided in discrete volumes, e.g., 15 mL, 20 mL, 25 mL, 30 nL. 35 mL, 40 mL, 45 mL, 50 mL, 55 mL, 60 mL, 65 mL, 70 mL, 75 mL, 80 mL, 85 mL, 90 mL, 95 mL, 100 mL, 150 mL, 200 mL, 250 mL, 300 mL, 350 mL, 400 mL, 450 mL, 500 mL, or the like. Such units can be provided so as to contain a specified number of cells, e.g., NK cells and/or ILC3 cells or NK cell and/or ILC3 cell populations in combination with other NK cells and/or ILC3 cells or perfusate cells, e.g., 1×10⁴, 5×10⁴, 1×10⁵, 5×10⁵, 1×10⁶, 5×10⁶, 1×10⁷, 5×10⁷, 1×10⁸, 5×10⁸ or more cells per milliliter, or 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹ or more cells per unit. In specific embodiments, the units can comprise about, at least about, or at most about 1×10⁴, 5×10⁴, 1×10⁵, 5×10⁵, 1×10⁶, 5×10⁶ or more NK cells per milliliter, or 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹ or more cells per unit. Such units can be provided to contain specified numbers of NK cells and/or ILC3 cells or NK cell and/or ILC3 cell populations and/or any of the other cells.

In the above embodiments, the NK cells and/or ILC3 cells or NK cell and/or ILC3 cell populations or combinations of NK cells and/or ILC3 cells or NK cell and/or ILC3 cell populations with other NK cells and/or ILC3 cells, perfusate cells or perfusate can be autologous to a recipient (that is, obtained from the recipient), or allogeneic to a recipient (that is, obtained from at last one other individual from said recipient).

In certain embodiments, each unit of cells is labeled to specify one or more of volume, number of cells, type of cells, whether the unit has been enriched for a particular type of cell, and/or potency of a given number of cells in the unit, or a given number of milliliters of the unit, that is, whether the cells in the unit cause a measurable suppression of proliferation of a particular type or types of tumor cell.

5.8.2. Combinations of NK Cells and/or ILC3 Cells with Adherent Placental Stem Cells

In other embodiments, the NK cells and/or ILC3 cells produced using the methods described herein, e.g., NK cell and/or ILC3 cell populations produced using the three-stage method described herein, either alone or in combination with placental perfusate or placental perfusate cells, are supplemented with isolated adherent placental cells, e.g., placental stem cells and placental multipotent cells as described, e.g., in Hariri U.S. Pat. Nos. 7,045,148 and 7,255,879, and in U.S. Patent Application Publication No. 2007/0275362, the disclosures of which are incorporated herein by reference in their entireties. In specific embodiments, NK cells and ILC3 cells, the NK cells and ILC3 cells are present in ratios as described herein. “Adherent placental cells” means that the cells are adherent to a tissue culture surface, e.g., tissue culture plastic. The adherent placental cells useful in the compositions and methods disclosed herein are generally not trophoblasts, embryonic germ cells or embryonic stem cells.

The NK cells and/or ILC3 cells produced using the methods described herein, e.g., NK cell and/or ILC3 cell populations, either alone or in combination with placental perfusate or placental perfusate cells can be supplemented with, e.g., 1×10⁴, 5×10⁴, 1×10⁵, 5×10⁵, 1×10⁶, 5×10⁶, 1×10⁷, 5×10⁷, 1×10⁸, 5×10⁸ or more adherent placental cells per milliliter, or 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹ or more adherent placental cells. The adherent placental cells in the combinations can be, e.g., adherent placental cells that have been cultured for, e.g., 1, 2, 3, 4, 5, 6, 7, 8, 9, 10, 12, 14, 16, 18, 20, 22, 24, 26, 28, 30, 32, 34, 36, 38, or 40 population doublings, or more.

Isolated adherent placental cells, when cultured in primary cultures or expanded in cell culture, adhere to the tissue culture substrate, e.g., tissue culture container surface (e.g., tissue culture plastic). Adherent placental cells in culture assume a generally fibroblastoid, stellate appearance, with a number of cytoplasmic processes extending from the central cell body. Adherent placental cells are, however, morphologically distinguishable from fibroblasts cultured under the same conditions, as the adherent placental cells exhibit a greater number of such processes than do fibroblasts. Morphologically, adherent placental cells are also distinguishable from hematopoietic stem cells, which generally assume a more rounded, or cobblestone, morphology in culture.

The isolated adherent placental cells, and populations of adherent placental cells, useful in the compositions and methods provided herein, express a plurality of markers that can be used to identify and/or isolate the cells, or populations of cells that comprise the adherent placental cells. The adherent placental cells, and adherent placental cell populations useful in the compositions and methods provided herein include adherent placental cells and adherent placental cell-containing cell populations obtained directly from the placenta, or any part thereof (e.g., amnion, chorion, amnion-chorion plate, placental cotyledons, umbilical cord, and the like). The adherent placental stem cell population, in one embodiment, is a population (that is, two or more) of adherent placental stem cells in culture, e.g., a population in a container, e.g., a bag.

The adherent placental cells generally express the markers CD73, CD105, and CD200, and/or OCT-4, and do not express CD34, CD38, or CD45. Adherent placental stem cells can also express HLA-ABC (MHC-1) and HLA-DR. These markers can be used to identify adherent placental cells, and to distinguish the adherent placental cells from other cell types. Because the adherent placental cells can express CD73 and CD105, they can have mesenchymal stem cell-like characteristics. Lack of expression of CD34, CD38 and/or CD45 identifies the adherent placental stem cells as non-hematopoietic stem cells.

In certain embodiments, the isolated adherent placental cells described herein detectably suppress cancer cell proliferation or tumor growth.

In certain embodiments, the isolated adherent placental cells are isolated placental stem cells. In certain other embodiments, the isolated adherent placental cells are isolated placental multipotent cells. In a specific embodiment, the isolated adherent placental cells are CD34⁻, CD10⁺ and CD105⁺ as detected by flow cytometry. In a more specific embodiment, the isolated CD34⁻, CD10⁺, CD105⁺ adherent placental cells are placental stem cells. In another more specific embodiment, the isolated CD34⁻, CD10⁺, CD105⁺ placental cells are multipotent adherent placental cells. In another specific embodiment, the isolated CD34⁻, CD10⁺, CD105⁺ placental cells have the potential to differentiate into cells of a neural phenotype, cells of an osteogenic phenotype, or cells of a chondrogenic phenotype. In a more specific embodiment, the isolated CD34⁻, CD10⁺, CD105⁺ adherent placental cells are additionally CD200⁺. In another more specific embodiment, the isolated CD34⁻, CD10⁺, CD105⁺ adherent placental cells are additionally CD90⁺ or CD45⁻, as detected by flow cytometry. In another more specific embodiment, the isolated CD34⁻, CD10⁺, CD105⁺ adherent placental cells are additionally CD90⁺ or CD45⁻, as detected by flow cytometry. In a more specific embodiment, the CD34⁻, CD10⁺, CD105⁺, CD200⁺ adherent placental cells are additionally CD90⁺ or CD45⁻, as detected by flow cytometry. In another more specific embodiment, the CD34⁻, CD10⁺, CD105⁺, CD200⁺ adherent placental cells are additionally CD90⁺ and CD45⁻, as detected by flow cytometry. In another more specific embodiment, the CD34⁻, CD10⁺, CD105⁺, CD200⁺, CD90⁺, CD45⁻ adherent placental cells are additionally CD80⁻ and CD86⁻, as detected by flow cytometry.

In one embodiment, the isolated adherent placental cells are CD200⁺, HLA-G⁺. In a specific embodiment, said isolated adherent placental cells are also CD73⁺ and CD105⁺. In another specific embodiment, said isolated adherent placental cells are also CD34⁻, CD38⁻ or CD45⁻. In a more specific embodiment, said isolated adherent placental cells are also CD34⁻, CD38⁻, CD45⁻, CD73⁺ and CD105⁺. In another embodiment, said isolated adherent placental cells produce one or more embryoid-like bodies when cultured under conditions that allow the formation of embryoid-like bodies.

In another embodiment, the isolated adherent placental cells are CD73⁺, CD105⁺, CD200⁺. In a specific embodiment of said populations, said isolated adherent placental cells are also HLA-G⁺. In another specific embodiment, said isolated adherent placental cells are also CD34⁻, CD38⁻ or CD45⁻. In another specific embodiment, said isolated adherent placental cells are also CD34⁻, CD38⁻ and CD45⁻. In a more specific embodiment, said isolated adherent placental cells are also CD34⁻, CD38⁻, CD45⁻, and HLA-G⁺. In another specific embodiment, said isolated adherent placental cells produce one or more embryoid-like bodies when cultured under conditions that allow the formation of embryoid-like bodies.

In another embodiment, the isolated adherent placental cells are CD200⁺, OCT-4⁺. In a specific embodiment, said isolated adherent placental cells are also CD73⁺ and CD105⁺. In another specific embodiment, said isolated adherent placental cells are also HLA-G⁺. In another specific embodiment, said isolated adherent placental cells are also CD34⁻, CD38⁻ and CD45⁻. In a more specific embodiment, said isolated adherent placental cells are also CD34⁻, CD38⁻, CD45⁻, CD73⁺, CD105⁺ and HLA-G⁺. In another specific embodiment, the isolated adherent placental cells also produce one or more embryoid-like bodies when cultured under conditions that allow the formation of embryoid-like bodies.

In another embodiment, the isolated adherent placental cells are CD73⁺, CD105⁺ and HLA-G⁺. In a specific embodiment, said isolated adherent placental cells are also CD34⁻, CD38⁻ or CD45⁻. In another specific embodiment, said isolated adherent placental cells also CD34⁻, CD38⁻ and CD45⁻. In another specific embodiment, said adherent stem cells are also OCT-4⁺. In another specific embodiment, said adherent stem cells are also CD200⁺. In a more specific embodiment, said adherent stem cells are also CD34⁻, CD38⁻, CD45⁻, OCT-4⁺ and CD200⁺.

In another embodiment, the isolated adherent placental cells are CD73⁺, CD105⁺ stem cells, wherein said cells produce one or more embryoid-like bodies under conditions that allow formation of embryoid-like bodies. In a specific embodiment, said isolated adherent placental cells are also CD34⁻, CD38⁻ or CD45⁻. In another specific embodiment, isolated adherent placental cells are also CD34⁻, CD38⁻ and CD45⁻. In another specific embodiment, isolated adherent placental cells are also OCT-4⁺. In a more specific embodiment, said isolated adherent placental cells are also OCT-4⁺, CD34⁻, CD38⁻ and CD45⁻.

In another embodiment, the adherent placental stem cells are OCT-4⁺ stem cells, wherein said adherent placental stem cells produce one or more embryoid-like bodies when cultured under conditions that allow the formation of embryoid-like bodies, and wherein said stem cells have been identified as detectably suppressing cancer cell proliferation or tumor growth.

In various embodiments, at least 10%, at least 20%, at least 30%, at least 40%, at least 50% at least 60%, at least 70%, at least 80%, at least 90%, or at least 95% of said isolated adherent placental cells are OCT-4⁺. In a specific embodiment of the above populations, said isolated adherent placental cells are also CD73⁺ and CD105⁺. In another specific embodiment, said isolated adherent placental cells are also CD34⁻, CD38⁻, or CD45⁻. In another specific embodiment, said stem cells are CD200⁺. In a more specific embodiment, said isolated adherent placental cells are also CD73⁺, CD105⁺, CD200⁺, CD34⁻, CD38⁻, and CD45⁻. In another specific embodiment, said isolated adherent placental cells have been expanded, for example, passaged at least once, at least three times, at least five times, at least 10 times, at least 15 times, or at least 20 times.

In a more specific embodiment of any of the above embodiments, the isolated adherent placental cells express ABC-p (a placenta-specific ABC transporter protein; see, e.g., Allikmets et al., Cancer Res. 58(23):5337-9 (1998)).

In another embodiment, the isolated adherent placental cells CD29⁺, CD44⁺, CD73⁺, CD90⁺, CD105⁺, CD200⁺, CD34⁻ and CD133⁻. In another embodiment, the isolated adherent placental cells constitutively secrete IL-6, IL-8 and monocyte chemoattractant protein (MCP-1).

Each of the above-referenced isolated adherent placental cells can comprise cells obtained and isolated directly from a mammalian placenta, or cells that have been cultured and passaged at least 1, 2, 3, 4, 5, 6, 7, 8, 9, 10, 12, 14, 16, 18, 20, 25, 30 or more times, or a combination thereof. Tumor cell suppressive pluralities of the isolated adherent placental cells described above can comprise about, at least, or no more than, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹ or more isolated adherent placental cells.

5.8.3. Compositions Comprising Adherent Placental Cell Conditioned Media

Also provided herein is the use of a composition comprising NK cells and/or ILC3 cells produced using the methods described herein, e.g., NK cell and/or ILC3 cell populations produced using the three-stage method described herein, and additionally conditioned medium, wherein said composition is tumor suppressive, or is effective in the treatment of cancer or viral infection. In specific embodiments, the NK cells and ILC3 cells are present in ratios as described herein. Adherent placental cells as described herein can be used to produce conditioned medium that is tumor cell suppressive, anti-cancer or anti-viral that is, medium comprising one or more biomolecules secreted or excreted by the cells that have a detectable tumor cell suppressive effect, anti-cancer effect or antiviral effect. In various embodiments, the conditioned medium comprises medium in which the cells have proliferated (that is, have been cultured) for at least 1, 2, 3, 4, 5, 6, 7, 8, 9, 10, 11, 12, 13, 14 or more days. In other embodiments, the conditioned medium comprises medium in which such cells have grown to at least 30%, 40%, 50%, 60%, 70%, 80%, 90% confluence, or up to 100% confluence. Such conditioned medium can be used to support the culture of a separate population of cells, e.g., placental cells, or cells of another kind. In another embodiment, the conditioned medium provided herein comprises medium in which isolated adherent placental cells, e.g., isolated adherent placental stem cells or isolated adherent placental multipotent cells, and cells other than isolated adherent placental cells, e.g., non-placental stem cells or multipotent cells, have been cultured.

Such conditioned medium can be combined with any of, or any combination of NK cells and/or ILC3 cells produced using the methods described herein, placental perfusate, or placental perfusate cells to form a composition that is tumor cell suppressive, anticancer or antiviral. In certain embodiments, the composition comprises less than half conditioned medium by volume, e.g., about, or less than about, 50%, 45%, 40%, 35%, 30%, 25%, 20%, 15%, 10%, 5%, 4%, 3%, 2%, or 1% by volume.

Thus, in one embodiment, provided herein is a composition comprising NK cells and/or ILC3 cells produced using the methods described herein and culture medium from a culture of isolated adherent placental cells, wherein said isolated adherent placental cells (a) adhere to a substrate; and (b) are CD34⁻, CD10⁺ and CD105⁺; wherein said composition detectably suppresses the growth or proliferation of tumor cells, or is anti-cancer or antiviral. In a specific embodiment, the isolated adherent placental cells are CD34⁻, CD10⁺ and CD105⁺ as detected by flow cytometry. In a more specific embodiment, the isolated CD34⁻, CD10⁺, CD105⁺ adherent placental cells are placental stem cells. In another more specific embodiment, the isolated CD34⁻, CD10⁺, CD105⁺ placental cells are multipotent adherent placental cells. In another specific embodiment, the isolated CD34⁻, CD10⁺, CD105⁺ placental cells have the potential to differentiate into cells of a neural phenotype, cells of an osteogenic phenotype, or cells of a chondrogenic phenotype. In a more specific embodiment, the isolated CD34⁻, CD10⁺, CD105⁺ adherent placental cells are additionally CD200⁺. In another more specific embodiment, the isolated CD34⁻, CD10⁺, CD105⁺ adherent placental cells are additionally CD90⁺ or CD45⁻, as detected by flow cytometry. In another more specific embodiment, the isolated CD34⁻, CD10⁺, CD105⁺ adherent placental cells are additionally CD90⁺ or CD45⁻, as detected by flow cytometry. In a more specific embodiment, the CD34⁻, CD10⁺, CD105⁺, CD200⁺ adherent placental cells are additionally CD90⁺ or CD45⁻, as detected by flow cytometry. In another more specific embodiment, the CD34⁻, CD10⁺, CD105⁺, CD200⁺ adherent placental cells are additionally CD90⁺ and CD45⁻, as detected by flow cytometry. In another more specific embodiment, the CD34⁻, CD10⁺, CD105⁺, CD200⁺, CD90⁺, CD45⁻ adherent placental cells are additionally CD80⁻ and CD86⁻, as detected by flow cytometry.

In another embodiment, provided herein is a composition comprising NK cells and/or ILC3 cells produced using the methods described herein, and culture medium from a culture of isolated adherent placental cells, wherein said isolated adherent placental cells (a) adhere to a substrate; and (b) express CD200 and HLA-G, or express CD73, CD105, and CD200, or express CD200 and OCT-4, or express CD73, CD105, and HLA-G, or express CD73 and CD105 and facilitate the formation of one or more embryoid-like bodies in a population of placental cells that comprise the placental stem cells when said population is cultured under conditions that allow formation of embryoid-like bodies, or express OCT-4 and facilitate the formation of one or more embryoid-like bodies in a population of placental cells that comprise the placental stem cells when said population is cultured under conditions that allow formation of embryoid-like bodies; wherein said composition detectably suppresses the growth or proliferation of tumor cells, or is anti-cancer or antiviral. In a specific embodiment, the composition further comprises a plurality of said isolated placental adherent cells. In another specific embodiment, the composition comprises a plurality of non-placental cells. In a more specific embodiment, said non-placental cells comprise CD34⁺ cells, e.g., hematopoietic progenitor cells, such as peripheral blood hematopoietic progenitor cells, cord blood hematopoietic progenitor cells, or placental blood hematopoietic progenitor cells. The non-placental cells can also comprise stem cells, such as mesenchymal stem cells, e.g., bone marrow-derived mesenchymal stem cells. The non-placental cells can also be one or more types of adult cells or cell lines. In another specific embodiment, the composition comprises an anti-proliferative agent, e.g., an anti-MIP-1a or anti-MIP-1β antibody.

In a specific embodiment, culture medium conditioned by one of the cells or cell combinations described above is obtained from a plurality of isolated adherent placental cells co-cultured with a plurality of tumor cells at a ratio of about 1:1, about 2:1, about 3:1, about 4:1, or about 5:1 isolated adherent placental cells to tumor cells. For example, the conditioned culture medium or supernatant can be obtained from a culture comprising about 1×10⁵ isolated adherent placental cells, about 1×10⁶ isolated adherent placental cells, about 1×10⁷ isolated adherent placental cells, or about 1×10⁸ isolated adherent placental cells, or more. In another specific embodiment, the conditioned culture medium or supernatant is obtained from a co-culture comprising about 1×10⁵ to about 5×10⁵ isolated adherent placental cells and about 1×10⁵ tumor cells; about 1×10⁶ to about 5×10⁶ isolated adherent placental cells and about 1×10⁶ tumor cells; about 1×10⁷ to about 5×10⁷ isolated adherent placental cells and about 1×10⁷ tumor cells; or about 1×10⁸ to about 5×10⁸ isolated adherent placental cells and about 1×10⁸ tumor cells.

5.9. Preservation of Cells

Cells, e.g., NK cells and/or ILC3 cells produced using the methods described herein, e.g., NK cell and/or ILC3 cell populations produced using the three-stage method described herein, or placental perfusate cells comprising hematopoietic stem cells or progenitor cells, can be preserved, that is, placed under conditions that allow for long-term storage, or under conditions that inhibit cell death by, e.g., apoptosis or necrosis.

Placental perfusate can be produced by passage of a cell collection composition through at least a part of the placenta, e.g., through the placental vasculature. The cell collection composition comprises one or more compounds that act to preserve cells contained within the perfusate. Such a placental cell collection composition can comprise an apoptosis inhibitor, necrosis inhibitor and/or an oxygen-carrying perfluorocarbon, as described in related U.S. Application Publication No. 20070190042, the disclosure of which is hereby incorporated by reference in its entirety.

In one embodiment, perfusate or a population of placental cells are collected from a mammalian, e.g., human, post-partum placenta by bringing the perfusate or population of cells into proximity with a cell collection composition comprising an inhibitor of apoptosis and an oxygen-carrying perfluorocarbon, wherein said inhibitor of apoptosis is present in an amount and for a time sufficient to reduce or prevent apoptosis in the population of placental cells, e.g., adherent placental cells, for example, placental stem cells or placental multipotent cells, as compared to a population of cells not contacted or brought into proximity with the inhibitor of apoptosis. For example, the placenta can be perfused with the cell collection composition, and placental cells, e.g., total nucleated placental cells, are isolated therefrom. In a specific embodiment, the inhibitor of apoptosis is a caspase inhibitor. In another specific embodiment, said inhibitor of apoptosis is a JNK inhibitor. In a more specific embodiment, said JNK inhibitor does not modulate differentiation or proliferation of adherent placental cells, e.g., adherent placental stem cells or adherent placental multipotent cells. In another embodiment, the cell collection composition comprises said inhibitor of apoptosis and said oxygen-carrying perfluorocarbon in separate phases. In another embodiment, the cell collection composition comprises said inhibitor of apoptosis and said oxygen-carrying perfluorocarbon in an emulsion. In another embodiment, the cell collection composition additionally comprises an emulsifier, e.g., lecithin. In another embodiment, said apoptosis inhibitor and said perfluorocarbon are between about 0° C. and about 25° C. at the time of bringing the placental cells into proximity with the cell collection composition. In another more specific embodiment, said apoptosis inhibitor and said perfluorocarbon are between about 2° C. and 10° C., or between about 2° C. and about 5° C., at the time of bringing the placental cells into proximity with the cell collection composition. In another more specific embodiment, said bringing into proximity is performed during transport of said population of cells. In another more specific embodiment, said bringing into proximity is performed during freezing and thawing of said population of cells.

In another embodiment, placental perfusate and/or placental cells can be collected and preserved by bringing the perfusate and/or cells into proximity with an inhibitor of apoptosis and an organ-preserving compound, wherein said inhibitor of apoptosis is present in an amount and for a time sufficient to reduce or prevent apoptosis of the cells, as compared to perfusate or placental cells not contacted or brought into proximity with the inhibitor of apoptosis. In a specific embodiment, the organ-preserving compound is UW solution (described in U.S. Pat. No. 4,798,824; also known as VIASPAN™; see also Southard et al., Transplantation 49(2):251-257 (1990) or a solution described in Stern et al., U.S. Pat. No. 5,552,267, the disclosures of which are hereby incorporated by reference in their entireties. In another embodiment, said organ-preserving composition is hydroxyethyl starch, lactobionic acid, raffinose, or a combination thereof. In another embodiment, the placental cell collection composition additionally comprises an oxygen-carrying perfluorocarbon, either in two phases or as an emulsion.

In another embodiment of the method, placental cells are brought into proximity with a cell collection composition comprising an apoptosis inhibitor and oxygen-carrying perfluorocarbon, organ-preserving compound, or combination thereof, during perfusion. In another embodiment, placental cells are brought into proximity with said cell collection compound after collection by perfusion.

Typically, during placental cell collection, enrichment and isolation, it is preferable to minimize or eliminate cell stress due to hypoxia and mechanical stress. In another embodiment of the method, therefore, placental perfusate or a population of placental cells is exposed to a hypoxic condition during collection, enrichment or isolation for less than six hours during said preservation, wherein a hypoxic condition is a concentration of oxygen that is less than normal blood oxygen concentration. In a more specific embodiment, said perfusate or population of placental cells is exposed to said hypoxic condition for less than two hours during said preservation. In another more specific embodiment, said population of placental cells is exposed to said hypoxic condition for less than one hour, or less than thirty minutes, or is not exposed to a hypoxic condition, during collection, enrichment or isolation. In another specific embodiment, said population of placental cells is not exposed to shear stress during collection, enrichment or isolation.

Cells, e.g., placental perfusate cells, hematopoietic cells, e.g., CD34⁺ hematopoietic stem cells; NK cells and/or ILC3 cells produced using the methods described herein; isolated adherent placental cells provided herein can be cryopreserved, e.g., in cryopreservation medium in small containers, e.g., ampoules or septum vials. In certain embodiments, cells provided herein are cryopreserved at a concentration of about 1×10⁴-5×10⁸ cells per mL. In specific embodiments, cells provided herein are cryopreserved at a concentration of about 1×10⁶-1.5×10⁷ cells per mL. In more specific embodiments, cells provided herein are cryopreserved at a concentration of about 1×10⁴, 5×10⁴, 1×10⁵, 5×10⁵, 1×10⁶, 5×10⁶, 1×10⁷, 1.5×10⁷ cells per mL.

Suitable cryopreservation medium includes, but is not limited to, normal saline, culture medium including, e.g., growth medium, or cell freezing medium, for example commercially available cell freezing medium, e.g., C2695, C2639 or C6039 (Sigma); CryoStor® CS2, CryoStor® CS5 or CryoStor® CS10 (BioLife Solutions). In one embodiment, cryopreservation medium comprises DMSO (dimethylsulfoxide), at a concentration of, e.g., about 1, 2, 3, 4, 5, 6, 7, 8, 9 or 10% (v/v). Cryopreservation medium may comprise additional agents, for example, methylcellulose, dextran, albumin (e.g., human serum albumin), trehalose, and/or glycerol. In certain embodiments, the cryopreservation medium comprises about 1%-10% DMSO, about 25%-75% dextran and/or about 20-60% human serum albumin (HSA). In certain embodiments, the cryopreservation medium comprises about 1%-10% DMSO, about 25%-75% trehalose and/or about 20-60% human HSA. In a specific embodiment, the cryopreservation medium comprises 5% DMSO, 55% dextran and 40% HSA. In a more specific embodiment, the cryopreservation medium comprises 5% DMSO, 55% dextran (10% w/v in normal saline) and 40% HSA. In another specific embodiment, the cryopreservation medium comprises 5% DMSO, 55% trehalose and 40% HSA. In a more specific embodiment, the cryopreservation medium comprises 5% DMSO, 55% trehalose (10% w/v in normal saline) and 40% HSA. In another specific embodiment, the cryopreservation medium comprises CryoStor® CS5. In another specific embodiment, the cryopreservation medium comprises CryoStor® CS10.

Cells provided herein can be cryopreserved by any of a variety of methods, and at any stage of cell culturing, expansion or differentiation. For example, cells provided herein can be cryopreserved right after isolation from the origin tissues or organs, e.g., placental perfusate or umbilical cord blood, or during, or after either the first, second, or third step of the methods outlined above. In certain embodiments, the hematopoietic cells, e.g., hematopoietic stem or progenitor cells are cryopreserved within about 1, 5, 10, 15, 20, 30, 45 minutes or within about 1, 2, 4, 6, 10, 12, 18, 20 or 24 hours after isolation from the origin tissues or organs. In certain embodiments, said cells are cryopreserved within 1, 2 or 3 days after isolation from the origin tissues or organs. In certain embodiments, said cells are cryopreserved after being cultured in a first medium as described above, for about 1, 2, 3, 4, 5, 6, 7, 8, 9, 10, 11, 12, 13, 14, 15, 16, 17, 18, 19, 20, 21, 22, 23, 24, 25, 26, 27 or 28 days. In some embodiments, said cells are cryopreserved after being cultured in a first medium as described above, for about 1, 2, 3, 4, 5, 6, 7, 8, 9, 10, 11, 12, 13, 14, 15, 16, 17, 18, 19, 20, 21, 22, 23, 24, 25, 26, 27 or 28 days, and in a second medium for about 1, 2, 3, 4, 5, 6, 7, 8, 9, 10, 11, 12, 13, 14, 15, 16, 17, 18, 19, 20, 21, 22, 23, 24, 25, 26, 27 or 28 days as described above. In some embodiments, when NK cells are made using a three-stage method described herein, said cells are cryopreserved after being cultured in a first medium about 1, 2, 3, 4, 5, 6, 7, 8, 9, 10, 11, 12, 13, 14, 15, 16, 17, 18, 19, 20, 21, 22, 23, 24, or 25 days; and/or after being cultured in a second medium about 1, 2, 3, 4, 5, 6, 7, 8, 9, 10, 11, 12, 13, 14, 15, 16, 17, 18, 19, 20, 21, 22, 23, 24, or 25 days; and/or after being cultured in a third medium about 1, 2, 3, 4, 5, 6, 7, 8, 9, 10, 11, 12, 13, 14, 15, 16, 17, 18, 19, 20, 21, 22, 23, 24, or 25 days. In a specific embodiment, NK cells and/or ILC3 cells are made using a three-stage method described herein, and said cells are cryopreserved after being cultured in a first medium for 10 days; after being cultured in a second medium for 4 days; and after being cultured in a third medium for 21 days.

In one aspect, provided herein is a method of cryopreserving a population of NK cells and/or ILC3 cells, e.g., NK cells and/or ILC3 cells produced by a three-stage method described herein. In one embodiment, said method comprises: culturing hematopoietic stem cells or progenitor cells, e.g., CD34⁺ stem cells or progenitor cells, in a first medium comprising a stem cell mobilizing agent and thrombopoietin (Tpo) to produce a first population of cells, subsequently culturing said first population of cells in a second medium comprising a stem cell mobilizing agent and interleukin-15 (IL-15), and lacking Tpo, to produce a second population of cells, and subsequently culturing said second population of cells in a third medium comprising IL-2 and IL-15, and lacking a stem cell mobilizing agent and LMWH, to produce a third population of cells, wherein the third population of cells comprises natural killer cells that are CD56+, CD3−, CD16− or CD16+, and CD94+ or CD94−, and wherein at least 70%, or at least 80%, of the natural killer cells are viable, and next, cryopreserving the NK cells in a cryopreservation medium. In certain embodiments, said first medium and/or said second medium lack leukemia inhibiting factor (LIF) and/or macrophage inflammatory protein-1 alpha (MIP-1α). In certain embodiments, said third medium lacks LIF, MIP-1α, and FMS-like tyrosine kinase-3 ligand (Flt-3L). In specific embodiments, said first medium and said second medium lack LIF and MIP-1α, and said third medium lacks LIF, MIP-1α, and Flt3L. In certain embodiments, none of the first medium, second medium or third medium comprises heparin, e.g., low-molecular weight heparin. In a specific embodiment, said cryopreservation step further comprises (1) preparing a cell suspension solution; (2) adding cryopreservation medium to the cell suspension solution from step (1) to obtain cryopreserved cell suspension; (3) cooling the cryopreserved cell suspension from step (3) to obtain a cryopreserved sample; and (4) storing the cryopreserved sample below −80° C. In certain embodiments, the method includes no intermediary steps.

In one embodiment, said method comprises: (a) culturing hematopoietic stem or progenitor cells in a first medium comprising a stem cell mobilizing agent and thrombopoietin (Tpo) to produce a first population of cells; (b) culturing the first population of cells in a second medium comprising a stem cell mobilizing agent and interleukin-15 (IL-15), and lacking Tpo, to produce a second population of cells; and (c) culturing the second population of cells in a third medium comprising IL-2 and IL-15, and lacking LMWH, to produce a third population of cells; wherein the third population of cells comprises natural killer cells that are CD56+, CD3−, and CD11a+ and next, cryopreserving the NK cells in a cryopreservation medium. In certain embodiments, said first medium and/or said second medium lack leukemia inhibiting factor (LIF) and/or macrophage inflammatory protein-1 alpha (MIP-1α). In certain embodiments, said third medium lacks LIF, MIP-1α, and FMS-like tyrosine kinase-3 ligand (Flt-3L). In specific embodiments, said first medium and said second medium lack LIF and MIP-1α, and said third medium lacks LIF, MIP-1α, and Flt3L. In certain embodiments, none of the first medium, second medium or third medium comprises heparin, e.g., low-molecular weight heparin. In a specific embodiment, said cryopreservation step further comprises (1) preparing a cell suspension solution; (2) adding cryopreservation medium to the cell suspension solution from step (1) to obtain cryopreserved cell suspension; (3) cooling the cryopreserved cell suspension from step (3) to obtain a cryopreserved sample; and (4) storing the cryopreserved sample below −80° C. In certain embodiments, the method includes no intermediary steps.

In one embodiment, said method comprises: (a) culturing hematopoietic stem or progenitor cells in a first medium comprising a stem cell mobilizing agent and thrombopoietin (Tpo) to produce a first population of cells; (b) culturing the first population of cells in a second medium comprising a stem cell mobilizing agent and interleukin-15 (IL-15), and lacking Tpo, to produce a second population of cells; and (c) culturing the second population of cells in a third medium comprising IL-2 and IL-15, and lacking each of stem cell factor (SCF) and LMWH, to produce a third population of cells; wherein the third population of cells comprises natural killer cells that are CD56+, CD3−, and CD11a+ and next, cryopreserving the NK cells in a cryopreservation medium. In certain embodiments, said first medium and/or said second medium lack leukemia inhibiting factor (LIF) and/or macrophage inflammatory protein-1 alpha (MIP-1α). In certain embodiments, said third medium lacks LIF, MIP-1α, and FMS-like tyrosine kinase-3 ligand (Flt-3L). In specific embodiments, said first medium and said second medium lack LIF and MIP-1α, and said third medium lacks LIF, MIP-1α, and Flt3L. In certain embodiments, none of the first medium, second medium or third medium comprises heparin, e.g., low-molecular weight heparin. In a specific embodiment, said cryopreservation step further comprises (1) preparing a cell suspension solution; (2) adding cryopreservation medium to the cell suspension solution from step (1) to obtain cryopreserved cell suspension; (3) cooling the cryopreserved cell suspension from step (3) to obtain a cryopreserved sample; and (4) storing the cryopreserved sample below −80° C. In certain embodiments, the method includes no intermediary steps.

In one embodiment, said method comprises: (a) culturing hematopoietic stem or progenitor cells in a first medium comprising a stem cell mobilizing agent and thrombopoietin (Tpo) to produce a first population of cells; (b) culturing the first population of cells in a second medium comprising a stem cell mobilizing agent and interleukin-15 (IL-15), and lacking Tpo, to produce a second population of cells; and (c) culturing the second population of cells in a third medium comprising IL-2 and IL-15, and lacking each of SCF, a stem cell mobilizing agent, and LMWH, to produce a third population of cells; wherein the third population of cells comprises natural killer cells that are CD56+, CD3−, and CD11a+ and next, cryopreserving the NK cells in a cryopreservation medium. In certain embodiments, said first medium and/or said second medium lack leukemia inhibiting factor (LIF) and/or macrophage inflammatory protein-1 alpha (MIP-1α). In certain embodiments, said third medium lacks LIF, MIP-1α, and FMS-like tyrosine kinase-3 ligand (Flt-3L). In specific embodiments, said first medium and said second medium lack LIF and MIP-1α, and said third medium lacks LIF, MIP-1α, and Flt3L. In certain embodiments, none of the first medium, second medium or third medium comprises heparin, e.g., low-molecular weight heparin. In a specific embodiment, said cryopreservation step further comprises (1) preparing a cell suspension solution; (2) adding cryopreservation medium to the cell suspension solution from step (1) to obtain cryopreserved cell suspension; (3) cooling the cryopreserved cell suspension from step (3) to obtain a cryopreserved sample; and (4) storing the cryopreserved sample below −80° C. In certain embodiments, the method includes no intermediary steps.

In one embodiment, said method comprises: (a) culturing hematopoietic stem or progenitor cells in a first medium comprising a stem cell mobilizing agent and thrombopoietin (Tpo) to produce a first population of cells; (b) culturing the first population of cells in a second medium comprising a stem cell mobilizing agent and interleukin-15 (IL-15), and lacking Tpo, to produce a second population of cells; (c) culturing the second population of cells in a third medium comprising IL-2 and IL-15, and lacking each of a stem cell mobilizing agent and LMWH, to produce a third population of cells; and (d) isolating CD11a+ cells from the third population of cells to produce a fourth population of cells; wherein the fourth population of cells comprises natural killer cells that are CD56+, CD3−, and CD11a+ and next, cryopreserving the NK cells in a cryopreservation medium. In certain embodiments, said first medium and/or said second medium lack leukemia inhibiting factor (LIF) and/or macrophage inflammatory protein-1 alpha (MIP-1α). In certain embodiments, said third medium lacks LIF, MIP-1α, and FMS-like tyrosine kinase-3 ligand (Flt-3L). In specific embodiments, said first medium and said second medium lack LIF and MIP-1α, and said third medium lacks LIF, MIP-1α, and Flt3L. In certain embodiments, none of the first medium, second medium or third medium comprises heparin, e.g., low-molecular weight heparin. In a specific embodiment, said cryopreservation step further comprises (1) preparing a cell suspension solution; (2) adding cryopreservation medium to the cell suspension solution from step (1) to obtain cryopreserved cell suspension; (3) cooling the cryopreserved cell suspension from step (3) to obtain a cryopreserved sample; and (4) storing the cryopreserved sample below −80° C. In certain embodiments, the method includes no intermediary steps.

In one embodiment, said method comprises: (a) culturing hematopoietic stem or progenitor cells in a first medium comprising a stem cell mobilizing agent and thrombopoietin (Tpo) to produce a first population of cells; (b) culturing the first population of cells in a second medium comprising a stem cell mobilizing agent and interleukin-15 (IL-15), and lacking Tpo, to produce a second population of cells; and (c) culturing the second population of cells in a third medium comprising IL-2 and IL-15, and lacking LMWH, to produce a third population of cells; wherein the third population of cells comprises ILC3 cells that are CD56+, CD3−, and CD11a− and next, cryopreserving the ILC3 cells in a cryopreservation medium. In certain embodiments, said first medium and/or said second medium lack leukemia inhibiting factor (LIF) and/or macrophage inflammatory protein-1 alpha (MIP-1α). In certain embodiments, said third medium lacks LIF, MIP-1α, and FMS-like tyrosine kinase-3 ligand (Flt-3L). In specific embodiments, said first medium and said second medium lack LIF and MIP-1α, and said third medium lacks LIF, MIP-1α, and Flt3L. In certain embodiments, none of the first medium, second medium or third medium comprises heparin, e.g., low-molecular weight heparin. In a specific embodiment, said cryopreservation step further comprises (1) preparing a cell suspension solution; (2) adding cryopreservation medium to the cell suspension solution from step (1) to obtain cryopreserved cell suspension; (3) cooling the cryopreserved cell suspension from step (3) to obtain a cryopreserved sample; and (4) storing the cryopreserved sample below −80° C. In certain embodiments, the method includes no intermediary steps.

In one embodiment, said method comprises: (a) culturing hematopoietic stem or progenitor cells in a first medium comprising a stem cell mobilizing agent and thrombopoietin (Tpo) to produce a first population of cells; (b) culturing the first population of cells in a second medium comprising a stem cell mobilizing agent and interleukin-15 (IL-15), and lacking Tpo, to produce a second population of cells; and (c) culturing the second population of cells in a third medium comprising a stem cell mobilizing agent, IL-2 and IL-15, and lacking LMWH, to produce a third population of cells; wherein the third population of cells comprises ILC3 cells that are CD56+, CD3−, and CD11a− and next, cryopreserving the ILC3 cells in a cryopreservation medium. In certain embodiments, said first medium and/or said second medium lack leukemia inhibiting factor (LIF) and/or macrophage inflammatory protein-1 alpha (MIP-1α). In certain embodiments, said third medium lacks LIF, MIP-1α, and FMS-like tyrosine kinase-3 ligand (Flt-3L). In specific embodiments, said first medium and said second medium lack LIF and MIP-1α, and said third medium lacks LIF, MIP-1α, and Flt3L. In certain embodiments, none of the first medium, second medium or third medium comprises heparin, e.g., low-molecular weight heparin. In a specific embodiment, said cryopreservation step further comprises (1) preparing a cell suspension solution; (2) adding cryopreservation medium to the cell suspension solution from step (1) to obtain cryopreserved cell suspension; (3) cooling the cryopreserved cell suspension from step (3) to obtain a cryopreserved sample; and (4) storing the cryopreserved sample below −80° C. In certain embodiments, the method includes no intermediary steps.

In one embodiment, said method comprises: (a) culturing hematopoietic stem or progenitor cells in a first medium comprising a stem cell mobilizing agent and thrombopoietin (Tpo) to produce a first population of cells; (b) culturing the first population of cells in a second medium comprising a stem cell mobilizing agent and interleukin-15 (IL-15), and lacking Tpo, to produce a second population of cells; and (c) culturing the second population of cells in a third medium comprising SCF, IL-2 and IL-15, and lacking LMWH, to produce a third population of cells; wherein the third population of cells comprises ILC3 cells that are CD56+, CD3−, and CD11a− and next, cryopreserving the ILC3 cells in a cryopreservation medium. In certain embodiments, said first medium and/or said second medium lack leukemia inhibiting factor (LIF) and/or macrophage inflammatory protein-1 alpha (MIP-1α). In certain embodiments, said third medium lacks LIF, MIP-1α, and FMS-like tyrosine kinase-3 ligand (Flt-3L). In specific embodiments, said first medium and said second medium lack LIF and MIP-1α, and said third medium lacks LIF, MIP-1α, and Flt3L. In certain embodiments, none of the first medium, second medium or third medium comprises heparin, e.g., low-molecular weight heparin. In a specific embodiment, said cryopreservation step further comprises (1) preparing a cell suspension solution; (2) adding cryopreservation medium to the cell suspension solution from step (1) to obtain cryopreserved cell suspension; (3) cooling the cryopreserved cell suspension from step (3) to obtain a cryopreserved sample; and (4) storing the cryopreserved sample below −80° C. In certain embodiments, the method includes no intermediary steps.

In one embodiment, said method comprises: (a) culturing hematopoietic stem or progenitor cells in a first medium comprising a stem cell mobilizing agent and thrombopoietin (Tpo) to produce a first population of cells; (b) culturing the first population of cells in a second medium comprising a stem cell mobilizing agent and interleukin-15 (IL-15), and lacking Tpo, to produce a second population of cells; and (c) culturing the second population of cells in a third medium comprising a stem cell mobilizing agent, SCF, IL-2 and IL-15, and lacking LMWH, to produce a third population of cells; wherein the third population of cells comprises ILC3 cells that are CD56+, CD3−, and CD11a− and next, cryopreserving the ILC3 cells in a cryopreservation medium. In certain embodiments, said first medium and/or said second medium lack leukemia inhibiting factor (LIF) and/or macrophage inflammatory protein-1 alpha (MIP-1α). In certain embodiments, said third medium lacks LIF, MIP-1α, and FMS-like tyrosine kinase-3 ligand (Flt-3L). In specific embodiments, said first medium and said second medium lack LIF and MIP-1α, and said third medium lacks LIF, MIP-1α, and Flt3L. In certain embodiments, none of the first medium, second medium or third medium comprises heparin, e.g., low-molecular weight heparin. In a specific embodiment, said cryopreservation step further comprises (1) preparing a cell suspension solution; (2) adding cryopreservation medium to the cell suspension solution from step (1) to obtain cryopreserved cell suspension; (3) cooling the cryopreserved cell suspension from step (3) to obtain a cryopreserved sample; and (4) storing the cryopreserved sample below −80° C. In certain embodiments, the method includes no intermediary steps.

In one embodiment, said method comprises: (a) culturing hematopoietic stem or progenitor cells in a first medium comprising a stem cell mobilizing agent and thrombopoietin (Tpo) to produce a first population of cells; (b) culturing the first population of cells in a second medium comprising a stem cell mobilizing agent and interleukin-15 (IL-15), and lacking Tpo, to produce a second population of cells; (c) culturing the second population of cells in a third medium comprising IL-2 and IL-15, and lacking each of a stem cell mobilizing agent and LMWH, to produce a third population of cells; and (d) isolating CD11a− cells from the third population of cells to produce a fourth population of cells; wherein the fourth population of cells comprises ILC3 cells that are CD56+, CD3−, and CD11a− and next, cryopreserving the ILC3 cells in a cryopreservation medium. In certain embodiments, said first medium and/or said second medium lack leukemia inhibiting factor (LIF) and/or macrophage inflammatory protein-1 alpha (MIP-1α). In certain embodiments, said third medium lacks LIF, MIP-1α, and FMS-like tyrosine kinase-3 ligand (Flt-3L). In specific embodiments, said first medium and said second medium lack LIF and MIP-1α, and said third medium lacks LIF, MIP-1α, and Flt3L. In certain embodiments, none of the first medium, second medium or third medium comprises heparin, e.g., low-molecular weight heparin. In a specific embodiment, said cryopreservation step further comprises (1) preparing a cell suspension solution; (2) adding cryopreservation medium to the cell suspension solution from step (1) to obtain cryopreserved cell suspension; (3) cooling the cryopreserved cell suspension from step (3) to obtain a cryopreserved sample; and (4) storing the cryopreserved sample below −80° C. In certain embodiments, the method includes no intermediary steps.

Cells provided herein can be cooled in a controlled-rate freezer, e.g., at about 0.1, 0.3, 0.5, 1, or 2° C./min during cryopreservation. In one embodiment, the cryopreservation temperature is about −80° C. to about −180° C., or about −125° C. to about −140° C. Cryopreserved cells can be transferred to liquid nitrogen prior to thawing for use. In some embodiments, for example, once the ampoules have reached about −90° C., they are transferred to a liquid nitrogen storage area. Cryopreserved cells can be thawed at a temperature of about 25° C. to about 40° C., more specifically can be thawed to a temperature of about 37° C. In certain embodiments, the cryopreserved cells are thawed after being cryopreserved for about 1, 2, 4, 6, 10, 12, 18, 20 or 24 hours, or for about 1, 2, 3, 4, 5, 6, 7, 8, 9, 10, 11, 12, 13, 14, 15, 16, 17, 18, 19, 20, 21, 22, 23, 24, 25, 26, 27 or 28 days. In certain embodiments, the cryopreserved cells are thawed after being cryopreserved for about 1, 2, 3, 4, 5, 6, 7, 8, 9, 10, 11, 12, 13, 14, 15, 16, 17, 18, 19, 20, 21, 22, 23, 24, 25, 26, 27 or 28 months. In certain embodiments, the cryopreserved cells are thawed after being cryopreserved for about 1, 2, 3, 4, 5, 6, 7, 8, 9 or 10 years.

Suitable thawing medium includes, but is not limited to, normal saline, plasmalyte culture medium including, for example, growth medium, e.g., RPMI medium. In certain embodiments, the thawing medium comprises one or more of medium supplements (e.g., nutrients, cytokines and/or factors). Medium supplements suitable for thawing cells provided herein include, for example without limitation, serum such as human serum AB, fetal bovine serum (FBS) or fetal calf serum (FCS), vitamins, human serum albumin (HSA), bovine serum albumin (BSA), amino acids (e.g., L-glutamine), fatty acids (e.g., oleic acid, linoleic acid or palmitic acid), insulin (e.g., recombinant human insulin), transferrin (iron saturated human transferrin), β-mercaptoethanol, stem cell factor (SCF), Fms-like-tyrosine kinase 3 ligand (Flt3-L), cytokines such as interleukin-2 (IL-2), interleukin-7 (IL-7), interleukin-15 (IL-15), thrombopoietin (Tpo) or heparin. In a specific embodiment, the thawing medium useful in the methods provided herein comprises RPMI. In another specific embodiment, said thawing medium comprises plasmalyte. In another specific embodiment, said thawing medium comprises about 0.5-20% FBS. In another specific embodiment, said thawing medium comprises about 1, 2, 5, 10, 15 or 20% FBS. In another specific embodiment, said thawing medium comprises about 0.5%-20% HSA. In another specific embodiment, said thawing medium comprises about 1, 2.5, 5, 10, 15, or 20% HSA. In a more specific embodiment, said thawing medium comprises RPMI and about 10% FBS. In another more specific embodiment, said thawing medium comprises plasmalyte and about 5% HSA.

The cryopreservation methods provided herein can be optimized to allow for long-term storage, or under conditions that inhibit cell death by, e.g., apoptosis or necrosis. In some embodiments, the post-thaw cells comprise greater than 60%, 65%, 70%, 75%, 80%, 85%, 90%, 95% or 98% of viable cells, as determined by, e.g., automatic cell counter or trypan blue method. In another embodiment, the post-thaw cells comprise about 0.5, 1, 5, 10, 15, 20 or 25% of dead cells. In another embodiment, the post-thaw cells comprise about 0.5, 1, 5, 10, 15, 20 or 25% of early apoptotic cells. In another embodiment, about 0.5, 1, 5, 10, 15 or 20% of post-thaw cells undergo apoptosis after 1, 2, 3, 4, 5, 6, 7, 8, 9, 10, 11, 12, 13, 14, 15, 16, 17, 18, 19, 20, 21, 22, 23, 24, 25, 26, 27 or 28 days after being thawed, e.g., as determined by an apoptosis assay (e.g., TO-PR03 or AnnV/PI Apoptosis assay kit). In certain embodiments, the post-thaw cells are re-cryopreserved after being cultured, expanded or differentiated using methods provided herein.

5.10. Compositions Comprising NK Cells and/or ILC3 Cells

5.10.1. NK Cells and/or ILC3 Cells Produced Using the Three-Stage Method

In some embodiments, provided herein is a composition, e.g., a pharmaceutical composition, comprising an isolated NK cell and/or ILC3 cell population produced using the three-stage method described herein. In a specific embodiment, said isolated NK cell and/or ILC3 cell population is produced from hematopoietic cells, e.g., hematopoietic stem or progenitor cells isolated from placental perfusate, umbilical cord blood, and/or peripheral blood. In another specific embodiment, said isolated NK cell and/or ILC3 cell population comprises at least 50% of cells in the composition. In another specific embodiment, said isolated NK cell and/or ILC3 cell population, e.g., CD3⁻CD56⁺ cells, comprises at least 80%, 85%, 90%. 95%, 98% or 99% of cells in the composition. In certain embodiments, no more than 5%, 10%, 15%, 20%, 25%, 30%, 35%, or 40% of the cells in said isolated NK cell and/or ILC3 cell population are CD3⁻CD56⁺ cells. In certain embodiments, said CD3⁻CD56⁺ cells are CD16⁻.

NK cell and/or ILC3 cell populations produced using the three-stage method described herein, can be formulated into pharmaceutical compositions for use in vivo. Such pharmaceutical compositions comprise a population of NK cells and/or ILC3 cells in a pharmaceutically-acceptable carrier, e.g., a saline solution or other accepted physiologically-acceptable solution for in vivo administration. Pharmaceutical compositions of the invention can comprise any of the NK cell and/or ILC3 cell populations described elsewhere herein.

The pharmaceutical compositions of the invention comprise populations of cells that comprise 50% viable cells or more (that is, at least 50% of the cells in the population are functional or living). Preferably, at least 60% of the cells in the population are viable. More preferably, at least 70%, 80%, 90%, 95%, or 99% of the cells in the population in the pharmaceutical composition are viable.

The pharmaceutical compositions of the invention can comprise one or more compounds that, e.g., facilitate engraftment; stabilizers such as albumin, dextran 40, gelatin, hydroxy ethyl starch, and the like.

When formulated as an injectable solution, in one embodiment, the pharmaceutical composition of the invention comprises about 1.25% HSA and about 2.5% dextran. Other injectable formulations, suitable for the administration of cellular products, may be used.

In one embodiment, the compositions, e.g., pharmaceutical compositions, provided herein are suitable for systemic or local administration. In specific embodiments, the compositions, e.g., pharmaceutical compositions, provided herein are suitable for parenteral administration. In specific embodiments, the compositions, e.g., pharmaceutical compositions, provided herein are suitable for injection, infusion, intravenous (IV) administration, intrafemoral administration, or intratumor administration. In specific embodiments, the compositions, e.g., pharmaceutical compositions, provided herein are suitable for administration via a device, a matrix, or a scaffold. In specific embodiments, the compositions, e.g., pharmaceutical compositions provided herein are suitable for injection. In specific embodiments, the compositions, e.g., pharmaceutical compositions, provided herein are suitable for administration via a catheter. In specific embodiments, the compositions, e.g., pharmaceutical compositions, provided herein are suitable for local injection. In more specific embodiments, the compositions, e.g., pharmaceutical compositions, provided herein are suitable for local injection directly into a solid tumor (e.g., a sarcoma). In specific embodiments, the compositions, e.g., pharmaceutical compositions, provided herein are suitable for injection by syringe. In specific embodiments, the compositions, e.g., pharmaceutical compositions, provided herein are suitable for administration via guided delivery. In specific embodiments, the compositions, e.g., pharmaceutical compositions, provided herein are suitable for injection aided by laparoscopy, endoscopy, ultrasound, computed tomography, magnetic resonance, or radiology.

In certain embodiments, the compositions, e.g., pharmaceutical compositions provided herein, comprising NK cells and/or ILC3 cells produced using the methods described herein, are provided as pharmaceutical grade administrable units. Such units can be provided in discrete volumes, e.g., 15 mL, 20 mL, 25 mL, 30 nL. 35 mL, 40 mL, 45 mL, 50 mL, 55 mL, 60 mL, 65 mL, 70 mL, 75 mL, 80 mL, 85 mL, 90 mL, 95 mL, 100 mL, 150 mL, 200 mL, 250 mL, 300 mL, 350 mL, 400 mL, 450 mL, 500 mL, or the like. Such units can be provided so as to contain a specified number of cells, e.g., NK cells and/or ILC3 cells, e.g., 1×10⁴, 5×10⁴, 1×10⁵, 5×10⁵, 1×10⁶, 5×10⁶, 1×10⁷, 5×10⁷, 1×10⁸, 5×10⁸ or more cells per milliliter, or 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹ or more cells per unit. In specific embodiments, the units can comprise about, at least about, or at most about 1×10⁴, 5×10⁴, 1×10⁵, 5×10⁵, 1×10⁶, 5×10⁶ or more NK cells and/or ILC3 cells per milliliter, or 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹ or more cells per unit. Such units can be provided to contain specified numbers of NK cells and/or ILC3 cells or NK cell and/or ILC3 cell populations and/or any of the other cells. In specific embodiments, the NK cells and ILC3 cells are present in ratios provided herein.

In another specific embodiment, said isolated NK cells and/or ILC3 cells in said composition are from a single individual. In a more specific embodiment, said isolated NK cells and/or ILC3 cells comprise NK cells and/or ILC3 cells from at least two different individuals. In another specific embodiment, said isolated NK cells and/or ILC3 cells in said composition are from a different individual than the individual for whom treatment with the NK cells and/or ILC3 cells is intended. In another specific embodiment, said NK cells have been contacted or brought into proximity with an immunomodulatory compound or thalidomide in an amount and for a time sufficient for said NK cells to express detectably more granzyme B or perforin than an equivalent number of natural killer cells, i.e. NK cells not contacted or brought into proximity with said immunomodulatory compound or thalidomide. In another specific embodiment, said composition additionally comprises an immunomodulatory compound or thalidomide. In certain embodiments, the immunomodulatory compound is a compound described below. See, e.g., U.S. Pat. No. 7,498,171, the disclosure of which is hereby incorporated by reference in its entirety. In certain embodiments, the immunomodulatory compound is an amino-substituted isoindoline. In one embodiment, the immunomodulatory compound is 3-(4-amino-1-oxo-1,3-dihydroisoindol-2-yl)-piperidine-2,6-dione; 3-(4′aminoisolindoline-1′-one)-1-piperidine-2,6-dione; 4-(amino)-2-(2,6-dioxo(3-piperidyl))-isoindoline-1,3-dione; or 4-Amino-2-(2,6-dioxopiperidin-3-yl)isoindole-1,3-dione. In another embodiment, the immunomodulatory compound is pomalidomide, or lenalidomide. In another embodiment, said immunomodulatory compound is a compound having the structure

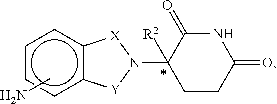

wherein one of X and Y is C═O, the other of X and Y is C═O or CH₂, and R² is hydrogen or lower alkyl, or a pharmaceutically acceptable salt, hydrate, solvate, clathrate, enantiomer, diastereomer, racemate, or mixture of stereoisomers thereof. In another embodiment, said immunomodulatory compound is a compound having the structure

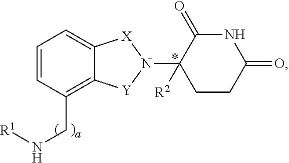

wherein one of X and Y is C═O and the other is CH₂ or C═O;

R¹ is H, (C₁-C₈)alkyl, (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl-(C₂-C₅)heteroaryl, C(O)R³, C(S)R³, C(O)OR⁴, (C₁-C₈)alkyl-N(R⁶)₂, (C₁-C₈)alkyl-OR⁵, (C₁-C₈)alkyl-C(O)OR⁵, C(O)NHR³, C(S)NHR³, C(O)NR³R^{3′}, C(S)NR³R^{3′} or (C₁-C₈)alkyl-O(CO)R⁵;

R² is H, F, benzyl, (C₁-C₈)alkyl, (C₂-C₈)alkenyl, or (C₂-C₈)alkynyl;

R³ and R^{3′} are independently (C₁-C₈)alkyl, (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₁-C₄)alkyl-(C₂-C₅)heteroaryl, (C₁-C₈)alkyl-N(R⁶)₂, (C₁-C₈)alkyl-OR⁵, (C₁-C₈)alkyl-C(O)OR⁵, (C₁-C₈)alkyl-O(CO)R⁵, or C(O)OR⁵;

R⁴ is (C₁-C₈)alkyl, (C₂-C₈)alkenyl, (C₂-C₈)alkynyl, (C₁-C₄)alkyl-OR⁵, benzyl, aryl, (C₀-C₄)alkyl-(C₁-C₆)heterocycloalkyl, or (C₀-C₄)alkyl-(C₂-C₅)heteroaryl;

R⁵ is (C₁-C₈)alkyl, (C₂-C₈)alkenyl, (C₂-C₈)alkynyl, benzyl, aryl, or (C₂-C₅)heteroaryl; each occurrence of R⁶ is independently H, (C₁-C₈)alkyl, (C₂-C₈)alkenyl, (C₂-C₈)alkynyl, benzyl, aryl, (C₂-C₅)heteroaryl, or (C₁-C₈)alkyl-C(O)O—R⁵ or the R⁶ groups can join to form a heterocycloalkyl group;

n is 0 or 1; and

* represents a chiral-carbon center;

or a pharmaceutically acceptable salt, hydrate, solvate, clathrate, enantiomer, diastereomer, racemate, or mixture of stereoisomers thereof. In another embodiment, said immunomodulatory compound is a compound having the structure

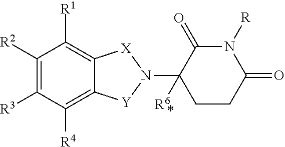

wherein:

one of X and Y is C═O and the other is CH₂ or C═O; R is H or CH₂OCOR′;

(i) each of R¹, R², R³, or R⁴, independently of the others, is halo, alkyl of 1 to 4 carbon atoms, or alkoxy of 1 to 4 carbon atoms or (ii) one of R¹, R², R³, or R⁴ is nitro or —NHR⁵ and the remaining of R¹, R², R³, or R⁴ are hydrogen;

R⁵ is hydrogen or alkyl of 1 to 8 carbons

R⁶ hydrogen, alkyl of 1 to 8 carbon atoms, benzo, chloro, or fluoro;

R′ is R⁷—CHR¹⁰—N(R⁸R⁹);

R⁷ is m-phenylene or p-phenylene or —(CₙH₂ₙ)— in which n has a value of 0 to 4;

each of R⁸ and R⁹ taken independently of the other is hydrogen or alkyl of 1 to 8 carbon atoms, or R⁸ and R⁹ taken together are tetramethylene, pentamethylene, hexamethylene, or —CH₂CH₂X₁CH₂CH₂— in which X₁ is —O—, —S—, or —NH—;

R¹⁰ is hydrogen, alkyl of to 8 carbon atoms, or phenyl; and

* represents a chiral-carbon center;

or a pharmaceutically acceptable salt, hydrate, solvate, clathrate, enantiomer, diastereomer, racemate, or mixture of stereoisomers thereof.

In another specific embodiment, the composition additionally comprises one or more anticancer compounds, e.g., one or more of the anticancer compounds described below.

In a more specific embodiment, the composition comprises NK cells and/or ILC3 cells from another source, or made by another method. In a specific embodiment, said other source is placental blood and/or umbilical cord blood. In another specific embodiment, said other source is peripheral blood. In more specific embodiments, the NK cell and/or ILC3 cell population in said composition is combined with NK cells and/or ILC3 cells from another source, or made by another method in a ratio of about 100:1, 95:5, 90:10, 85:15, 80:20, 75:25, 70:30, 65:35, 60:40, 55:45: 50:50, 45:55, 40:60, 35:65, 30:70, 25:75, 20:80, 15:85, 10:90, 5:95, 100:1, 95:1, 90:1, 85:1, 80:1, 75:1, 70:1, 65:1, 60:1, 55:1, 50:1, 45:1, 40:1, 35:1, 30:1, 25:1, 20:1, 15:1, 10:1, 5:1, 1:1, 1:5, 1:10, 1:15, 1:20, 1:25, 1:30, 1:35, 1:40, 1:45, 1:50, 1:55, 1:60, 1:65, 1:70, 1:75, 1:80, 1:85, 1:90, 1:95, 1:100, or the like.

In another specific embodiment, the composition comprises an NK cell and/or ILC3 cell population produced using the three-stage method described herein and either isolated placental perfusate or isolated placental perfusate cells. In a more specific embodiment, said placental perfusate is from the same individual as said NK cell and/or ILC3 cell population. In another more specific embodiment, said placental perfusate comprises placental perfusate from a different individual than said NK cell and/or ILC3 cell population. In another specific embodiment, all, or substantially all (e.g., greater than 90%, 95%, 98% or 99%) of cells in said placental perfusate are fetal cells. In another specific embodiment, the placental perfusate or placental perfusate cells, comprise fetal and maternal cells. In a more specific embodiment, the fetal cells in said placental perfusate comprise less than about 90%, 80%, 70%, 60% or 50% of the cells in said perfusate. In another specific embodiment, said perfusate is obtained by passage of a 0.9% NaCl solution through the placental vasculature. In another specific embodiment, said perfusate comprises a culture medium. In another specific embodiment, said perfusate has been treated to remove erythrocytes. In another specific embodiment, said composition comprises an immunomodulatory compound, e.g., an immunomodulatory compound described below, e.g., an amino-substituted isoindoline compound. In another specific embodiment, the composition additionally comprises one or more anticancer compounds, e.g., one or more of the anticancer compounds described below.

In another specific embodiment, the composition comprises an NK cell and/or ILC3 cell population and placental perfusate cells. In a more specific embodiment, said placental perfusate cells are from the same individual as said NK cell and/or ILC3 cell population. In another more specific embodiment, said placental perfusate cells are from a different individual than said NK cell and/or ILC3 cell population. In another specific embodiment, the composition comprises isolated placental perfusate and isolated placental perfusate cells, wherein said isolated perfusate and said isolated placental perfusate cells are from different individuals. In another more specific embodiment of any of the above embodiments comprising placental perfusate, said placental perfusate comprises placental perfusate from at least two individuals. In another more specific embodiment of any of the above embodiments comprising placental perfusate cells, said isolated placental perfusate cells are from at least two individuals. In another specific embodiment, said composition comprises an immunomodulatory compound. In another specific embodiment, the composition additionally comprises one or more anticancer compounds, e.g., one or more of the anticancer compounds described below.

5.11. Uses of NK Cells and/or ILC3 Cells Produced Using the Three-Stage Method

The NK cells and/or ILC3 cells produced using the methods described herein, e.g., NK cell and/or ILC3 cell produced according to the three-stage method described herein, provided herein can be used in methods of treating individuals having cancer, e.g., individuals having solid tumor cells and/or blood cancer cells, or persons having a viral infection. In some such embodiments, an effective dosage of NK cells and/or ILC3 cells produced using the methods described herein ranges from 1×10⁴ to 5×10⁴, 5×10⁴ to 1×10⁵, 1×10⁵ to 5×10⁵, 5×10⁵ to 1×10⁶, 1×10⁶ to 5×10⁶, 5×10⁶ to 1×10⁷, or more cells/kilogram body weight. The NK cells and/or ILC3 cells produced using the methods described herein, can also be used in methods of suppressing proliferation of tumor cells.

5.11.1. Treatment of Individuals Having Cancer

In one embodiment, provided herein is a method of treating an individual having a cancer, for example, a blood cancer or a solid tumor, comprising administering to said individual a therapeutically effective amount of NK cells produced using the methods described herein, e.g., NK cell populations produced using the three-stage method described herein. In one embodiment, provided herein is a method of treating an individual having a cancer, for example, a blood cancer or a solid tumor, comprising administering to said individual a therapeutically effective amount of ILC3 cells produced using the methods described herein, e.g., ILC3 cell populations produced using the three-stage method described herein. In certain embodiments, the individual has a deficiency of natural killer cells, e.g., a deficiency of NK cells active against the individual's cancer. In a specific embodiment, the method additionally comprises administering to said individual isolated placental perfusate or isolated placental perfusate cells, e.g., a therapeutically effective amount of placental perfusate or isolated placental perfusate cells. In another specific embodiment, the method comprises additionally administering to said individual an effective amount of an immunomodulatory compound, e.g., an immunomodulatory compound described above, or thalidomide. As used herein, an “effective amount” is an amount that, e.g., results in a detectable improvement of, lessening of the progression of, or elimination of, one or more symptoms of a cancer from which the individual suffers.

Administration of an isolated population of NK cells and/or ILC3 cells or a pharmaceutical composition thereof may be systemic or local. In specific embodiments, administration is parenteral. In specific embodiments, administration of an isolated population of NK cells and/or ILC3 cells or a pharmaceutical composition thereof to a subject is by injection, infusion, intravenous (IV) administration, intrafemoral administration, or intratumor administration. In specific embodiments, administration of an isolated population of NK cells and/or ILC3 cells or a pharmaceutical composition thereof to a subject is performed with a device, a matrix, or a scaffold. In specific embodiments, administration an isolated population of NK cells and/or ILC3 cells or a pharmaceutical composition thereof to a subject is by injection. In specific embodiments, administration an isolated population of NK cells and/or ILC3 cells or a pharmaceutical composition thereof to a subject is via a catheter. In specific embodiments, the injection of NK cells and/or ILC3 cells is local injection. In more specific embodiments, the local injection is directly into a solid tumor (e.g., a sarcoma). In specific embodiments, administration of an isolated population of NK cells and/or ILC3 cells or a pharmaceutical composition thereof to a subject is by injection by syringe. In specific embodiments, administration of an isolated population of NK cells and/or ILC3 cells or a pharmaceutical composition thereof to a subject is via guided delivery. In specific embodiments, administration of an isolated population of NK cells and/or ILC3 cells or a pharmaceutical composition thereof to a subject by injection is aided by laparoscopy, endoscopy, ultrasound, computed tomography, magnetic resonance, or radiology.

In a specific embodiment, the cancer is a blood cancer, e.g., a leukemia or a lymphoma. In more specific embodiments, the cancer is an acute leukemia, e.g., acute T cell leukemia, acute myelogenous leukemia (AML), acute promyelocytic leukemia, acute myeloblastic leukemia, acute megakaryoblastic leukemia, precursor B acute lymphoblastic leukemia, precursor T acute lymphoblastic leukemia, Burkitt's leukemia (Burkitt's lymphoma), or acute biphenotypic leukemia; a chronic leukemia, e.g., chronic myeloid lymphoma, chronic myelogenous leukemia (CML), chronic monocytic leukemia, chronic lymphocytic leukemia (CLL)/Small lymphocytic lymphoma, or B-cell prolymphocytic leukemia; hairy cell lymphoma; T-cell prolymphocytic leukemia; or a lymphoma, e.g., histiocytic lymphoma, lymphoplasmacytic lymphoma (e.g., Waldenström macroglobulinemia), splenic marginal zone lymphoma, plasma cell neoplasm (e.g., plasma cell myeloma, plasmacytoma, a monoclonal immunoglobulin deposition disease, or a heavy chain disease), extranodal marginal zone B cell lymphoma (MALT lymphoma), nodal marginal zone B cell lymphoma (NMZL), follicular lymphoma, mantle cell lymphoma, diffuse large B cell lymphoma, mediastinal (thymic) large B cell lymphoma, intravascular large B cell lymphoma, primary effusion lymphoma, T cell large granular lymphocytic leukemia, aggressive NK cell leukemia, adult T cell leukemia/lymphoma, extranodal NK/T cell lymphoma, nasal type, enteropathy-type T cell lymphoma, hepatosplenic T cell lymphoma, blastic NK cell lymphoma, mycosis fungoides (Sezary syndrome), a primary cutaneous CD30-positive T cell lymphoproliferative disorder (e.g., primary cutaneous anaplastic large cell lymphoma or lymphomatoid papulosis), angioimmunoblastic T cell lymphoma, peripheral T cell lymphoma, unspecified, anaplastic large cell lymphoma, a Hodgkin's lymphoma or a nodular lymphocyte-predominant Hodgkin's lymphoma. In another specific embodiment, the cancer is multiple myeloma or myelodysplastic syndrome.

In certain other specific embodiments, the cancer is a solid tumor, e.g., a carcinoma, such as an adenocarcinoma, an adrenocortical carcinoma, a colon adenocarcinoma, a colorectal adenocarcinoma, a colorectal carcinoma, a ductal cell carcinoma, a lung carcinoma, a thyroid carcinoma, a nasopharyngeal carcinoma, a melanoma (e.g., a malignant melanoma), a non-melanoma skin carcinoma, or an unspecified carcinoma; a desmoid tumor; a desmoplastic small round cell tumor; an endocrine tumor; an Ewing sarcoma; a germ cell tumor (e.g., testicular cancer, ovarian cancer, choriocarcinoma, endodermal sinus tumor, germinoma, etc); a hepatosblastoma; a hepatocellular carcinoma; a neuroblastoma; a non-rhabdomyosarcoma soft tissue sarcoma; an osteosarcoma; a retinoblastoma; a rhabdomyosarcoma; or a Wilms tumor. In another embodiment, the solid tumor is pancreatic cancer or breast cancer. In other embodiments, the solid tumor is an acoustic neuroma; an astrocytoma (e.g., a grade I pilocytic astrocytoma, a grade II low-grade astrocytoma; a grade III anaplastic astrocytoma; or a grade IV glioblastoma multiforme); a chordoma; a craniopharyngioma; a glioma (e.g., a brain stem glioma; an ependymoma; a mixed glioma; an optic nerve glioma; or a subependymoma); a glioblastoma; a medulloblastoma; a meningioma; a metastatic brain tumor; an oligodendroglioma; a pineoblastoma; a pituitary tumor; a primitive neuroectodermal tumor; or a schwannoma. In another embodiment, the cancer is prostate cancer. In another embodiment, the cancer is liver cancer. In another embodiment, the cancer is lung cancer. In another embodiment, the cancer is renal cancer.

In certain embodiments, the individual having a cancer, for example, a blood cancer or a solid tumor, e.g., an individual having a deficiency of natural killer cells, is an individual that has received a bone marrow transplant before said administering. In certain embodiments, the bone marrow transplant was in treatment of said cancer. In certain other embodiments, the bone marrow transplant was in treatment of a condition other than said cancer. In certain embodiments, the individual received an immunosuppressant in addition to said bone marrow transplant. In certain embodiments, the individual who has had a bone marrow transplant exhibits one or more symptoms of graft-versus-host disease (GVHD) at the time of said administration. In certain other embodiments, the individual who has had a bone marrow transplant is administered said cells before a symptom of GVHD has manifested.

In certain specific embodiments, the individual having a cancer, for example, a blood cancer, has received at least one dose of a TNFα inhibitor, e.g., ETANERCEPT® (Enbrel), prior to said administering. In specific embodiments, said individual received said dose of a TNFα inhibitor within 1, 2, 3, 4, 5, 6, 7, 8, 9, 10, 11 or 12 months of diagnosis of said cancer. In a specific embodiment, the individual who has received a dose of a TNFα inhibitor exhibits acute myeloid leukemia. In a more specific embodiment, the individual who has received a dose of a TNFα inhibitor and exhibits acute myeloid leukemia further exhibits deletion of the long arm of chromosome 5 in blood cells. In another embodiment, the individual having a cancer, for example, a blood cancer, exhibits a Philadelphia chromosome.

In certain other embodiments, the cancer, for example, a blood cancer or a solid tumor, in said individual is refractory to one or more anticancer drugs. In a specific embodiment, the cancer is refractory to GLEEVEC® (imatinib mesylate).

In certain embodiments, the cancer, for example, a blood cancer, in said individual responds to at least one anticancer drug; in this embodiment, placental perfusate, isolated placental perfusate cells, isolated natural killer cells, e.g., placental natural killer cells, e.g., placenta-derived intermediate natural killer cells, isolated combined natural killer cells, or NK cells described herein, and/or combinations thereof, and optionally an immunomodulatory compound, are added as adjunct treatments or as a combination therapy with said anticancer drug. In certain other embodiments, the individual having a cancer, for example, a blood cancer, has been treated with at least one anticancer drug, and has relapsed, prior to said administering. In certain embodiments, the individual to be treated has a refractory cancer. In one embodiment, the cancer treatment method with the cells described herein protects against (e.g., prevents or delays) relapse of cancer. In one embodiment, the cancer treatment method described herein results in remission of the cancer for 1 month or more, 2, 3, 4, 5, 6, 7, 8, 9, 10, 11, or 12 months or more, 1 year or more, 2 years or more, 3 years or more, or 4 years or more.

In one embodiment, provided herein is a method of treating an individual having multiple myeloma, comprising administering to the individual (1) lenalidomide; (2) melphalan; and (3) NK cells, wherein said NK cells are effective to treat multiple myeloma in said individual. In a specific embodiment, said NK cells are cord blood NK cells, or NK cells produced from cord blood hematopoietic cells, e.g., hematopoietic stem cells. In another embodiment, said NK cells have been produced by a three-stage method described herein for producing NK cells. In another embodiment, said lenalidomide, melphalan, and/or NK cells are administered separately from each other. In certain specific embodiments of the method of treating an individual with multiple myeloma, said NK cells are produced by a method comprising: culturing hematopoietic stem cells or progenitor cells, e.g., CD34⁺ stem cells or progenitor cells, in a first medium comprising a stem cell mobilizing agent and thrombopoietin (Tpo) to produce a first population of cells, subsequently culturing said first population of cells in a second medium comprising a stem cell mobilizing agent and interleukin-15 (IL-15), and lacking Tpo, to produce a second population of cells, and subsequently culturing said second population of cells in a third medium comprising IL-2 and IL-15, and lacking a stem cell mobilizing agent and LMWH, to produce a third population of cells, wherein the third population of cells comprises natural killer cells that are CD56+, CD3−, CD16− or CD16+, and CD94+ or CD94−, and wherein at least 70%, or at least 80%, of the natural killer cells are viable. In certain embodiments, said first medium and/or said second medium lack leukemia inhibiting factor (LIF) and/or macrophage inflammatory protein-1 alpha (MIP-1α). In certain embodiments, said third medium lacks LIF, MIP-1α, and FMS-like tyrosine kinase-3 ligand (Flt-3L). In specific embodiments, said first medium and said second medium lack LIF and MIP-1α, and said third medium lacks LIF, MIP-1α, and Flt3L. In certain embodiments, none of the first medium, second medium or third medium comprises heparin, e.g., low-molecular weight heparin.

In another embodiment, provided herein is a method of treating an individual having acute myelogenous leukemia (AML), comprising administering to the individual NK cells (optionally activated by pretreatment with IL2 alone, or IL-15 alone, IL2 and IL12 and IL18, IL12 and IL15, IL12 and IL18, IL2 and IL12 and IL15 and IL18, or IL2 and IL15 and IL18), wherein said NK cells are effective to treat AML in said individual. In a specific embodiment, the isolated NK cell population produced using the three-stage methods described herein has been pretreated with one or more of IL2, IL12, IL18, or IL15 prior to said administering. In a specific embodiment, said NK cells are cord blood NK cells, or NK cells produced from cord blood hematopoietic cells, e.g., hematopoietic stem cells. In another embodiment, said NK cells have been produced by a three-stage method described herein for producing NK cells. In certain specific embodiments of the method of treating an individual with AML, said NK cells are produced by a three-stage method, as described herein. In a particular embodiment, the AML to be treated by the foregoing methods comprises refractory AML, poor-prognosis AML, or childhood AML. Methods known in the art for administering NK cells for the treatment of refractory AML, poor-prognosis AML, or childhood AML may be adapted for this purpose; see, e.g., Miller et al., 2005, Blood 105:3051-3057; Rubnitz et al., 2010, J Clin Oncol. 28:955-959, each of which is incorporated herein by reference in its entirety. In certain embodiments, said individual has AML that has failed at least one non-natural killer cell therapeutic against AML. In specific embodiments, said individual is 65 years old or greater, and is in first remission. In specific embodiments, said individual has been conditioned with fludarabine, cytarabine, or both prior to administering said natural killer cells.

In one embodiment, provided herein is a method of treating an individual having multiple myeloma, comprising administering to the individual (1) lenalidomide; (2) melphalan; and (3) ILC3 cells, wherein said ILC3 cells are effective to treat multiple myeloma in said individual. In a specific embodiment, said ILC3 cells are cord blood ILC3 cells, or ILC3 cells produced from cord blood hematopoietic cells, e.g., hematopoietic stem cells. In another embodiment, said ILC3 cells have been produced by a three-stage method described herein for producing ILC3 cells. In another embodiment, said lenalidomide, melphalan, and/or ILC3 cells are administered separately from each other. In certain specific embodiments of the method of treating an individual with multiple myeloma, said ILC3 cells are produced by a method comprising: culturing hematopoietic stem cells or progenitor cells, e.g., CD34⁺ stem cells or progenitor cells, in a first medium comprising a stem cell mobilizing agent and thrombopoietin (Tpo) to produce a first population of cells, subsequently culturing said first population of cells in a second medium comprising a stem cell mobilizing agent and interleukin-15 (IL-15), and lacking Tpo, to produce a second population of cells, and subsequently culturing said second population of cells in a third medium comprising IL-2 and IL-15, and lacking a stem cell mobilizing agent and LMWH, to produce a third population of cells, wherein the third population of cells comprises natural killer cells that are CD56+, CD3−, CD16− or CD16+, and CD94+ or CD94−, and wherein at least 70%, or at least 80%, of the natural killer cells are viable. In certain embodiments, said first medium and/or said second medium lack leukemia inhibiting factor (LIF) and/or macrophage inflammatory protein-1 alpha (MIP-1α). In certain embodiments, said third medium lacks LIF, MIP-1α, and FMS-like tyrosine kinase-3 ligand (Flt-3L). In specific embodiments, said first medium and said second medium lack LIF and MIP-1α, and said third medium lacks LIF, MIP-1α, and Flt3L. In certain embodiments, none of the first medium, second medium or third medium comprises heparin, e.g., low-molecular weight heparin.

In another embodiment, provided herein is a method of treating an individual having acute myelogenous leukemia (AML), comprising administering to the individual ILC3 cells (optionally activated by pretreatment with IL2 and IL12 and IL18, IL12 and IL15, IL12 and IL18, IL2 and IL12 and IL15 and IL18, or IL2 and IL15 and IL18), wherein said ILC3 cells are effective to treat AML in said individual. In a specific embodiment, the ILC3 cell population produced using the three-stage methods described herein has been pretreated with one or more of IL2, IL12, IL18, or IL15 prior to said administering. In a specific embodiment, said ILC3 cells are cord blood ILC3 cells, or ILC3 cells produced from cord blood hematopoietic cells, e.g., hematopoietic stem cells. In another embodiment, said ILC3 cells have been produced by a three-stage method described herein for producing ILC3 cells. In certain specific embodiments of the method of treating an individual with AML, said ILC3 cells are produced by a three-stage method, as described herein. In a particular embodiment, the AML to be treated by the foregoing methods comprises refractory AML, poor-prognosis AML, or childhood AML.

Methods known in the art for administering ILC3 cells for the treatment of refractory AML, poor-prognosis AML, or childhood AML may be adapted for this purpose; see, e.g., Miller et al., 2005, Blood 105:3051-3057; Rubnitz et al., 2010, J Clin Oncol. 28:955-959, each of which is incorporated herein by reference in its entirety. In certain embodiments, said individual has AML that has failed at least one non-natural killer cell therapeutic against AML. In specific embodiments, said individual is 65 years old or greater, and is in first remission. In specific embodiments, said individual has been conditioned with fludarabine, cytarabine, or both prior to administering said natural killer cells.

In other specific embodiments of the method of treating an individual with AML, said NK cells are produced by a method comprising: culturing hematopoietic stem cells or progenitor cells, e.g., CD34⁺ stem cells or progenitor cells, in a first medium comprising a stem cell mobilizing agent and thrombopoietin (Tpo) to produce a first population of cells, subsequently culturing said first population of cells in a second medium comprising a stem cell mobilizing agent and interleukin-15 (IL-15), and lacking Tpo, to produce a second population of cells, and subsequently culturing said second population of cells in a third medium comprising IL-2 and IL-15, and lacking a stem cell mobilizing agent and LMWH, to produce a third population of cells, wherein the third population of cells comprises natural killer cells that are CD56+, CD3−, CD16− or CD16+, and CD94+ or CD94−, and wherein at least 70%, or at least 80%, of the natural killer cells are viable. In certain embodiments, said first medium and/or said second medium lack leukemia inhibiting factor (LIF) and/or macrophage inflammatory protein-1 alpha (MIP-1α). In certain embodiments, said third medium lacks LIF, MIP-1α, and FMS-like tyrosine kinase-3 ligand (Flt-3L). In specific embodiments, said first medium and said second medium lack LIF and MIP-1α, and said third medium lacks LIF, MIP-1α, and Flt3L. In certain embodiments, none of the first medium, second medium or third medium comprises heparin, e.g., low-molecular weight heparin.

In another embodiment, provided herein is a method of treating an individual having chronic lymphocytic leukemia (CLL), comprising administering to the individual a therapeutically effective dose of (1) lenalidomide; (2) melphalan; (3) fludarabine; and (4) NK cells, e.g., NK cells produced by a three-stage method described herein, wherein said NK cells are effective to treat said CLL in said individual. In a specific embodiment, said NK cells are cord blood NK cells, or NK cells produced from cord blood hematopoietic stem cells. In another embodiment, said NK cells have been produced by a three-stage method described herein for producing NK cells. In a specific embodiment of any of the above methods, said lenalidomide, melphalan, fludarabine, and expanded NK cells are administered to said individual separately. In certain specific embodiments of the method of treating an individual with CLL, said NK cells are produced by a method comprising: culturing hematopoietic stem cells or progenitor cells, e.g., CD34⁺ stem cells or progenitor cells, in a first medium comprising a stem cell mobilizing agent and thrombopoietin (Tpo) to produce a first population of cells, subsequently culturing said first population of cells in a second medium comprising a stem cell mobilizing agent and interleukin-15 (IL-15), and lacking Tpo, to produce a second population of cells, and subsequently culturing said second population of cells in a third medium comprising IL-2 and IL-15, and lacking a stem cell mobilizing agent and LMWH, to produce a third population of cells, wherein the third population of cells comprises natural killer cells that are CD56+, CD3−, CD16− or CD16+, and CD94+ or CD94−, and wherein at least 70%, or at least 80%, of the natural killer cells are viable. In certain embodiments, said first medium and/or said second medium lack leukemia inhibiting factor (LIF) and/or macrophage inflammatory protein-1 alpha (MIP-1α). In certain embodiments, said third medium lacks LIF, MIP-1α, and FMS-like tyrosine kinase-3 ligand (Flt-3L). In specific embodiments, said first medium and said second medium lack LIF and MIP-1α, and said third medium lacks LIF, MIP-1α, and Flt3L. In certain embodiments, none of the first medium, second medium or third medium comprises heparin, e.g., low-molecular weight heparin.

5.11.2. Suppression of Tumor Cell Proliferation

Further provided herein is a method of suppressing the proliferation of tumor cells, comprising bringing NK cells produced using the methods described herein, e.g., NK cell populations produced using the three-stage method described herein, into proximity with the tumor cells, e.g., contacting the tumor cells with NK cells produced using the methods described herein. A plurality of the NK cells can thus be used in the method of suppressing the proliferation of the tumor cells comprising bringing a therapeutically effective amount of the NK cell population into proximity with the tumor cells, e.g., contacting the tumor cells with the cells in the NK cell population. Optionally, isolated placental perfusate or isolated placental perfusate cells is brought into proximity with the tumor cells and/or NK cells produced using the methods described herein. In another specific embodiment, an immunomodulatory compound, e.g., an immunomodulatory compound described above, or thalidomide is additionally brought into proximity with the tumor cells and/or NK cells produced using the methods described herein, such that proliferation of the tumor cells is detectably reduced compared to tumor cells of the same type not brought into proximity with NK cells produced using the methods described herein. Optionally, isolated placental perfusate or isolated placental perfusate cells are brought into proximity with the tumor cells and/or NK cells produced using the methods described herein that have been contacted or brought into proximity with an immunomodulatory compound.

Also provided herein is a method of suppressing the proliferation of tumor cells, comprising bringing ILC3 cells produced using the methods described herein, e.g., ILC3 cell populations produced using the three-stage method described herein, into proximity with the tumor cells, e.g., contacting the tumor cells with ILC3 cells produced using the methods described herein. A plurality of the ILC3 cells can thus be used in the method of suppressing the proliferation of the tumor cells comprising bringing a therapeutically effective amount of the ILC3 cell population into proximity with the tumor cells, e.g., contacting the tumor cells with the cells in the ILC3 cell population. Optionally, isolated placental perfusate or isolated placental perfusate cells is brought into proximity with the tumor cells and/or ILC3 cells produced using the methods described herein. In another specific embodiment, an immunomodulatory compound, e.g., an immunomodulatory compound described above, or thalidomide is additionally brought into proximity with the tumor cells and/or ILC3 cells produced using the methods described herein, such that proliferation of the tumor cells is detectably reduced compared to tumor cells of the same type not brought into proximity with ILC3 cells produced using the methods described herein. Optionally, isolated placental perfusate or isolated placental perfusate cells are brought into proximity with the tumor cells and/or ILC3 cells produced using the methods described herein that have been contacted or brought into proximity with an immunomodulatory compound.

As used herein, in certain embodiments, “contacting,” or “bringing into proximity,” with respect to cells, in one embodiment encompasses direct physical, e.g., cell-cell, contact between placental perfusate, placental perfusate cells, natural killer cells, e.g., NK cell populations produced according to the three-stage method described herein, ILC3 cells, e.g., ILC3 cell populations produced according to the three-stage method described herein, and/or isolated combined natural killer cells and the tumor cells. In another embodiment, “contacting” encompasses presence in the same physical space, e.g., placental perfusate, placental perfusate cells, natural killer cells, e.g., placental intermediate natural killer cells, natural killer cells described herein, e.g., NK cell populations produced according to the three-stage method described herein, ILC3 cells described herein, e.g., ILC3 cell populations produced according to the three-stage method described herein, and/or isolated combined natural killer cells are placed in the same container (e.g., culture dish, multiwell plate) as tumor cells. In another embodiment, “contacting” placental perfusate, placental perfusate cells, combined natural killer cells, placental intermediate natural killer cells, or natural killer cells described herein, e.g., NK cell populations produced according to the three-stage method described herein or ILC3 cells described herein, e.g., ILC3 cell populations produced according to the three-stage method described herein, and tumor cells is accomplished, e.g., by injecting or infusing the placental perfusate or cells, e.g., placental perfusate cells, combined natural killer cells, natural killer cells, e.g., placental intermediate natural killer cells, or ILC3 cells, into an individual, e.g., a human comprising tumor cells, e.g., a cancer patient. “Contacting,” in the context of immunomodulatory compounds and/or thalidomide, means, e.g., that the cells and the immunomodulatory compound and/or thalidomide are directly physically contacted with each other, or are placed within the same physical volume (e.g., a cell culture container or an individual).

In a specific embodiment, the tumor cells are blood cancer cells, e.g., leukemia cells or lymphoma cells. In more specific embodiments, the cancer is an acute leukemia, e.g., acute T cell leukemia cells, acute myelogenous leukemia (AML) cells, acute promyelocytic leukemia cells, acute myeloblastic leukemia cells, acute megakaryoblastic leukemia cells, precursor B acute lymphoblastic leukemia cells, precursor T acute lymphoblastic leukemia cells, Burkitt's leukemia (Burkitt's lymphoma) cells, or acute biphenotypic leukemia cells; chronic leukemia cells, e.g., chronic myeloid lymphoma cells, chronic myelogenous leukemia (CML) cells, chronic monocytic leukemia cells, chronic lymphocytic leukemia (CLL)/Small lymphocytic lymphoma cells, or B-cell prolymphocytic leukemia cells; hairy cell lymphoma cells; T-cell prolymphocytic leukemia cells; or lymphoma cells, e.g., histiocytic lymphoma cells, lymphoplasmacytic lymphoma cells (e.g., Waldenström macroglobulinemia cells), splenic marginal zone lymphoma cells, plasma cell neoplasm cells (e.g., plasma cell myeloma cells, plasmacytoma cells, monoclonal immunoglobulin deposition disease, or a heavy chain disease), extranodal marginal zone B cell lymphoma (MALT lymphoma) cells, nodal marginal zone B cell lymphoma (NMZL) cells, follicular lymphoma cells, mantle cell lymphoma cells, diffuse large B cell lymphoma cells, mediastinal (thymic) large B cell lymphoma cells, intravascular large B cell lymphoma cells, primary effusion lymphoma cells, T cell large granular lymphocytic leukemia cells, aggressive NK cell leukemia cells, adult T cell leukemia/lymphoma cells, extranodal NK/T cell lymphoma—nasal type cells, enteropathy-type T cell lymphoma cells, hepatosplenic T cell lymphoma cells, blastic NK cell lymphoma cells, mycosis fungoides (Sezary syndrome), primary cutaneous CD30-positive T cell lymphoproliferative disorder (e.g., primary cutaneous anaplastic large cell lymphoma or lymphomatoid papulosis) cells, angioimmunoblastic T cell lymphoma cells, peripheral T cell lymphoma—unspecified cells, anaplastic large cell lymphoma cells, Hodgkin lymphoma cells or nodular lymphocyte-predominant Hodgkin lymphoma cells. In another specific embodiment, the tumor cells are multiple myeloma cells or myelodysplastic syndrome cells.

In specific embodiments, the tumor cells are solid tumor cells, e.g., carcinoma cells, for example, adenocarcinoma cells, adrenocortical carcinoma cells, colon adenocarcinoma cells, colorectal adenocarcinoma cells, colorectal carcinoma cells, ductal cell carcinoma cells, lung carcinoma cells, thyroid carcinoma cells, nasopharyngeal carcinoma cells, melanoma cells (e.g., malignant melanoma cells), non-melanoma skin carcinoma cells, or unspecified carcinoma cells; desmoid tumor cells; desmoplastic small round cell tumor cells; endocrine tumor cells; Ewing sarcoma cells; germ cell tumor cells (e.g., testicular cancer cells, ovarian cancer cells, choriocarcinoma cells, endodermal sinus tumor cells, germinoma cells, etc.); hepatosblastoma cells; hepatocellular carcinoma cells; neuroblastoma cells; non-rhabdomyosarcoma soft tissue sarcoma cells; osteosarcoma cells; retinoblastoma cells; rhabdomyosarcoma cells; or Wilms tumor cells. In another embodiment, the tumor cells are pancreatic cancer cells or breast cancer cells. In other embodiments, the solid tumor cells are acoustic neuroma cells; astrocytoma cells (e.g., grade I pilocytic astrocytoma cells, grade II low-grade astrocytoma cells; grade III anaplastic astrocytoma cells; or grade IV glioblastoma multiforme cells); chordoma cells; craniopharyngioma cells; glioma cells (e.g., brain stem glioma cells; ependymoma cells; mixed glioma cells; optic nerve glioma cells; or subependymoma cells); glioblastoma cells; medulloblastoma cells; meningioma cells; metastatic brain tumor cells; oligodendroglioma cells; pineoblastoma cells; pituitary tumor cells; primitive neuroectodermal tumor cells; or schwannoma cells. In another embodiment, the tumor cells are prostate cancer cells.

As used herein, “therapeutically beneficial” and “therapeutic benefits” include, but are not limited to, e.g., reduction in the size of a tumor; lessening or cessation of expansion of a tumor; reducing or preventing metastatic disease; reduction in the number of cancer cells in a tissue sample, e.g., a blood sample, per unit volume; the clinical improvement in any symptom of the particular cancer or tumor said individual has, the lessening or cessation of worsening of any symptom of the particular cancer the individual has, etc.

5.11.3. Treatment of Cancers Using NK Cells and/or ILC3 Cells and Other Anticancer Agents

Treatment of an individual having cancer using the NK cells produced using the methods described herein, e.g., NK cell populations produced using the three-stage method described herein, can be part of an anticancer therapy regimen that includes one or more other anticancer agents. Likewise, treatment of an individual having cancer using the ILC3 cells produced using the methods described herein, e.g., ILC3 cell populations produced using the three-stage method described herein, can be part of an anticancer therapy regimen that includes one or more other anticancer agents. In addition or alternatively, treatment of an individual having cancer using the NK cells and/or ILC3 cells produced using the methods described herein can be used to supplement an anticancer therapy that includes one or more other anticancer agents. Such anticancer agents are well-known in the art and include anti-inflammatory agents, immumodulatory agents, cytotoxic agents, cancer vaccines, chemotherapeutics, HD AC inhibitors (e.g., HDAC6i (ACY-241)), and siRNAs. Specific anticancer agents that may be administered to an individual having cancer, e.g., an individual having tumor cells, in addition to the NK cells produced using the methods described herein and optionally perfusate, perfusate cells, natural killer cells other than NK cells produced using the methods described herein include, but are not limited to: acivicin; aclarubicin; acodazole hydrochloride; acronine; adozelesin; adriamycin; adrucil; aldesleukin; altretamine; ambomycin; ametantrone acetate; amsacrine; anastrozole; anthramycin; asparaginase (e.g., from Erwinia chrysan; Erwinaze); asperlin; avastin (bevacizumab); azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; celecoxib (COX-2 inhibitor); Cerubidine; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflomithine hydrochloride; elsamitrucin; Elspar; enloplatin; enpromate; epipropidine; epirubicin hydrochloride; erbulozole; esorubicin hydrochloride; estramustine; estramustine phosphate sodium; etanidazole; etoposide; etoposide phosphate; Etopophos; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; Idamycin; idarubicin hydrochloride; ifosfamide; ilmofosine; iproplatin; irinotecan; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; Proleukin; Purinethol; puromycin; puromycin hydrochloride; pyrazofurin; Rheumatrex; riboprine; safingol; safingol hydrochloride; semustine; simtrazene; sparfosate sodium; sparsomycin; spirogermanium hydrochloride; spiromustine; spiroplatin; streptonigrin; streptozocin; sulofenur; Tabloid; talisomycin; tecogalan sodium; taxotere; tegafur; teloxantrone hydrochloride; temoporfin; teniposide; teroxirone; testolactone; thiamiprine; thioguanine; thiotepa; tiazofurin; tirapazamine; Toposar; toremifene citrate; trestolone acetate; Trexall; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; and zorubicin hydrochloride.

Other anti-cancer drugs include, but are not limited to: 20-epi-1,25 dihydroxyvitamin D3; 5-azacytidine; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptosar (also called Campto; irinotecan) camptothecin derivatives; capecitabine; carboxamide-amino-triazole; carboxyamidotriazole; CaRestM3; CARN 700; cartilage derived inhibitor; carzelesin; casein kinase inhibitors (ICOS); castanospermine; CC-122; CC-220; CC-486; cecropinB; cetrorelix; chlorlns; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidenmin B; deslorelin; dexamethasone; dexifosfamide; dexrazoxane; dexverapamil; diaziquone; didemninB; didox; diethylnorspermine; dihydro-5-azacytidine; dihydrotaxol, 9-; dioxamycin; diphenyl spiromustine; docetaxel; docosanol; dolasetron; doxifluridine; doxorubicin; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine (e.g., Fludara); fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; homoharringtonine (HHT, omacetaxine mepesuccinate); hypericin; ibandronic acid; idarubicin; idoxifene; idramantone; ilmofosine; ilomastat; imatinib (e.g., GLEEVEC®), imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MTF inhibitor; mifepristone; miltefosine; mirimostim; mitoguazone; mitolactol; mitomycin analogues; mitonafide; mitotoxin fibroblast growth factor-saporin; mitoxantrone; mofarotene; molgramostim; anti-EGFR antibody (e.g., Erbitux (cetuximab)); anti-CD19 antibody; anti-CD20 antibody (e.g., rituximab); anti-CS-1 antibody (e.g., elotuzumab (BMS/AbbVie)); anti-CD38 antibody (e.g., daratumumab (Genmab/Janssen Biotech); anti-CD138 antibody (e.g., indatuximab (Biotest AG Dreieich)); anti-PD-1 antibody; anti-PD-L1 antibody (e.g., durvalumab (AstraZeneca)); anti-NKG2A antibody (e.g., monalizumab (IPH2201; Innate Pharma)); anti-DLL4 antibody (e.g., demcizumab (Oncomed/Celgene)); anti-DLL4 and anti-VEGF bispecific antibody; anti-RSP03 antibody; anti-TIGIT antibody; ICOS agonist antibody; anti-disialoganglioside (GD2) antibody (e.g., monoclonal antibody 3F8 or chl4.18); anti-ErbB2 antibody (e.g., herceptin); human chorionic gonadotrophin; monophosphoryl lipid A+myobacterium cell wall sk; mopidamol; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; nilutamide; nisamycin; nitric oxide modulators; nitroxide antioxidant; nitrullyn; oblimersen (GENASENSE®); O⁶-benzylguanine; octreotide; okicenone; oligonucleotides; onapristone; ondansetron; ondansetron; oracin; oral cytokine inducer; ormaplatin; osaterone; oxaliplatin (e.g., Floxatin); oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rohitukine; romurtide; roquinimex; rubiginone Bl; ruboxyl; safingol; saintopin; SarCNU; sarcophytol A; sargramostim; Sdi 1 mimetics; semustine; senescence derived inhibitor 1; sense oligonucleotides; signal transduction inhibitors; sizofiran; sobuzoxane; sodium borocaptate; sodium phenylacetate; solverol; somatomedin binding protein; sonermin; sparfosic acid; spicamycinD; spiromustine; splenopentin; spongistatin 1; squalamine; stipiamide; stromelysin inhibitors; sulfinosine; superactive vasoactive intestinal peptide antagonist; suradista; suramin; swainsonine; tallimustine; tamoxifen methiodide; tauromustine; tazarotene; tecogalan sodium; tegafur; tellurapyrylium; telomerase inhibitors; temoporfin; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; Vectibix (panitumumab)velaresol; veramine; verdins; verteporfin; vinorelbine; vinxaltine; vitaxin; vorozole; Welcovorin (leucovorin); Xeloda (capecitabine); zanoterone; zeniplatin; zilascorb; and zinostatin stimalamer.

Treatment of an individual having cancer using the NK cells produced using the methods described herein, e.g., NK cell populations produced using the three-stage method described herein, can be part of an anticancer therapy regimen that includes one or more immune checkpoint modulator. In certain embodiments, the immune checkpoint modulator modulates an immune checkpoint molecule such as CD28, OX40, Glucocorticoid-Induced Tumour-necrosis factor Receptor-related protein (GITR), CD137 (4-1BB), CD27, Herpes Virus Entry Mediator (HVEM), T cell Immunoglobulin and Mucin-domain containing-3 (TIM-3), Lymphocyte-Activation Gene 3 (LAG-3), Cytotoxic T-Lymphocyte-associated Antigen-4 (CTLA-4), V-domain Immunoglobulin Suppressor of T cell Activation (VISTA), B and T Lymphocyte Attenuator (BTLA), PD-1, and/or PD-L1. In certain embodiments, the immune checkpoint molecule is an antibody or antigen-binding fragment thereof.

In certain embodiments, the immune checkpoint modulator is an agonist of an immune checkpoint molecule. In certain embodiments, the immune checkpoint molecule is CD28, OX40, Glucocorticoid-Induced Tumour-necrosis factor Receptor-related protein (GITR), CD137 (4-1BB), CD27, ICOS (CD278); Inducible T-cell Costimulator) and/or Herpes Virus Entry Mediator (HVEM). In certain embodiments, the immune checkpoint modulator is an antibody or antigen-binding fragment thereof.

In certain embodiments, the immune checkpoint modulator is an antagonist of an immune checkpoint molecule. In certain embodiments, the immune checkpoint molecule is T cell Immunoglobulin and Mucin-domain containing-3 (TIM-3), Lymphocyte-Activation Gene 3 (LAG-3), Cytotoxic T-Lymphocyte-associated Antigen-4 (CTLA-4), V-domain Immunoglobulin Suppressor of T cell Activation (VISTA), B and T Lymphocyte Attenuator (BTLA), PD-1, and/or PD-L1. In certain embodiments, the immune checkpoint modulator is an antibody or antigen-binding fragment thereof.

In certain embodiments, the immune checkpoint modulator is an antibody or antigen-binding fragment thereof. In certain embodiments, the antibody or antibody-binding fragment thereof binds PD-1. In certain embodiments, the antibody or antibody-binding fragment thereof that binds PD-1 is nivolumab (OPDIVO® BMS-936558, MDX-1106, ONO-4538; Bristol-Myers Squibb, Ono Pharmaceuticals, Inc.), pembrolizumab (KEYTRUDA®, lambrolizumab, MK-3475; Merck), pidilizumab (CT-011; Curetech, Medivation); MEDI0680 (AMP-514; MedImmune, AstraZeneca); PDR-001 (Novartis), SHR1210, or INCSHR1210; Incyte, Jiangsu Hengrui). In certain embodiments, the antibody or antigen-binding fragment thereof binds PD-L1. In certain embodiments, the antibody or antigen-binding fragment thereof that binds PD-L1 is durvalumab (MEDI4736; MedImmune, AstraZeneca), BMS-936559 (MDX-1105; Bristol-Myers Squibb), avelumab (MSB0010718C; Merck Serono, Pfizer), or atezolizumab (MPDL-3280A; Genentech, Roche). In certain embodiments, the antibody or antibody-binding fragment thereof binds LAG-3. In certain embodiments, the antibody or antibody-binding fragment thereof that binds LAG-3 is BMS-986016 (Bristol-Myers Squibb), GSK2831781 (GlaxoSmithKline), or LAG525 (Novartis). In certain embodiments, the antibody or antibody-binding fragment thereof binds CTLA-4. In certain embodiments, the antibody or antibody-binding fragment thereof that binds CTLA-4 is ipilimumab (YERVOY™, BMS-734016, MDX010, MDX-101; Bristol-Myers Squibb), or tremelimumab (CP-675,206; MedImmune, AstraZeneca). In certain embodiments, the antibody or antibody-binding fragment thereof binds OX40. In certain embodiments, the antibody or antibody-binding fragment thereof that binds OX40 is MEDI6469 (MedImmune, AstraZeneca), MEDI0562 (MedImmune, AstraZeneca), or KHK4083 (Kyowa Hakko Kirin). In certain embodiments, the antibody or antibody-binding fragment thereof binds GITR. In certain embodiments, the antibody or antibody-binding fragment thereof that binds GITR is TRX518 (Leap Therapeutics) or MEDI1873 (MedImmune, AstraZeneca). In certain embodiments, the antibody or antibody-binding fragment thereof binds CD137 (4-1BB). In certain embodiments, the antibody or antibody-binding fragment thereof that binds CD137 (4-1BB) is PF-2566 (PF-05082566; Pfizer), or urelumab (BMS-663513; Bristol-Myers Squibb). In certain embodiments, the antibody or antibody-binding fragment thereof binds CD27. In certain embodiments, the antibody or antibody-binding fragment thereof that binds CD27 is varilumab (CDX-1127; Celldex Therapies).

In certain embodiments, treatment of an individual having cancer using the NK cells produced using the methods described herein, e.g., NK cell populations produced using the three-stage method described herein, is part of an anticancer therapy regimen that includes lenalidomide or pomalidomide. In certain embodiments, treatment of an individual having cancer using the NK cells produced using the methods described herein, e.g., NK cell populations produced using the three-stage method described herein, is part of an anticancer therapy regimen that includes an HD AC inhibitor. In certain embodiments, treatment of an individual having cancer using the NK cells produced using the methods described herein, e.g., NK cell populations produced using the three-stage method described herein, is part of an anticancer therapy regimen that includes an anti-CS-1 antibody. In certain embodiments, treatment of an individual having cancer using the NK cells produced using the methods described herein, e.g., NK cell populations produced using the three-stage method described herein, is part of an anticancer therapy regimen that includes an anti-CD38 antibody. In certain embodiments, treatment of an individual having cancer using the NK cells produced using the methods described herein, e.g., NK cell populations produced using the three-stage method described herein, is part of an anticancer therapy regimen that includes an anti-CD138 antibody. In certain embodiments, treatment of an individual having cancer using the NK cells produced using the methods described herein, e.g., NK cell populations produced using the three-stage method described herein, is part of an anticancer therapy regimen that includes an anti-PD-1 antibody. In certain embodiments, treatment of an individual having cancer using the NK cells produced using the methods described herein, e.g., NK cell populations produced using the three-stage method described herein, is part of an anticancer therapy regimen that includes an anti-PD-L1 antibody. In certain embodiments, treatment of an individual having cancer using the NK cells produced using the methods described herein, e.g., NK cell populations produced using the three-stage method described herein, is part of an anticancer therapy regimen that includes an anti-NKG2A antibody. In certain embodiments, treatment of an individual having cancer using the NK cells produced using the methods described herein, e.g., NK cell populations produced using the three-stage method described herein, is part of an anticancer therapy regimen that includes an anti-CD20 antibody (e.g., rituximab; RITUXAN®). In certain embodiments, treatment of an individual having cancer using the NK cells produced using the methods described herein, e.g., NK cell populations produced using the three-stage method described herein, is part of an anticancer therapy regimen that includes CC-122. In certain embodiments, treatment of an individual having cancer using the NK cells produced using the methods described herein, e.g., NK cell populations produced using the three-stage method described herein, is part of an anticancer therapy regimen that includes CC-220. In certain embodiments, treatment of an individual having cancer using the NK cells produced using the methods described herein, e.g., NK cell populations produced using the three-stage method described herein, is part of an anticancer therapy regimen that includes an anti-DLL4 antibody (e.g., demcizumab). In certain embodiments, treatment of an individual having cancer using the NK cells produced using the methods described herein, e.g., NK cell populations produced using the three-stage method described herein, is part of an anticancer therapy regimen that includes an anti-DLL4 and anti-VEGF bispecific antibody. In certain embodiments, treatment of an individual having cancer using the NK cells produced using the methods described herein, e.g., NK cell populations produced using the three-stage method described herein, is part of an anticancer therapy regimen that includes an anti-RSP03 antibody. In certain embodiments, treatment of an individual having cancer using the NK cells produced using the methods described herein, e.g., NK cell populations produced using the three-stage method described herein, is part of an anticancer therapy regimen that includes an anti-TIGIT antibody. In certain embodiments, treatment of an individual having cancer using the NK cells produced using the methods described herein, e.g., NK cell populations produced using the three-stage method described herein, is part of an anticancer therapy regimen that includes an ICOS agonist antibody. In certain embodiments, treatment of an individual having cancer using the NK cells produced using the methods described herein, e.g., NK cell populations produced using the three-stage method described herein, is part of an anticancer therapy regimen that includes homoharringtonine (e.g., omacetaxine mepesuccinate).

In some embodiments, treatment of an individual having cancer using the NK cells produced using the methods described herein is part of an anticancer therapy regimen for antibody-dependent cell-mediated cytotoxicity (ADCC). In some embodiments, treatment of an individual having cancer using the ILC3 cells produced using the methods described herein is part of an anticancer therapy regimen for antibody-dependent cell-mediated cytotoxicity (ADCC). In one embodiment, the ADCC regimen comprises administration of one or more antibodies (e.g., an antibody described in the foregoing paragraph) in combination with NK cells and/or ILC3 cells produced using the methods described herein. Several types of cancer can be treated using such ADCC methods, including but not limited to acute lymphoblastic leukemia (ALL) or other B-cell malignancies (lymphomas and leukemias), neuroblastoma, melanoma, breast cancers, and head and neck cancers. In specific embodiments, the ADCC therapy comprises administration of one or more of the following antibodies anti-EGFR antibody (e.g., Erbitux (cetuximab)), anti-CD19 antibody, anti-CD20 antibody (e.g., rituximab), anti-disialoganglioside (GD2) antibody (e.g., monoclonal antibody 3F8 or chl4.18), or anti-ErbB2 antibody (e.g., herceptin), in combination with NK cells and/or ILC3 cells produced using the methods described herein. In one embodiment, the ADCC regimen comprises administration of an anti-CD33 antibody in combination with NK cells and/or ILC3 cells produced using the methods described herein. In one embodiment, the ADCC regimen comprises administration of an anti-CD20 antibody in combination with NK cells and/or ILC3 cells produced using the methods described herein. In one embodiment, the ADCC regimen comprises administration of an anti-CD138 antibody in combination with NK cells and/or ILC3 cells produced using the methods described herein. In one embodiment, the ADCC regimen comprises administration of an anti-CD32 antibody in combination with NK cells and/or ILC3 cells produced using the methods described herein.

5.11.4. Treatment of Viral Infection

In another embodiment, provided herein is a method of treating an individual having a viral infection, comprising administering to said individual a therapeutically effective amount of NK cells produced using the methods described herein, e.g., NK cell populations produced using the three-stage method described herein. In another embodiment, provided herein is a method of treating an individual having a viral infection, comprising administering to said individual a therapeutically effective amount of ILC3 cells produced using the methods described herein, e.g., ILC3 cell populations produced using the three-stage method described herein. In certain embodiments, the individual has a deficiency of natural killer cells, e.g., a deficiency of NK cells or other innate lymphoid cells active against the individual's viral infection. In certain specific embodiments, said administering additionally comprises administering to the individual one or more of isolated placental perfusate, isolated placental perfusate cells, isolated natural killer cells, e.g., placental natural killer cells, e.g., placenta-derived intermediate natural killer cells, isolated combined natural killer cells, and/or combinations thereof. In certain specific embodiments, the NK cells and/or ILC3 cells produced using the methods described herein are contacted or brought into proximity with an immunomodulatory compound, e.g., an immunomodulatory compound above, or thalidomide, prior to said administration. In certain other specific embodiments, said administering comprises administering an immunomodulatory compound, e.g., an immunomodulatory compound described above, or thalidomide, to said individual in addition to said NK cells and/or ILC3 cells produced using the methods described herein, wherein said amount is an amount that, e.g., results in a detectable improvement of, lessening of the progression of, or elimination of, one or more symptoms of said viral infection. In specific embodiments, the viral infection is an infection by a virus of the Adenoviridae, Picornaviridae, Herpesviridae, Hepadnaviridae, Flaviviridae, Retroviridae, Orthomyxoviridae, Paramyxoviridae, Papilommaviridae, Rhabdoviridae, or Togaviridae family. In more specific embodiments, said virus is human immunodeficiency virus (HIV).coxsackievirus, hepatitis A virus (HAV), poliovirus, Epstein-Barr virus (EBV), herpes simplex type 1 (HSV1), herpes simplex type 2 (HSV2), human cytomegalovirus (CMV), human herpesvirus type 8 (HHV8), herpes zoster virus (varicella zoster virus (VZV) or shingles virus), hepatitis B virus (HBV), hepatitis C virus (HCV), hepatitis D virus (HDV), hepatitis E virus (HEV), influenza virus (e.g., influenza A virus, influenza B virus, influenza C virus, or thogotovirus), measles virus, mumps virus, parainfluenza virus, papillomavirus, rabies virus, or rubella virus.

In other more specific embodiments, said virus is adenovirus species A, serotype 12, 18, or 31; adenovirus species B, serotype 3, 7, 11, 14, 16, 34, 35, or 50; adenovirus species C, serotype 1, 2, 5, or 6; species D, serotype 8, 9, 10, 13, 15, 17, 19, 20, 22, 23, 24, 25, 26, 27, 28, 29, 30, 32, 33, 36, 37, 38, 39, 42, 43, 44, 45, 46, 47, 48, 49, or 51; species E, serotype 4; or species F, serotype 40 or 41.

In certain other more specific embodiments, the virus is Apoi virus (APOIV), Aroa virus (AROAV), bagaza virus (BAGV), Banzi virus (BANV), Bouboui virus (BOUV), Cacipacore virus (CPCV), Carey Island virus (CIV), Cowbone Ridge virus (CRV), Dengue virus (DENV), Edge Hill virus (EHV), Gadgets Gully virus (GGYV), Ilheus virus (ILHV), Israel turkey meningoencephalomyelitis virus (ITV), Japanese encephalitis virus (JEV), Jugra virus (JUGV), Jutiapa virus (JUTV), kadam virus (KADV), Kedougou virus (KEDV), Kokobera virus (KOKV), Koutango virus (KOUV), Kyasanur Forest disease virus (KFDV), Langat virus (LGTV), Meaban virus (MEAV), Modoc virus (MODV), Montana myotis leukoencephalitis virus (MMLV), Murray Valley encephalitis virus (MVEV), Ntaya virus (NTAV), Omsk hemorrhagic fever virus (OHFV), Powassan virus (POWV), Rio Bravo virus (RBV), Royal Farm virus (RFV), Saboya virus (SABV), St. Louis encephalitis virus (SLEV), Sal Vieja virus (SVV), San Perlita virus (SPV), Saumarez Reef virus (SREV), Sepik virus (SEPV), Tembusu virus (TMUV), tick-borne encephalitis virus (TBEV), Tyuleniy virus (TYUV), Uganda S virus (UGSV), Usutu virus (USUV), Wesselsbron virus (WESSV), West Nile virus (WNV), Yaounde virus (YAOV), Yellow fever virus (YFV), Yokose virus (YOKV), or Zika virus (ZIKV).

In other embodiments, the NK cells produced using the methods described herein, and optionally placental perfusate and/or perfusate cells, are administered to an individual having a viral infection as part of an antiviral therapy regimen that includes one or more other antiviral agents. Specific antiviral agents that may be administered to an individual having a viral infection include, but are not limited to: imiquimod, podofilox, podophyllin, interferon alpha (IFNα), reticolos, nonoxynol-9, acyclovir, famciclovir, valaciclovir, ganciclovir, cidofovir; amantadine, rimantadine; ribavirin; zanamavir and oseltaumavir; protease inhibitors such as indinavir, nelfinavir, ritonavir, or saquinavir; nucleoside reverse transcriptase inhibitors such as didanosine, lamivudine, stavudine, zalcitabine, or zidovudine; and non-nucleoside reverse transcriptase inhibitors such as nevirapine, or efavirenz.

5.11.5. Other Treatment Uses for ILC3 Cells

Provided herein are ILC3 cells that can be used in all the methods as provided herein. Exemplary methods in which ILC3 cells can be used are disclosed in the following aspects.

In another aspect, provided herein is a method of repairing the gastrointestinal tract after chemotherapy comprising administering to an individual a plurality of ILC3 cells, wherein the ILC3 cells are by a three-stage method described herein.

In another aspect, provided herein is a method of protecting an individual against radiation comprising administering to an individual a plurality of ILC3 cells, wherein the ILC3 cells are produced by a three-stage method described herein. In certain aspects, said ILC3 cells are used as an adjunct to bone marrow transplantation.

In another aspect, provided herein is a method of reconstituting the thymus of an individual comprising administering to an individual a plurality of ILC3 cells, wherein the ILC3 cells are produced by a three-stage method described herein.

In another aspect, provided herein is a method of promoting protective immunity to pathogens in an individual comprising administering to an individual a plurality of ILC3 cells, wherein the ILC3 cells are produced by a three-stage method described herein. In certain aspects, promoting protective immunity to pathogens is performed to treat intestinal infection. In certain aspects, promoting protective immunity to pathogens is performed to prevent intestinal infection. In certain aspects, the intestinal infection is Citrobacter rodentium.

In another aspect, provided herein is a method of tumor rejection comprising administering to an individual a plurality of ILC3 cells, wherein the ILC3 cells have been produced by a three-stage method described herein.

In another aspect, provided herein is a method of maintaining tissue integrity during organogenesis comprising administering to an individual a plurality of ILC3 cells, wherein the ILC3 cells have been produced by a three-stage method described herein.

In another aspect, provided herein is a method of tissue repair comprising administering to an individual a plurality of ILC3 cells, wherein the ILC3 cells have been produced by a three-stage method described herein.

In another aspect, provided herein is a method of regulation of inflammation comprising administering to an individual a plurality of ILC3 cells, wherein the ILC3 cells have been produced by a three-stage method described herein.

5.11.6. Administration

Determination of the number of cells, e.g., placental perfusate cells, e.g., nucleated cells from placental perfusate, combined natural killer cells, ILC3 cells, and/or isolated natural killer cells, e.g., NK cell populations produced using the three-stage method described herein, and determination of the amount of an immunomodulatory compound, e.g., an immunomodulatory compound, or thalidomide, can be performed independently of each other.

Administration of an isolated population of NK cells and/or ILC3 cells or a pharmaceutical composition thereof may be systemic or local. In specific embodiments, administration is parenteral. In specific embodiments, administration of an isolated population of NK cells and/or ILC3 cells or a pharmaceutical composition thereof to a subject is by injection, infusion, intravenous (IV) administration, intrafemoral administration, or intratumor administration. In specific embodiments, administration of an isolated population of NK cells and/or ILC3 cells or a pharmaceutical composition thereof to a subject is performed with a device, a matrix, or a scaffold. In specific embodiments, administration an isolated population of NK cells and/or ILC3 cells or a pharmaceutical composition thereof to a subject is by injection. In specific embodiments, administration an isolated population of NK cells and/or ILC3 cells or a pharmaceutical composition thereof to a subject is via a catheter. In specific embodiments, the injection of NK cells and/or ILC3 cells is local injection. In more specific embodiments, the local injection is directly into a solid tumor (e.g., a sarcoma). In specific embodiments, administration of an isolated population of NK cells and/or ILC3 cells or a pharmaceutical composition thereof to a subject is by injection by syringe. In specific embodiments, administration of an isolated population of NK cells and/or ILC3 cells or a pharmaceutical composition thereof to a subject is via guided delivery. In specific embodiments, administration of an isolated population of NK cells and/or ILC3 cells or a pharmaceutical composition thereof to a subject by injection is aided by laparoscopy, endoscopy, ultrasound, computed tomography, magnetic resonance, or radiology.

5.11.6.1. Administration of Cells

In certain embodiments, NK cells and/or ILC3 cells produced using the methods described herein, e.g., NK cell and/or ILC3 cell populations produced using the three-stage method described herein, are used, e.g., administered to an individual, in any amount or number that results in a detectable therapeutic benefit to the individual, e.g., an effective amount, wherein the individual has a viral infection, cancer, or tumor cells, for example, an individual having tumor cells, a solid tumor or a blood cancer, e.g., a cancer patient. Such cells can be administered to such an individual by absolute numbers of cells, e.g., said individual can be administered at about, at least about, or at most about, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, or 1×10¹¹ NK cells and/or ILC3 cells produced using the methods described herein. In other embodiments, NK cells and/or ILC3 cells produced using the methods described herein can be administered to such an individual by relative numbers of cells, e.g., said individual can be administered at about, at least about, or at most about, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, or 1×10¹¹ NK cells and/or ILC3 cells produced using the methods described herein per kilogram of the individual. In other embodiments, NK cells and/or ILC3 cells produced using the methods described herein can be administered to such an individual by relative numbers of cells, e.g., said individual can be administered at about, at least about, or at most about, 1×10⁵, 5×10⁵, 1×10⁶, 5×10⁶, 1×10⁷, 5×10⁷, 1×10⁸, or 5×10⁸ NK cells and/or ILC3 cells produced using the methods described herein per kilogram of the individual. NK cells and/or ILC3 cells produced using the methods described herein can be administered to such an individual according to an approximate ratio between a number of NK cells and/or ILC3 cells produced using the methods described herein, and optionally placental perfusate cells and/or natural killer cells other than NK cells and/or ILC3 cells produced using the methods described herein, and a number of tumor cells in said individual (e.g., an estimated number). For example, NK cells and/or ILC3 cells produced using the methods described herein can be administered to said individual in a ratio of about, at least about or at most about 1:1, 1:1, 3:1, 4:1, 5:1, 6:1, 7:1, 8:1, 9:1, 10:1, 15:1, 20:1, 25:1, 30:1, 35:1, 40:1, 45:1, 50:1, 55:1, 60:1, 65:1, 70:1, 75:1, 80:1, 85:1, 90:1, 95:1 or 100:1 to the number of tumor cells in the individual. The number of tumor cells in such an individual can be estimated, e.g., by counting the number of tumor cells in a sample of tissue from the individual, e.g., blood sample, biopsy, or the like. In specific embodiments, e.g., for solid tumors, said counting is performed in combination with imaging of the tumor or tumors to obtain an approximate tumor volume. In a specific embodiment, an immunomodulatory compound or thalidomide, e.g., an effective amount of an immunomodulatory compound or thalidomide, are administered to the individual in addition to the NK cells and/or ILC3 cells produced using the methods described herein, optionally placental perfusate cells and/or natural killer cells other than NK cells and/or ILC3 cells produced using the methods described herein.

In certain embodiments, the method of suppressing the proliferation of tumor cells, e.g., in an individual; treatment of an individual having a deficiency in the individual's natural killer cells; or treatment of an individual having a viral infection; or treatment of an individual having cancer, e.g., an individual having tumor cells, a blood cancer or a solid tumor, comprises bringing the tumor cells into proximity with, or administering to said individual, a combination of NK cells and/or ILC3 cells produced using the methods described herein and one or more of placental perfusate and/or placental perfusate cells. In specific embodiments, the method additionally comprises bringing the tumor cells into proximity with, or administering to the individual, an immunomodulatory compound or thalidomide.

In a specific embodiment, for example, treatment of an individual having a deficiency in the individual's natural killer cells (e.g., a deficiency in the number of NK cells or in the NK cells' reactivity to a cancer, tumor or virally-infected cells); or treatment of an individual having a cancer or a viral infection, or suppression of tumor cell proliferation, comprises bringing said tumor cells into proximity with, or administering to said individual, NK cells and/or ILC3 cells produced using the methods described herein supplemented with isolated placental perfusate cells or placental perfusate. In specific embodiments, about 1×10⁴, 5×10⁴, 1×10⁵, 5×10⁵, 1×10⁶, 5×10⁶, 1×10⁷, 5×10⁷, 1×10⁸, 5×10⁸ or more NK cells produced using the methods described herein per milliliter, or 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹ or more NK cells produced using the methods described herein are supplemented with about, or at least about, 1×10⁴, 5×10⁴, 1×10⁵, 5×10⁵, 1×10⁶, 5×10⁶, 1×10⁷, 5×10⁷, 1×10⁸, 5×10⁸ or more isolated placental perfusate cells per milliliter, or 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹ or more isolated placental perfusate cells. In other more specific embodiments, about 1×10⁴, 5×10⁴, 1×10⁵, 5×10⁵, 1×10⁶, 5×10⁶, 1×10⁷, 5×10⁷, 1×10⁸, 5×10⁸ or more NK cells produced using the methods described herein or 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹ or more NK cells produced using the methods described herein are supplemented with about, or at least about, 1, 2, 3, 4, 5, 6, 7, 8, 9, 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950 or 1000 mL of perfusate, or about 1 unit of perfusate.

In another specific embodiment, treatment of an individual having a deficiency in the individual's natural killer cells; treatment of an individual having cancer; treatment of an individual having a viral infection; or suppression of tumor cell proliferation, comprises bringing the tumor cells into proximity with, or administering to the individual, NK cells and/or ILC3 cells produced using the methods described herein, wherein said cells are supplemented with adherent placental cells, e.g., adherent placental stem cells or multipotent cells, e.g., CD34⁻, CD10⁺, CD105⁺, CD200⁺ tissue culture plastic-adherent placental cells. In specific embodiments, the NK cells and/or ILC3 cells produced using the methods described herein are supplemented with about 1×10⁴, 5×10⁴, 1×10⁵, 5×10⁵, 1×10⁶, 5×10⁶, 1×10⁷, 5×10⁷, 1×10⁸, 5×10⁸ or more adherent placental stem cells per milliliter, or 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹ or more adherent placental cells, e.g., adherent placental stem cells or multipotent cells.

In another specific embodiment, treatment of an individual having a deficiency in the individual's natural killer cells; treatment of an individual having cancer; treatment of an individual having a viral infection; or suppression of tumor cell proliferation, is performed using an immunomodulatory compound or thalidomide in combination with NK cells and/or ILC3 cells produced using the methods described herein, wherein said cells are supplemented with conditioned medium, e.g., medium conditioned by CD34⁻, CD10⁺, CD105⁺, CD200⁺ tissue culture plastic-adherent placental cells, e.g., 0.1, 0.2, 0.3, 0.4, 0.5, 0.6, 0.1, 0.8, 0.9, 1, 2, 3, 4, 5, 6, 7, 8, 9, 10 mL of stem cell-conditioned culture medium per unit of perfusate, or per 10⁴, 10⁵, 10⁶, 10⁷, 10⁸, 10⁹, 10¹⁰, or 10¹¹ NK cells and/or ILC3 cells produced using the methods described herein. In certain embodiments, the tissue culture plastic-adherent placental cells are the multipotent adherent placental cells described in U.S. Pat. Nos. 7,468,276 and 8,057,788, the disclosures of which are incorporated herein by reference in their entireties. In another specific embodiment, the method additionally comprises bringing the tumor cells into proximity with, or administering to the individual, an immunomodulatory compound or thalidomide.

In another specific embodiment, treatment of an individual having a deficiency in the individual's natural killer cells; treatment of an individual having cancer; treatment of an individual having a viral infection; or suppression of tumor cell proliferation, in which said NK cells and/or ILC3 cells produced using the methods described herein are supplemented with placental perfusate cells, the perfusate cells are brought into proximity with interleukin-2 (IL-2) for a period of time prior to said bringing into proximity. In certain embodiments, said period of time is about, at least, or at most 1, 2, 3, 4, 5, 6, 7, 8, 9, 10, 12, 14, 16, 18, 20, 22, 24, 26, 28, 30, 32, 34, 36, 38, 40, 42, 44, 46 or 48 hours prior to said bringing into proximity.

The NK cells and/or ILC3 cells produced using the methods described herein and optionally perfusate or perfusate cells, can be administered once to an individual having a viral infection, an individual having cancer, or an individual having tumor cells, during a course of anticancer therapy; or can be administered multiple times, e.g., once every 1, 2, 3, 4, 5, 6, 7, 8, 9, 10, 11, 12, 13, 14, 15, 16, 17, 18, 19, 20, 21, 22 or 23 hours, or once every 1, 2, 3, 4, 5, 6 or 7 days, or once every 1, 2, 3, 4, 5, 6, 7, 8, 9, 10, 24, 36 or more weeks during therapy. In embodiments in which cells and an immunomodulatory compound or thalidomide are used, the immunomodulatory compound or thalidomide, and cells or perfusate, can be administered to the individual together, e.g., in the same formulation; separately, e.g., in separate formulations, at approximately the same time; or can be administered separately, e.g., on different dosing schedules or at different times of the day. Similarly, in embodiments in which cells and an antiviral compound or anticancer compound are used, the antiviral compound or anticancer compound, and cells or perfusate, can be administered to the individual together, e.g., in the same formulation; separately, e.g., in separate formulations, at approximately the same time; or can be administered separately, e.g., on different dosing schedules or at different times of the day. The NK cells and/or ILC3 cells produced using the methods described herein and perfusate or perfusate cells, can be administered without regard to whether NK cells and/or ILC3 cells produced using the methods described herein, perfusate, or perfusate cells have been administered to the individual in the past.

### 6. KITS

Provided herein is a pharmaceutical pack or kit comprising one or more containers filled with one or more of the compositions described herein, e.g., a composition comprising NK cells and/or ILC3 cells produced by a method described herein, e.g., NK cell and/or ILC3 cell populations produced using the three-stage method described herein. Optionally associated with such container(s) can be a notice in the form prescribed by a governmental agency regulating the manufacture, use or sale of pharmaceuticals or biological products, which notice reflects approval by the agency of manufacture, use or sale for human administration.

The kits encompassed herein can be used in accordance with the methods described herein, e.g., methods of suppressing the growth of tumor cells and/or methods of treating cancer, e.g., hematologic cancer, and/or methods of treating viral infection. In one embodiment, a kit comprises NK cells and/or ILC3 cells produced by a method described herein or a composition thereof, in one or more containers. In a specific embodiment, provided herein is a kit comprising an NK cell and/or ILC3 cell population produced by a three-stage method described herein, or a composition thereof.

### 7. EXAMPLES

### 7.1. Example 1: Three-Stage Method of Producing Natural Killer Cells from Hematopoietic Stem or Progenitor Cells

CD34⁺ cells are cultured in the following medium formulations for the indicated number of days, and aliquots of cells are taken for assessment of cell count, cell viability, characterization of natural killer cell differentiation and functional evaluation.

Stage 1 medium: 90% Stem Cell Growth Medium (SCGM) (CellGro®), 10% Human Serum-AB, supplemented with 25 ng/mL or 250 ng/mL recombinant human thrombopoietin (TPO), 25 ng/mL recombinant human Flt3L, 27 ng/mL recombinant human stem cell factor (SCF), 25 ng/mL recombinant human IL-7, 0.05 ng/mL or 0.025 ng/mL recombinant human IL-6, 0.25 ng/mL or 0.125 ng/mL recombinant human granulocyte colony-stimulating factor (G-CSF), 0.01 ng/mL or 0.025 ng/mL recombinant human granulocyte-macrophage colony-stimulating factor (GM-CSF), 0.10% gentamicin, and 1 to 10 μm StemRegenin-1 (SR-1) or other stem cell mobilizing agent.

Stage 2 medium: 90% SCGM, 10% Human Serum-AB, supplemented with 25 ng/mL recombinant human Flt3L, 27 ng/mL recombinant human SCF, 25 ng/mL recombinant human IL-7, 20 ng/mL recombinant human IL-15, 0.05 ng/mL or 0.025 ng/mL recombinant human IL-6, 0.25 ng/mL or 0.125 ng/mL recombinant human granulocyte colony-stimulating factor (G-CSF), 0.01 ng/mL or 0.025 ng/mL recombinant human granulocyte-macrophage colony-stimulating factor (GM-CSF), 0.10% gentamicin, and 1 to 10 μm SRI or other stem cell mobilizing agent.

Stage 3 medium: 90% STEMMACS™, 10% Human Serum-AB, 0.025 mM 2-mercaptoethanol (55 mM), supplemented with 22 ng/mL recombinant human SCF, 1000 U/mL recombinant human IL-2, 20 ng/mL recombinant human IL-7, 20 ng/mL recombinant human IL-15, 0.05 ng/mL or 0.025 ng/mL recombinant human IL-6, 0.25 ng/mL or 0.125 ng/mL recombinant human granulocyte colony-stimulating factor (G-CSF), 0.01 ng/mL or 0.025 ng/mL recombinant human granulocyte-macrophage colony-stimulating factor (GM-CSF), and 0.10% gentamicin.

Cells are seeded at Day 0 at 3×10⁴ cells/mL in Stage 1 media, and cells are tested for purity by a CD34+ and CD45+ count and viability by 7AAD staining. At Day 5 cells are counted and seeded to a concentration of 1×10⁵ cells/mL with Stage 1 medium. At Day 7 cells are counted and seeded to a concentration of 1×10⁵ cells/mL with Stage 1 medium.

At Day 10, cells are counted and seeded to a concentration of 1×10⁵ cells/mL in Stage 2 medium. At Day 12, cells are counted and seeded to a concentration of 3×10⁵ cells/mL in Stage 2 medium. At Day 14, cells are counted and seeded in Stage 3 medium. Cells are maintained in Stage 3 media until day 35.

Alternatively, the following protocol is used through Day 14: Cells seeded at Day 0 at 7.5×10³ cells/mL in Stage 1 media, and cells are tested for purity by a CD34+ and CD45+ count and viability by 7AAD staining. At Day 7 cells are counted and seeded to a concentration of 3×10⁵ cells/mL with Stage 1 medium. At Day 9 cells are counted and seeded to a concentration of 3×10⁵ cells/mL with Stage 2 medium. At Day 12, cells are counted and seeded to a concentration of 3×10⁵ cells/mL in Stage 2 medium. At Day 14, cells are counted and seeded to a concentration of 3×10⁵ cells/mL in Stage 2 medium.

Seeding of cells into at passage is performed either by dilution of the culture with fresh media or by centrifugation of cells and resuspension/addition of fresh media.

For harvest, cells are spun at 400×g for seven minutes, followed by suspension of the pellet in an equal volume of Plasmalyte A. The suspension is spun at 400×g for seven minutes, and the resulting pellet is suspended in 10% HSA (w/v), 60% Plasmalyte A (v/v) at the target cell concentration. The cells are then strained through a 70 μm mesh, the final container is filled, an aliquot of the cells are tested for viability, cytotoxicity, purity, and cell count, and the remainder is packaged.

### 7.2. Example 2: Selection of Stem Cell Mobilizing Agents for the Expansion of NK Cells

The following compounds were investigated for their ability to promote the expansion of NK cell populations in vitro:
4-(2-((2-(benzo[b]thiophen-3-yl)-6-(isopropylamino)pyrimidin-4-yl)amino)ethyl)phenol) (“CRL1”)

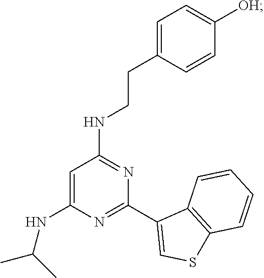 [SMILES: CC(C)Nc1cc(NCCc2ccc(O)cc2)nc(-c2csc3ccccc23)n1]

4-(2-((2-(benzo[b]thiophen-3-yl)-7-isopropylthieno[3,2-d]pyrimidin-4-yl)amino)ethyl)phenol)) (“CRL2”)

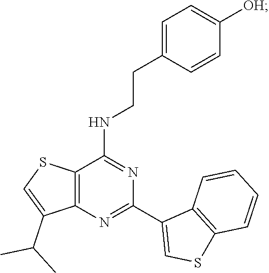 [SMILES: CC(C)c1csc2c(NCCc3ccc(O)cc3)nc(-c3csc4ccccc34)nc12]

4-(2-((2-(benzo[b]thiophen-3-yl)-7-isopropyl-6,7-dihydro-5H-pyrrolo[2,3-d]pyrimidin-4-yl)amino)ethyl)phenol (“CRL3”)

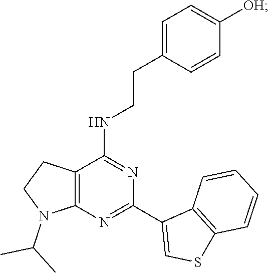 [SMILES: CC(C)N1CCc2c(NCCc3ccc(O)cc3)nc(-c3csc4ccccc34)nc21]

2-(benzo[b]thiophen-3-yl)-4-((4-hydroxyphenethyl)amino)-7-isopropyl-5,7-dihydro-6H-pyrrolo[2,3-d]pyrimidin-6-one (“CRL4”)

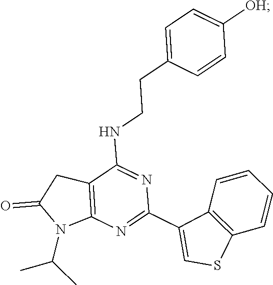 [SMILES: CC(C)N1C(=O)Cc2c(NCCc3ccc(O)cc3)nc(-c3csc4ccccc34)nc21]

3-((2-(benzo[b]thiophen-3-yl)-9-isopropyl-9H-purin-6-yl)oxy)propanamide (“CRL5”)

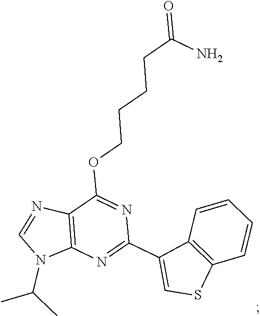 [SMILES: CC(C)n1cnc2c(OCCCCC(N)=O)nc(-c3csc4ccccc34)nc21]

4-(2-((2-(benzo[b]thiophen-3-yl)-8-(dimethylamino)pyrimido[5,4-d]pyrimidin-4-yl)amino)ethyl)phenol (“CRL6”)

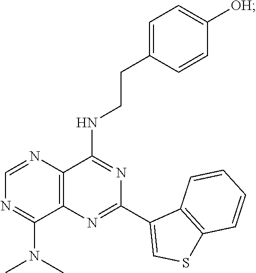 [SMILES: CN(C)c1ncnc2c(NCCc3ccc(O)cc3)nc(-c3csc4ccccc34)nc12]

5-(2-((2-(1H-indol-3-yl)ethyl)amino)-6-(sec-butylamino)pyrimidin-4-yl)nicotinonitrile (“CRL7”)

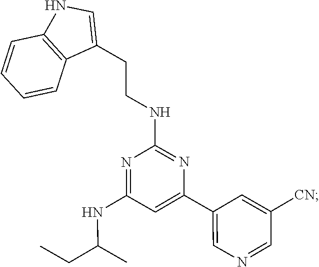 [SMILES: CCC(C)Nc1cc(-c2cncc(C#N)c2)nc(NCCc2c[nH]c3ccccc23)n1]

N-(2-(1H-indol-3-yl)ethyl)-2-methyl-6-phenylthieno[2,3-d]pyrimidin-4-amine (“CRL8”)

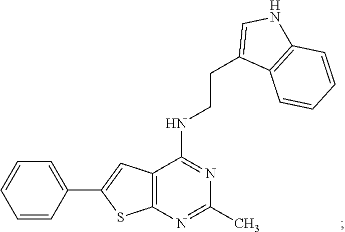 [SMILES: Cc1nc(NCCc2c[nH]c3ccccc23)c2cc(-c3ccccc3)sc2n1]

N-(2-(1H-indol-3-yl)ethyl)-6-(4-fluorophenyl)thieno[2,3-d]pyrimidin-4-amine (“CRL9”)

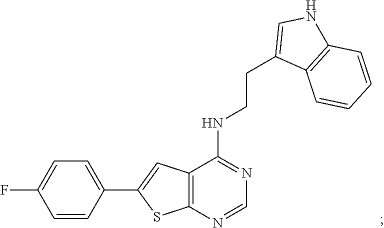 [SMILES: Fc1ccc(-c2cc3c(NCCc4c[nH]c5ccccc45)ncnc3s2)cc1]

3-(2-(benzo[b]thiophen-3-yl)-9-isopropyl-6-oxo-6,9-dihydro-1H-purin-1-yl)propanamide (“CRL10”)

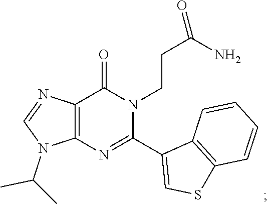 [SMILES: CC(C)n1cnc2c(=O)n(CCC(N)=O)c(-c3csc4ccccc34)nc21]

N-(2-(1H-indol-3-yl)ethyl)-2-(5-fluoropyridin-3-yl)quinazolin-4-amine (“CRL11”)

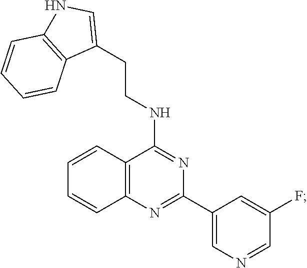 [SMILES: Fc1cncc(-c2nc(NCCc3c[nH]c4ccccc34)c3ccccc3n2)c1]

5-(4-((2-(1H-indol-3-yl)ethyl)amino)quinazolin-2-yl)nicotinonitrile (“CRL12”)

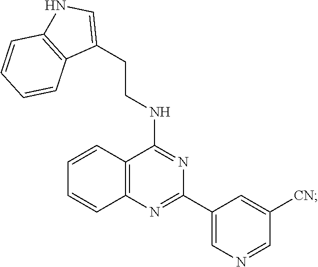 [SMILES: N#Cc1cncc(-c2nc(NCCc3c[nH]c4ccccc34)c3ccccc3n2)c1]

N⁴-(2-(1H-indol-3-yl)ethyl)-N²-(sec-butyl)quinazoline-1,4-diamine (“CRL13”)

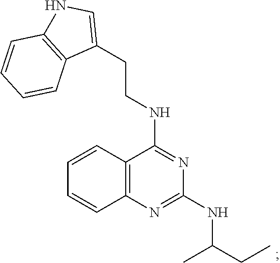 [SMILES: CCC(C)Nc1nc(NCCc2c[nH]c3ccccc23)c2ccccc2n1]

2-(benzo[b]thiophen-3-yl)-4-((4-hydroxyphenethyl)amino)-7-isopropyl-7H-pyrrolo[2,3-d]pyrimidine-5-carbonitrile (“CRL14”)

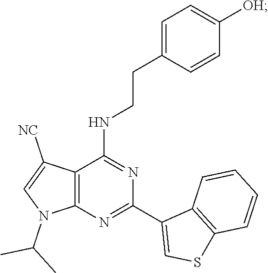 [SMILES: CC(C)n1cc(C#N)c2c(NCCc3ccc(O)cc3)nc(-c3csc4ccccc34)nc21]

N-(2-(1H-indol-3-yl)ethyl)-6-(benzo[b]thiophen-3-yl)-3-isopropylimidazo[1,5-a]pyrazin-8-amine (“CRL15”)

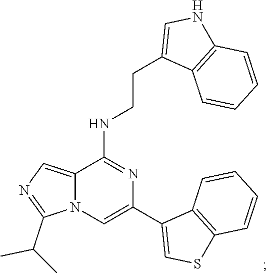 [SMILES: CC(C)c1ncc2c(NCCc3c[nH]c4ccccc34)nc(-c3csc4ccccc34)cn12]

4-(2-((6-(benzo[b]thiophen-3-yl)-3-isopropylimidazo[1,5-a]pyrazin-8-yl)amino)ethyl)phenol (“CRL16”)

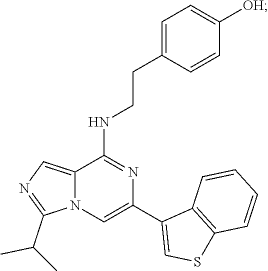 [SMILES: CC(C)c1ncc2c(NCCc3ccc(O)cc3)nc(-c3csc4ccccc34)cn12]

5-(4-((2-(1H-indol-3-yl)ethyl)amino)-7-isopropylthieno[3,2-d]pyrimidin-2-yl)nicotinonitrile (“CRL17”)

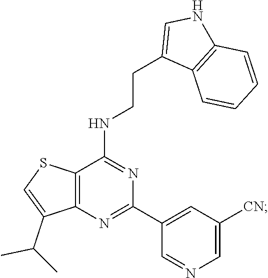 [SMILES: CC(C)c1csc2c(NCCc3c[nH]c4ccccc34)nc(-c3cncc(C#N)c3)nc12]

N-(2-(1H-indol-3-yl)ethyl)-2-(5-fluoropyridin-3-yl)-7-isopropylthieno[3,2-d]pyrimidin-4-amine (“CRL18”)

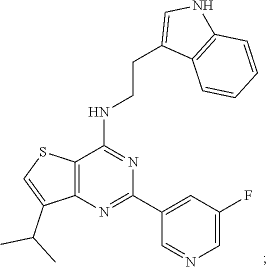 [SMILES: CC(C)c1csc2c(NCCc3c[nH]c4ccccc34)nc(-c3cncc(F)c3)nc12]

N-(2-(1H-indol-3-yl)ethyl)-2-(5-fluoropyridin-3-yl)furo[3,2-d]pyrimidin-4-amine (“CRL19”)

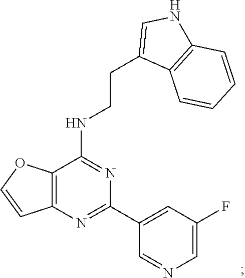 [SMILES: Fc1cncc(-c2nc(NCCc3c[nH]c4ccccc34)c3occc3n2)c1]

N-(2-(1H-indol-3-yl)ethyl)-2-(5-methyl pyridin-3-yl)furo[3,2d]pyrimidin-4-amine (“CRL20”)

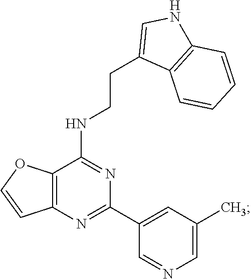 [SMILES: Cc1cncc(-c2nc(NCCc3c[nH]c4ccccc34)c3occc3n2)c1]

N-(2-(1H-indol-3-yl)ethyl)-7-isopropyl-2-(5-methylpyridin-3-yl)thieno[3,2-d]pyrimidin-4-amine (“CRL21”)

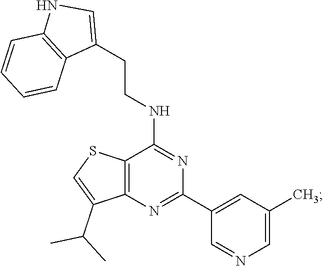 [SMILES: Cc1cncc(-c2nc(NCCc3c[nH]c4ccccc34)c3scc(C(C)C)c3n2)c1]

and
5-(4-((2-(1H-indol-3-yl)ethyl)amino)furo[3,2-d]pyrimidin-2-yl)nicotinonitrile (“CRL22”)

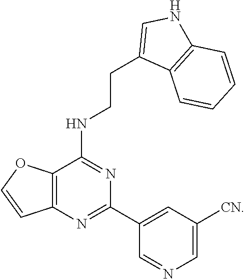 [SMILES: N#Cc1cncc(-c2nc(NCCc3c[nH]c4ccccc34)c3occc3n2)c1]

### 7.3. Example 3: Characterization of Three-Stage NK Cells

### Methods

UCB CD34+ cells were cultivated in presence of cytokines including thrombopoietin, SCF, Flt3 ligand, IL-7, IL-15 and IL-2 for 35 days to produce three-stage NK cells, as described in Example 1. Multi-color flow cytometry was used to determine the phenotypic characteristics of three-stage NK cells.

For biological testing, the compounds were provided to culture to evaluate their effects on NK cell expansion and differentiation. Specifically, donors of CD34+ cells (StemCell Technology) were thawed and expanded in vitro following NK culture protocol. During the first 14 days of the culture, each CRL compounds was dissolved in DMSO and added to the culture at 10 μM concentration. SRI (at 10 μM) served as a positive control compound, while DMSO alone without any compound served as a negative control. At the end of the culture on Day 35, cell expansion, natural killer (NK) cell differentiation and cytotoxicity of the cells against K562 tumor cell line were characterized. Due to the large number of the compounds, the testing was performed in two experiments, CRL1-11 and CRL 12-22. The same donors were used for each experiment. Positive and negative controls were also included in both experiments.

### Results

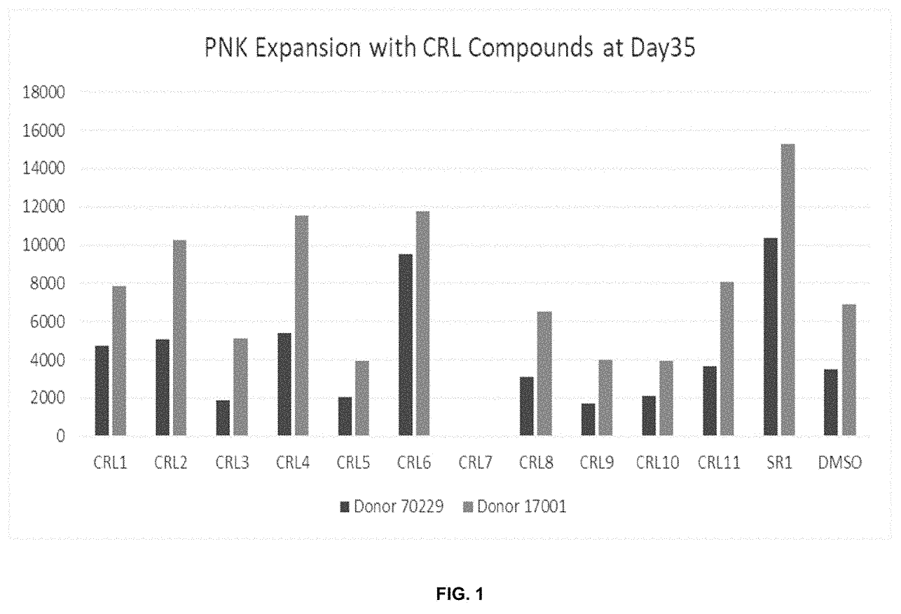

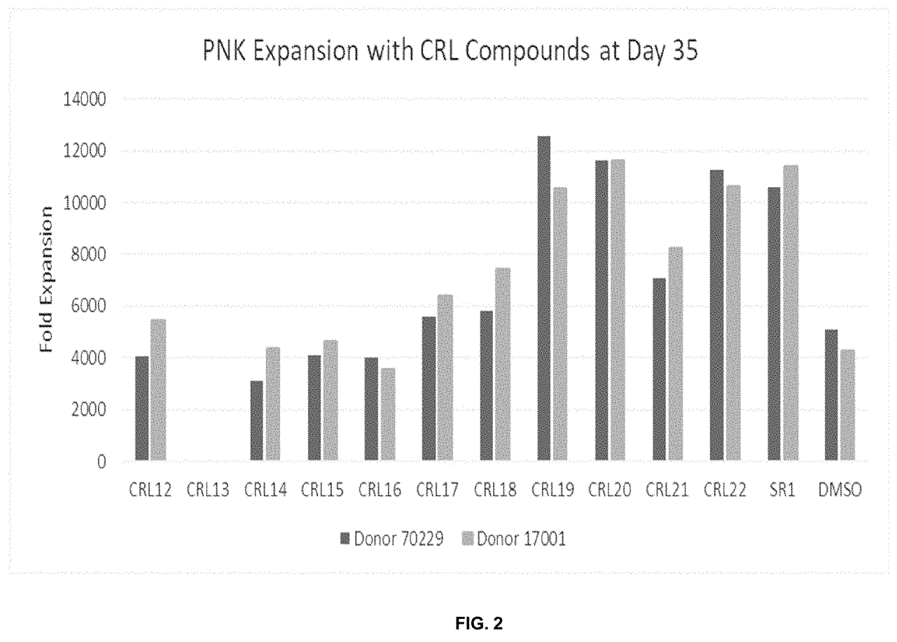

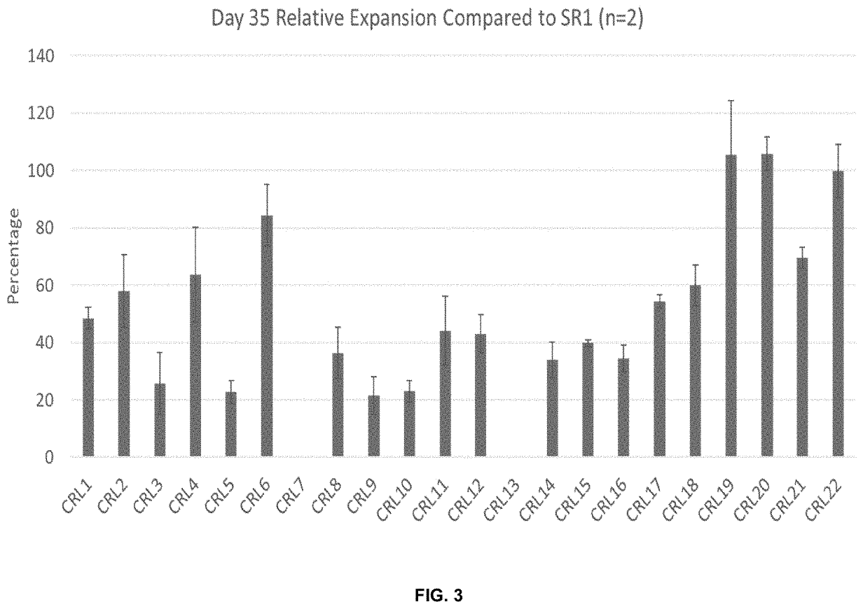

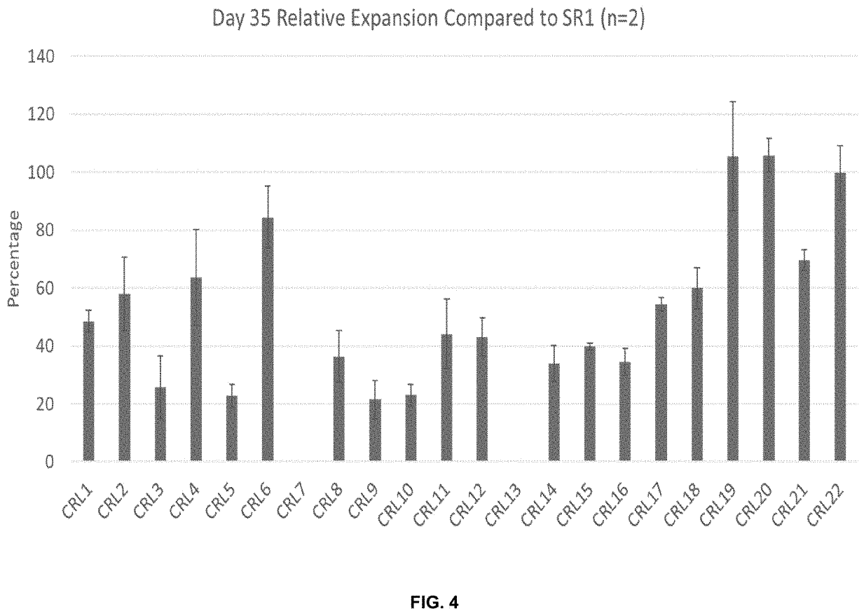

Cell expansion data showed that 20 out of the 22 compounds supported NK expansion at 10 μM concentration. Except for CRL7 and CRL13, the rest of the compounds all resulted in a NK expansion of 2,000˜15,000 fold over 35 days (FIG. 1 and FIG. 2). Among all the compounds, CRL 19, 20 and 22 supported cell expansion the best, and they demonstrated a similar level of expansion compared to SRI at Day 35 (FIG. 3). CD34 cell expansion at Day 14 of the culture showed a similar trend that most of the compounds supported CD34 cells expansion, and CRL19, 20 and 22 achieved the highest CD34 cell expansion at Day 14 (FIG. 4).

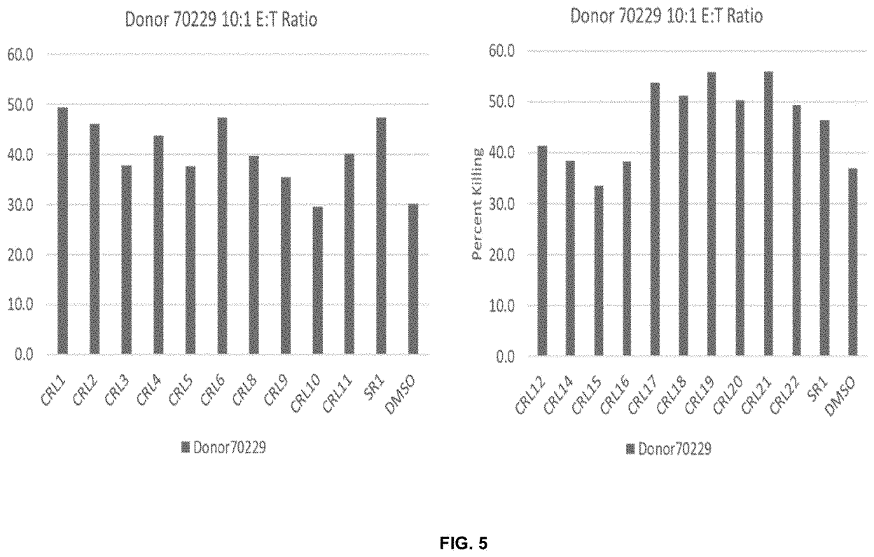

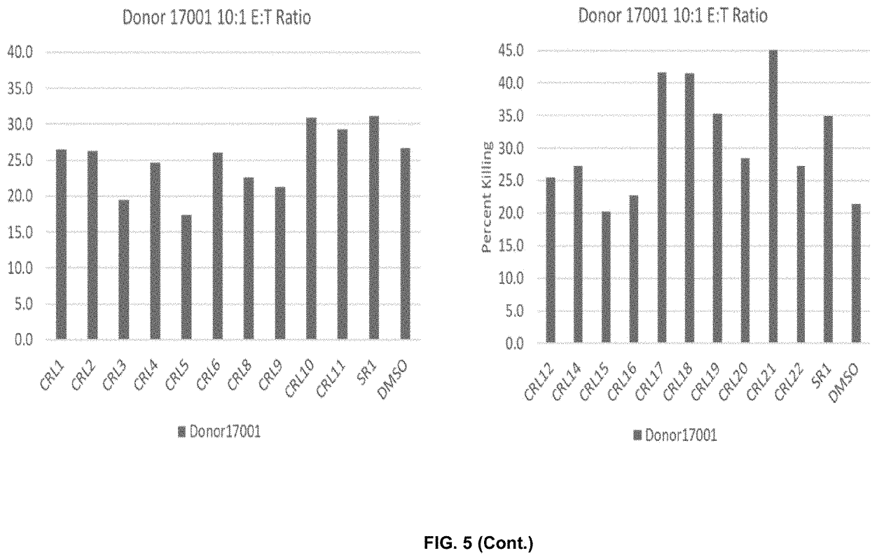

Cytotoxicity assay was run using compound cultured cells against K562 tumor cells at 10:1 effector to target ratio (FIG. 5) to evaluate cell functions. The results showed that the cells cultured with compounds killed 30-60% of K562 cells at 10:1 E:T ratio, indicating that the cells present NK functions. For both donors, cells cultured with CRL17, 18, 19 and 21 demonstrated similar or greater killing activities compared to those cultured with SRI.

### Conclusions:

In summary, we found that all the compounds except CRL7 and CRL13 supported PNK-007 expansion and differentiation. Expansion with the compounds ranged from 2,000˜15, 000 fold over 35 days, and the culture achieved more than 70% of NK cells. Among these compounds, CRL 19, 20 and 22 demonstrated very similar expansion, differentiation and cytotoxicity profiles as SRI for PNK-007 culture. CRL 17, 18, and 21 resulted in slightly less expansion compared to SRI but increased CD56+/CD11a+ subpopulation, and also increased killing activities of the cells.

### 7.4 Example 4: Further Characterization of Three-Stage NK Cells

### Methods

Cells: Frozen PBMC were acquired from Stem Cell Technologies. Peripheral blood derived NKs (PB-NK) cells were isolated from fresh blood of healthy donors using the Human NK Cell Enrichment Kit (Stem Cell Technologies) according to manufacturer's instructions. CYNK cells were generated from umbilical cord blood-derived CD34+ stem cells (Ref: Zhang et al. J Immunother Cancer. 2015). Briefly, the CD34+ cells were cultivated in the presence of cytokines including thromobopoietin, SCF, Flt3 ligand, IL-7, IL-15 and IL-2 for 35 days. PBNK and CYNK cells were cryopreserved until analysis.

Magnetic-activated cell sorting: PNK cells were stained with PE Mouse Anti-Human CD11a (BD) and CD11a+ PNK cells concentrated using anti-PE MicroBeads according to manufacturer's instructions (Miltenyi Biotec).

Single cell RNA sequencing: CYNK cells were combined with PB-NK at 1:1 ratio and gene expression analyzed on single cell level using 10× Genomics Chromium platform and Illumina sequencing. Bioinformatics analysis utilized 10× Genomics Cell Ranger analysis pipeline.

Flow Cytometry: Cryopreserved cells were rapidly thawed in a 37° C. water bath and washed once in RPMI1640+10% hiFBS (heat inactivated Fetal Bovine Serum, Gibco), followed by LIVE/DEAD™ Fixable Aqua Stain in PBS. Cells were washed with FACS buffer (PBS+2% FBS) followed by incubation in blocking solution (Brilliant Stain buffer, Mouse IgG2a isotype k control and Human BD Fc Block (all from BD)). Cells were washed with FACS buffer and incubated with fluorophore-coupled antibodies in FACS buffer for 25 min on ice. Cells were washed with FACS buffer before analysis on Fortessa X20 flow cytometer (BD).

qRT-PCR: RNA was isolated from cells using Quick-RNA Miniprep kit (Qiagen) according to the manufacturer's instructions. cDNA was synthesized using Superscript IV Reverse Transcriptase (Thermo Fisher Scientific) in a standard reaction. RT-PCR was performed using Taqman Gene expression assays (Applied Biosystems). Expression levels were calculated relative to GAPDH (Hs02758991) using the ΔΔCt method.

### Results

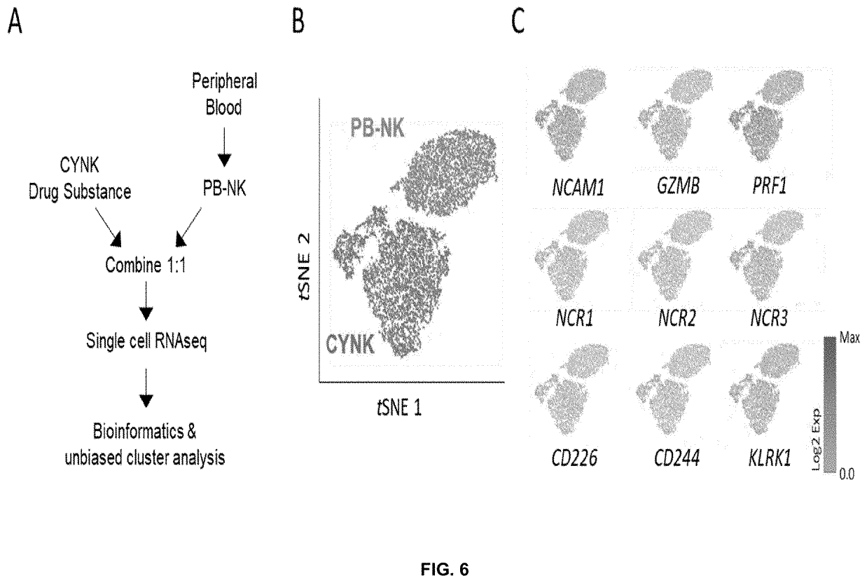

CYNIC cells efficiently kill various tumor cell lines in vitro, however, the mechanisms CYNK cells use to induce cell death remains poorly understood (ref). To elucidate on the activating NK cell receptors, the intracellular signaling pathways and molecular mechanisms CYNK cells employ to carry out their functional roles, we used single-cell RNA sequencing (scRNAseq) as an unbiased approach to compare CYNK cells to peripheral blood NK cells (PB-NK) (FIG. 6A). Unbiased transcriptional clustering revealed two distinct signatures differentiating between CYNK and PB-NK cells (FIG. 6B). Tables 1 and 2 list top 50 upregulated genes per cluster in PB-NK and CYNK cells, respectively. The gene set expressed higher in PB-NK cells included genes associated with NK cell functional roles, including FGFBP2, granzymes (GZMH, GZMM), CXCR4, KLRF1, KLF2, IFNG (Table 1).

□ FGFBP2, encoding fibroblast growth factor-binding protein, is known to be secreted by cytotoxic lymphocytes.
□ Granzymes are a group of serine proteases which are stored in the cytotoxic granules of NK cells and cytotoxic T lymphocytes (ref). While GzmA and GzmB induce target cell death upon release to their cytoplasm and have been extensively studied, less is known about the functional role of GzmH, GzmK and GzmM.
□ CXCR4 regulates NK cell homing to bone marrow.
□ KLRF1 encodes NKp80, an activating C-type lectin-like immunoreceptor that is activated upon binding to activation-induced C-type lectin (AICL), inducing NK cell cytotoxicity and cytokine secretion.
□ Transcription factor KLF2 that regulates both NK cell proliferation and survival.
□ NK cell-derived IFN-γ (IFNG gene) is a key immunoregulatory factor secreted from activated NK cells that promotes adaptive immune response by modulating dendritic cell and T cell responses.

TABLE 1
Top 50 upregulated genes per PB-NK cluster.

| | | Feature | CYNK | PB-NK | PB-NK Log2 | PB-NK P- |
|---|---|---|---|---|---|---|
| | Feature ID | Name | Average | Average | Fold Change | Value |
| 1 | ENSG00000137441 | FGFBP2 | 0.099352 | 2.935962 | 4.88363 | 4.09E−78 |
| 2 | ENSG00000100450 | GZMH | 0.136708 | 2.484828 | 4.182845 | 2.49E−58 |
| 3 | ENSG00000276085 | CCL3L3 | 0.072152 | 1.251852 | 4.115143 | 2.13E−49 |
| 4 | ENSG00000197540 | GZMM | 0.134235 | 1.982728 | 3.883559 | 1.40E−50 |
| 5 | ENSG00000121966 | CXCR4 | 0.403236 | 5.935725 | 3.879087 | 9.19E−51 |
| 6 | ENSG00000169554 | ZEB2 | 0.127877 | 1.860789 | 3.861967 | 7.03E−50 |
| 7 | ENSG00000127528 | KLF2 | 0.172475 | 1.92761 | 3.481483 | 1.86E−40 |
| 8 | ENSG00000189067 | LITAF | 0.297791 | 3.231559 | 3.439184 | 1.06E−39 |
| 9 | ENSG00000069667 | RORA | 0.101913 | 1.055542 | 3.371425 | 3.26E−37 |
| 10 | ENSG00000145220 | LYAR | 0.142448 | 1.306592 | 3.196402 | 2.39E−33 |
| 11 | ENSG00000125107 | CNOT1 | 0.208595 | 1.809824 | 3.116348 | 3.39E−32 |
| 12 | ENSG00000111537 | IFNG | 0.193317 | 1.639941 | 3.083863 | 1.11E−29 |
| 13 | ENSG00000158050 | DUSP2 | 0.40774 | 3.322164 | 3.025836 | 4.12E−30 |
| 14 | ENSG00000110046 | ATG2A | 0.190226 | 1.508942 | 2.987028 | 3.39E−29 |
| 15 | ENSG00000173762 | CD7 | 0.492697 | 3.641922 | 2.885402 | 1.77E−27 |
| 16 | ENSG00000141682 | PMAIP1 | 0.252398 | 1.820017 | 2.849558 | 6.51E−26 |
| 17 | ENSG00000078304 | PPP2R5C | 0.381864 | 2.591665 | 2.762207 | 6.15E−25 |
| 18 | ENSG00000153234 | NR4A2 | 0.399174 | 2.622622 | 2.715393 | 5.59E−24 |
| 19 | ENSG00000152518 | ZFP36L2 | 0.856899 | 5.585388 | 2.703993 | 4.72E−24 |
| 20 | ENSG00000145675 | PIK3R1 | 0.325168 | 2.078618 | 2.675822 | 2.70E−23 |
| 21 | ENSG00000150045 | KLRF1 | 0.191285 | 1.177103 | 2.620822 | 4.78E−22 |
| 22 | ENSG00000255198 | SNHG9 | 0.516983 | 2.951818 | 2.512937 | 1.34E−20 |
| 23 | ENSG00000125148 | MT2A | 0.51504 | 2.913311 | 2.499426 | 9.06E−20 |
| 24 | ENSG00000116741 | RGS2 | 0.203737 | 1.147279 | 2.492865 | 1.51E−19 |
| 25 | ENSG00000153922 | CHD1 | 0.252574 | 1.350762 | 2.418474 | 9.42E−19 |
| 26 | ENSG00000120129 | DUSP1 | 2.078529 | 9.865317 | 2.24638 | 2.58E−16 |
| 27 | ENSG00000143924 | EML4 | 0.256284 | 1.150299 | 2.165756 | 7.80E−15 |
| 28 | ENSG00000128016 | ZFP36 | 2.22866 | 9.777355 | 2.132849 | 1.32E−14 |
| 29 | ENSG00000163874 | ZC3H12A | 0.261759 | 1.120475 | 2.097382 | 7.47E−14 |
| 30 | ENSG00000105993 | DNAJB6 | 0.6506 | 2.667169 | 2.035058 | 2.98E−13 |
| 31 | ENSG00000126524 | SBDS | 0.534822 | 2.185078 | 2.030148 | 3.57E−13 |
| 32 | ENSG00000125347 | IRF1 | 1.450448 | 5.812277 | 2.002193 | 7.32E−13 |
| 33 | ENSG00000157514 | TSC22D3 | 1.103379 | 4.30409 | 1.963373 | 2.57E−12 |
| 34 | ENSG00000184205 | TSPYL2 | 0.592137 | 2.247746 | 1.924086 | 1.14E−11 |
| 35 | ENSG00000146278 | PNRC1 | 1.362312 | 5.156149 | 1.919832 | 7.77E−12 |
| 36 | ENSG00000135070 | ISCA1 | 0.27898 | 1.043084 | 1.90227 | 2.06E−11 |
| 37 | ENSG00000171223 | JUNB | 4.09462 | 15.11622 | 1.883884 | 2.20E−11 |
| 38 | ENSG00000156232 | WHAMM | 0.316425 | 1.146147 | 1.856513 | 7.14E−11 |
| 39 | ENSG00000164327 | RICTOR | 0.318279 | 1.101977 | 1.791406 | 3.85E−10 |
| 40 | ENSG00000118503 | TNFAIP3 | 0.550807 | 1.902316 | 1.787777 | 3.93E−10 |
| 41 | ENSG00000120616 | EPC1 | 0.562199 | 1.846066 | 1.714953 | 2.17E−09 |
| 42 | ENSG00000167508 | MVD | 0.309448 | 1.00722 | 1.702322 | 4.11E−09 |
| 43 | ENSG00000013441 | CLK1 | 0.690164 | 2.216412 | 1.682859 | 4.62E−09 |
| 44 | ENSG00000188042 | ARL4C | 0.437325 | 1.388136 | 1.666056 | 8.18E−09 |
| 45 | ENSG00000162924 | REL | 0.553809 | 1.736208 | 1.648145 | 1.14E−08 |
| 46 | ENSG00000005483 | KMT2E | 0.79402 | 2.460289 | 1.631225 | 1.47E−08 |
| 47 | ENSG00000119801 | YPEL5 | 0.966141 | 2.98202 | 1.625617 | 1.70E−08 |
| 48 | ENSG00000123505 | AMD1 | 0.558578 | 1.664102 | 1.574595 | 6.03E−08 |
| 49 | ENSG00000159388 | BTG2 | 0.751541 | 2.22132 | 1.563151 | 7.55E−08 |
| 50 | ENSG00000010404 | IDS | 0.723193 | 2.128073 | 1.556757 | 8.48E−08 |

Top differentially expressed genes in CYNK cluster that are encode factors associated with NK cell functional role include surface receptors and co-receptors (CD96, NCR3, CD59, KLRC1), TNFSF10, immune checkpoint genes (TNFRSF18, TNFRSF4, HAVCR2), NK cell receptor adaptor molecule genes (FCER1G and LAT2) (Table 2).

TABLE 2
Top 50 upregulated genes per CYNK cluster.

| | | Feature | PBNK | CYNK | CYNK Log2 | CYNK P- |
|---|---|---|---|---|---|---|
| | Feature ID | Name | Average | Average | Fold Change | Value |
| 1 | ENSG00000102471 | NDFIP2 | 0.077391 | 1.45981 | 4.230949 | 1.69E−22 |
| 2 | ENSG00000242258 | LINC00996 | 0.063046 | 1.183921 | 4.222944 | 5.04E−22 |
| 3 | ENSG00000172005 | MAL | 0.057005 | 1.03529 | 4.173813 | 1.35E−21 |
| 4 | ENSG00000108702 | CCL1 | 0.078524 | 1.334494 | 4.080611 | 5.11E−09 |
| 5 | ENSG00000198125 | MB | 0.10193 | 1.683947 | 4.041355 | 1.45E−20 |
| 6 | ENSG00000128040 | SPINK2 | 0.087962 | 1.233641 | 3.804242 | 7.88E−19 |
| 7 | ENSG00000166920 | C15orf48 | 0.078901 | 1.018246 | 3.683547 | 6.40E−18 |
| 8 | ENSG00000134072 | CAMK1 | 0.151762 | 1.932724 | 3.667647 | 2.13E−18 |
| 9 | ENSG00000134545 | KLRC1 | 0.509273 | 4.740451 | 3.217889 | 9.47E−16 |
| 10 | ENSG00000121858 | TNFSF10 | 0.295975 | 2.682764 | 3.178801 | 6.44E−15 |
| 11 | ENSG00000186891 | TNFRSF18 | 1.182011 | 10.09017 | 3.093605 | 6.96E−15 |
| 12 | ENSG00000008517 | IL32 | 4.345617 | 37.08234 | 3.093395 | 6.60E−15 |
| 13 | ENSG00000042493 | CAPG | 0.369213 | 3.112494 | 3.074529 | 9.91E−15 |
| 14 | ENSG00000235576 | AC092580.4 | 0.44736 | 3.660475 | 3.031759 | 2.23E−14 |
| 15 | ENSG00000163191 | S100A11 | 0.41527 | 3.364804 | 3.017543 | 2.42E−14 |
| 16 | ENSG00000186827 | TNFRSF4 | 0.135529 | 1.097816 | 3.01448 | 1.91E−13 |
| 17 | ENSG00000074800 | ENO1 | 2.166202 | 16.05066 | 2.889567 | 1.86E−13 |
| 18 | ENSG00000158869 | FCER1G | 0.734274 | 5.393877 | 2.876632 | 2.43E−13 |
| 19 | ENSG00000118971 | CCND2 | 0.457175 | 3.324621 | 2.861636 | 3.21E−13 |
| 20 | ENSG00000205426 | KRT81 | 0.169883 | 1.187806 | 2.803005 | 3.69E−12 |
| 21 | ENSG00000243927 | MRPS6 | 0.358643 | 2.29304 | 2.675597 | 6.10E−12 |
| 22 | ENSG00000182718 | ANXA2 | 0.206125 | 1.282389 | 2.635118 | 3.48E−11 |
| 23 | ENSG00000125384 | PTGER2 | 0.175546 | 1.08713 | 2.628037 | 4.29E−11 |
| 24 | ENSG00000124767 | GLO1 | 0.214053 | 1.289543 | 2.588793 | 6.50E−11 |
| 25 | ENSG00000135077 | HAVCR2 | 0.175924 | 1.031051 | 2.548543 | 1.51E−10 |
| 26 | ENSG00000103490 | PYCARD | 0.183097 | 1.070527 | 2.545209 | 1.34E−10 |
| 27 | ENSG00000086730 | LAT2 | 0.178566 | 1.04156 | 2.541707 | 1.53E−10 |
| 28 | ENSG00000141526 | SLC16A3 | 0.282006 | 1.622835 | 2.523282 | 1.73E−10 |
| 29 | ENSG00000103187 | COTL1 | 0.894342 | 5.013779 | 2.486834 | 1.45E−10 |
| 30 | ENSG00000067225 | PKM | 1.099712 | 6.145949 | 2.482453 | 1.11E−10 |
| 31 | ENSG00000177156 | TALDO1 | 0.196687 | 1.084745 | 2.46115 | 4.23E−10 |
| 32 | ENSG00000153283 | CD96 | 0.368458 | 2.029162 | 2.460314 | 1.66E−10 |
| 33 | ENSG00000204475 | NCR3 | 0.640272 | 3.472457 | 2.438804 | 2.31E−10 |
| 34 | ENSG00000170442 | KRT86 | 0.257845 | 1.372733 | 2.410873 | 1.02E−09 |
| 35 | ENSG00000117632 | STMN1 | 0.468878 | 2.413499 | 2.36315 | 1.22E−09 |
| 36 | ENSG00000227507 | LTB | 3.831437 | 19.41653 | 2.341609 | 1.09E−09 |
| 37 | ENSG00000130429 | ARPC1B | 0.570053 | 2.846585 | 2.31957 | 1.27E−09 |
| 38 | ENSG00000162704 | ARPC5 | 0.347317 | 1.717418 | 2.30484 | 1.66E−09 |
| 39 | ENSG00000088832 | FKBP1A | 0.40017 | 1.978205 | 2.304629 | 1.60E−09 |
| 40 | ENSG00000102265 | TIMP1 | 0.385447 | 1.902345 | 2.302248 | 1.96E−09 |
| 41 | ENSG00000113088 | GZMK | 0.290312 | 1.403201 | 2.27168 | 1.37E−08 |
| 42 | ENSG00000085063 | CD59 | 0.215186 | 1.035997 | 2.265377 | 7.12E−09 |
| 43 | ENSG00000102144 | PGK1 | 1.405879 | 6.735348 | 2.260328 | 2.92E−09 |
| 44 | ENSG00000148908 | RGS10 | 0.217451 | 1.014713 | 2.220352 | 1.33E−08 |
| 45 | ENSG00000196405 | EVL | 1.186164 | 5.50471 | 2.214345 | 5.41E−09 |
| 46 | ENSG00000128340 | RAC2 | 1.063092 | 4.917253 | 2.209516 | 5.72E−09 |
| 47 | ENSG00000100097 | LGALS1 | 4.427539 | 20.46621 | 2.208968 | 6.05E−09 |
| 48 | ENSG00000139626 | ITGB7 | 0.50059 | 2.285445 | 2.19016 | 8.54E−09 |
| 49 | ENSG00000196230 | TUBB | 1.062715 | 4.838214 | 2.186651 | 1.22E−08 |
| 50 | ENSG00000171314 | PGAM1 | 0.670096 | 3.046436 | 2.18433 | 8.56E−09 |

To better understand how the cytotoxic response is initiated in CYNK cells, we specifically analyzed the expression of manually chosen genes encoding well characterized proteins leading from target detection to a cytolytic response, with main focus on NK cell receptors and adaptor molecule (Table 3). Differential gene expression analysis showed high expression of the two key cytotoxic molecules perforin (PRF1) and granzyme B (GZMB) in CYNK cells. Similarly, most receptors that were differentially expressed between CYNK and PB-NK cells, with the exception of KLRF1 (encoding NKp80), were higher expressed on CYNK cells. Expression of selected NK cell effector and receptor genes is visualized on tSNE plots in FIG. 6C. Elevated expression of genes encoding components of the NK cell cytotoxic machinery correlate well with the high cytotoxic activity of CYNK cells against a broad range of target cells.

TABLE 3
Top differentially expressed genes encoding factors regulating NK cell cytolytic function.
Genes that had <1 count per cell across the entire cluster were excluded.

| | | | | | | CYNK | |
|---|---|---|---|---|---|---|---|
| | | | | | | Log2 | |
| | | Feature | | CYNK | PBNK | Fold | CYNK P- |
| | Feature ID | Name | Alias | Average | Average | Change | Value |
| 1 | ENSG00000134545 | KLRC1 | NKG2A, | 4.740451 | 0.509273 | 3.217889 | 9.47E−16 |
| | | | CD159a | | | | |
| 2 | ENSG00000121858 | TNFSF10 | TRAIL | 2.682764 | 0.295975 | 3.178801 | 6.44E−15 |
| 3 | ENSG00000186891 | TNFRSF18 | GITR | 10.09017 | 1.182011 | 3.093605 | 6.96E−15 |
| 4 | ENSG00000186827 | TNFRSF4 | CD134, | 1.097816 | 0.135529 | 3.014481 | 1.91E−13 |
| | | | OX40 | | | | |
| 5 | ENSG00000135077 | HAVCR2 | TIM-3 | 1.031051 | 0.175924 | 2.548543 | 1.51E−10 |
| 6 | ENSG00000153283 | CD96 | Tactile | 2.029162 | 0.368458 | 2.460314 | 1.66E−10 |
| 7 | ENSG00000204475 | NCR3 | CD337, | 3.472457 | 0.640272 | 2.438804 | 2.31E−10 |
| | | | NKp30 | | | | |
| | | | MAC-IP, | | | | |
| 8 | ENSG00000085063 | CD59 | MIRL, protectin | 1.035997 | 0.215186 | 2.265377 | 7.12E−09 |
| 9 | ENSG00000139626 | ITGB7 | | 2.285445 | 0.50059 | 2.19016 | 8.54E−09 |
| 10 | ENSG00000180644 | PRF1 | | 3.589295 | 0.887169 | 2.016259 | 8.95E−08 |
| 11 | ENSG00000100453 | GZMB | | 11.6194 | 3.515453 | 1.725026 | 4.27E−06 |
| 12 | ENSG00000100385 | IL2RB | | 2.568753 | 0.956632 | 1.424929 | 0.000126 |
| 13 | ENSG00000205809 | KLRC2 | NKG2C, | 1.419451 | 0.784861 | 0.854636 | 0.026587 |
| | | | CD159c | | | | |
| 14 | ENSG00000111796 | KLRB1 | CD161 | 18.74844 | 10.45953 | 0.842324 | 0.027995 |
| 15 | ENSG00000150045 | KLRF1 | NKp80 | 0.191285 | 1.177103 | −2.62082 | 4.78E−22 |

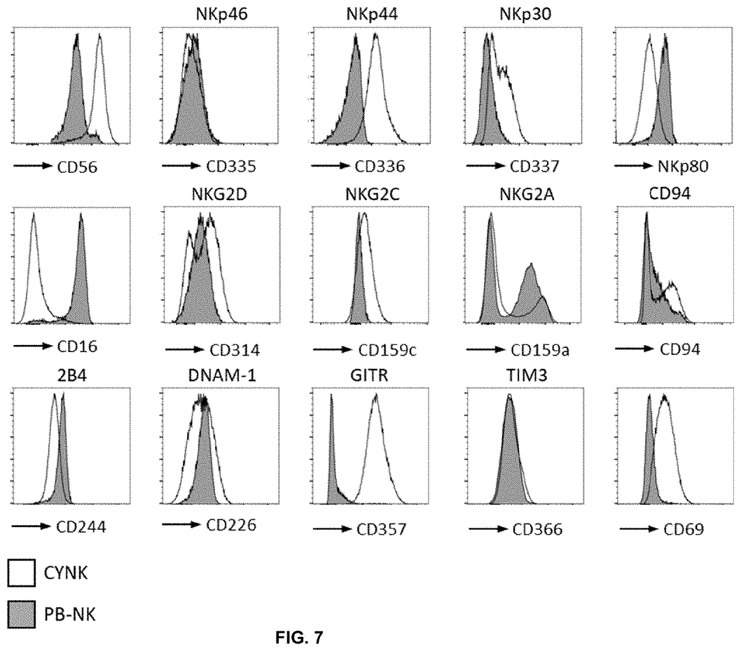

We next analyzed the transcriptional profile of CYNK and PB-NK cells by quantitative real-time PCR (qRT-PCR) focusing on selected NK cell-associated genes that were highly and/or differentially expressed in the scRNAseq dataset (FIG. 7). RNA was extracted from freshly thawed naïve cells post isolation or culture. qRT-PCR demonstrated high expression of CD69, KLRK1 and KLRB1 relative to the housekeeping gene GAPDH in both CYNK and PB-NK cells, whereas, KLRK1 and KLRB1, encoding for NKG2D and CD161/KLRB1, respectively, were significantly higher expressed in PB-NK cells. Significant differential expression of NKp80, encoded by KLRF1 gene, earlier seen by scRNAseq (Table 3), was confirmed by qRT-PCR. Similarly, KLRD1 was higher expressed on PB-NK compared to CYNK cells. Together, the data show higher expression of the inhibitory killer cell lectin-like receptor (KLRB1, KLRD1, KLRF1) expression on PB-NK cells when compared to CYNK cells. The two C-type lectin receptor genes KLRC1 and KLRC2, encoding the inhibitory NKG2A and the activating NKG2C, were higher expressed in CYNK cells. Of the natural cytotoxicity receptors (NCRs), only NCR2 (encoding NKp44) was differentially expressed with high expression in CYNK cells and almost no expression in PB-NK cells. Two co-activating NK cell receptor genes CD244 (2B4) and CD226 (DNAM-1) were slightly higher expressed in PB-NK compared to CYNK cells. Alongside the typical ligand-activated NK cell receptor genes, we also analyzed the expression of FCGR3A encoding an Fc receptor CD16 that is required for antibody-dependent cell-mediated cytotoxicity. Whereas scRNAseq data demonstrated no significant differential expression of FCGR3A, by qRT-PCR it was highly expressed in the PB-NK cells and at a very low level in CYNK cells. The expression of two genes TNFRSF18 and TNFSF10 that were highly differentially expressed by scRNAseq and elevated in the CYNK cluster, were also analyzed by qRT-PCR. The PCR data confirms high expression of these genes encoding for GITR and TRAIL, respectively, on CYNK cells relative to low level expression in PB-NK cells.

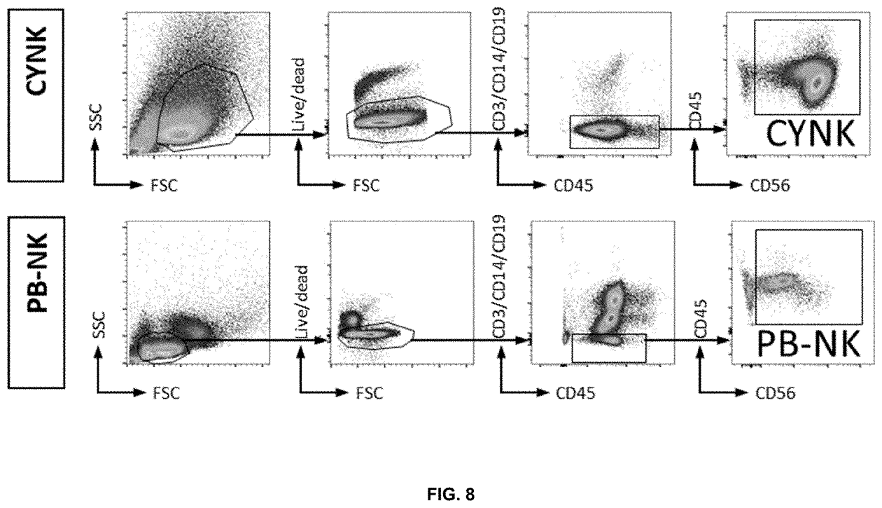

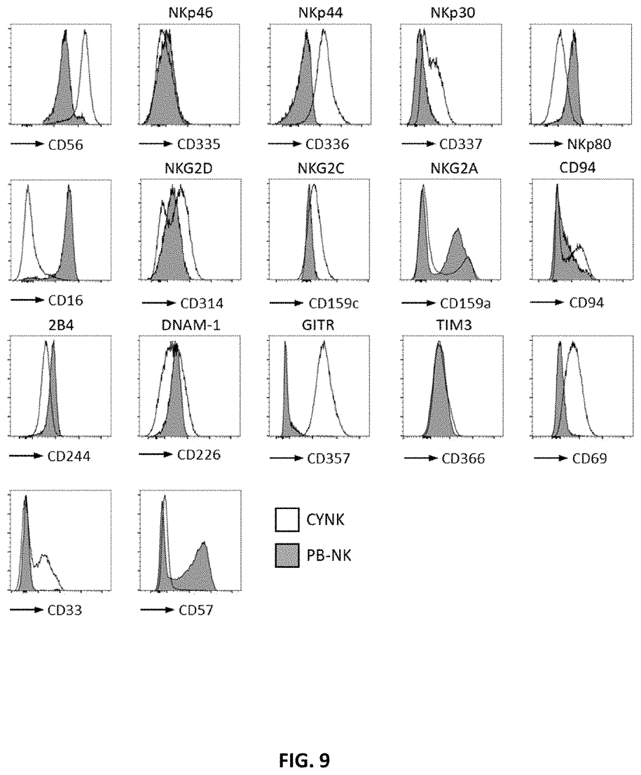

Lastly, we characterized CYNK cells relative to PB-NK by surface protein expression using flow cytometry. Antibodies targeting various NK cell receptors were chosen based on the transcriptional characterization by scRNAseq and qRT-PCR (Tables 1-3, GIG. 6 and FIG. 7). NK cells express high level of the NK cell marker CD56 and lack the expression of T cell, B cell and myeloid cell markers CD3, CD19 and CD14, respectively (FIG. 8). Whereas a majority of PB-NK cells express CD56 at a low level, a small subset of PB-NK cells express CD56 at a level seen in CYNK cells (FIG. 9). NCR analysis demonstrated a high expression of NKp44 in CYNK cells, whereas NKp44 was expressed at a low level in PB-NK, corresponding well to our transcriptional analysis (FIG. 7). NKp80, on the other hand, was expressed on PB-NK cell and little on CYNK, also confirming the transcriptional data of KLRF1 expression (Table 1 and FIG. 7). CD16 was virtually not expressed on CYNK cells, whereas the majority of PB-NK cells expressed CD16 at a high level. CD16 protein expression, therefore, also corresponds well to transcriptional analysis (Table 1 and FIG. 7). The expression of killer cell lectin-like receptors was comparable between CYNK and PB-NK cells, with CYNK cells demonstrating higher mean fluorescence intensity compared to PB-NK cells for NKG2D, NKG2C, CD94 (NKG2C) and NKG2A. GITR, a checkpoint inhibitor molecule, encoded by TNFRSF18, was not expressed on PB-NK cells but highly on all CYNK cells, correlating well to qRT-PCR data.

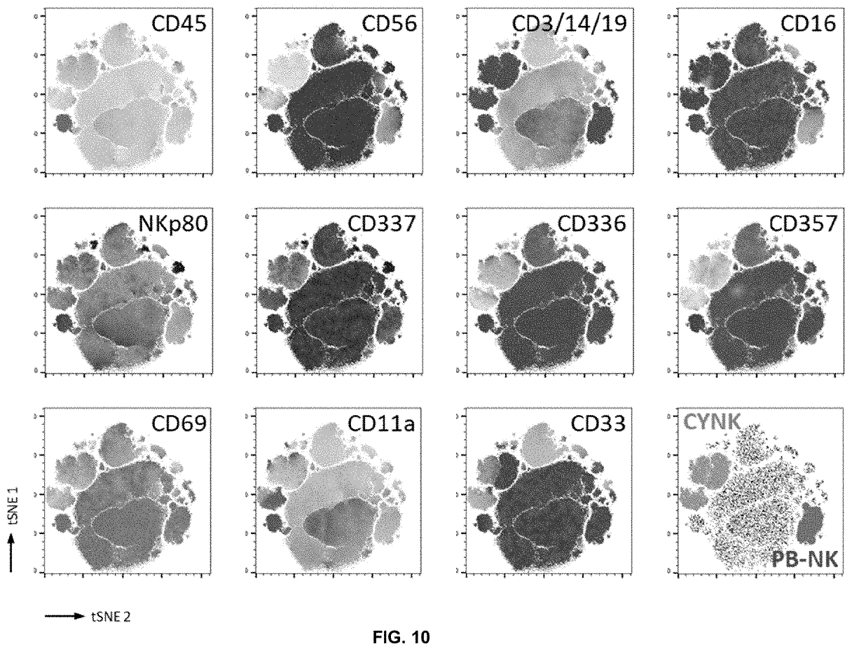

We used the flow cytometry dataset (FIG. 8 and FIG. 9) to perform an unbiased analysis of the surface marker expression on CYNK and PB-NK cell populations (FIG. 10).

Antibody-stained CYNK and PBMC cells were mixed for acquisition and analyzed by flow cytometry. It is evident from the tSNE plots that CYNK and PB-NK cells cluster separately from each other and other peripheral blood cells when looking at the localization of CD56- and CD3/CD14/CD19-positive cells on the plot. High expression of NKp44 (CD336) and GITR (CD357) enable the identification of CYNK cells as GITR is virtually not expressed in any cell type in the PBMC subsets. PB-NK cells on the other hand, highly express CD16 and NKp80 that are not expressed on CYNK cells. Altogether, we have identified cell surface markers that allow to distinguish CYNK cells from PB-NK with high confidence.

### 7.5 Example 5: Treatment of Coronavirus Infections with CYNK Cells

### 1. Summary

Celularity is developing CYNK-001, previously designated as PNK-007, for the treatment of coronavirus disease 2019 (COVID-19). CYNK-001 is an allogeneic, culture-expanded natural killer (NK) cell population derived from human placental hematopoietic stem cells. CYNK-001 is formulated for intravenous (IV) administration and is currently being studied in three ongoing clinical trials: Phase 1 study under IND 016792 in patients who have relapsed and/or refractory AML, Phase 1/2 study under IND 017030 for multiple myeloma (MM), Phase 1 study under IND 019486 for glioblastoma multiforme (GBM).

COVID-19 is an outbreak of respiratory disease caused by a novel coronavirus (SARS-CoV-2) that was first detected in Wuhan City, China. At the time of writing of this document, the virus had spread to 41 countries on five continents. A lack of specific antiviral treatment for COVID-19 has resulted in many critically ill patients and considerable mortality, highlighting an urgent need for clinically effective solutions.

CYNK-001 is well characterized in respect to key cellular attributes: identity, morphology, immunophenotype, and functionality. CYNK-001 cells morphologically appear as large granular lymphocytes, and they are roughly spherical in shape with an average cell diameter of 9.5±0.1 μm. The majority (≥85%) of CYNK-001 cells are identified as CD56⁺ and contain very low to non-detectable levels of CD3⁺ T cells (≤1.0%) or CD19⁺ B cells (≤1.0%), as measured by flow cytometry. CYNK-001 cells additionally express activating receptors including NKG2D⁺, NKp46⁺, NKp30⁺ and DNAM-1⁺.

CYNK-001 demonstrates a range of biological activities expected of NK cells, including expression of perforin and granzyme B cytotoxic granules, cytolytic activity against hematological tumor cell lines and GBM solid tumor cell lines, and secretion of immunomodulatory cytokines, such as IFN-γ, TNF-α and GM-CSF in the presence of tumors cell lines.

In support of CYNK-001 for the treatment of COVID-19, our in vitro study showed that CYNK-001 express the NK cell activating receptors involved in the recognition of stressed and/or virus infected cells, suggesting that CYNK-001 could provide a benefit to the COVID-19 patients in terms of limiting SARS-CoV-2 replication and disease progression through elimination of the infected cells. Our data also show that CYNK-001 is positive for CXCR3 transcript that is known to direct NK cells to the site of infection, including coronavirus or Influenza infection in the lung. Complemented by our earlier data demonstrating localization of CYNK-001 to the lungs of mice post infusion, CXCR3 expression by CYNK-001 suggests that CYNK-001 could home to sites of SARS-CoV-2 infection in the lower airway, in a CXCL10-dependent manner. Finally, we analyzed whether CYNK-001 expressed the receptors co-opted by SARS-CoV-2 during infection. Angiotensin-converting enzyme 2 (ACE2) and Transmembrane protease, serine 2 (TMPRSS2) mediate the entry of SARS-CoV and SARS-CoV-2 on target cells but are not expressed on CYNK-001 cells, suggesting that CYNK-001 cells do not get infected by the virus.

The data outlined in this report support the clinical development of CYNK-001 for the treatment of COVID-19.

### 2. Introduction

Celularity is developing CYNK-001, previously designated as PNK-007, for the treatment of coronavirus disease 2019 (COVID-19). CYNK-001 is an allogeneic culture-expanded natural killer (NK) cell population derived from human placental hematopoietic stem cells. CYNK-001 is formulated for intravenous (IV) administration and is currently being studied in three ongoing clinical trials: Phase 1 study under IND 016792 in patients who have relapsed and/or refractory AML, Phase 1/2 study under IND 017030 for multiple myeloma (MM), Phase 1 study under IND 019486 for glioblastoma multiforme (GBM).

CYNK-001 consists of culture-expanded NK cells which are harvested and washed in Plasma-Lyte A, then packaged at 30×10⁶ cells/mL in a total volume of 20-mL of cryopreservation solution containing 10% (w/v) HSA, 5.5% (w/v) Dextran 40, 0.21% NaCl (w/v), 32% (v/v) Plasma-Lyte A, and 5% (v/v) dimethyl sulfoxide (DMSO). It is filled into the container closure, frozen using a controlled rate freezer, and cryopreserved. When required for administration by a site, CYNK-001 is shipped in the vapor phase of liquid nitrogen (LN2) to the designated clinical site, where it is processed for dose preparation in a standardized manner just prior to IV or intratumoral administration.

CYNK-001 is well characterized with respect to key cellular attributes: identity, morphology, immunophenotype, and functionality. CYNK-001 cells morphologically appear as large granular lymphocytes, and they are roughly spherical in shape with an average cell diameter of 9.5±0.1 μm. CYNK-001 cells are identified as CD56⁺ and CD3′ (≥85%) and contain very low to non-detectable levels of CD3⁺ T cells (≤1.0%) or CD19⁺ B cells (≤1.0%), as measured by flow cytometry.

CYNK-001 demonstrates a range of biological activities expected of NK cells, including the expression of perforin and granzyme B cytotoxic granules, cytolytic activity against hematological tumor cell lines and GBM solid tumor cell lines, and secretion of immunomodulatory cytokines such as IFN-γ, TNF-α and GM-CSF in the presence of tumors cell lines. CYNK-001 additionally express the NK activating receptors, including NKG2D⁺, NKp46⁺, NKp30⁺ and DNAM-1⁺ (CELU-RES-2019-001, CELU-RES-2019-002, CELU-RES-2019-003).

COVID-19 is an outbreak of a respiratory disease caused by a novel coronavirus (SARS-CoV-2) that was first detected in Wuhan City, China (Chen. 2020: Huang. 2020). According to the World Health Organization (WHO) on Feb. 26, 2020, there had been 81,109 confirmed cases globally, leading to 2,764 deaths. The causes of death include the complications from viral pneumonia, acute respiratory distress syndrome (ARDS) and multi-organ failure. SARS-CoV-2 was rapidly characterized as a novel member of the betacoronavirus genus, closely related to several bat coronaviruses as well as the severe acute respiratory syndrome coronavirus (SARS-CoV) (Wrapp, 2020). Compared to SARS-CoV, SARS-CoV-2 appears to be more readily transmitted from human to human, spreading to multiple continents and leading to the WHO declaration of a Public Health Emergence of International Concern (PHEIC) on Jan. 30, 2020. The lack of specific antiviral treatment for COVID-19 has resulted in many critically ill patients who do not respond to available treatments. Therefore, there is an urgent need to identify clinically effective solutions.

NK cells are innate immune cells with an important role in early host response against various pathogens. Multiple NK cell receptors are involved in the recognition of infected cells, including NKG2D, DNAM-1 and the natural cytotoxicity receptors NKp30, NKp44 and NKp46, which bind common stress ligands or pathogen-associated molecules (Cook, 2014). NK cells kill their target cells by cytotoxic molecules perforin and granzymes, and via death receptor-mediated apoptosis (Loh, 2005). In addition to their cytotoxic functions, NK cells are important for priming adaptive immunity by the secretion of various chemokines and cytokines, including IFN-g (Lanier. 2008).

Studies in humans and mice have established that there is robust activation of NK cells during viral infection, regardless of the virus class (Ivanova, 2014), and that the depletion of or a defect in NK cells aggravates viral pathogenesis (Littwitz, 2013; Bukowski, 1983; Gazit, 2006; Nogusa, 2008; Stein-Streilein, 1986). The important role of NK cells in virus control is illustrated by the diverse mechanisms human viruses, exemplified by CMV, have evolved to evade the NK cell recognition pathways (Lanier, 2008). In murine and human CMV infection, NK cell-mediated anti-viral activity is dependent on IFN-g secretion and perforin-dependent lysis of infected cells (Loh, 2005; Wu, 2015). HIV-1 infection in pregnancy is inhibited by decidual NK cells (Quillay, 2016) and hepatitis C virus infection is controlled by NK cells in the liver (Guidotti, 2006). NK cells have a major role in the early control of lung infections with pathogenic organisms. Timely NK cell-mediated cytotoxicity and IFN-g production limit diverse respiratory bacterial, fungal and viral infections (Ivanova, 2014).

NK cells sense the environment using a broad repertoire of surface receptors that can differentiate between normal and malignant cells (cancerous or infected) by binding to stress ligands and viral antigens. In particular, the stress ligand-induced NKG2D-MICA/B pathway has been shown to be important for NK cell activation and recognition of infected cells in multiple viral infections, including coronaviruses (Walsh, 2008; Lanier, 2008). Various viral glycoproteins expressed by enveloped viruses, including coronaviruses (Zeng, 2008), are specifically recognized by the natural cytotoxicity receptors NKp30, NKp44, and NKp46 (Cook, 2014). NK cell cytolytic activity against Influenza virus is triggered by the recognition of viral haemagglutinin by NKp46 receptor, but also induced by antibody-dependent cell-mediated cytotoxicity (ADCC) (Mandelboim, 2001). In infected tissue microenvironment, NK cell activation leads to increased activating receptor expression and their cytotoxic responses are strongly potentiated by type I IFNs produced by dendritic cells and infected epithelial cells, also enabling subsequent priming and T cell activation and memory (Lanier, 2008).

It was shown that coronavirus infection stimulates the recruitment of NK cells to control infection. Research following the SARS-CoV outbreak revealed that SARS-CoV infection in a mouse model resulted in acute expression of CCL5, CXCL10, and CCL3 chemokines in lung epithelial cells (Law, 2007). In a separate study, NK cells migrated to coronavirus-infected organs in a CXCL10 dependent manner that was associated with reduced coronavirus titers. Anti-viral activity and NK cell homing to the tissue correlated with IFN-g secretion (Trifilo, 2004).

A study of NK cells from peripheral blood of patients with SARS coronavirus (SARS-CoV) was evaluated for the number of NK cells, as it was previously noted that patients with lower NK cells in the HIV population were susceptible to retrovirus resistance. It was noted that patients with SARS coronavirus had significantly lower counts of NK cells in their peripheral blood compared to patients with mycoplasma pneumonia and healthy adults. It was unclear as to why the number was lower. It was hypothesized that either the NK cells had died as a direct attack from the virus or the NK cells were redistributed to targeted organs, such as the lungs (National Research Project of SARS, 2004). Hematological abnormalities such as thrombocytopenia and lymphopenia were common in both SARS-CoV and MERS-CoV patients. Thrombocytopenia and lymphopenia may be predictive of fatal outcome in MERS-CoV patients (Yin, 2018). Based on these observations, it is hypothesized that adoptive NK cell therapy may provide the antiviral activities in those with SARS-CoV-2 infection.

This report presents the characterization of CYNK-001 cells including the assessment of NK cell receptors, on RNA and protein level, related to anti-coronavirus activities. The data outlined in this report, along with previously demonstrated PNK-007 safety data in phase 1 AML and MM trials, and the cited literature, support the development of CYNK-001 for the treatment of COVID-19.

### 3. Purpose/Study Objectives

The purpose of this study was to evaluate the possibility of using CYNK-001 for anti-coronavirus purpose. Single-cell RNAseq and flow cytometric analysis were performed to characterize CYNK-001 cells and evaluate their potential to recognize virus-infected cells, thereby, providing a rationale for the clinical development of CYNK-001 for the treatment of COVID-19.

### 4. Purpose/Study Objectives

The purpose of this study was to evaluate the possibility of using CYNK-001 for anti-coronavirus purpose. Single-cell RNAseq and flow cytometric analysis were performed to characterize CYNK-001 cells and evaluate their potential to recognize virus-infected cells, thereby, providing a rationale for the clinical development of CYNK-001 for the treatment of COVID-19.

### 6. Methods

### Single-Cell RNA Sequencing

Frozen PNK-007 cells were processed for single-cell capture on barcoded beads followed by library preparation using the 10× Genomics® Chromium™ platform (10× Genomics, Pleasanton, Calif., USA). Sequencing was performed on Illumina HiSeq. Differential gene expression analysis was performed using 10× Genomics® Cell Ranger™ single-cell RNA-seq pipeline and data analyzed using Loupe Cell Browser.

### Flow Cytometry

CYNK-001 cells from six different donors (donor IDs: 2000108365, 2000109063, 2000109106, 2000111822, 2000112315, 2000113036), with NK cell purity [symbol] 90% CD56+CD3−, were used in this assay. Frozen CYNK-001 cells were thawed and washed m staining buffer (PBS (10010-023, Gibco) containing 10% FBS (10082-147, Gibco)). 1×106 CYNK-001 cells were stained with Aqua live dead (L34966, Invitrogen) in PBS and then blocked with a solution containing Mouse IgG2a, κ Isotype Control (555571, BD), Fc Block (564219, BD) and BD Horizon Brilliant Stain Buffer (563794, BD). Fluorophore-conjugated antibodies from BD and Biolegend were diluted in staining buffer according to manufacturer's instructions. The following antibodies were used: CD226 (Clone: DX11 559789, BD), CD337 (Clone: p30-15 563385, BD), CD335 (Clone: 9E2 563230, BD), CD56 (Clone: 5.1H11 362510, BioLegend), CD3 (Clone: SK7 560176, BD), CD14 (Clone: MφP9, 641394, BD), CD19 (Clone: SJ25C1 641395, BD), CD336 (Clone: p44-8 744300, BD), CD314 (Clone: BAT221 130-092-673, Miltenyi Biotech). Samples were acquired on Cytek Aurora flow cytometer (Cytek, CA, US) and data analyzed on Flowjo Software (BD).

### 7. Results

### CYNK-001 Characterization

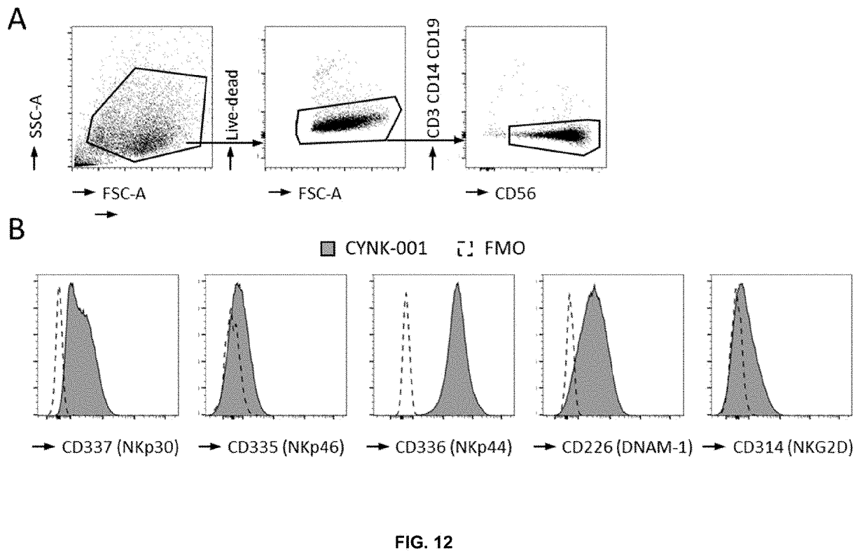

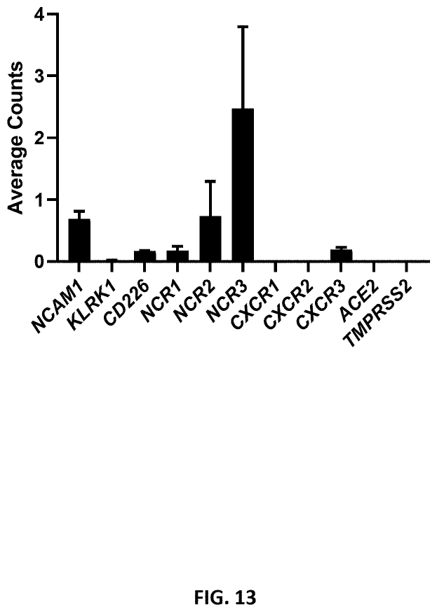

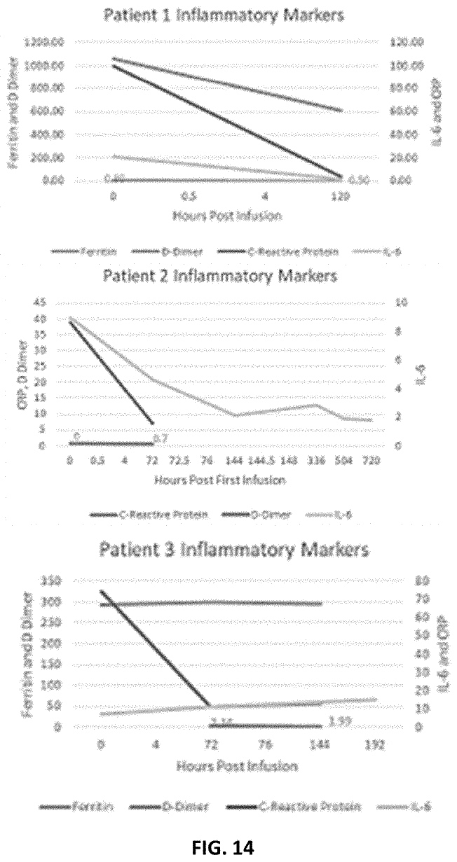

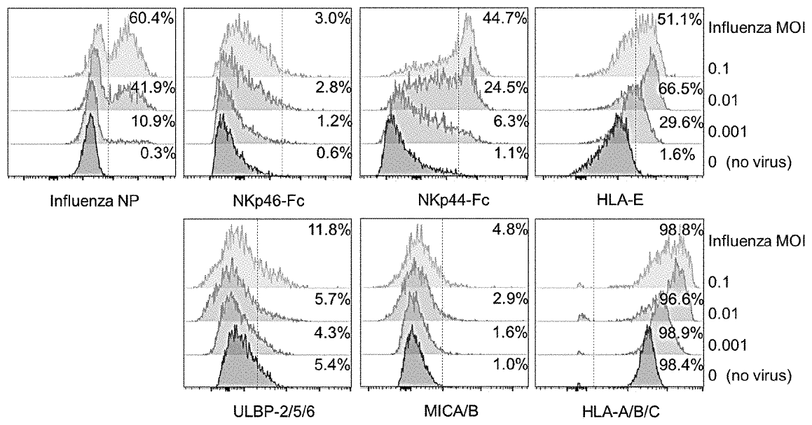

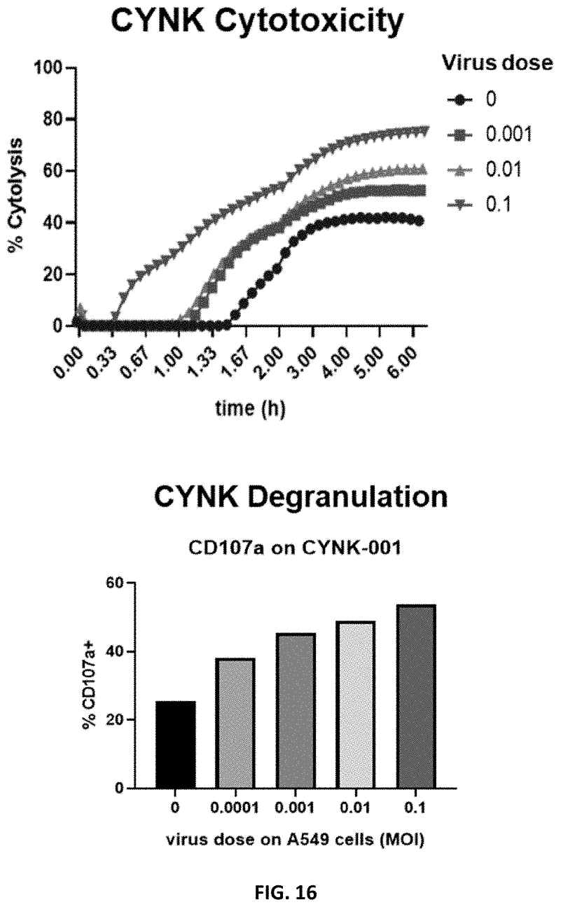

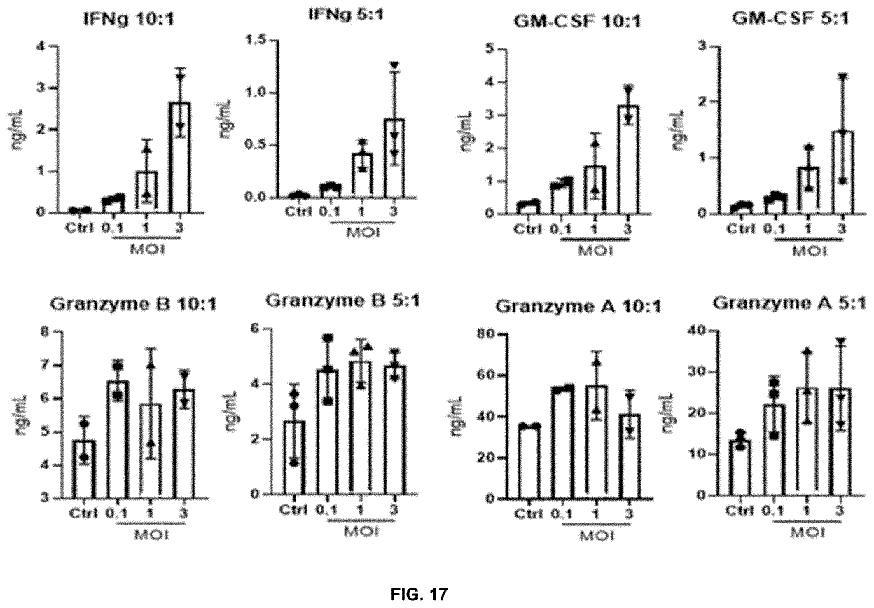

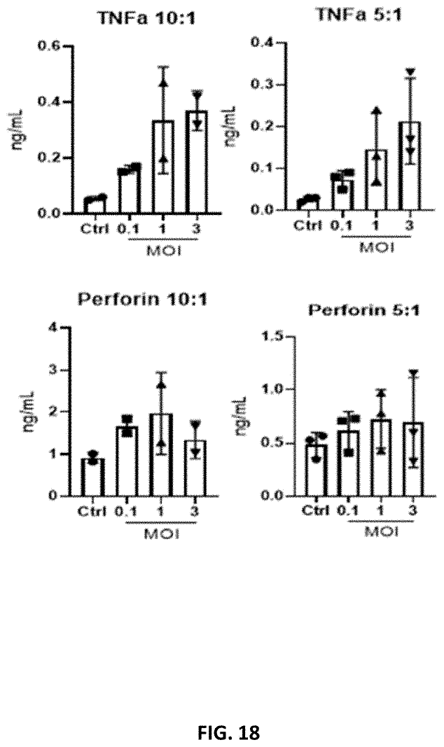

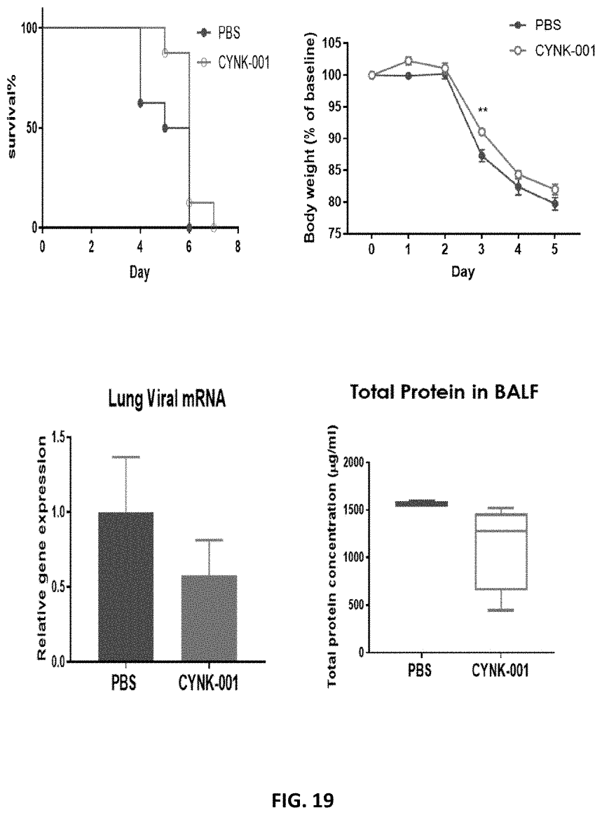

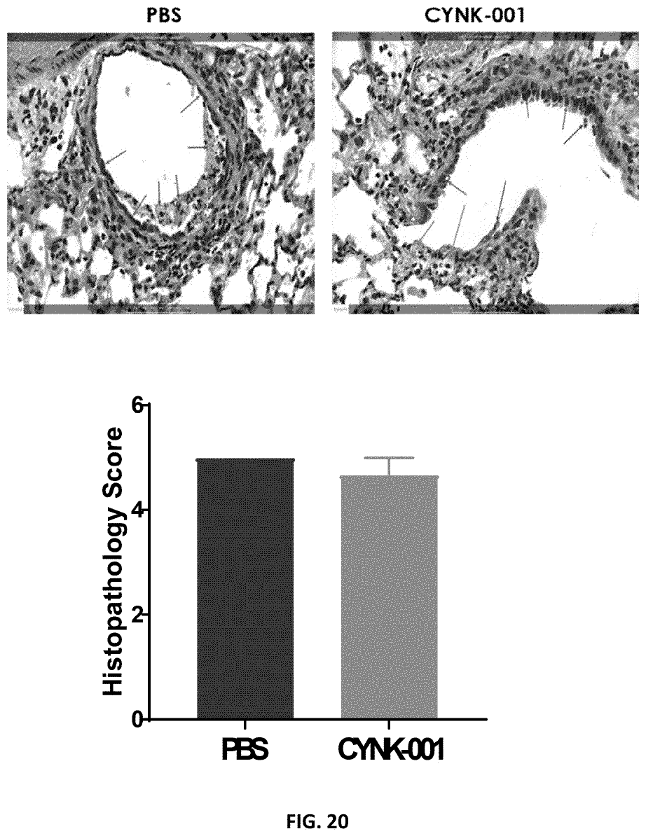

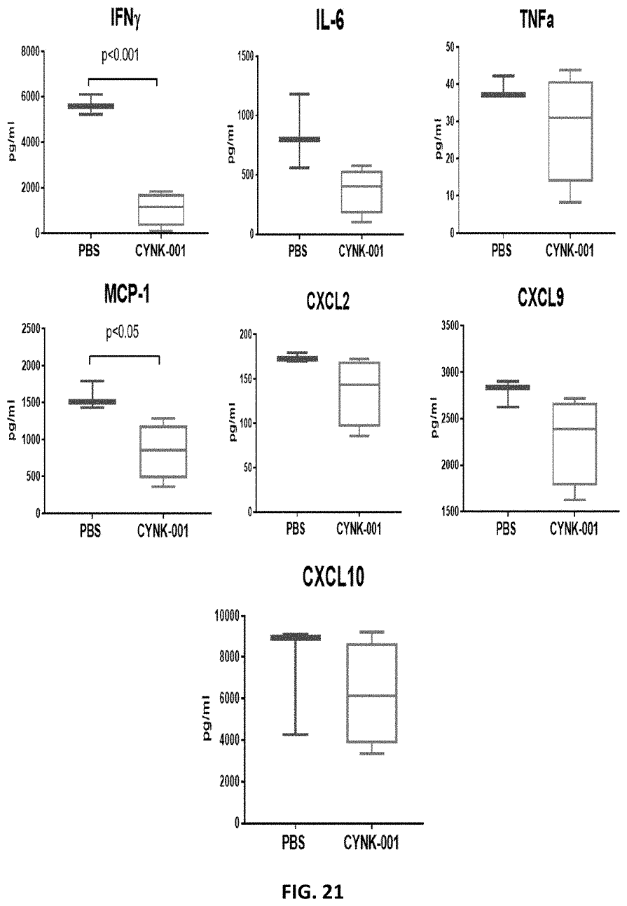

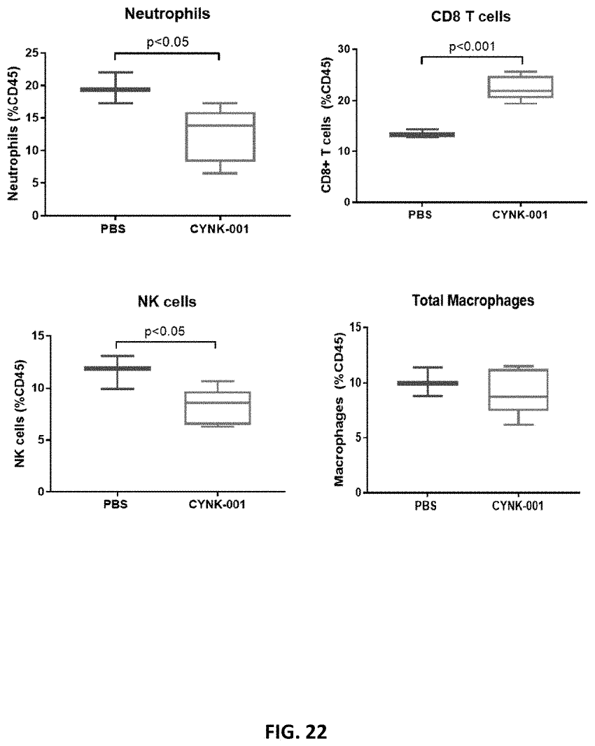

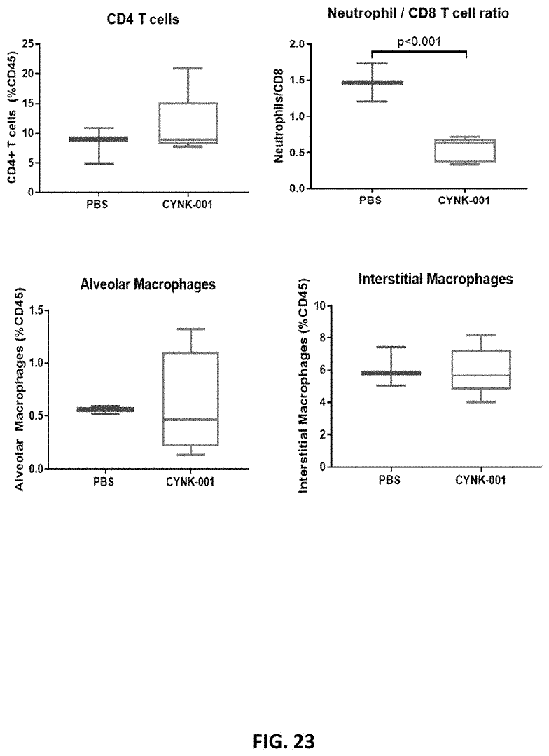

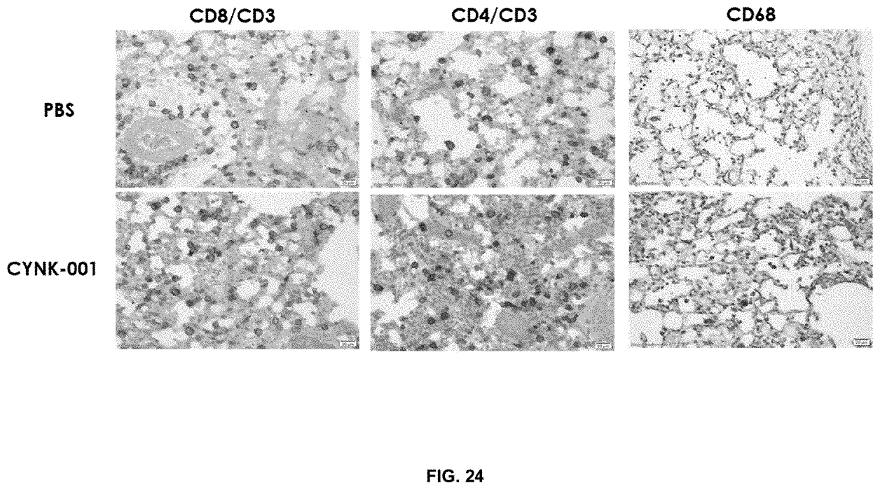

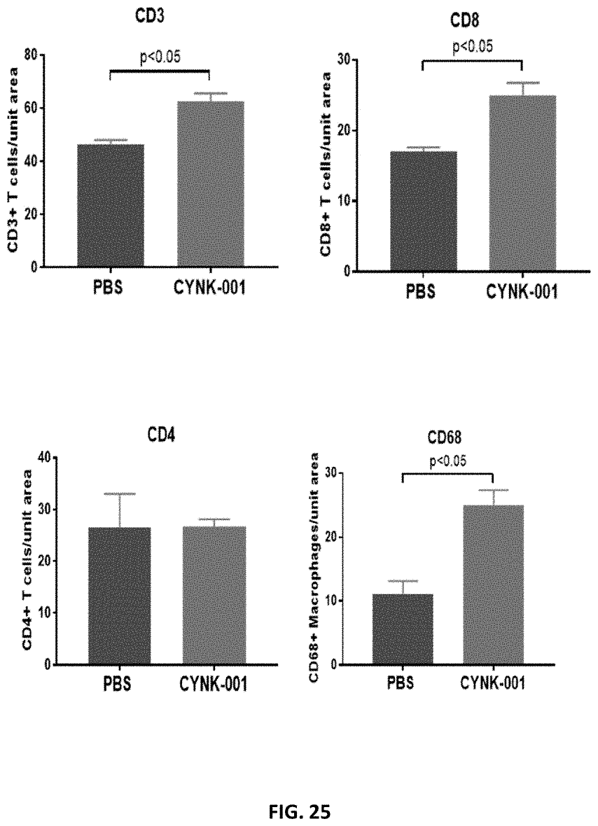

CYNK-001 are human placental hematopoietic stem cell-derived NK cells that express the dominant NK cell marker CD56 and lack lineage markers such as CD3, CD14 and CD19 (FIG. 12). CYNK-001 cells express the NK cell activating receptors NKG2D, DNAM1, NKp30, NKp46, and NKp44 that recognize stressed and virus-infected cells (Walsh, 2008; Lanier, 2008; Zeng, 2008; Cook, 2014) ((FIGS. 12A-12B, (FIG. 13, and Table 4). Whereas data on the pathogenic coronaviruses is limited, copious studies on the general mechanisms NK cells use to recognize infected cells in the context of diverse viral pathogens, allows us to predict that SARS-CoV-2 infected cells would express stress antigen molecules as in other viral infections, rendering them sensitive to CYNK-001 recognition and subsequent elimination.

Regarding the homing to infected tissues, Celularity has shown that CYNK-001 cells have immediate localization to the lungs following intravenous injection in the non-obese diabetic (NOD)-scid IL2Rgammanull (NSG) immune deficient mouse (IND 016792, CELU-2018-003; CELU-2019-001). It has been shown that CXCR3 expression on NK cells is involved in NK cell trafficking to the lung in Influenza virus infection (Carlin, 2018; Scharenberg, 2019). CXCR3 is also involved in CXCL10-directed NK cell homing to coronavirus infected tissues (Trifilo, 2004). Single-cell RNA sequencing (scRNAseq) demonstrated that CYNK-001 cells highly express the CXCR3 transcript ((FIG. 13). Together, the data suggest that CYNK cells have the potential to be efficacious and retained in the lungs given the heightened local biodistribution and chemoattraction to CXCL10.

NK cells can become infected with viral pathogens, therefore, either contributing to virus dissemination or resulting in decreased innate immune responses (Mao, 2009). Whereas SARS-CoV-2 uses ACE2 and the cellular protease TMPRSS2 for entry into target cells (Hoffmann, 2020), CYNK-001 do not express the transcript of either of the entry proteins, strongly suggesting that they do not get infected by SARS-CoV-2 ((FIG. 13).

TABLE 4
Expression level of selected receptors on CYNK-001 cells. Freshly
thawed CYNK-001 cells were stained with fluorophore-conjugated
antibodies recognizing indicated NK cell markers. Gating demonstrated
in FIG. 12A. CYNK-001 are defined as live CD3− CD14−
CD19− (Lineage, Lin) CD56+ cells. Data compiled of 6 donors.

| | | Gated on CD3− CD14- CD19−/CD56+ | | | | |
|---|---|---|---|---|---|---|
| Sample: | Lin-CD56+ | NKp30 | NKp46 | NKp44 | DNAM1 | NKG2D |
| Average | 90.10% | 37.80% | 40.80% | 96.20% | 70.40% | 43.60% |
| SD | 5.64% | 26.50% | 24.00% | 5.09% | 28.40% | 26.90% |

In the context of decreased circulating NK cells in patients with SARS-CoV and MERS-CoV infections, and the incidence of lymphopenia associated with SARS-CoV-2 infection (Chen, 2020; Wang, 2020; Huang, 2020), patients with SARS-CoV-2 may benefit from the application of adoptive NK cell therapy such as CYNK-001 to provide antiviral activities.

CYNK-001 cells were characterized to address their potential anti-viral role in the context of the novel SARS-CoV-2 outbreak. In vitro studies showed that CYNK-001 express the NK cell activating receptors involved in the recognition of stressed and/or virus infected cells, suggesting that CYNK-001 could provide a benefit to COVID-19 patients in terms of limiting SARS-CoV-2 replication and disease progression. Our earlier data demonstrating localization of CYNK-001 to the lungs of mice post infusion, suggests that CYNK-001 could reach the dominant infection site of SARS-CoV-2, the lower airways. Expression of CXCR3 on CYNK-001 is hypothesized to retain CYNK-001 in the infected lung in response to local chemokines induced by SARS-CoV-2. Finally, we analyzed whether CYNK-001 expressed the SARS-CoV-2 receptor. Two proteins, ACE2 and TMPRSS2, described to mediate the entry of SARS-CoV and SARS-CoV-2 to their target cells are not expressed on CYNK-001 cells, suggesting that CYNK-001 cells do not get infected by the virus.

In conclusion, extensive published data from preclinical and clinical studies characterizing the role on NK cells in the control of viral infections, together with our data on CYNK-001 cells, suggest that COVID-19 patients could benefit from CYNK-001 treatment and support the development of CYNK-001 for the treatment of COVID-19.

### REFERENCES

Bukowski J F, Woda B A, Habu S, Okumura K, Welsh R M. Natural killer cell depletion enhances virus synthesis and virus-induced hepatitis in vivo. J Immunol. 1983; 131(3):1531-1538.
Carlin L E, Hemann E A, Zacharias Z R, Heusel J W, Legge K L. Natural Killer Cell Recruitment to the Lung During Influenza A Virus Infection Is Dependent on CXCR3, CCR5, and Virus Exposure Dose. Front Immunol, 2018; 9:781. Published 2018 Apr. 17. doi: 10.3389/fimmu.2018.00781
Chen N, Zhou M, Dong X, Qu J, Gong F, Han Y, et al. Epidemiological and clinical characteristics of 99 cases of 2019 novel coronavirus pneumonia in Wuhan, China: a descriptive study. Lancet. 2020 Feb. 15; 395(10223):507-513.
Cook, K. D., S. N. Waggoner, and J. K. Whitmire, NK cells and their ability to modulate T cells during virus infections. Crit Rev Immunol, 2014. 34(5): p. 359-88.
Gazit R, Gruda R, Elboim M, et al. Lethal influenza infection in the absence of the natural killer cell receptor gene Ncr1. Nat Immunol, 2006; 7(5):517-523. doi: 10.1038/ni 1322
Guidotti L G, Chisari F V. Immunobiology and pathogenesis of viral hepatitis. Annu Rev Pathol. 2006; 1:23-61.
Hoffmann M, Kleine-Weber H, Kruger N, Muller M, Drosten C, Pohlmann S. The novel coronavirus 2019 (2019-nCoV) uses the SARS-coronavirus receptor ACE2 and the cellular protease TMPRSS2 for entry into target cells. bioRxiv preprint doi: https://doi.org/10.1101/2020.01.31.929042.
Huang C, Wang Y, Li X, Ren L, Zhao J, Hu Y, et al. Clinical features of patients infected with 2019 novel coronavirus in Wuhan, China. Lancet, 2020 Feb. 15; 395(10223):497-506.
Ivanova D, Krempels R, Ryfe J, Weitzman K, Stephenson D, Gigley J P. NK cells in mucosal defense against infection. Biomed Res Int, 2014; 2014:413982.
Lanier, L. L., Evolutionary struggles between NK cells and viruses. Nat Rev Immunol, 2008. 8(4): p. 259-68.
Law A H, Lee D C, Cheung B K, Yim H C, Lau A S. Role for nonstructural protein 1 of severe acute respiratory syndrome coronavirus in chemokine dysregulation. J Virol, 2007 January; 81(1): 416-22.
Littwitz E, Francois S, Dittmer U, Gibbert K. Distinct roles of NK cells in viral immunity during different phases of acute Friend retrovirus infection. Retrovirology, 2013 Nov. 1; 10:127. doi: 10.1186/1742-4690-10-127. PubMed PMID: 24182203; PubMed Central PMCID: PMC3826539.
Loh J, Chu D T, O'Guin A K, Yokoyama W M, Virgin H W 4th. Natural killer cells utilize both perforin and gamma interferon to regulate murine cytomegalovirus infection in the spleen and liver. J Virol, 2005 January; 79(1):661-7.
Mandelboim O, Lieberman N, Lev M, Paul L, Amon T I, Bushkin Y, et al. Recognition of haemagglutinins on virus-infected cells by NKp46 activates lysis by human NK cells. Nature, 2001 Feb. 22; 409(6823): 1055-60.
Mao H, Tu W, Qin G, et al. Influenza virus directly infects human natural killer cells and induces cell apoptosis. J Virol, 2009; 83(18):9215-9222. doi:10.1128/JVI.00805-09
Nogusa S, Ritz B W, Kassim S H, Jennings S R, Gardner E M. Characterization of age-related changes in natural killer cells during primary influenza infection in mice. Mech Ageing Dev. 2008; 129(4):223-230. doi:10.1016/j.mad.2008.01.003
Quillay H, El Costa H, Duriez M, Marlin R, Cannou C, Madec Y, et al. NK cells control HIV-1 infection of macrophages through soluble factors and cellular contacts in the human decidua. Retrovirology, 2016 Jun. 6; 13(1):39.
Scharenberg M, Vangeti S, Kekäläinen E, et al. Influenza A Virus Infection Induces Hyperresponsiveness in Human Lung Tissue-Resident and Peripheral Blood NK Cells. Front Immunol, 2019; 10:1116. Published 2019 May 17. doi:10.3389/fimmu.2019.01116
Stein-Streilein J, Guffee J. In vivo treatment of mice and hamsters with antibodies to asialo GM1 increases morbidity and mortality to pulmonary influenza infection. J Immunol. 1986 Feb. 15; 136(4): 1435-41.
Trifdo M J, Montalto-Morrison C, Stiles L N, Hurst K R, Hardison J L, Manning J E, et al. CXC chemokine ligand 10 controls viral infection in the central nervous system: evidence for a role in innate immune response through recruitment and activation of natural killer cells. J Virol, 2004 January; 78(2):585-94.
Walsh K B, Lodoen M B, Edwards R A, Lanier L L, Lane T E. Evidence for differential roles for NKG2D receptor signaling in innate host defense against coronavirus-induced neurological and liver disease. J Virol, 2008; 82(6):3021-3030. doi:10.1128/JVI.02032-07
Wang C, Horby P W, Hayden F G, Gao G F. A novel coronavirus outbreak of global health concern. Lancet, 2020 Feb. 15; 395(10223):470-473.
Wrapp D, Wang N, Corbett K S, Goldsmith J A, Hsieh C L, Abiona O, et al. Cryo-EM structure of the 2019-nCoV spike in the prefusion conformation. Science, 2020 Feb. 19. pii: eabb2507. doi: 10.1126/science.abb2507. [Epub ahead of print] PubMedPMID: 32075877.
Wu Z, Sinzger C, Reichel J J, Just M, Mertens T. Natural killer cells can inhibit the transmission of human cytomegalovirus in cell culture by using mechanisms from innate and adaptive immune responses. J Virol, 2015 March; 89(5):2906-17. doi: 10.1128/JVI.03489-14.
Yin Y, Wunderink R G. MERS, SARS and other coronaviruses as causes of pneumonia. Respirology, 2018 February; 23(2): 130-137.
Zeng Q, Langereis M A, van Vliet A L, Huizinga E G, de Groot R J. Structure of coronavirus hemagglutinin-esterase offers insight into corona and influenza virus evolution. Proc Natl Acad Sci USA, 2008 Jul. 1; 105(26):9065-9.

### 7.6 Example 6: Treatment of Coronavirus Infections with CYNK Cells

A Phase I/II study of human placental hematopoietic stem cell derived natural killer cells (CYNK-001) for the treatment of adults with coronavirus disease (COVID-19)

### Phase I:

The primary objectives of the Phase I portion of the study is to evaluate the safety, tolerability, and efficacy of multiple CYNK-001 intravenous (IV) infusions administered with an initial dose of 150×106 cells on Day 1 followed by second and third doses each of 600×106 cells on Days 4 and 7 in subjects with COVID-19.

Overall, if the safety stopping rules are not met and if efficacy is demonstrated in at least 2 out of the 14 subjects by Day 15 of CYNK-001 infusion, the study will move forward to the Phase II portion of the study.

### Phase II:

The primary objective of the Phase II portion is to evaluate the efficacy of CYNK-001 on subjects with COVID-19 by using the Ordinal Scale for Clinical Improvement (OSCI) defined by the World Health Organization (WHO).

The secondary objectives of the Phase II portion are: To determine safety and tolerability of CYNK-001 as measured by the frequency and severity of adverse events (AEs) using CTCAE, version 5.0; and To evaluate the overall clinical benefit of receiving CYNK-001 for COVID-19 as measured by rate of clinical improvement by OSCI, time to and rate of clinical improvement by NEWS2 Score, medical discharge, hospital utilization, and all-cause mortality rate, time to and rate of clearance of SARS-CoV-2, time to and rate of pulmonary clearance, duration of hospitalization, supplemental oxygen-free days, proportion of subjects requiring ventilation, SOFA score, and radiologic evaluation score.

### Study Population

SARS-CoV-2 positive subjects experiencing symptom(s)/clinical sign(s) of COVID-19 illness or having positive disease-related radiologic evaluation (chest x-ray/CT scan).

### Duration of Treatment:

Subject will receive up to 3 doses of CYNK-001 on Days 1, 4, and 7. A minimum of 2 doses is required for efficacy assessment. After the first dose, subsequent infusions on Days 4 and 7 will be provided only if no toxicity of Grade 3 and above (either related or unrelated to CYNK-001) is observed.

### Study Design:

The study will include a Phase I portion wherein a total of 14 subjects will be enrolled to assess the safety and efficacy of CYNK-001. To evaluate the safety for potential dose limiting toxicities (DLTs), this phase will enroll 3 subjects initially treated with CYNK-001. The safety data for these 3 subjects will be evaluated 24 hours after the final dose was provided to the 3rd subject.

If deemed safe, the remaining 11 subjects will be enrolled and monitored per the safety stopping rule until Study Day 15 where the first CYNK-001 infusion occurs on Day 1. If any DLT is observed in the first three subjects, the DMC will be convened for a recommendation. For the remaining 11 subjects, DMC will be convened if the safety stopping rule is met. Overall, in case of 2 out of the 6 subjects had experienced DLTs in the Phase I portion of this study, the DMC will be convened for safety evaluation. Subjects treated in the Phase I portion of the study will be treated in the inpatient setting.

The starting dose of 150×106 cells was selected as a desensitizing dose on Day 1 followed by 600×106 cells on Days 4 and 7. The DLTs will be evaluated through Study Day 28 following the first dose of CYNK-001 infusion (Day 1). Once CYNK-001 is deemed safe per the stopping rules and if efficacy is established in at least 2 out of the 14 subjects by Study Day 15, Phase II portion of the study will be initiated.

The Phase II portion of the study is a randomized, open-label, multi-site study. Subjects will be randomized into either CYNK-001 or Control group with best supportive care alone as defined by the institutional practice by 1:1 ratio. All subjects in both Phase I and Phase II will receive the best supportive care. Subjects treated in the Phase II portion of the study may be treated in the outpatient setting, if deemed safe based on Phase I data review and recommendation by the DMC.

The study is divided into 3 study periods:

### Screening Period

The Screening Period is defined as the period from signing the informed consent to just prior to the administration of CYNK-001. Due to the critical nature of this infection, this period may be less than a day. Upon giving written informed consent, all screening/baseline assessments will be completed. Some procedures that occur as part of standard of care in medical evaluation may be completed prior to the date of informed consent, according to institutional practices, and therefore do not need to be repeated. Chest x-rays, CT scans, rRT-PCR (or other approved test based on institutional practices for baseline assessment) viral testing, and blood work should occur often as outlined in the Table of Events.

During the Screening period, after having signed an Informed Consent Form (ICF), subjects will be assessed for eligibility for the study. Eligibility must be confirmed prior to proceeding to the treatment period. This information will need to be gathered and entered into the Electronic Data Capture (EDC). Subject eligibility will be based on investigator assessment using the Inclusion/Exclusion criteria provided as part of the study. The Screening Period is followed by a Treatment Period.

### Treatment Period (Day 1-Day 28)

The treatment period begins with the administration of study drug on Study Day 1 (in the inpatient setting for subjects treated in Phase I). For those subjects who are allocated to treatment with CYNK 001, the initial dose will consist of 150×106 cells on Day 1 followed by second and third doses each of 600×106 cells administered intravenously (IV) on Days 4 and 7. Subjects will receive a minimum of two and up to three CYNK-001 infusions. CYNK-001 infusions will occur on Study Days 1, 4, and 7. After the first dose, subsequent infusions on Days 4 and 7 will be provided only if no toxicity of Grade 3 and above (either related or unrelated to CYNK 001) is observed for each subject. If any such [symbol] Grade 3 toxicity is observed, the second and third doses will be delayed up to 48 hours until the noted event is resolved or reduced to Grade 1 toxicity level.

Subjects who were treated in the Phase 1 portion of the study may be discharged on the day following the final planned CYNK-001 infusion (i.e., discharged on Study Day 8, where infusions occur on Days 1, 4, and 7 or discharged on Study Day 5 if only two doses were received on Days 1 and 4.)

As part of discharge criteria, the study team should assess for further monitoring of subjects who may be experiencing toxicity of Grade 3 and above (either related or unrelated to CYNK-001) at the time of discharge and consult with the treating physician. The decision to discharge a subject should be in consultation with the treating physician and clinical site study team. Physicians should follow best clinical practice to determine appropriate timing of hospital discharge.

Upon discharge, plans should be made for appropriately delegated staff to have telephone contact with subjects every day between hospital discharge through at least Day 15 visit for safety/AE monitoring. The discharge plan should include follow-up visit schedule as well as planned telehealth visit schedule.

Upon discharge, subjects will be provided with a thermometer, pulse oximeter, and blood pressure monitor for at home collection of temperature, oxygen saturation, and blood pressure with written instructions on their use as well as expectations for self-monitoring. These daily measurements will be reported via telephone to the clinical staff during each daily telehealth visit. During these daily telehealth visits, subjects are to report any new symptoms or worsening of symptoms associated with previously identified adverse events and will also provide the study team with daily vital sign measurements. Any vital signs outside of normal range will be escalated to the clinical site study team and evaluated for appropriate management.

The subject will be asked during each call to report any new or worsening symptoms that could be consistent with adverse events since the previous visit or telephone call. The investigator (or appropriately delegated study staff) will determine if medical attention or an unscheduled clinic visit is required. Each telephone call should be carefully documented with date and time of the call in the source documents and reported as appropriate.

Consultation between the Medical Monitor and appropriately delegated site staff should occur every other day after hospital discharge up to Day 15 visit for ongoing review of the subject's clinical status. This communication may occur via telephone contact or written message (i.e., email) and should be documented accordingly.

Consultation with the Medical Monitor is required prior to each CYNK-001 infusion if there is:

an increase in supplemental oxygen of greater than or equal to 50% from baseline (for first CYNK-001 infusion) or from level at prior CYNK-001 infusion (for second and third CYNK 001 infusion) resulting in oxygen use of greater than 8 L.—or—a change in the mode of supplemental oxygen delivery with the intention to deliver oxygen more efficiently.

The Medical Monitor will escalate to the Sponsor clinical and safety teams as appropriate per GCP guidelines to advise if a subject who is experiencing rapid worsening of disease should proceed to first or subsequent CYNK-001 infusion.

Subjects treated in the Phase II portion of the study will be treated either in the inpatient or outpatient setting, as determined after review of data from the Phase I portion of the study and based on DMC recommendation. The level of outpatient monitoring of subjects treated in the Phase II portion will be determined based on review of Phase I data and DMC recommendation.

A de-escalation dose (Dose −1) will be initiated based on the study safety stopping rules and dose-limiting toxicities. Dose de-escalation is defined as reducing the frequency of the doses (without reducing the total number of cells given per dose) by providing doses only on Days 1 and 7 due to any potential safety concerns per DMC recommendation.

On each day of CYNK-001 infusion, subjects will be pre-medicated and post-medicated with acetaminophen 650 mg orally (PO) and diphenhydramine 25 mg (PO/IV) at least 30 minutes prior to and approximately 4 hours following the end of the CYNK-001 infusion. Meperidine may also be administered to control rigors, if clinically indicated. Subjects must be monitored for at least 4 hours after completion of each CYNK-001 infusion.

All subjects (even in control arm) should meet the inclusion/exclusion criteria.

The control arm subjects in the Phase II portion of the study will receive the best supportive care as defined by the institutional practice without CYNK-001. All subjects in both Phase I and Phase II will receive the best supportive care.

Additional testing including blood, nasopharyngeal and oropharyngeal (optional) swabs, and sputum (optional) may be collected for research purposes. In some cases, customary standard of care procedures may be conducted more frequently for the purpose of this clinical study.

Information will need to be gathered and entered in the EDC associated with the medical management of the subject.

### Follow-Up Period (Day 29 to 6 Months):

The follow-up period is defined from Study Day 29 to 6 months. The Subjects will be followed at 3 months, and 6 months or until loss to follow-up, death, or withdrawal from study whichever occurs first.

The study will be conducted in compliance with ICH Good Clinical Practices (GCPs) and in concordance with local Health Authority regulations.

The End of Trial is defined as either the date of the last visit of the last subject to complete the post treatment follow-up, or the date of receipt of the last data point from the last subject that is required for primary, secondary and/or exploratory analysis, as specified in the protocol, whichever is the later date.

Dose Limiting Toxicity (DLT) Definition

Known pathologies associated with COVID-19 will be carefully considered and differentiated from potential CYNK-001-related effects in order to identify CYNK-001 related toxicities. Adverse events occurring up to Study Day 28 where the first dose of CYNK-001 infusion occurs on Study Day 1 will be included in the dose-limiting toxicity (DLT) determination.

A DLT is defined as the development of any new (not pre-existing) events:

Grade 4 or 5 event in any organ system
Grade 4>24 hours (Due to known organ damage associated with the COVID-19) in the following organ systems: Cardiac, Pulmonary, Hepatic, Renal, Central Nervous System (CNS)
Grade 3 or above allergic reaction that is suspected to be related to CYNK-001.
Grade 3 or above Graft versus Host Disease (GvHD) event occurring within the first 28 days following CYNK-001 infusion (to Study Day 28).
Grade 3 or above Cytokine Release Syndrome (CRS) event occurring within the 28 days following the first CYNK-001 infusion (to Study Day 28).
All above events to be identified in discussion with the clinical study Medical Monitor and reportable to Drug Safety team.

The events will be assessed for the first 3 subjects in Phase I and per the study stopping rule definition for the remaining subjects. Any such findings will be forwarded to the DMC for recommendation, review and confirmation as to whether or not the maximal tolerated dose (MTD) has been exceeded. If the MTD is confirmed by the DMC, no further CYNK-001 administration will occur within that dose level or at any higher dose level.

During Phase II portion of the study, DMC will be convened at midpoint (after 18 subjects have received CYNK-001 treatment) to evaluate safety for adverse event of interest such as shock, ARDS, and death in the treatment group versus the control group.

MTD is defined as the highest CYNK-001 dose level wherein it was deemed safe per the defined stopping rules or if the DMC recommends stopping the study due to DLTs suspected to be related to CYNK-001.

Number of subjects (planned): This study will enroll up to 86 subjects in total, with 14 subjects in the Phase I portion and up to 72 subjects with a 1:1 randomization ratio to either CYNK-001 or the best supportive care control arm as defined by the institutional practice in the Phase II portion of the study.

Investigational product, dosage and mode of administration: CYNK-001 is manufactured in a cryopreserved formulation that is thawed and diluted at the clinical site prior to dose preparation and direct infusion. CYNK-001 is packaged at 30.0+/−9.0×106 cells/mL in a total volume of 20 mL cryopreservation solution containing 10% (w/v) human serum albumin (HSA), 5.5% (w/v) Dextran 40, 0.21% sodium chloride (NaCl) (w/v), 32% (v/v) Plasma Lyte A, and 5% (v/v) dimethyl sulfoxide (DMSO). It is filled into the container closure, frozen using a controlled rate freezer, and the cryopreserved product is stored in vapor phase of liquid nitrogen (LN2). Prior to releasing to the site, all release and characterization testing will be complete. When required for administration by a site, CYNK-001 is shipped in vapor phase LN2 to the designated clinical site where it will be processed for dose preparation in a standardized manner just prior to intravenous (IV) administration.

CYNK-001 dosage and mode of administration: Dose Level 1: CYNK-001 with an initial Dose of 150×106 cells on Day 1 followed by 600×106 cells on Days 4 and 7 (second and third doses). After the first dose, subsequent infusions on Days 4 and 7 will be provided only if no toxicity of Grade 3 and above (either related or unrelated to CYNK-001) is observed for each subject. If any such [symbol] Grade 3 toxicity is observed, the second and third doses will be delayed up to 48 hours until the noted event is resolved or reduced to Grade 1 toxicity level.

Dose de-escalation Level −1: Dose de-escalation is defined as reducing the frequency of the doses (without reducing the total number of cells given per dose) by providing doses only on Days 1 and 7 for any potential safety concerns per DMC recommendation. CYNK-001 with an initial dose of 150×106 cells on Day 1 followed by 600×106 cells on Day 7.

CYNK-001 is to be administered IV using a gravity IV administration set with a 16- to 22-gauge (or equivalent) needle or catheter with no filters. A central line may be used to infuse CYNK-001 after confirming that the catheter diameter is 16- to 22-gauge (or equivalent) needle. For substantial deviation from this catheter diameter consultation with the medical monitor is required. The recommended infusion rate is approximately 240 mL per hour.

Subjects will receive pre- and post-medication of acetaminophen (650 mg PO) and diphenhydramine (25 mg PO/IV) at least 30 minutes prior to and approximately 4 hours after each CYNK-001 infusion.

Vital signs will be taken prior to, approximately 30 minutes after the start, and approximately 4 hours after the completion of each infusion. Subjects must be monitored for at least 4 hours after completion of each CYNK-001 infusion.

Dose Modifications: Dose adjustments may occur if clinically indicated by the treating physician. In general, the following should be followed:

Dose reductions are not permitted in this study. If DLTs safety concerns are observed, the dose de-escalation treatment with reducing frequency of doses will be implemented per DMC recommendation.

Should dose delays for CYNK-001 be required: Day 1 will be the date of initial dose. Day 4 dose may be delayed up to 48 hours.

For non-safety reasons: If delayed longer than 48 hours, Day 4 dose will be skipped, and the subject will receive the Day 7 dose. If the Day 4 dose is given within 48 hours, the Day 7 dose will be delayed for three days from the actual day of when Day 4 dose was given (i.e., if Day 4 dose is given on Day 5, then Day 7 dose will be given on Day 8)

For safety reasons: Day 4 dose could be delayed if [symbol] Grade 3 toxicity is observed after the first dose. In such cases, Day 4 dose is provided only if the event is resolved or reduced to Grade 1 toxicity level. If the toxicity did not resolve within 48 hours, the Day 4 dose will be skipped. If the Day 4 dose is given within 48 hours, the Day 7 dose will be delayed for three days from the actual day of when Day 4 dose given

If the subject has worsening of illness, study medication will be stopped.

Day 7 dose may be delayed up to 48 hours:

For non-safety reasons: If delayed longer than 48 hours, the subject will not receive additional therapy.

For safety reasons: If Day 4 dose was delayed but given within 48 hours of planned Day 4, then the Day 7 dose will be delayed for three days from the actual day of when Day 4 dose was given. If Day 4 dose was given as planned, Day 7 dose could be delayed if [symbol] Grade 3 toxicity is observed after the second dose. If Day 4 dose was skipped, Day 7 dose could be delayed if [symbol] Grade 3 toxicity is still observed after the first dose. In such cases, Day 7 dose is provided only if the event is resolved or reduced to Grade 1 toxicity level. If the toxicity did not resolve within 48 hours of the scheduled day, the Day 7 dose will not be administered.

If the subject has worsening of illness, study medication will be stopped.

All subjects who receive any amount of CYNK-001 will be followed to 6 months or until loss to follow-up, death, or withdrawal from study, whichever occurs first.

Statistical methods: Statistical Overview: The objectives of the Phase I portion are to evaluate the safety and efficacy (for lack of efficacy) of CYNK-001.

The overall clinical benefit in the Phase II portion of the study will be evaluated by comparing therapeutic effect of CYNK-001 versus the control group (best supportive care alone). Safety and tolerability by adverse events, labs, vital signs, etc. will also be evaluated.

Efficacy Analysis: Phase I efficacy data will be summarized by descriptive analyses. The efficacy endpoint used in the lack of efficacy tests is the responses at Day 15 of the Ordinal Scale for Clinical Improvement (OSCI). The study may be terminated if less than 2 subjects have efficacy responses in Phase I.

Phase II efficacy data will be analyzed based on Intention to Treat (ITT) population which include all randomized subjects. The primary endpoints of the study is time to clinical improvement by OSCI. The secondary endpoints include clinical status by OSCI, time to and rate of clinical improvement by NEWS2 Score, medical discharge, hospital utilization, and all-cause mortality rate, time to and rate of clearance of SARS-CoV-2, time to and rate of pulmonary clearance, duration of hospitalization, supplemental oxygen-free days, proportion of subjects requiring ventilation, SOFA score, and radiologic evaluation score.

For time to event data, Kaplan-Meier estimates for medians and 2-sided 95% CIs will be calculated. Stratified log-rank test will be used to test the difference between treatment groups. Stratification is based on randomization factor (age). For event rate data, the point estimates and the 2-sided 95% CIs will be calculated. Fisher's exact test will be used to test the difference between groups. For ordinal data, the outcome will be analyzed by using Mann-Whitney-Wilcoxon-Test.

Safety Analysis: Safety analysis will be based on the safety population which includes all subjects who are treated by any amount of CYNK-001 or who enroll into the control group. Descriptive statistics will be provided for AEs, vital sign measurements, physical examination findings, clinical laboratory test results, infusion site assessments, and concomitant medications and procedures.

Sample Size: Phase T Fourteen (14) subjects will be treated by CYNK-001 in the Phase I portion. Based on clinical judgement, this sample size is appropriate to evaluate the safety of CYNK-001 and to evaluate the lack of efficacy. This number is not based on power calculation.

Phase II: The primary efficacy endpoint is time to clinical improvement by OSCI.

As a preliminary proof of concept study without relevant data for the new coronavirus disease, 1-sided α of 0.05 will be used in the sample size consideration. With a sample size of 36 for each group (72 in total with 1:1 randomization ratio), a reduction of 50% for the time to event efficacy of CYNK-001 comparing with control can be detected with a power of at least 81% (assuming the time to clinical improvement is 8 days or earlier for CYNK-001 group, and 16 days or earlier for the control group with the same reduction of 50%) by using Log-rank test. This estimation is based on the study design with a maximum follow up of 28 days for each subject for primary analysis.

Among several coronaviruses that are pathogenic to humans, most cause mild clinical symptoms mainly represented as a respiratory tract infection, with two exceptions: the severe acute respiratory syndrome (SARS-CoV) and the Middle East respiratory syndrome (MERS-CoV) (Yin, 2018). A novel coronavirus emerged in the end of 2019 in Wuhan, China causing respiratory illness in people and has demonstrated rapid and effective person-to-person transmission, even from asymptomatic patients. The virus, initially called nCoV-2019, was identified by the Chinese Center for Disease Control and Prevention from a throat swab from a patient, and subsequently named SARS-CoV-2, that causes a coronavirus disease (COVID-19) (Chen, 2020).

Infection with SARS Coronavirus 2 (SARS-CoV-2) can lead to heterogeneous clinical manifestations, from asymptomatic infection to multi-organ system failure and need for intensive care support (Huang, 2020). Strategies to inhibit viral replication and reduce inflammation incited by SARS-CoV-2 (Xu 2020, Horby, 2020, Gritti, 2020, Guaraldi, 2020) are successful in selected cases. People with COVID-19 can seek medical care to help relieve symptoms, however even with appropriate early medical intervention, the illness can escalate to pneumonia, ARDS and in some cases requires intensive care. At the time of writing this protocol amendment, although most cases have presented in China, COVID-19 has been identified in over 217 countries and territories globally with more than 54 million cases and 1,280,860 fatalities (WHO, November 2020). In the US, more than 10 million confirmed cases have been identified in November 2020. The spread from carrier who may not show symptoms might be possible but people are more contagious when they are most symptomatic (CPC, 2020). A paper published on 21 Feb. 2020 concluded that a familial cluster of 5 patients with COVID-19 pneumonia had contact before their symptom onset with an asymptomatic family member who had traveled from epidemic center of Wuhan with presumption that asymptomatic carrier transmission of COVID-19 (Bai 2020).

Three recent papers have provided some insight into the initial presenting symptoms and the progression of the illness to pneumonia, in some cases escalating to severe pulmonary and other organ distress, which could be fatal (Huang, 2020; Chen, 2020; Wang. 2020). Chen et al presented 99 cases of patients infected with SARS-CoV-2, noting that the mean age was 55 and 68% were male. Of these cases 49% had been exposed to the Huanan Seafood Market, which is considered to be the source for the infection. Common symptoms (above 20% incidence) included shortness of breath, cough and fever, with 15% of patients exhibiting all three of these symptoms. However, it was noted that 90% of patients presented with more than one sign or symptom. ARDS was noted in 17% of these patients and with acute respiratory injury at 8%. Acute renal injury, septic shock or ventilator-associated pneumonia was rare (under 5%). Radiological findings noted unilateral pneumonia in 25% and bilateral pneumonia in 75% of the patients. Oxygen therapy was administered to 76% of these patients, and antibiotic, antifungal and antiviral therapy was used frequently. Intravenous immunoglobulin therapy was administered to 27% and glucocorticoids to 19%. At the time of data cut-off of this paper the mortality rate of the 99 patients infected by SARS-CoV-2 was 11%, with 58% remaining in hospital and 31% being discharged. Of these 99 patients, 23/99 (23%) were admitted to ICU, oxygen therapy was administered to 76%, invasive mechanical intervention 4% (range 3-20 days), non-invasive mechanical intervention 13% (range 4-22 days), continuous renal replacement therapy 9%, extracorporeal membrane oxygenation 3%. Treatment with antibiotics, antifungal, and antiviral therapy received in 71%, 15% and 76% respectively. Glucocorticoids were administered in 19% and intravenous immunoglobulin therapy in 27% (Chen, 2020).

Wang et al presented 138 cases of patients with confirmed SARS-CoV-2-infected pneumonia, noting the median age was 56 years and 75% were men. Hospital-associated transmission was suspected as the presumed mechanism of infection for affected health care professionals (29%) and hospitalized patients (12.3%). Common symptoms included fever (98.6%), dry cough (59.4%), lymphopenia (70.3%), elevated lactate dehydrogenase (39.9%). Chest computed tomographic scans showed bilateral patchy shadows or ground glass opacity in the lungs of all patients. Most patients received antiviral therapy (89.9%) and many received antibacterial therapy (64.4%), and glucocorticoid therapy (44.9%). Thirty-six (26.1%) patients were transferred to ICU. The median time for first symptom to dyspnea was 5 days, to hospital admission was 7 days, and to ARDS was 8 days. Compared with patients not treated in the ICU (73.9%), patients treated in the ICU were older and more likely to have underlying comorbidities. Of the 36 patients in the ICU, 4 (11%) received high-flow oxygen therapy, 15 (41.7%) received noninvasive ventilation, and 17 (47.2%) received invasive ventilation (with 4 of them being switched to Extracorporeal membrane oxygenation). At the time of data cutoff of this paper, the article reports that 47 patients (34.1%) were discharged and 6 died (overall mortality, 4.3%), but the remaining patients are still hospitalized. Among those discharged alive (34.1%), the median hospital stay was 10 days (Wang, 2020).

The COVID-19 epidemic continues and is unlikely to wane anytime soon; clinical management and mortality rate warrants need for new therapeutic approaches to medically support the patients and prevent mortality.

The World Health Organization (WHO) has released an interim guidance dated 28 Jan. 2020 on the clinical management of severe acute respiratory infection when SARS-CoV-2 infection is suspected (WHO, 2020), The Chinese government had released the diagnosis and treatment plan (provisional 6th edition) dated 19 Feb. 2020 for Novel coronavirus pneumonia (https://www.chinalawtranslate.com/en/diagnostic-and-treatment-plan-6).

Real-time Reverse Transcriptase Polymerase-Chain-Reaction (rRT-PCR) assays for in vitro qualitative detection of SARS-CoV-2 in respiratory specimens and sera have been developed for identification of the COVID-19 infection. The CDC developed a new laboratory test kit for use in testing patient specimens called “Centers for Disease Control and Prevention (CDC) COVID-19 Real-Time Reverse Transcriptase (RT)-PCR Diagnostic Panel.” The kits were shipped to qualified international laboratories internationally, however the US FDA issued the Emergency Use Authorization of this test on 4 Feb. 2020 for use in the US.

The CDC recommends collecting and testing upper respiratory specimens (i.e., nasopharyngeal and oropharyngeal swabs), and lower respiratory tract (LRT) specimens (i.e., sputum). Sera testing is also an option as well as bronchoalveolar lavage (CDC, 2020). Note: as new testing has been developed to detect SARS-CoV-2, the use of alternate SARS-CoV-2 testing by other approved methods is permitted where institutional practice allows.

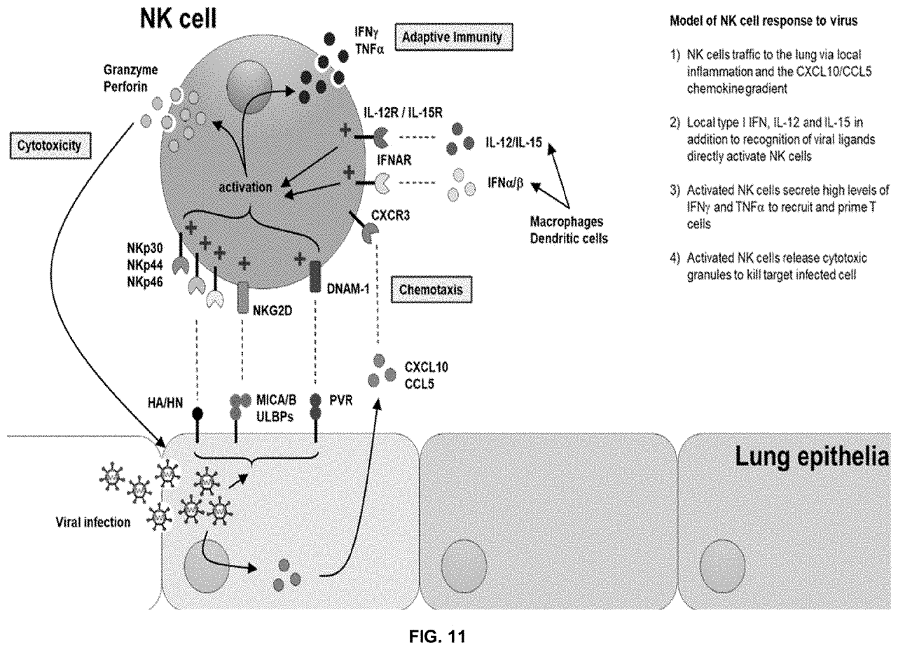

NK cells are innate immune cells with an important role in early host response against various pathogens. Multiple NK cell receptors are involved in the recognition of infected cells, including NKG2D, DNAM-1 and the natural cytotoxicity receptors NKp30, NKp44 and NKp46, which bind common stress ligands or pathogen-associated molecules (see FIG. 11) (Cook, 2014). NK cells kill their target cells by cytotoxic molecules perforin and granzymes, and via death receptor-mediated apoptosis (Loh, 2005). In addition to their cytotoxic functions, NK cells are important for priming adaptive immunity by the secretion of various chemokines and cytokines, including IFN-g. The important role of NK cells in virus control is illustrated by the diverse mechanisms human viruses have evolved to evade the NK cell recognition pathways, especially exemplified by CMV (Lanier, 2008).

Studies in humans and mice have established that there is robust activation of NK cells during viral infection, regardless of the virus class (Ivanova, 2014), and that the depletion of NK cells aggravates viral pathogenesis (Littwitz, 2013; Gazit 2006; Nogusa, 2008; Stein-Streilein, 1986. In murine and human CMV infection, NK cell-mediated anti-viral activity is dependent on IFN-g secretion and perforin-dependent lysis of infected cells (Loh, 2005; Wu, 2015). HIV-1 infection in pregnancy is inhibited by decidual NK cells (Quillay, 2016) and hepatitis C virus infection is controlled by NK cells in the liver (Guidotti, 2006). NK cells have a major role in the early control of lung infections with pathogenic organisms. Timely NK cell-mediated cytotoxicity and IFN-g production limit diverse respiratory bacterial, fungal and viral infections (Ivanova, 2014).

NK cells sense the environment using a broad repertoire of surface receptors that can differentiate between normal and malignant cells (cancerous or infected) by binding to stress ligands and viral antigens. In particular, the stress ligand-induced NKG2D-MICA/B pathway has been shown to be important for NK cell activation and recognition of infected cells in multiple viral infections, including coronaviruses (Walsh, 2008; Lanier, 2008). Various viral glycoproteins expressed by enveloped viruses, including coronaviruses (Zeng, 2008), are specifically recognized by the natural cytotoxicity receptors NKp30, NKp44, and NKp46 (Cook, 2014). NK cell cytolytic activity against Influenza virus is triggered by the recognition of viral haemagglutinin by NKp46 receptor, but also induced by antibody-dependent cell-mediated cytotoxicity (ADCC) (Mandelboim, 2001). In infected tissue microenvironment, NK cell activation leads to increase activating receptor expression and their cytotoxic responses are strongly potentiated by type I IFNs produced by dendritic cells and infected epithelial cells, also enabling subsequent priming and T cell activation and memory (Lanier, 2008).

It was shown that coronavirus infection stimulates the recruitment of NK cells to control infection. Research following the SARS-CoV outbreak revealed that SARS-CoV infection in a mouse model resulted in acute expression of CCL5, CXCL10, and CCL3 chemokines in lung epithelial cells (Law, 2007). In a separate study, NK cells migrated to coronavirus-infected organs in a CXCL10 dependent manner and was associated with reduced coronavirus titers. Anti-viral activity accompanied NK cell homing to the tissue and IFN-g secretion (Trifilo, 2004).

A study of NK cells from peripheral blood of patients with SARS coronavirus (SARS-CoV) was evaluated for the number of NK cells, as it was previously noted that patients with lower NK cells in the HIV population were susceptible to retrovirus resistance. It was noted that patients with SARS coronavirus had significantly lower counts of NK cells in their peripheral blood compared to patients with mycoplasma pneumonia and healthy adults. It was unclear as to why the number was lower. It was hypothesized that either the NK cells had died as a direct attack from the virus or the NK cells were redistributed to targeted organs, such as the lungs (National Research Project of SARS, 2004). Hematological abnormalities such as thrombocytopenia and lymphopenia were common in both SARS-CoV and MERS-CoV patients. Thrombocytopenia and lymphopenia may be predictive of fatal outcome in MERS-CoV patients (Yin, 2018). Based on these observations, it is hypothesized that adoptive NK cell therapy may provide the antiviral activities in those with SARS-CoV-2 infection.

CYNK-001 is the only cryopreserved allogeneic, off-the-shelf NK cell therapy being developed from placental hematopoietic stem cells as a potential treatment option for various hematologic cancers and solid tumors. NK cells are a unique class of immune cells, innately capable of targeting cancer cells and virus infecting cells and interacting with adaptive immunity. CYNK-001 cells derived from the placenta are intrinsically safe and versatile, currently being investigated as a treatment for acute myeloid leukemia (AML), multiple myeloma (MM), and glioblastoma multiforme (GBM).

CYNK-001 are human placental hematopoietic stem cell-derived NK cells that express the dominant NK cells marker CD56 and lack lineage markers such as CD3, CD14 and CD19. These cells demonstrate a range of biological activities expected of NK cells, including expression of perforin and granzyme B, cytolytic activity against hematological tumor cell lines, and secretion of immunomodulatory cytokines such as IFN-γ in the presence of tumor cells. CYNK-001 cells express NKG2D and CD94, as well as NK activating receptors DNAM1, NKp30, NKp46, and NKp44 that have been shown to be key in the recognition of virus-infected cells (Walsh, 2008; Lanier, 2008; Zeng, 2008; Cook, 2014). Whereas data on the pathogenic coronaviruses is limited, copious amount of studies on the general mechanisms NK cells use to recognize infected cells in the context of diverse viral pathogens, allows us to predict that SARS-CoV-2 infected cells would express stress antigen molecules as in other viral infections, rendering them sensitive to CYNK-001 recognition and subsequent elimination. Regarding homing to infected tissues, it has been shown that CYNK-001 cells have immediate biodistribution in the lungs following intravenous injection in preclinical models (IND 016792, CELU-2018-003; CELU-2019-001). CYNK-001 cells also express CXCR3, the receptor involved in homing to CXCL10 following coronavirus infection. Consequently, CYNK cells have the potential to be efficacious and retained in the lungs given the heightened local biodistribution and chemoattraction to CXCL10.

This study is the first study that will evaluate the safety and potential efficacy of CYNK-001 in subjects with SARS-CoV2. The study will be comprised of Screening Period, Treatment Period, and Follow-up Period. The Treatment Period will include CYNK-001 cells along with clinical care. For the Phase II control arm, treatment period will include Best Supportive Care.

HLA matching and KIR mismatching will not be used in the selection of CYNK-001 for an individual subject. However, these data will be collected for retrospective analysis.

PNK-007 was distributed as fresh formulated cells just in time to subjects for their treatment. This just in time formulation required transition for further development of this product which resulted in a cryopreserved product for shipment to sites. The results of testing based on identity, purity, viability, fold expansion during manufacturing and performance of the Drug Products using a qualified cytotoxicity assay demonstrated comparability between PNK-007 and CYNK-001.

PNK-007, is an allogeneic, off the shelf cell therapy enriched for CD56+/CD3− NK cells expanded from placental CD34+ cells has previously been used in the treatment of acute myeloid leukemia (PNK-007-AML-001) and multiple myeloma (PNK-007-MM-001). PNK-007 is dosed based on subject weight (e.g., 10⁶ cells/kg) so the volume of the infusion scales with the subject weight (approximately 2 mL/kg). Each unit of PNK-007 was custom filled based on the subject weight, so that a full unit delivers the appropriate cell dose.

A total of 10 subjects were treated with a single infusion of PNK-007 (range 1×10⁶ cells/kg to 10×10⁶ cells/kg) followed by 5 or 6 total rhIL-2 injections every other day starting on day of PNK-007 infusion to facilitate PNK-007 expansion. A conditioning treatment of cyclophosphamide for 2 days and fludarabine for 5 days with both ending 2 days prior to the PNK-007 infusion was given to favor NK cell expansion. Cell therapy regimens have historically included systemic lymphodepletion to improve cell expansion, persistence, and efficacy for CAR-T in leukemias (Brentjens, 2011) as well as for haploidentical NK cells in AML (Miller, 2005). In the PNK-007-AML-001 study, four subjects were treated in the highest dose administered, 10×10⁶ cells/kg PNK-007, with an actual dose infused ranging from 5.86×10⁸ to 8.49×10⁸ total cells associated with subject weight ranges from 59.3 kg to 83.1 kg. One dose limiting toxicity of CRS was experienced on Day 14 at the 10×10⁶ cells/kg dose which was managed with appropriate treatment regimen. This subject's weight was 77.2 kg resulting in a dose of ˜7.7×10⁸ cells.

For the 15 subjects who received treatment on the PNK-007-MM-001 study, 9 subjects were allocated to receive 10×10⁶ cells/kg dose, the actual dose infused of PNK-007 ranged from 6.47×10⁸ cells to 1.08×10⁹ cells with subject weight ranges from 66.7 kg to 111.6 kg. For the 6 subjects who were allocated to receive 30×10⁶ cells/kg dose, the actual dose infused of PNK-007 ranged from 1.51×10⁹ cells to 2.92×10⁹ cells with weight ranges from 51.5 kg to 99.8 kg. All 15 subjects received a single infusion of PNK-007, with 12/15 subjects also receiving rhIL-2 to facilitate expansion. No dose limiting toxicities were experienced.

Of the 25 subjects treated with PNK-007, HLA matching and KIR mismatching was not used in the selection of product for an individual subject. From the retrospective data collected and samples analyzed, there was no allo-reactivity identified based on the absence of anti-HLA alloantibodies at all measured timepoints. The majority of the subjects treated in these two studies were also receiving prophylactic antibiotics, antivirals and/or antifungals as part of a prophylactic treatment plan associated with their clinical care. To date there have been no identified drug interactions with these products.

CYNK-001 was well tolerated by NOD SCID Gamma (NSG) mice after three weekly repeat IV administration at the dose of 10×10⁶ cells/animal (400×10⁶ cells/kg). Histopathology results showed that CYNK-001 was not associated with any treatment-related abnormal pathological effect. CYNK-001 cells were detected in the lung, liver, spleen, kidney, and bone marrow, and persisted up to 7 days.

The dose selection for this study population was selected at 600×10⁶ cells per dose, noting that this would be anticipated to be approximately 10×10⁶ cells/kg based on average weight of the Chinese population. It was reported by Huang et al the median time from onset of symptoms to acute respiratory distress syndrome was 9.0 days (8.0-14.0) in 41 Wuhan COVID-19 patients, therefore we propose to have three repeat doses given within 7 days (Huang. 2020). It is also noted that the subjects in this study will not be receiving rhIL-2 to facilitate expansion. Data from the Huang et al indicated elevated basal levels of systemic IL-2 and IL-15 in COVID-19 patients which would support CYNK-001 expansion.

CYNK-001 is the only cryopreserved allogeneic, off-the-shelf NK cell therapy being developed from placental hematopoietic stem cells as a potential treatment option for various hematologic cancers and solid tumors. NK cells are a unique class of immune cells, innately capable of targeting cancer cells and virus infecting cells and interacting with adaptive immunity. CYNK-001 cells derived from the placenta are intrinsically safe and versatile, currently being investigated as a treatment for acute myeloid leukemia (AML), multiple myeloma (MM), and glioblastoma multiforme (GBM).

CYNK-001 are human placental hematopoietic stem cell-derived NK cells that express the dominant NK cells marker CD56 and lack lineage markers such as CD3, CD14 and CD19. These cells demonstrate a range of biological activities expected of NK cells, including expression of perforin and granzyme B, cytolytic activity against hematological tumor cell lines, and secretion of immunomodulatory cytokines such as IFN-γ in the presence of tumor cells. CYNK-001 cells express NKG2D and CD94, as well as NK activating receptors DNAM1, NKp30, NKp46, and NKp44 that have been shown to be key in the recognition of virus-infected cells (Walsh, 2008; Lanier, 2008; Zeng, 2008; Cook, 2014). Whereas data on the pathogenic coronaviruses is limited, copious amount of studies on the general mechanisms NK cells use to recognize infected cells in the context of diverse viral pathogens, allows us to predict that SARS-CoV-2 infected cells would express stress antigen molecules as in other viral infections, rendering them sensitive to CYNK-001 recognition and subsequent elimination. Regarding homing to infected tissues, it has been shown that CYNK-001 cells have immediate biodistribution in the lungs following intravenous injection in preclinical models (IND 016792, CELU-2018-003; CELU-2019-001). CYNK-001 cells also express CXCR3, the receptor involved in homing to CXCL10 following coronavirus infection. Consequently, CYNK cells have the potential to be efficacious and retained in the lungs given the heightened local biodistribution and chemoattraction to CXCL10.

This study is the first study that will evaluate the safety and potential efficacy of CYNK-001 in subjects with SARS-CoV2. The study will be comprised of Screening Period, Treatment Period, and Follow-up Period. The Treatment Period will include CYNK-001 cells along with clinical care. For the Phase II control arm, treatment period will include Best Supportive Care.

HLA matching and KIR mismatching will not be used in the selection of CYNK-001 for an individual subject. However, these data will be collected for retrospective analysis.

PNK-007 was distributed as fresh formulated cells just in time to subjects for their treatment. This just in time formulation required transition for further development of this product which resulted in a cryopreserved product for shipment to sites. The results of testing based on identity, purity, viability, fold expansion during manufacturing and performance of the Drug Products using a qualified cytotoxicity assay demonstrated comparability between PNK-007 and CYNK-001.

PNK-007, is an allogeneic, off the shelf cell therapy enriched for CD56+/CD3− NK cells expanded from placental CD34+ cells has previously been used in the treatment of acute myeloid leukemia (PNK-007-AML-001) and multiple myeloma (PNK-007-MM-001). PNK-007 is dosed based on subject weight (e.g., 10⁶ cells/kg) so the volume of the infusion scales with the subject weight (approximately 2 mL/kg). Each unit of PNK-007 was custom filled based on the subject weight, so that a full unit delivers the appropriate cell dose.

A total of 10 subjects were treated with a single infusion of PNK-007 (range 1×10⁶ cells/kg to 10×10⁶ cells/kg) followed by 5 or 6 total rhIL-2 injections every other day starting on day of PNK-007 infusion to facilitate PNK-007 expansion. A conditioning treatment of cyclophosphamide for 2 days and fludarabine for 5 days with both ending 2 days prior to the PNK-007 infusion was given to favor NK cell expansion. Cell therapy regimens have historically included systemic lymphodepletion to improve cell expansion, persistence, and efficacy for CAR-T in leukemias (Brentjens, 2011) as well as for haploidentical NK cells in AML (Miller, 2005). In the PNK-007-AML-001 study, four subjects were treated in the highest dose administered, 10×10⁶ cells/kg PNK-007, with an actual dose infused ranging from 5.86×10⁸ to 8.49×10⁸ total cells associated with subject weight ranges from 59.3 kg to 83.1 kg. One dose limiting toxicity of CRS was experienced on Day 14 at the 10×10⁶ cells/kg dose which was managed with appropriate treatment regimen. This subject's weight was 77.2 kg resulting in a dose of ˜7.7×10⁸ cells.

For the 15 subjects who received treatment on the PNK-007-MM-001 study, 9 subjects were allocated to receive 10×10⁶ cells/kg dose, the actual dose infused of PNK-007 ranged from 6.47×10⁸ cells to 1.08×10⁹ cells with subject weight ranges from 66.7 kg to 111.6 kg. For the 6 subjects who were allocated to receive 30×10⁶ cells/kg dose, the actual dose infused of PNK-007 ranged from 1.51×10⁹ cells to 2.92×10⁹ cells with weight ranges from 51.5 kg to 99.8 kg. All 15 subjects received a single infusion of PNK-007, with 12/15 subjects also receiving rhIL-2 to facilitate expansion. No dose limiting toxicities were experienced.

Of the 25 subjects treated with PNK-007, HLA matching and KIR mismatching was not used in the selection of product for an individual subject. From the retrospective data collected and samples analyzed, there was no allo-reactivity identified based on the absence of anti-HLA alloantibodies at all measured timepoints. The majority of the subjects treated in these two studies were also receiving prophylactic antibiotics, antivirals and/or antifungals as part of a prophylactic treatment plan associated with their clinical care. To date there have been no identified drug interactions with these products.

CYNK-001 was well tolerated by NOD SCID Gamma (NSG) mice after three weekly repeat IV administration at the dose of 10×10⁶ cells/animal (400×10⁶ cells/kg). Histopathology results showed that CYNK-001 was not associated with any treatment-related abnormal pathological effect. CYNK-001 cells were detected in the lung, liver, spleen, kidney, and bone marrow, and persisted up to 7 days.

The dose selection for this study population was selected at 600×10⁶ cells per dose, noting that this would be anticipated to be approximately 10×10⁶ cells/kg based on average weight of the Chinese population. It was reported by Huang et al the median time from onset of symptoms to acute respiratory distress syndrome was 9.0 days (8.0-14.0) in 41 Wuhan COVID-19 patients, therefore we propose to have three repeat doses given within 7 days (Huang, 2020). It is also noted that the subjects in this study will not be receiving rhIL-2 to facilitate expansion. Data from the Huang et al indicated elevated basal levels of systemic IL-2 and IL-15 in COVID-19 patients which would support CYNK-001 expansion.

### Trial Objectives and Purpose

### Primary Objective

Phase I: The primary objectives of the Phase I portion of the study is to evaluate the safety, tolerability, and efficacy of multiple CYNK-001 intravenous (IV) infusions administered at an initial dose of 150×10⁶ cells dose on Day 1 followed by 600×10⁶ cells doses on Days 4 and 7 in subjects with COVID-19.

Overall, if the safety stopping rules are not met and if efficacy is demonstrated in at least 2 out of the 14 subjects by Day 15 of CYNK-001 infusion (as defined by at least one “Patient State” category of improvement on the Ordinal Scale for Clinical Improvement (OSCI), the study will move forward to the Phase II portion of the study.

Phase II: The primary objective of the Phase II portion is to evaluate the efficacy of CYNK-001 on subjects with COVID-19 by using the OSCI defined by the World Health Organization (WHO).

### Secondary Objectives

The secondary objectives of the Phase II portion are: To determine safety and tolerability of CYNK-001 as measured by the frequency and severity of AEs using CTCAE, version 5.0; and To evaluate the overall clinical benefit of receiving CYNK-001 for COVID-19 as measured by rate of clinical improvement by OSCI, time to and rate of clinical improvement by NEWS2 Score, medical discharge, hospital utilization, and all-cause mortality rate, time to and rate of clearance of SARS-CoV-2, time to and rate of pulmonary clearance, duration of hospitalization, supplemental oxygen-free days, proportion of subjects requiring ventilation, SOFA score, and radiologic evaluation score.

### Exploratory Objectives

Exploratory objectives include detection of SARS-CoV-2 via rRT-PCR in various specimen types, cytokine and chemokine measurement, and immune monitoring, alloreactivity measurement.

Study Endpoint Descriptions

TABLE 5
Study Endpoints

| | | | |
|---|---|---|---|
| Endpoint | Name | Description | Timeframe |

Phase 1

| | | | |
|---|---|---|---|
| Phase 1 | Safety | Frequency and severity of adverse | DLTs assessed |
| Primary | | events, changes in vital signs, | from Study Day 1 |
| | | laboratory assessments, Performance | through Day 28 |
| | | Status assessment, and | |
| | | immunological and inflammation | |
| | | assessments. | |

| | | |
|---|---|---|
| Futility Check for go/no go | Efficacy as measured by clinical | Study Day 15 |
| decision to move to Phase 2: | improvement by OSCI. At least 2 | |
| | out of 14 subjects must achieve at | |
| | least 1 “Patient State” category | |
| | improvement in Ordinal Score | |
| | (OSCI) | |

Phase 2

| | | | |
|---|---|---|---|
| Phase 2 | Time to clinical | Time to clinical improvement | Study Day 28 |
| Primary | improvement by | measured by OSCI | |
| | OSCI | | |
| Phase 2 | Clinical status by | Ordinal scale by OSCI | Study Day 28 |
| Secondary | OSCI | | |
| | Rate of clinical | Proportion of subjects who achieved | Study Day 28 |
| | improvement by | clinical improvements by OSCI | |
| | OSCI | | |
| | Time to clinical | Time to clinical improvement | Study Day 28 |
| | improvement by | measured by NEWS2 Score | |
| | NEWS2 | | |
| | Rate of clinical | Proportion of subjects who achieved | Study Day 28 |
| | improvement by | clinical symptom improvement by | |
| | NEWS2 | NEWS2 Score | |
| | Rate of clearance | Proportion of subjects with clearance | Study Day 28 |
| | of SARS-CoV-2 | of SARS-CoV-2 from mucosal | |
| | | specimens (nasopharyngeal swab, | |
| | | oropharyngeal swab if available) | |
| Phase 2 | Time to clearance | Clearance of SARS-CoV-2 by | Study Day 28 |
| Secondary | of SARS-CoV-2 | rRT-PCR testing of mucosal samples | |
| Phase 2 | | (nasopharyngeal swab, | |
| Secondary | | oropharyngeal swab if available); | |
| | | negative results should be confirmed | |
| | | by same sample type at least 24 | |
| | | hours after the first negative result | |
| | Time to | Time to disappearance of virus from | Study Day 28 |
| | pulmonary | LRT specimen where it has | |
| | clearance | previously been found (induced | |
| | | sputum if available, endotracheal | |
| | | aspirate if available) | |
| | Rate to pulmonary | Proportion of subjects who had | Study Day 28 |
| | clearance | disappearance of virus from LRT | |
| | | specimens where it has previously | |
| | | been found. | |
| | Duration of | Duration of hospitalization from | Study Day 28 |
| | hospitalization | time from hospitalization to medical | |
| | | discharge | |
| | Supplemental | For subjects requiring supplemental | Study Day 28 |
| | oxygen-free days | oxygen, days with supplemental | |
| | | oxygen-free days | |
| | Proportion of | Proportion of subjects who need | Study Day 28 |
| | subjects requiring | invasive or non-invasive ventilation | |
| | ventilation | | |
| | SOFA Score | Sequential Organ Failure | Study Day 28 |
| | | Assessment (SOFA) score | |
| | Radiologic | Chest x-ray and/or CT scan results | Study Day 28 |
| | Evaluation Score | will be evaluated and scored | |
| | All-cause | Proportion of subjects who died | up to Study Day 28 |
| | Mortality rate | | and Month 6 |
| | Safety | Frequency and severity of adverse | Month 6 |
| | | events, changes in vital signs, | |
| | | laboratory assessments, Performance | |
| | | Status assessment, and | |
| | | immunological and inflammation | |
| | | assessments. | |

Exploratory

| | | | |
|---|---|---|---|
| Exploratory | Cytokine and | Cytokine and chemokine assessment | Month 6 |
| (Phase 1 | Chemokine | measured serially over the course of | |
| and 2) | Assessment | the study | |
| | Immune | As measured by chimerism and/or | Month 6 |
| | Monitoring | other differentiating methodologies | |
| | | of donor-derived natural killer cells | |
| | | for those treated with CYNK-001. | |
| | | Immune profiling and | |
| | | immunophenotyping including | |
| | | alloreactivity | |
| | Detection of | Detection of SARS-CoV-2 via | Month 6 |
| | SARS-CoV-2 in | rRT-PCR in various specimens | |
| | various specimen | including peripheral blood | |
| | types | | |

### Investigational Plan

### Overall Study Design

The proposed study will evaluate the safety and the clinical efficacy of CYNK-001 in SARS-CoV-2 positive subjects as measured by clearance of the SARS-CoV-2 and improvement in clinical symptoms as measured by OSCI and NEWS2 scores.

The study will include a Phase I portion wherein a total of 14 subjects will be enrolled to assess the safety and efficacy of CYNK-001. To evaluate the safety for potential DLTs, this phase will enroll 3 subjects initially treated with CYNK-001. The safety data for these 3 subjects will be evaluated 24 hours after the final dose was provided to the 3^{rd} subject. If deemed safe, the remaining 11 subjects will be enrolled and monitored per the safety stopping rule until Day 15 after the first CYNK-001 infusion. If any DLT is observed in the first three subjects, the DMC will be convened for recommendation. For the remaining 11 subjects, DMC will be convened if the safety stopping rule is met. Overall, in case of 2 out of the 6 subjects had experienced DLTs in the Phase I portion of this study, the DMC will be convened for safety evaluation.

The Phase II portion of the study is a randomized, open-label, multi-site study. Subjects will be randomized to either CYNK-001 group or Control group (best supportive care) with 1:1 ratio stratified by age (<45 vs. ≥45 years old). All subjects in both Phase I and Phase II will receive the best supportive care. Best supportive care will be inclusive of biologic immunomodulatory therapy aimed at reducing morbidity from cytokine release syndrome, such as IL-6 (tocilizumab or siltuximab) or GM-CSF inhibitors.

During Phase II portion of the study, DMC will be convened at midpoint (after 18 subjects have received treatment) to evaluate safety for adverse event of interest such as shock, ARDS, and death in the treatment group versus the control group.

The study is divided into 3 study periods: Screening Period, Treatment Period, and Follow-up Period, each with associated evaluations and procedures that must be performed at specific timepoints.

Subject participation is dependent on slot availability based on time of entry into the study.

### Screening Period

The Screening Period is defined as the period from signing the informed consent to just prior to the administration of CYNK-001. Due to the critical nature of this infection, this period may be less than a day. Upon giving written informed consent, all screening/baseline assessments will be completed. Some procedures that occur as part of standard of care in medical evaluation may be completed prior to the date of informed consent, according to institutional practices, and therefore do not need to be repeated. Chest x-rays and blood work should occur often as outlined in the Table of Events.

During the Screening period, after having signed an ICF, subjects will be assessed for eligibility for the study. Eligibility must be confirmed prior to proceeding to the treatment period. This information will need to be gathered and entered into the EDC. Subject eligibility will be based on investigator assessment using the Inclusion/Exclusion criteria provided as part of the study. The Screening Period is followed by a Treatment Period.

### Treatment Period (Day 1 to Day 28)

The treatment period begins with the administration of study drug on Study Day 1. For those subjects who are allocated treatment to CYNK-001, the initial dose will consist of 150×10⁶ cells followed by the second and third doses each of 600×10⁶ cells administered intravenously (IV). Subjects will receive a minimum of two and up to three CYNK-001 infusions. CYNK-001 infusions will occur on Study Days 1, 4, and 7. After the first dose, subsequent infusions on Days 4 and 7 will be provided only if no toxicity of Grade 3 and above (either related or unrelated to CYNK-001) is observed for each subject. If any such ≥Grade 3 toxicity is observed, the second and third doses will be delayed up to 48 hours until the noted event is resolved or reduced to Grade 1 toxicity level.

Subjects treated in Phase I will receive all planned CYNK-001 infusions in the inpatient setting and may be discharged one day following the final planned CYNK-001 infusion (i.e., discharged on Study Day 8, where infusions occur on Days 1, 4, and 7). If any dose is delayed, subjects will remain inpatient until one day following the final CYNK-001 infusion.

As part of discharge criteria, the study team should assess for further monitoring of subjects who may be experiencing toxicity of Grade 3 and above (either related or unrelated to CYNK-001) at the time of discharge and consult with the treating physician. The decision to discharge a subject should be in consultation with the treating physician. Physicians should follow best clinical practice to determine appropriate timing of hospital discharge.

Upon discharge, plans should be made for appropriately delegated staff to have telephone contact with subjects every day between hospital discharge through at least Day 15 visit for safety/AE monitoring.

Subjects will be provided with a thermometer, pulse oximeter, and blood pressure monitor for at home collection of temperature, oxygen saturation, and blood pressure with written instructions on their use as well as expectations for self-monitoring. These daily measurements will be reported via telephone to the clinical staff during each daily telehealth visit. During these daily telehealth visits, subjects are to report any new symptoms or worsening of symptoms associated with previously identified adverse events.

The subject will be asked during each call to report any new or worsening symptoms that could be consistent with adverse events since the previous visit or telephone call. The investigator (or appropriately delegated study staff) will determine if medical attention or an unscheduled clinic visit is required. Each telephone call should be carefully documented with date and time of the call in the source documents and reported as appropriate.

Consultation between the Medical Monitor and appropriately delegated site staff should occur every other day after hospital discharge up to Day 15 visit for ongoing review of the subject's clinical status. This communication may occur via telephone contact or written message (i.e., email) and should be documented accordingly.

Subjects treated in the Phase II portion of the study will be treated either in the inpatient or outpatient setting, as determined after review of data from the Phase I portion of the study and based on DMC recommendation. The level of outpatient monitoring of subjects treated in the Phase II portion will be determined based on review of Phase I data and DMC recommendation.

Consultation with the Medical Monitor is required prior to each CYNK-001 infusion if there is:
An increase in supplemental oxygen of greater than or equal to 50% from baseline (for first CYNK-001 infusion) or from level at prior CYNK-001 infusion (for second and third CYNK-001 infusion) resulting in oxygen use of greater than 8 L.—OR—
A change in the mode of supplemental oxygen delivery with the intention to deliver oxygen more efficiently.
The Medical Monitor will escalate to the Sponsor clinical and safety teams as appropriate per GCP guidelines to advise if a subject who is experiencing rapid worsening of disease should proceed to first or subsequent CYNK-001 infusion.

A de-escalation Dose level −1 will be initiated based on the study stopping rules and dose-limiting toxicities. Dose de-escalation is defined as reducing the frequency of the doses (without reducing the total number of cells given per dose) by providing doses only on Days 1 and 7 due to potential safety concerns per the DMC recommendation.

On each day of CYNK-001 infusion, subjects will be pre-medicated and post-medicated with acetaminophen 650 mg orally (per oral, PO) and diphenhydramine 25 mg (PO/IV) at least 30 minutes prior to and approximately 4 hours following the end of the CYNK-001 infusion. Meperidine may also be administered to control rigors, if clinically indicated. Subjects must be monitored for at least 4 hours after completion of each CYNK-001 infusion.

If subject has been found to be SARS-CoV-2 negative by rRT-PCR or the third dose is skipped per protocol allowances, and has received two doses of CYNK-001, the third dose is not mandatory. If the subject does not receive the third dose, the subject may be discharged one day following the final planned dose at the discretion of the treating physician based on clinical status.

All subjects (even in control arm) should meet the inclusion/exclusion criteria.

The control arm subjects in the Phase II portion of the study will receive the best supportive care as defined by the institutional practice without CYNK-001. All subjects in both Phase I and Phase II will receive the best supportive care.

Additional testing including blood, nasopharyngeal and oropharyngeal (optional) swabs, and sputum (optional) may be collected for research purposes. In some cases, customary standard of care procedures may be conducted more frequently for the purposes of this clinical study.

Information will need to be gathered and entered into the EDC associated with the medical management of the subject.

### Follow Up Period

The follow-up period is defined from Day 29 to Month 6. Subjects will be followed at 3 months, and 6 months, or until loss to follow-up, death, or withdrawal from study, whichever occurs first.

The study will be conducted in compliance with ICH Good Clinical Practices (GCPs) and in concordance with local Health Authority regulations.

The End of Trial is defined as either the date of the last visit of the last subject to complete the post treatment follow-up, or the date of receipt of the last data point from the last subject that is required for primary, secondary and/or exploratory analysis, as specified in the protocol, whichever is the later date.

### Hospital Discharge

Hospital discharge is defined as the day a hospitalized subject is discharged.

Hospital Discharge visit should be performed if a hospitalized subject is discharged on any day other than a scheduled study visit day.

Subjects who were treated in the Phase 1 portion of the study may be discharged on the day following the final planned CYNK-001 infusion (i.e., discharged on Study Day 8, where infusions occur on Days 1, 4, and 7 or discharged on Study Day 5 if only two doses were received on Days 1 and 4.)

As part of discharge criteria, the study team should assess for further monitoring of subjects who may be experiencing toxicity of Grade 3 and above (either related or unrelated to CYNK-001) at the time of discharge and consult with the treating physician and clinical site study team. The decision to discharge a subject should be in consultation with the treating physician. Physicians should follow best clinical practice to determine appropriate timing of hospital discharge.

Upon discharge, plans should be made for appropriately delegated staff to have telephone contact with subjects every day between hospital discharge through at least Day 15 visit for safety/AE monitoring. The discharge plan should include follow-up visit schedule as well as planned telehealth visit schedule.

Upon discharge, subjects will be provided with a thermometer, pulse oximeter, and blood pressure monitor for at home collection of temperature, oxygen saturation, and blood pressure with written instructions on their use as well as expectations for self-monitoring. These daily measurements will be reported via telephone to the clinical staff during each daily telehealth visit. During these daily telehealth visits, subjects are to report any new symptoms or worsening of symptoms associated with previously identified adverse events and will also provide the study team with daily vital sign measurements. Any vital signs outside of normal range will be escalated to the clinical site study team and evaluated for appropriate management.

The subject will be asked during each call to report any new or worsening symptoms that could be consistent with adverse events since the previous visit or telephone call. The investigator (or appropriately delegated study staff) will determine if medical attention or an unscheduled clinic visit is required. Each telephone call should be carefully documented with date and time of the call in the source documents and reported as appropriate.

Alternative person to contact in case of possible emergency or for planned daily calls from clinical site staff should also be collected prior to hospital discharge.

Consultation between the Medical Monitor and appropriately delegated site staff should occur every other day after hospital discharge up to Day 15 visit for ongoing review of the subject's clinical status. This communication may occur via telephone contact or written message (i.e., email) and should be documented accordingly.

### Early Termination

Early Termination is defined as a subject who does not complete the 6-month Follow up visit. Early termination may be due to medical discharge without follow-up, loss to follow-up, withdrawal by a subject, or death.

### Number of Subjects

This study will enroll up to 86 subjects, with 14 subjects in the Phase I portion and up to 72 subjects in the Phase II portion with 36 subjects given CYNK-001 and other 36 subjects who will be treated with the best supportive care only.

### Dose Limiting Toxicity (DLT) Definition

Known pathologies associated with COVID-19 will be carefully considered and differentiated from potential CYNK-001-related effects in order to identify CYNK-001 related toxicities. Adverse events occurring up to Day 28 from the first dose of the CYNK-001 infusion will be included in the dose-limiting toxicity (DLT) determination.

A DLT is defined as the development of any new (not pre-existing) events:
Grade 4 or 5 event in any organ system
Grade 4>24 hour (Due to known organ damage associated with the COVID-19) in the following organ systems:
   Cardiac
   Pulmonary
   Hepatic
   Renal
Central Nervous System (CNS)
Grade 3 or above allergic reaction that is suspected to be related to CYNK-001.
Grade 3 or above GvHD event occurring within the first 28 days following CYNK-001 infusion (to Study Day 28).
Grade 3 or above CRS event occurring within the first 28 days following the first CYNK-001 infusion (to Study Day 28).
   All above events to be identified in discussion with the clinical study Medical Monitor and reportable to Drug Safety.

The events will be assessed for the first 3 subjects in Phase I and per the study stopping rules for the remaining subjects. Any such findings will be forwarded to the DMC for recommendation, review and confirmation as to whether or not the maximal tolerated dose (MTD) has been exceeded. If the MTD is confirmed by the DMC, no further CYNK-001 administration will occur within that dose level or at any higher dose level. For the remaining 11 subjects, DMC will be convened if the safety stopping rule is met. Overall, in case of 2 out of the 6 subjects had experienced DLTs in the Phase I portion of this study, the DMC will be convened for safety evaluation.

During Phase II portion of the study, DMC will be convened at midpoint (after 18 subjects have received treatment) to evaluate safety for adverse event of interest such as shock, ARDS, and death in the treatment group versus the control group.

MTD is defined as the highest CYNK-001 dose level wherein it was deemed safe per the defined stopping rules or if the DMC recommends stopping the study due to DLTs suspected to be related to CYNK-001.

### Treatment Assignment

Phase I: Upon confirmation of eligibility during the Screening Period, eligible subjects will be sequentially assigned to the CYNK-001 group at the time of eligibility based on treatment slot availability.

Dose Level cohorts: Dose Level 1: CYNK-001 with an initial IV dose of 150×10⁶ cells on Day 1 followed by 600×10⁶ cells CYNK-001 IV on Days 4 and 7. After the first dose, subsequent infusions on Days 4 and 7 will be provided only if no toxicity of ≥Grade 3 (either related or unrelated to CYNK-001) is observed for each subject. If any such ≥Grade 3 toxicity is observed, the second and third doses will be delayed up to 48 hours until the noted event is resolved or reduced to Grade 1 toxicity level. Dose de-escalation Level −1: CYNK-001 with an initial IV dose of 150×10⁶ cells on Day 1 followed by 600×10⁶ cells CYNK-001 IV on Day 7 will be implemented due to potential safety concerns per the PMC recommendation. A total of 14 subjects will be treated in the Phase I portion of the study.

Phase II: For the Phase II portion of the study, up to 72 subjects will be randomized into the study with 1:1 ratio to either CYNK-001 or the control arm (best supportive care). The Phase II portion of the study is a randomized, open-label, multi-site study. Subjects will be randomized into either CYNK-001 group or Control group with 1:1 ratio stratified by age (<45 vs. ≥45 years old).

### Dose Adjustment Criteria

Dose adjustments may occur if clinically indicated by the treating physician. In general, the following should be followed:
Dose reductions are not permitted in this study. If any DLTs were observed, the dose de-escalation treatment with reducing frequency of doses will be implemented per the DMC recommendation.
Should dose delays for CYNK-001 be required:
   Day 1 will be the date of initial dose
   Day 4 dose may be delayed up to 48 hours
      For non-safety reasons: If delayed longer than 48 hours, Day 4 dose will be skipped, and the subject will receive the Day 7 dose. If the Day 4 dose is given within 48 hours, the Day 7 dose will be delayed for three days from the actual day of when Day 4 dose was given (Ex: if Day 4 dose is given on Day 5, then Day 7 dose will be given on Day 8)
      For safety reasons: Day 4 dose could be delayed if ≥Grade 3 toxicity is observed after the first dose. In such cases, Day 4 dose is provided only if the event is resolved or reduced to Grade 1 toxicity level. If the toxicity did not resolve within 48 hours, the Day 4 dose will be skipped. If the Day 4 dose is given within 48 hours, the Day 7 dose will be delayed for three days from the actual day of when Day 4 dose given.
      If the subject has worsening of illness, study medication will be stopped.
   Day 7 dose may be delayed up to 48 hours:
      For non-safety reasons: If delayed longer than 48 hours, the subject will not receive additional therapy.
      For safety reasons: If Day 4 dose was delayed but given within 48 hours of planned Day 4, then the Day 7 dose will be delayed for three days from the actual day of when Day 4 dose was given.
      If Day 4 dose was given as planned, Day 7 dose could be delayed if ≥Grade 3 toxicity is observed after the second dose. If Day 4 dose was skipped, Day 7 dose could be delayed if ≥Grade 3 toxicity is still observed after the first dose. In such cases, Day 7 dose is provided only if the event is resolved or reduced to Grade 1 toxicity level. If the toxicity did not resolve within 48 hours of the scheduled day, the Day 7 dose will not be administered.
      If the subject has worsening of illness, study medication will be stopped.
   All subjects who receive any amount of CYNK-001 will be followed to 6 months or until loss to follow-up, death, or withdrawal from study, whichever occurs first.
   If subject has been found to be SARS-CoV-2 negative by rRT-PCR or the third dose is skipped per protocol allowances, and has received two doses of CYNK-001, the third dose is not mandatory. The subject may be discharged one day following the final planned dose of CYNK-001.

Consultation with the Medical Monitor is required prior to each CYNK-001 infusion if there is:
An increase in supplemental oxygen of greater than or equal to 50% from baseline (for first CYNK-001 infusion) or from level at prior CYNK-001 infusion (for second and third CYNK-001 infusion) resulting in oxygen use of greater than 8 L.
—or—
A change in the mode of supplemental oxygen delivery with the intention to deliver oxygen more efficiently.
The Medical Monitor will escalate to the Sponsor clinical and safety teams as appropriate per GCP guidelines to advise if a subject who is experiencing rapid worsening of disease should proceed to first or subsequent CYNK-001 infusion.

### Duration of Study Participation

### Treatment Discontinuation

Discontinuation from study medication does not mean discontinuation from the study, and remaining study procedures should be completed as indicated by the study protocol. If a clinically significant finding is identified (including but not limited to changes from baseline) after enrollment, the investigator or qualified designee will determine if any change in participant management is needed. Any new clinically relevant finding will be reported as an AE. The following events are considered sufficient reasons for discontinuing a subject from study medication:
If any clinical AE, laboratory abnormality, or other medical condition or situation occurs such that continued treatment with study medication would not be in the best interest of the participant.
If the participant meets an exclusion criterion (either newly developed or not previously recognized) that precludes further study participation. Discussion with the Medical Monitor is recommended.
Worsening of illness which requires discontinuation of study medication at the discretion of the treating physician.
Subject withdrawal from treatment (subject no longer wants to receive study medication but is willing to have additional data collected), which must be documented in subject's medical record. It must be confirmed in documented communications whether or not AEs are leading the subject's choice to withdraw from the study medication.
Death
Protocol violation; discussion with the Medical Monitor is recommended.
Pregnancy
Loss to follow-up
Completion of study treatment according to the study protocol.
   Reason for study treatment discontinuation must be recorded in the electronic case report form (eCRF) and source documents.

### Study Discontinuation

The following events are considered sufficient reasons for discontinuing a subject from the study:
Screen failure
Subject withdrawal from study (subject no longer wants to participate in the study and is willing to have additional data collected), which must be documented in subject's medical record. It must be confirmed in documented communications whether or not AEs are leading the subject's choice to withdraw from the study.
Significant study intervention non-compliance
Death
Loss to follow-up
Protocol violation; discussion with the Medical Monitor is recommended
   Reason for study discontinuation must be recorded in the eCRF and source documents.

### Subject Withdrawal

Subjects may withdraw voluntarily from the study at any time upon request. Information related to the subject withdrawal must be well documented in the source document, including the documentation associated with any AEs the subject may or may not be experiencing at the time of the withdrawal.

Subjects who withdraw from the study after a single dose of CYNK-001 treatment will not be replaced.

### Criteria for Study Termination

The study may be terminated for the following reasons:
Study is completed as planned.
The study is terminated based on lack of evidence of therapeutic benefit.

Celularity also reserves the right to terminate this study prematurely at any time for reasonable medical or administrative reasons. Any premature discontinuation will be appropriately documented according to local requirements (e.g., IRB/EC, regulatory authorities, and others as applicable).

In addition, the Investigator or Celularity has the right to discontinue a single site at any time during the study for medical or administrative reasons such as:
Unsatisfactory enrollment
GCP non-compliance
Inaccurate or incomplete data collection
Falsification of records
Failure to adhere to the study protocol
Number of subjects not following the study protocol plan surpasses the statistical drop-out rate assumption resulting in the study being underpowered.

### End of Trial

The End of Trial is defined as either the date of the last visit of the last subject to complete the post-treatment follow-up, or the date of receipt of the last data point from the last subject that is required for primary, secondary and/or exploratory analysis, as specified in the protocol, whichever is the later date.

Efficacy Endpoint Definitions
Time to Clinical Improvement by OSCI: defined as the time from the date of randomization to the first date of clinical improvement measured by OSCI. Clinical improvement is defined as an improvement at least by cross category (or Patient State improvement, such as from Hospitalized Severe Disease to Hospitalized Mild Disease). Subjects who do not have clinical improvement on or before Study Day 28 will be censored at Study Day 28.
Clinical Status by OSCI: defined by the Ordinal Scale score of 0 to 8 by OSCI.
Rate of Clinical Improvement by OSCI: defined as the proportion of subjects who achieved clinical improvement by OSCI.
Time to Clearance of SARS-CoV-2: defined as the time from the date of randomization to the clearance of SARS-CoV-2 by rRT-PCR by two negative results at least 24 hours apart, with the first negative as the start date of clearance. Specimens included are nasopharyngeal swab and oropharyngeal swab (if available). Subjects who do not have clearance on or before Day 28 will be censored at Day 28.
Rate of Clearance of SARS-CoV-2: defined as the proportion of subjects with “negative” measurement of COVID-19 by rRT-PCR.
Time to Clinical Improvement by NEWS2: defined as the time from the date of randomization to the first date of clinical improvement. Clinical improvement is defined as improvement of clinical symptoms as measured by the NEWS2 Score. Subjects who do not have clinical improvement on or before Study Day 28 will be censored at Study Day 28.
Rate of Clinical Improvement by NEWS2: defined as the proportion of subjects who achieved clinical improvement.
Time to Pulmonary Clearance: defined as the time of randomization to the date of pulmonary clearance. This is defined as disappearance of virus from LRT-specimen where it has previously been found (induced sputum if available, endotracheal aspirate if available). Subjects who do not have pulmonary clearance on or before Study Day 28 will be censored at Study Day 28.
Pulmonary Clearance Rate: defined as the proportion of subjects who achieve pulmonary clearance.
Duration of Hospitalization: defined as date of randomization to the date of medical discharge. Subjects who are not discharged on or before Study Day 28 will be censored at Study Day 28.
Ventilatory Support: For those subjects requiring ventilatory support or supplemental oxygen during the treatment period:
   Supplemental oxygen-free days
   Proportion of subjects developing respiratory failure requiring invasive or noninvasive mechanical ventilation
SOFA Score: For those subjects evaluated by Sequential Organ Failure Assessment (SOFA) scores from ICU admission through ICU discharge (for subjects requiring intensive care). Mean arterial pressure should be measured with an arterial line.
   Organ support, according to the number of days within the 28 days starting from Day 1 when subjects do not receive specific forms of support:
a. Supplemental oxygen-free days
b. Renal replacement therapy-free days
c. Vasopressor-free days
d. Invasive or non-invasive mechanical ventilation free days
e. Organ support-free days (that is, days free of invasive mechanical ventilation, renal replacement therapy and vasopressors)
f. Extracorporeal circulation support-free days

Mortality Rate: defined as the proportion of subjects who died by any cause.

### Table of Events

TABLE 6
Table of Events

| | | | | | | | | | | Hospital | |
|---|---|---|---|---|---|---|---|---|---|---|---|
| | | Treatment Period | | | | | | Follow-up period ^{w} | | Dis- | |
| | Screen | | Day 4 | Day 7 | Day 15 | Day 21 | Day 28 | Day 90 | Day 180 | charge^{v} | Early |
| | Base- | Day | (+2 | (+2 | (±2 | (±2 | (± | (±14 | (±14 | (if | Termi- |
| Event | line ^{a} | 1^{k} | days)^{k} | days)^{k} | days) | days) | 2 days) | days) | days) | applicable) | nation ^{b} |

Study Entry and General Assessments

| | | | | | | | | | | | |
|---|---|---|---|---|---|---|---|---|---|---|---|
| Study Informed Consent | X | — | — | — | — | — | — | — | — | — | — |
| Inclusion/Exclusion | X | — | — | — | — | — | — | — | — | — | — |
| Assessment | | | | | | | | | | | |
| Pre-ICF rRT-PCR (or | X | — | — | — | — | — | — | — | — | — | — |
| other approved test per | | | | | | | | | | | |
| institutional practice) | | | | | | | | | | | |
| results confirming | | | | | | | | | | | |
| SARS-CoV-2 | | | | | | | | | | | |
| Demographics/Medical | X | — | — | — | — | — | — | — | — | — | — |
| history | | | | | | | | | | | |

Treatment and Study Required concomitant medications

| Phase II only: | X | — | — | — | — | — | — | — | — | — | — |
|---|---|---|---|---|---|---|---|---|---|---|---|
| Randomization to | | | | | | | | | | | |
| CYNK-001 or Best | | | | | | | | | | | |
| Supportive Care (1:1 | | | | | | | | | | | |
| ratio) after treatment | | | | | | | | | | | |
| eligibility is confirmed | | | | | | | | | | | |
| CYNK-001 IV | — | X | X | X^{e} | — | — | — | — | — | — | — |
| infusion^{c,d} | | | | | | | | | | | |
| Best supportive care - | — | Control group to receive Best | | | | | | — | — | — | — |
| Control group (Phase II | | Supportive Care only | | | | | | | | | |
| only) | | | | | | | | | | | |
| Acetaminophen 650 mg | — | X^{fk} | X^{fk} | X^{efk} | — | — | — | — | — | — | — |
| PO pre- and post- | | | | | | | | | | | |
| medication^{d} | | | | | | | | | | | |
| Diphenhydramine 25 mg | — | X^{fk} | X^{fk} | X^{efk} | — | — | — | — | — | — | — |
| PO/IV pre- and post- | | | | | | | | | | | |
| medication^{d} | | | | | | | | | | | |
| 4-hour safety monitoring | — | X^{f} | X^{f} | X^{f} | — | — | — | — | — | — | — |
| after each infusion^{f} | | | | | | | | | | | |

CYNK-001 Safety Laboratory Assessments and Procedures

| Adverse events^{g} | Continuous starting after informed | | | | | | | | | | |
|---|---|---|---|---|---|---|---|---|---|---|---|
| | consent, until last study visit. | | | | | | | | | | |
| 12-lead ECG | X | If clinically indicated | | | | | X | If clinically indicated | | | |
| Infusion site assessment | — | X^{k} | X^{k} | X^{k} | — | — | X | — | — | — | X |
| Pregnancy test for | L | — | — | — | — | — | X | — | — | — | Lⁱ |
| FCBP ^{h} | | | | | | | | | | | |
| C-Reactive protein, | — | L ^{jk} | L ^{jk} | L ^{jk} | L ^{jk} | L ^{jk} | L ^{jk} | — | — | — | — |
| ferritin, D-dimer, | | | | | | | | | | | |
| procalcitonin | | | | | | | | | | | |

| | | | | | | | |
|---|---|---|---|---|---|---|---|
| Coagulation profile (PT, | — | L^{k} | If clinically indicated | — | — | — | Lⁱ |
| INR, PTT, aPTT, | | | | | | | |
| fibrinogen) | | | | | | | |

| | | |
|---|---|---|
| Electroencephalography | — | If clinically indicated |
| (EEG) ^{j} | | |

| | |
|---|---|
| Urinalysis (dipstick) | If clinically indicated |

Clinical Data and Laboratory Information Collection

| Physical Examination^{u} | X | X^{k} | X^{k} | X^{k} | X | X | X | X | X | X | X |
|---|---|---|---|---|---|---|---|---|---|---|---|
| Discharge Plan (if | — | — | — | Upon discharge, plans should be made for appropriately delegated | | | | | | X | — |
| applicable) ^{lmv} | | | | staff to have telephone contact with subjects every day between | | | | | | | |
| | | | | hospital discharge through at least Day 15 visit for safety/AE | | | | | | | |
| | | | | monitoring. The discharge plan should include follow-up visit | | | | | | | |
| | | | | schedule as well as planned telehealth visit schedule. | | | | | | | |
| | | | | Consultation between the Medical Monitor and appropriately | | | | | | | |
| | | | | delegated site staff should occur every other day after hospital | | | | | | | |
| | | | | discharge up to Day 15 visit for ongoing review of the subject's | | | | | | | |
| | | | | clinical status. This communication may occur via telephone contact | | | | | | | |
| | | | | or written message (i.e. email) and should be documented | | | | | | | |
| | | | | accordingly. | | | | | | | |
| Daily Telephone calls to | | | Daily telephone calls | | | | | | | | |
| subject after discharge to | | | between hospital discharge | | | | | | | | |
| Day 15 | | | and Study Day 15. Subjects | | | | | | | | |
| | | | will self-report AEs, worsening | | | | | | | | |
| | | | of symptoms, and vitals (BP, | | | | | | | | |
| | | | oxygen saturation, and | | | | | | | | |
| | | | temperature) | | | | | | | | |
| Karnofsky Performance | X | X^{k} | X^{k} | X^{k} | X | X | X | X | X | X | X |
| Status | | | | | | | | | | | |
| Hospital Utilization ^{m} | | Continuous collection where applicable, starting after | | | | | | | | | |
| | | informed consent signature, until last study visit. | | | | | | | | | |
| Infection Symptom | X | X^{k} | X^{k} | X^{k} | X | X | X | X | X | X | X |
| Assessment^{g} | | | | | | | | | | | |
| SOFA score (for subjects | Continuous starting after informed consent until | | | | | | | | | | |
| requiring intensive care) | last study visit while in intensive care | | | | | | | | | | |
| Height | X | — | — | — | — | — | — | — | — | — | — |
| Vital Signs (Weight, BP, | X | X ^{nk} | X ^{nk} | X ^{nk} | X | X | X | X | X | X | X |
| Temp, Pulse, Respiration | | | | | | | | | | | |
| rate, Oxygen Saturation | | | | | | | | | | | |
| (SpO2)^{o}) ⁿ | | | | | | | | | | | |
| Supplemental Oxygen | X | X^{k} | X^{k} | X^{k} | X | X | X | X | X | X | X |
| level^{xo} | | | | | | | | | | | |
| Prior/Concomitant | | Prior collection relevant to medications administered for | | | | | | | | | |
| Medications and | | infection management and comorbidity control. Concomitant collection | | | | | | | | | |
| Procedures | | to be continuous starting after informed consent until last study visit. | | | | | | | | | |
| Hematology Panel^{p} | L | L^{k} | L^{k} | L^{k} | L | L | L | L | L | L | L |
| Serum Chemistry Panel^{q} | L | L^{k} | L^{k} | L^{k} | L | L | L | L | L | L | L |
| Chest X-ray | X | X^{k} | X^{k} | X^{k} | X | X | X | X | X | X | X |

| | | |
|---|---|---|
| Chest CT Scan | — | As clinically indicated |

Biomarkers

| | | | | | | | | | | | |
|---|---|---|---|---|---|---|---|---|---|---|---|
| IL-6 ^{j} | — | C^{k} | C^{k} | C^{k} | C | C | C | C | C | C | C |
| HLA type | — | C^{dk} | — | — | — | — | — | — | — | — | — |
| rRT-PCR Nasopha- | — | C^{k} | C^{k} | C^{k} | C | C | C | C | C | C | C |
| ryngeal swab | | | | | | | | | | | |
| Optional rRT-PCR | — | O^{k} | O^{k} | O^{k} | O | O | O | O | O | O | O |
| Oropharyngeal swab | | | | | | | | | | | |
| Optional rRT-PCR | — | O^{k} | O^{k} | O^{k} | O | O | O | O | O | O | O |
| Sputum | | | | | | | | | | | |
| rRT-PCR Serum^{t} | — | C^{k} | C^{k} | C^{k} | C | C | C | C^{t} | C^{t} | C | C |
| rRT-PCR Endotracheal | — | C | C | C | C | C | C | C | C | C | C |
| aspirate (for intubated | | | | | | | | | | | |
| subjects only) | | | | | | | | | | | |
| Immune Monitoring ^{r} | — | C^{k} | C^{k} | C^{k} | C | C | C | C | C | O | O |
| Serum Collection: | — | C^{k} | C^{k} | C^{k} | C | C | C | C | C | O | O |
| Cytokine evaluations and | | | | | | | | | | | |
| Panel Reactive antibodies | | | | | | | | | | | |
| (including anti-HLA) ^{sk} | | | | | | | | | | | |

Abbreviations: AE = adverse event; aPTT = Activated partial thromboplastin time; BP = Blood pressure; C = Central Lab, D = Day; ET = Early Termination; ECG = electrocardiogram; eCRF = electronic case report form; EEG =electroencephalography; FCBP = Female of childbearing potential; FEV1 = forced expiratory volume in one second; FVC = forced vital capacity: FEV1/FVC = ratio of forced expiratory volume in one second/forced vital capacity; HLA = human leukocyte antigen; ICF = Informed consent form; IL-6 = interleukin-6; INR = International Normalized Ratio; IV = Intravenously; L = Local Lab, NK = Natural Killer; O = optional assessment; PO = by mouth; PRA = panel reactive antibodies; PT = prothrombin time; PTT = partial thromboplastin time; Temp = temperature; rRT-PCR = Real-time Reverse-Transcriptase Polymerase Chain Reaction; SOFA = sequential organ failure assessment; SpO2 = Oxygen saturation; X = required assessment.
^{a} There is no pre-defined limit on the baseline period. In some cases, subject may be confirmed to have SARS-CoV-2 and receive initial dose of CYNK-001 within 24 hours. Subject must have clinically confirmed SARS-CoV-2 by rRT-PCR (or other approved test to detect SARS-CoV-2 per institutional practice) to qualify for undergoing informed consent process. All subsequent after baseline SARS-CoV-2 testing must be done by rRT-PCR.
^{b} Early termination is defined as a subject who does not complete the 6-month Follow-up Visit. This could be due to medical discharge without follow-up, loss to follow-up, withdrawal by the subject or death.
^{c}CYNK-001 will be administered at an initial dose of 150 × 10⁶ cells on Day 1 followed by 600 × 10⁶ cells on Days 4 and 7. Subjects should be monitored for at least 4 hours following completion of CYNK-001 infusion. CYNK-001 infusion should be paused or discontinued if there are any signs of an infusion site reaction or signs of an allergic reaction to the study drug. In the case an infusion site reaction or allergic reaction is suspected, the CYNK-001 infusion should be stopped, and vital signs should be taken. Treatment should be provided as clinically indicated and the patient should be monitored for at least 4 hours after the infusion.
^{d}For those subjects allocated to receive treatment with CYNK-001.
^{e}If subject has been found to be SARS-CoV-2 negative by rRT-PCR or the third dose is skipped per protocol allowances and has received two doses of CYNK-001, Day 7 CYNK-001 infusion (third planned dose) is not mandatory. If subject does not receive third dose, subject may be discharged one day following the final planned dose.
^{f}Acetaminophen 650 mg PO and Diphenhydramine 25 mg PO/IV should be administered at least 30 minutes prior to and 4 hours after completion of CYNK-001 infusion. Subjects should be monitored for at least 4 hours after each infusion.
^{g} Adverse events should be collected from the time the subject signs informed consent until their last study visit. Given that symptom associated with infection are critical to the study endpoints, symptoms associated with COVID-19 should be documented at baseline and graded according to the Common Terminology Criteria for Adverse Events 5.0. Symptom worsening and improvement should be captured in the EDC. Symptom worsening to be reported as an adverse event.
^{h} Pregnancy testing for females of childbearing potential should following institutional practices. Either blood or urine negative pregnancy test result is required to initiate initial CYNK-001 infusion and for post-treatment Day 28 test.
ⁱIf early termination is within 6 months from the first CYNK-001 infusion.
^{j} Testing completed for the purposes of Cytokine Release Syndrome (CRS) Monitoring. Careful consideration of possible CRS versus COVID-19 infection should be noted and addressed in source documentation. If CYNK-001 infusion is delayed, all lab tests including IL-6 should be collected on day of actual infusion. If IL-6 central analysis is delayed due to infusion delays or CRS is suspected up to Day 28, additional local IL-6 monitoring should occur.
^{k}Prior to CYNK-001 infusion, as applicable. Note: If CYNK-001 infusion is delayed per protocol, laboratory and exploratory collections and safety assessments are to be collected/performed on day of actual infusion. If any CYNK-001 infusion is skipped per protocol, laboratory and exploratory collections and safety assessments should be performed on originally scheduled infusion day when feasible.
^{l} Where applicable, a Discharge plan should be discussed with the subject to account for activities associated with the clinical trial following hospital discharge, doctor appointments in the outpatient setting and allowances to make contact via telephone to check on the status of the subject. Alternative person to contact in case of possible emergency or for planned daily calls from clinical site staff should also be collected. The discharge plan should include follow-up visit schedule as well as planned telehealth visit schedule. As part of discharge criteria, the study team should assess for further monitoring of subjects who may be experiencing toxicity of Grade 3 and above (either related or unrelated to CYNK-001) at the time of discharge and consult with the treating physician. The decision to discharge a subject should be in consultation with the treating physician. Physicians should follow best clinical practice to determine appropriate timing of hospital discharge.
^{m} Hospital utilization should document the nature of the admission and care unit used for clinical management of the subject. Discharge should be captured where applicable from hospital unit and/or hospital facility.
ⁿ Vital signs to be collected from medical record during hospital admission. Once subject is discharged the vital signs should be collected in the outpatient facility where the subject is seen. On each day of CYNK-001 infusion, vital signs to be collected pre-infusion, approximately 30 minutes after the start of infusion, and approximately 4 hours after the completion of infusion. On non-infusion days where multiple vital signs are collected throughout the day, vital signs should be captured in the EDC once per day and the worst vitals should be recorded.
Note:
Subjects must be monitored for at least 4 hours after completion of each CYNK-001 infusion.
^{o}Oxygen saturation levels to be collected from medical record during hospital admission where applicable. Once subject is discharged the oxygen saturation should be collected in the outpatient facility where the subject is seen. Oxygen therapy is permitted and should be carefully documented, including daily level of oxygenation requirements from time of hospital admission to time of hospital discharge.
^{p}Hematology panel including complete blood count (CBC) with differential, including red blood cell (RBC) count, hemoglobin, hematocrit, white blood cell (WBC) count (with differential), platelet count, and mean platelet volume (MPV).
^{q}Chemistry panel including sodium, potassium, calcium, carbon dioxide (bicarbonate) CO2 chloride, blood urea nitrogen (BUN), creatinine, lactate dehydrogenase (LDH), glucose, albumin, total protein, alkaline phosphatase, bilirubin (total and direct), aspartate aminotransferase/serum glutamic oxaloacetic transaminase (AST/SGOT), alanine aminotransferase/serum glutamic pyruvic transaminase.
^{r} Peripheral blood to be collected in 3 × 10 mL green top sodium heparin tubes, 1 × 4 mL green top sodium heparin tube, and 1 × 6 mL ACD tube
^{s} 4 × 500 ul and 1 × 1 ml aliquots of serum will be separated from 1 × 8.5 mL blood collected in marbled red top serum separation tubes and immediately frozen for subsequent cytokine correlates and anti-HLA/anti-PRA analysis.
^{t}1 mL serum will be separated from 1 × 3.5 mL blood collected in marbled red top serum separation tubes and immediately frozen for subsequent testing for SARS-CoV-2. During follow up, serum collection is optional if previous serum testing tested negative for SARS-CoV-2.
^{u}Physical examinations should be well documented in source documents, allowing for subject's overall wellbeing and illness related symptoms to be monitored for improvement and/or worsening.
^{v}Hospital Discharge visit should be performed if a hospitalized subject is discharged on any day other than a scheduled study visit day.
^{w} If institutional practice prevents COVID-19 patients from returning to the institution for research purposes, follow up visits may occur by telephone for clinical status and adverse event monitoring/reporting.
^{x}Consultation with the Medical Monitor is required prior to each CYNK-001 infusion if 1) there is an increase in supplemental oxygen of greater than or equal to 50% from baseline (for first CYNK-001 infusion) or from level at prior CYNK-001 infusion (for second and third CYNK-001 infusion) resulting in oxygen use of greater than 8 L -or- there is a change in the mode of supplemental oxygen delivery with the intention to deliver oxygen more efficiently. The Medical Monitor will escalate to the Sponsor clinical and safety teams as appropriate per GCP guidelines to advise if a subject who is experiencing rapid worsening of disease should proceed to first or subsequent CYNK-001 infusion.

### Selection and Withdrawal of Subjects

Subject Inclusion Criteria
1. Subject has confirmed positivity for SARS-CoV-2 as measured by rRT-PCR or other approved test to detect SARS-CoV-2 per institutional practice.
2. Subject is experiencing any symptom/clinical sign of COVID-19 illness or has a positive disease-related chest x-ray/CT scan at screening.
3. Subject is ≥18 years of age at the time of signing the Study informed consent form (ICF).
4. Subject understands and voluntarily signs the Study ICF prior to any study-related assessments/procedures are conducted.
5. Subject is willing and able to adhere to the study schedule and other protocol requirements.
6. SpO2≥88% on room air; oxygen is permitted as delivered by nasal cannula and/or face mask at any flow rate to achieve this SpO2. Subjects must have an SpO2≥92% if on supplementary oxygen.
   (Note: Once eligibility is confirmed to initiate CYNK-001 treatment, an increase of supplemental oxygen of greater than or equal to 50% from baseline/screening resulting in oxygen use of greater than 8 L—or—a change in mode of supplemental oxygen delivery with the intention to deliver oxygen more frequently requires consultation with the medical monitor)
7. Ability to be off immunosuppressive drugs for 3 days prior to infusion, unless clinically indicated. Steroids are permitted if clinically indicated and at the discretion of the treating physician. If clinically indicated, careful consideration should be taken regarding the timing and tapering of high-dose steroids.
8. Female of childbearing potential (FCBP)* must not be pregnant and agree to not becoming pregnant for at least 28 days following the last infusion of CYNK-001. FCBP must agree to use an adequate method of contraception during the treatment period. a. *FCBP is a female who: 1) has achieved menarche at some point, 2) has not undergone a hysterectomy or bilateral oophorectomy or 3) has not been naturally postmenopausal (amenorrhea following cancer therapy does not rule out childbearing potential) for at least 24 consecutive months (i.e., has had menses at any time in the preceding 24 consecutive months).
9. Male subjects must agree to use a condom during sexual contact for at least 28 days following the last infusion of CYNK-001, even if he has undergone a successful vasectomy.

Subject Exclusion Criteria
1. Subject requires supplemental oxygen delivered by mechanical ventilation, either invasive or bilevel positive airway pressure.
2. Subject admitted to Intensive Care Unit/Pulmonary Acute Care Unit designated area with severe pulmonary pneumonia, ARDS or Sepsis.
3. Subject is pregnant or breastfeeding.
4. Subject has a history of chronic asthma requiring ongoing medical therapy or other chronic pulmonary disease that, at the discretion of the treating physician, would contraindicate participation in this study.
5. Subject has any other organ dysfunction [Common Terminology Criteria for AEs (CTCAE) Version 5.0 Grade 3] that will interfere with the administration of the therapy according to this protocol.
6. Subject has inadequate organ function as defined below at time of Treatment Eligibility Period:
   a) Subject has aspartate aminotransferase (AST), alanine aminotransferase (ALT), or alkaline phosphatase≥5× the upper limit of normal (ULN). (It is anticipated that the infection may impact liver.)
   b) Estimated glomerular filtration rate (eGFR)<30 mL/min/1.73 m2 as calculated using the Modification of Diet in Renal Disease Study equation (Levey, 2006) or history of an abnormal eGFR <60. A decline of >15 mL/min/1.73 m2 below normal in the past year prior to infection. (It is anticipated that the infection may impact renal function.)
   c) Subject has a bilirubin level >2 mg/dL (unless subject has known Gilbert's Syndrome).
7. Subject has a known sensitivity or allergy to treatment additives or diluent substances of dimethyl sulfoxide (DMSO), PlasmaLyte A or human serum albumin (HSA). Please refer to investigational brochure (IB).
8. Subject has active autoimmune disease other than controlled connective tissue disorder or those who are not on active therapy.
9. Subject is immunocompromised, has known human immunodeficiency virus (HIV) positivity, or has actively been treated with immunosuppressive products prior to being infected with SARS-CoV-2.
10. Subject has known active malignancy, unless the subject has been free of disease for ≥3 years from the date of signing the ICF. Exceptions include the following noninvasive malignancies:
   a. Basal cell carcinoma of the skin
   b. Squamous cell carcinoma of the skin
   c. Carcinoma in situ of the cervix
   d. Carcinoma in situ of the breast
   e. Incidental histological finding of prostate cancer (TNM stage of T1a or T1b)
11. Detection of other respiratory viruses from mucosal surfaces that, at the investigator's discretion, would interfere with the study treatment plan; detection of another respiratory virus is not in itself an exclusion criteria unless the investigator believes it would interfere with administration of CYNK-001.
12. Subjects must not have a history of unconsciousness or hemoptysis within 2 weeks of signing ICF.
13. Subjects must not have end stage liver disease and/or cirrhosis.
14. Subject has any significant medical condition, laboratory abnormality, or psychiatric illness that would prevent the subject from participating in the study.
15. Subject has any condition including the presence of laboratory abnormalities which places the subject at unacceptable risk if he or she were to participate in the study.
16. Subject has any condition that confounds the ability to interpret data from the study

### Treatment of Subjects

### Description of Study Drug

### CYNK-001

CYNK-001 is an allogeneic off the shelf cell therapy enriched for CD56⁺/CD3′ NK cells culture-expanded from human placental CD34⁺ cells. Culture-expanded cells are harvested, washed in PlasmaLyte A and then packaged at 30.0+/−9.0×10⁶ cells/mL in a total volume of 20-mL of cryopreservation solution containing 10% (w/v) HSA, 5.5% (w/v) Dextran 40, 0.21% NaCl (w/v), 32% (v/v) PlasmaLyte A, and 5% (v/v) DMSO. It is filled into the container closure, frozen using a controlled rate freezer, and cryopreserved. Prior to releasing to the site, all release and characterization testing will be complete. When administration is required by a site, CYNK-001 is shipped in vapor phase LN2 to the designated clinical site where it will be processed for dose preparation in a standardized manner just prior to IV administration.

On Study Days 1, 4, 7, subjects will receive acetaminophen 650 mg orally (per os, PO) and diphenhydramine 25 mg PO/IV prior to CYNK-001 infusion, followed by CYNK-001 infusion, and acetaminophen 650 mg PO and diphenhydramine 25 mg PO/IV at least 30 minutes prior to each CYNK-001 infusion and approximately 4 hours after completion of each CYNK-001 infusion.

CYNK-001 will be administered at an initial dose of 150×10⁶ cells followed by 600×10⁶ cells on Days 4 and 7, administered IV, using a gravity IV administration set with a 16- to 22-gauge (or equivalent) needle or catheter with no filters. A central line may be used to infuse CYNK-001 after confirming that the catheter diameter is 16- to 22-gauge (or equivalent) needle. For substantial deviation from this catheter diameter, consultation with the Medical Monitor is required. The recommended infusion rate is approximately 240 mL per hour. No other medications or blood products should be in the IV line at the time of CYNK-001 infusion. Vital signs should be taken pre-CYNK-001 infusion, approximately 30 minutes after the start of infusion, and approximately 4 hours after the completion of infusion. Additional vital signs should be taken if clinically indicated and any abnormal clinically significant findings should be documented. Immediately following the infusion, the infusion line will be flushed with 30 to 60 mL of normal saline.

The CYNK-001 infusion should be paused or discontinued if there are any signs of an infusion site reaction or signs of an allergic reaction to the study drug. In the case an infusion site reaction or allergic reaction is suspected, the CYNK-001 infusion should be stopped, and vital signs should be taken. Treatment should be provided as clinically indicated and the patient should be monitored for at least 4 hours after the infusion.

TABLE 7
Investigational Product

| | Investigational Product |
|---|---|
| Product Name: | CYNK-001 |
| Dosage Form: | CYNK-001 is a monodispersed cell suspension |
| | formulated in a cryopreserved solution and shipped |
| | in vapor phase liquid nitrogen. |
| Unit Dose | 600 × 10⁶ cells/bag |
| Route of | Intravenous (IV) |
| Administration | |
| Physical | The CYNK-001 Drug Product (DP) consists of CYNK- |
| Description | 001 cells formulated at 30.0 ± 9.0 × 10⁶ cells/mL in |
| | 10% (w/v) HSA, 5.5% (w/v) Dextran 40, 0.21% (w/v) |
| | NaCl, 32% (v/v) PlasmaLyte A, and 5% (v/v) DMSO. |
| | The straw-colored DP is filled at 20 ± 2.0 mL into a |
| | Saint Gobain KryoSure freezing bag. These bags are |
| | frozen in a controlled rate freezer, then stored and |
| | shipped in vapor phase liquid nitrogen. |
| Manufacturer | Designated Manufacturing facility for Celularity Inc. |

### CYNK-001 Overdose

On a per dose basis, an overdose is defined as the following amount over the protocol-specified dose of CYNK-001 assigned to a given subject, regardless of any associated AEs or sequalae:

CYNK-001: 30% over the assigned protocol-specified dose of 600×10⁶ cells.

On a schedule or frequency basis, an overdose is defined as anything more frequent than the protocol required schedule or frequency.

Complete data about drug administration, including any overdose, regardless of whether the overdose was accidental or intentional, should be reported in the eCRF.

### Concomitant Medications

Over the course of this study, additional medications may be required to manage aspects of the disease state of the subjects, including side effects from trial treatments or clinical worsening. Supportive care, including but not limited to antiemetic medications, may be administered at the discretion of the physician. Use of high-dose steroids (greater than or equal to 0.5 mg/kg per day prednisone equivalent) is not recommended during the Treatment Period, unless clinically indicated and at the discretion of the treating physician. High-dose steroids have been shown to interfere with the effectiveness of some adoptive cell therapies. If clinically indicated, careful consideration should be taken regarding the timing and tapering of high-dose steroids.

All concomitant treatments, including blood and blood products, used from 28 days prior to the first CYNK-001 infusion until completion of the study must be reported on the eCRF. All treatments administered for the purposes of providing care of COVID-19 including name and number of doses must be reported on the eCRF.

For information regarding other drugs that may interact with CYNK-001 and affect CYNK-001 activity, please see the IB.

### Permitted Concomitant Medications

All subjects are to receive standard medical care for COVID-19 signs and symptoms, unless contraindicated.

During the Treatment Period and Follow-Up Period, the following Concomitant Medications are permitted:

Prophylactic antibiotic and antifungal medication are permitted at the discretion of the treating physician. These treatments must be identified as prophylactic in the physical examination source documents.

Diphenhydramine and acetaminophen are permitted to be used as indicated before and after CYNK-001 administration and as clinically indicated.

Meperidine is permitted for the control of rigors and as clinically indicated.

Steroids are permitted if clinically indicated and at the treating physician's discretion during the treatment period. If clinically indicated, careful consideration should be taken regarding the timing and tapering of high-dose steroids.

Blood product transfusions may occur as clinically indicated greater than 24 hours before or greater than 24 hours after CYNK-001 infusion.

Supplemental oxygen therapy is permitted if clinically indicated.
Note: Per NIH COVID-19 Treatment Guidelines dated 17 Dec. 2020, the optimal target SpO2 in adults with COVID-19 is between 92% and 96%. (https://www.covid19treatmentguidelines.nih.gov/critical-care/oxygenation-and-ventilation/; NIH COVID-19 Treatment Guidelines: Oxygenation and Ventilation. 17 Dec. 2020)

Concomitant therapy with potential activity against COVID-19 is permitted.

### Prohibited Concomitant Medications

Blood product transfusions should not occur within 24 hours prior to and/or 24 hours after CYNK-001 infusion, unless clinically indicated.

### Required Concomitant Medications

The best supportive care treatments must be identified in the source documents and documented that they are administered for COVID-19. Prophylactic use of any treatment during the study must be documented as prophylactic treatment.

Subjects should receive adequate medical therapy for control of hypertension, diabetes, and any other chronic medical conditions for which they require ongoing care.

Acetaminophen and diphenhydramine are required concomitant medications to be administered prior to and following each CYNK-001 infusion.

In some cases, tocilizumab, an anti-IL-6R-antibody, may be required to treat toxicities such as Cytokine Release Syndrome (CRS). Please refer to currently approved Actemra® package insert (Actemra, 2019). The recommended dose to intervene in subjects with CRS is 8 mg/kg; however, dosing is at the discretion of the treating physician. Other similarly available immune-modulatory drugs (targeted biologics), but not corticosteroids or more broadly-acting immunosuppressants) could be considered per physician discretion.

### Treatment Compliance

CYNK-001 is to be administered IV at the clinical study site. Study personnel will review the dosing treatment allocation and ensure treatment is administered according to the subject's treatment plan. Treatment compliance will be noted on the appropriate CRFs and source records based on administration records.
Dose reductions are not permitted in this study.
Dose delays are permitted.
Concomitant therapies
   Blood product transfusions should not occur within 24 hours prior to and/or 24 hours after CYNK-001 infusion.
   Use of steroids is permissible if clinically indicated and at the discretion of the treating physician.

### Study Drug Materials and Management

Celularity will supply CYNK-001 for IV administration. Subjects will receive CYNK-001 according to the protocol specified treatment plan.

Commercially available acetaminophen and diphenhydramine will be used. Site should obtain commercially available product through the local hospital pharmacy or licensed distributor.

Tocilizumab or similarly available immune-modulatory drugs (targeted biologics, but not corticosteroids or more broadly-acting immunosuppressants) should be available on site for administration of at least 3 doses soon after an order has been placed in the event of suspected CRS requiring treatment.

### Study Drug

CYNK-001, human placental hematopoietic stem cell derived natural killer cells, consists of culture-expanded cells which are harvested, washed in PlasmaLyte A and then packaged at 30.0+/−9.0×10⁶ cells/mL in a total volume of 20 mL of cryopreservation solution containing 10% (w/v) HSA, 5.5% (w/v) Dextran 40, 0.21% NaCl (w/v), 32% (v/v) PlasmaLyte A, and 5% (v/v) DMSO. It is filled into the container closure, frozen using a controlled rate freezer, and cryopreserved.

### Study Drug Packaging and Labeling

CYNK-001 investigational product is packaged in 50 mL bags that is designed as a closed system for freezing, thawing, and transfer of sterile contents. The bags used are made from high quality USP Class VI fluorinated ethylene propylene (FEP) material. Each bag is independently labeled with the product identifier, lot number, volume, required storage temperature, and bag number. Each bag is loaded into a protective aluminum cassette. Each cassette is labeled with the same information listed on the bag within.

### Study Drug Storage

CYNK-001 investigational product will be shipped in a qualified shipping configuration that will maintain and track cryogenic temperature data and critical chain of custody events.

Depending on the clinical site's needs, investigational product will either:
Be shipped, per dose, directly to the clinical site, then will be either moved directly from the cryogenic shipper to the clinical sites qualified LN2 freezer OR be left in the cryogenic shipper to be removed prior to dose preparation. Shippers are qualified for up to ten days of dynamic hold time. If any delay in dosing occurs beyond 48 hours of receipt of the shipper, the investigational product will need to be transferred by trained personnel to a qualified onsite LN2 freezer maintaining cryogenic conditions.
Be shipped with all doses included. Once receipt of the shipper has occurred at the site, the shipper will be unloaded by trained personnel and investigational product transferred into a qualified onsite LN2 freezer.

Storage of investigational product at cryogenic temperatures below −150° Celsius, is required to maintain the stability of CYNK-001. Storage of CYNK-001 is required to be in a qualified LN2 freezer that maintains these cryogenic conditions. The optimal temperature range from storage is between −150° Celsius and −200° Celsius.

The onsite freezer must maintain temperature monitoring that can be accessible to the investigator(s) or designee upon request. Temperature monitoring must also include alarms in the event of a malfunction in temperature recording or a temperature deviation above −150° Celsius. If a malfunction or deviation occurs, the investigator(s) or designee and Sponsor are required to be notified immediately. The investigator(s) or designee are encouraged to consult with the Sponsor on how to proceed with the impacted product. The impacted product in question should be quarantined per the sites standard operating procedure until direction from investigator(s) or designee on how to proceed is determined. In the event of a malfunction in temperature recording or a deviation from acceptable temperature occurs, a root cause analysis should be conducted and be available to the investigator(s) or designee.

### Study Drug Preparation

Each CYNK-001 bag will be the combination of 600×10⁶ cell CYNK-001 Drug Product Bag(s) and 40 mL of 10% HSA in PlasmaLyte A. The Dose Administration Table below provides the required number of CYNK-001 Drug Product Bags and volume of 10% HSA in PlasmaLyte A to reach the protocol specified intended dose.

TABLE 8
Dose Administration

| | | Number of CYNK-001 | Volume of 10% HSA in |
|---|---|---|---|
| Dose | Dose (Cells) | Drug Product Bags | PlasmaLyte A (mL) |
| Initial | 150 × 10⁶ | 1 | 10 |
| Second | 600 × 10⁶ | 1 | 40 |
| & Third | | | |

Preparation must be performed by an institutional qualified and study designated site staff member. Use aseptic technique.

Preparation of Diluent Solution Bag
1. Insert the appropriate dispensing pins into the septum of the 25% HSA stock solution bottle and the port on the PlasmaLyte A bag.
2. Obtain the transfer pack that will serve as the Diluent Solution Bag and insert the appropriate dispensing pin.
3. Using a syringe, remove 50 mL of PlasmaLyte A from the PlasmaLyte A Bag.
4. Attach the syringe to the Diluent Solution Bag and dispense its contents into the bag.
5. Repeat steps 3 and 4 to transfer a total of 150 mL of PlasmaLyte A into the Diluent Solution Bag.
6. Using a new syringe, remove the entire 100 mL contents of the 25% HSA solution and dispense it into the Diluent Solution Bag.
7. Thoroughly mix the Diluent Solution Bag, now containing 10% HSA in PlasmaLyte A, by gently massaging the bag and inverting slowly multiple times.

### Thaw and Dilution of CYNK-001 Drug Product

Aseptic connections in this section may be performed by either the tube welding or spike method.
1. Wearing appropriate personal protective equipment, obtain CYNK-001 cassettes from LN2 dry shipper or Sponsor authorized and approved storage freezer. Transfer the cassettes between the dry shipper and freezer or designated area for thaw on dry ice.
2. Carefully remove the CYNK-001 Drug Product Bags from the cassettes. Inspect the bags for any breaks or cracks prior to thawing.
3. Thaw the CYNK-001 Drug Product bag at 37° Celsius using a water bath or use the dry thaw method until there is no visible ice in the drug product bag. Remove the bag immediately once complete thaw has been achieved and record the thaw timepoint. The product should be in ambient conditions until infusion for up to 4 hours. If there is any delay in infusion, the product should be stored at 2-8° C. for up to 8 hours.
4. Using a syringe, draw 20-mL of diluent from the Diluent Solution Bag.
5. Attach the syringe containing 20-mL of diluent to the CYNK-001 Drug Product Bag and dispense the contents into the bag.
6. Gently massage the CYNK-001 Drug Product Bag to break up any cell aggregates.
7. Using the same syringe, draw up the entire content of the CYNK-001 Drug Product Bag taking care to remove cells from the corners and near the ports.
8. Dispense the contents of the syringe slowly into the CYNK-001 Infusion Bag.
9. Using a new syringe draw 20 mL of diluent from the Diluent Solution Bag.
10. Attach the syringe to the empty CYNK-001 Drug Product Bag and dispense contents into the bag.
11. Rinse the bag with the diluent to ensure there are no residual cells and draw the solution into the syringe.
12. Dispense the contents into the CYNK-001 Infusion Bag while slowly massaging the bag to ensure adequate mixing.
13. INITIAL DESENSITIZING DOSE ONLY (otherwise skip to Step 14)
For the Initial desensitizing dose (1.5×10⁸ cells), draw 15 mL from the prepared CYNK-001 infusion bag using a syringe and transfer the contents of the syringe to another new CYNK-001 infusion bag. Inspect the contents of the bag for visible clumps. The product is now ready for infusion.
14. FOR SECOND AND THIRD DOSES ONLY
For the second and third doses (6×10⁸ cells each), inspect the contents of the prepared CYNK-001 Infusion Bag for any visible clumps. The product is now ready for infusion.

CYNK-001 Administration
1. Spike an IV Administration Set without a filter into one of the spike ports on the bottom of the bag and prime the line.
2. Attach administration set Luer adapter to a 16- to 22-gauge needle (or equivalent) to the subject, or an existing port. If attached to an existing administration set, it is acceptable to maintain Keep Vein Open (KVO) flow of normal saline.
3. Adjust flow rate on administration set to infuse subject at a rate of approximately 240 mL per hour using a gravity administration set.
4. Immediately following the infusion, flush the line with 30 to 60 mL of normal saline.
Note: Initial desensitizing dose may be administered as a slow IV push over approximately 4 minutes, followed by 10-20 mL saline flush administered as a slow IV push over approximately 4 minutes if institutional practice allows.

CYNK-001 infusion should be paused or discontinued if there are any signs of an infusion site reaction or signs of an allergic reaction to the study drug. In the case an infusion site reaction or allergic reaction is suspected, the CYNK-001 infusion should be stopped, and vital signs should be taken. Treatment should be provided as clinically indicated and the patient should be monitored for at least 4 hours after the infusion.

### Assessment of Efficacy

This study will explore the potential clinical efficacy of CYNK-001 by evaluating:
OSCI: OSCI will be recorded daily in hospital and outpatient by phone contact.
Clearance of SARS-CoV-2: defined as the time from the date of randomization to the clearance of SARS-CoV-2 by rRT-PCR by two negative results at least 24 hours apart. Specimens included are nasopharyngeal swab and optional oropharyngeal swab.
Pulmonary Clearance: defined as the time from randomization to the date of pulmonary clearance. This is defined as disappearance of virus from LRT specimen where it has previously been found (induced sputum if available, endotracheal aspirate if available).
Duration of Hospitalization: defined as the date of hospitalization to the date of medical discharge.
Ventilatory Support: For those subjects requiring ventilatory support or supplemental oxygen during the treatment period:
   Supplemental oxygen-free days
   The development of respiratory failure requiring invasive or noninvasive mechanical ventilation
SOFA Score: For those subjects evaluated by Sequential Organ Failure Assessment (SOFA) scores at ICU admission through ICU discharge (for subjects requiring intensive care; mean arterial pressure to be measured with an arterial line).
   Organ support, according to the number of days within the 28 days starting Day 1 when subjects do not receive specific forms of support:
a. Supplemental oxygen-free days
b. Renal replacement therapy-free days
c. Vasopressor-free days
d. Invasive or non-invasive mechanical ventilation free days
e. Organ support-free days (that is, days free of invasive mechanical ventilation, renal replacement therapy and vasopressors)
f. Extracorporeal circulation support-free days
Mortality: defined as the death within 28 days and 6 months of any cause.
NEWS2 Score: NEWS2 Score will be calculated based on data collected in the EDC for assessment of clinical symptoms including respiration, oxygen saturation, blood pressure, pulse, consciousness, and temperature (Royal College of Physicians, 2017).
Radiologic Evaluation: Chest x-ray and/or CT scans will be evaluated at timepoints outlined in the Table of Events Table 5. In an effort to apply objective, semi-quantitative methods for radiologic evaluation, the following binary scoring system will be implemented:
   Score A: Chest x-ray or CT scan: normal (score 0) versus abnormal (score 1)
   Score B: Pleural Effusion: absence (score 0) versus presence (score 1)
Radiologic Evaluation Score: the sum of Score A and B.

### Exploratory Assessments

The translational and biomarker assays for this study will require obtaining peripheral blood and serum, sputum as indicated in the Table of Events, Table 5.
Interleukin-6 (IL-6), will be measured as a possible biomarker of CRS for confirmatory evaluations
Immune phenotyping including NK cell measurement
HLA typing for those subjects treated with CYNK-001
Serum Collection: cytokine and Anti-human leukocyte antigen (HLA) testing and anti-panel reactive antibodies (PRA) antibodies
rRT-PCR testing of nasopharyngeal swab, oropharyngeal swab (optional), sputum (optional), serum, endotracheal aspirate (if available)

### LIST OF REFERENCES

Actemra®. [Package Insert], South San Francisco, United States of America: Genentech, Inc; 2019. Available from: https://www.accessdata.fda.gov/drugsatfda_docs/label/2019/125276s120s1211bl.pdf
Bai Y, Lingsheng Y, Tao Wei, Tian F et al. Presumed Asymptomatic carrier transmission of COVID-19, 2020 February.
Brentjens R J, Riviere I, Park J H et al. Safety and persistence of adoptively transferred autologous CD-19-targeted T cells in patients with relapsed or chemotherapy refractory B-cell leukemias, 2011. 118(18): 4817-4828.
Centers for Disease Control and Prevention, Respiratory Viruses Branch, Division of Viral Diseases. Real-time RT-PCR panel for detection 2019-novel coronavirus, 2020 (https://www.cdc.gov/coronavirus/index.html)
Chen N, Zhou M, Dong X, Qu J, Gong F, Han Y et al. Epidemiological and clinical characteristics of 99 cases of 2019 novel coronavirus pneumonia in Wuhan, China: a descriptive study. Lancet 2020 Jan. 30. pii: S0140-6736(20)30211-7. doi: 10.1016/S0140-6736(20)30211-7. [Epub ahead of print]
Cook, K. D., S. N. Waggoner, and J. K. Whitmire, NK cells and their ability to modulate T cells during virus infections. Crit Rev Immunol, 2014. 34(5): p. 359-88.
Davila M L, Riviere I, Wang X, Bartido S, Park J, Curran K, et al. Efficacy and toxicity management of 19-28z CAR T cell therapy in B cell acute lymphoblastic leukemia. Sci Transl Med, 2014 Feb. 19; 6(224):224ra25.
Gazit R, Gruda R, Elboim M, Arnon T I, Katz G, Achdout H, Hanna J, Qimron U, Landau G, Greenbaum E, Zakay-Rones Z, Porgador A, Mandelboim O. Lethal influenza infection in the absence of the natural killer cell receptor gene Ncr1. Nat Immunol, 2006 May; 7(5):517-23.
Gritti G, Raimondi F, Ripamonti D, et al. Use of siltuximab in patients with COVID-19 pneumonia requiring ventilatory support. medRxiv 2020: 2020.04.01.20048561.
Guaraldi G, Meschiari M, Cozzi-Lepri A, et al. Tocilizumab in patients with severe COVID-19: a retrospective cohort study. The Lancet Rheumatology 2020.
Guidotti L G, Chisari F V. Immunobiology and pathogenesis of viral hepatitis. Annu Rev Pathol. 2006; 1:23-61.
Harris A C, Young R, Devine S, Hogan W, Ayuk F, Bunworasate U, et al. International, multicenter standardization of acute graft-versus-host disease clinical data collection: a report from the Mount Sinai acute GVHD International Consortium. Biol Blood Marrow Transplant 2016; 22(1):4-10.
Horby P, Lim W S, Emberson J, et al. Effect of Dexamethasone in Hospitalized Patients with COVID-19: Preliminary Report. medRxiv 2020: 2020.06.22.20137273.
Huang C, Wang Y, Li X, Ren L, Zhao J, Hu Y, et al. Clinical features of patients infected with 2019 novel coronavirus in Wuhan, China. The Lancet 2020; 395(10223): 497-506.
Ivanova D, Krempels R, Ryfe J, Weitzman K, Stephenson D, Gigley J P. NK cells in mucosal defense against infection. Biomed Res Int, 2014; 2014:413982.
Jones A E, Trzeciak S, Kline J A. The sequential organ failure assessment score for predicting outcome in patients with severe sepsis and evidence of hypoperfusion at the time of emergency department presentation. Crit Care Med, 2009 May; 37(5): 1649-54.
Lanier, L L. Evolutionary struggles between NK cells and viruses. Nat Rev Immunol, 2008. 8(4): p. 259-68.
Law, A. H., et al., Role for nonstructural protein 1 of severe acute respiratory syndrome coronavirus in chemokine dysregulation. J Virol, 2007. 81(1): 416-22.
Lee D W, Gardner R, Porter D L, Louis C U, Ahmed N, Jensen M, et al. Current concepts in the diagnosis and management of cytokine release syndrome. Blood, 2014 Jul. 10; 124(2):188-95. Erratum in: Blood, 2015; 126(8):1048.
Lee D, Santomasso B, Locke F, Ghobadi A, Turtle C, Brudno J et all. ASTCT Consensus Grading for Cytokine Release Syndrome and Neurologic Toxicity Associated with Immune Effector Cells. Biology of Blood and Marrow Transplantation 2019; 25(4): 625-638.
Levey A S, Coresh J, Greene T, Stevens L A, Zhang Y L, Hendriksen S, et al. Using standardized serum creatinine values in the modification of diet in renal disease study equation for estimating glomerular filtration rate. Ann Intern Med 2006; 145:247-54.
Littwitz E, Francois S, Dittmer U, Gibbert K. Distinct roles of NK cells in viral immunity during different phases of acute Friend retrovirus infection. Retrovirology, 2013 Nov. 1; 10:127. doi: 10.1186/1742-4690-10-127. PubMed PMID: 24182203; PubMed Central PMCID: PMC3826539.
Loh, J., et al., Natural killer cells utilize both perforin and gamma interferon to regulate murine cytomegalovirus infection in the spleen and liver. J Virol, 2005. 79(1): p. 661-7.
Mandelboim O, Lieberman N, Lev M, Paul L, Arnon T I, Bushkin Y, et al. Recognition of haemagglutinins on virus-infected cells by NKp46 activates lysis by human NK cells. Nature. 2001 Feb. 22; 409(6823): 1055-60.
Miller J S, Soignier Y et al. Successful adoptive transfer and in vivo expansion of human haploidentical NK cells in patients with cancer, 2005; 105:3051-3057.
Mor V, Laliberte L, Morris J N, Wiemann M. The Kamofsky performance status scale. 1984 Cancer 53: 2002-7.
Nogusa S, Ritz B W, Kassim S H, Jennings S R, Gardner E M. Characterization of age-related changes in natural killer cells during primary influenza infection in mice. Mech Ageing Dev. 2008 April; 129(4):223-30.
National Research Project for SARS, Beijing Group. The Involvement of Natural Killer Cells in the Pathogenesis of Severe Acute Respiratory Syndrome. Am J Clin Pathol 2004; 121:507-511.
National Early Warning Score (NEWS) 2: Standardising the assessment of acute-illness severity in the NHS. Royal College of Physicians; 2017.
National Institutes of Health (NIH). COVID-19 Treatment Guidelines: Oxygenation and Ventilation. Last updated: Dec. 17, 2020. Available from: https://www.covid19treatmentguidelines.nih.gov/critical-care/oxYgenation-and-ventilation/Research involving individuals with questionable capacity to consent: points to consider. NIH. November 2009. Retrieved from: https://grants.nih.gov/grants/policy/questionablecapacity.htm.
Stein-Streilein J, Guffee J. In vivo treatment of mice and hamsters with antibodies to asialo GM1 increases morbidity and mortality to pulmonary influenza infection. J Immunol. 1986 Feb. 15; 136(4): 1435-41.
Trifllo, M. J., et al., CXC chemokine ligand 10 controls viral infection in the central nervous system: evidence for a role in innate immune response through recruitment and activation of natural killer cells. J Virol, 2004. 78(2): p. 585-94.
Quillay H, El Costa H, Duriez M, Marlin R, Cannou C, Madec Y, et al. NK cells control HIV-1 infection of macrophages through soluble factors and cellular contacts in the human decidua. Retrovirology, 2016 Jun. 6; 13(1):39.
Walsh K B, Lodoen M B, Edwards R A, Lanier L L, Lane T E. Evidence for differential roles for NKG2D receptor signaling in innate host defense against coronavirus-induced neurological and liver disease. J Virol, 2008 March; 82(6):3021-30.
Wang D, Hu B, Hu C, Zhu F, Lui X et al. Clinical Characteristics of 138 Hospitalized Patients with 2019 Novel Coronavirus-Infected Pneumonia in Wuhan, China. Jama. Doi:10.1001/jama.2020.1585.
WHO—Clinical management of severe acute respiratory infection when Novel coronavirus (2019-nCoV) infection is suspected: Interim Guidance—World Health Organization, 28 Jan. 2020.
WHO—R&D Blueprint novel Coronavirus COVID-19 therapeutic trial synopsis—World Health Organization, 18 Feb. 2020.
Wu Z, Sinzger C, Reichel J J, Just M, Mertens T. Natural killer cells can inhibit the transmission of human cytomegalovirus in cell culture by using mechanisms from innate and adaptive immune responses. J Virol, 2015 March; 89(5):2906-17. doi: 10.1128/JVI.03489-14.
Xu X, Han M, Li T, et al. Effective treatment of severe COVID-19 patients with tocilizumab. Proc Natl Acad Sci USA 2020; 117(20): 10970-5.
Yin Y, Wunderink R G. MERS, SARS and other coronaviruses as causes of pneumonia. Respirology 2018; 23: 130-37.
Zeng Q., et al., Structure of coronavirus hemagglutinin-esterase offers insight into corona and influenza virus evolution. Proc Natl Acad Sci USA, 2008. 105(26): p. 9065-9.

### 7.7 Example 7: Treatment of Patients with Coronavirus Infections with CYNK Cells

### Background

CYNK-001 is a cryopreserved, allogeneic, off-the-shelf natural killer (NK) cell investigational product derived from placental CD34+ cells. CYNK-001 exhibits cytotoxicity against various cancer cell types as well as virally infected cells and secretes immunomodulatory cytokines upon target activation. This is the first study to evaluate the safety and potential efficacy of CYNK-001 to treat patients (pts) with SARS-CoV-2, previously investigated in only solid tumor and hematologic malignancies.

### Methods

Placental CD34+ cells were cultured in the presence of cytokines for 35 days to generate CYNK-001 under the cGMP conditions. Pts with a positive RT-PCR test for SARS-CoV-2 from the nasopharynx and having moderate to severe illness, not requiring intensive care support or mechanical ventilation, were eligible. All enrolled pts received best supportive care. In the Phase 1 trial focused on safety of administration, a total of 14 pts will receive up to 3 CYNK-001 infusions on Days 1 (1.5e8 cells), 4 (6e8 cells), 7 (6e8 cells). Efficacy was measured by SARS-CoV-2 clearance as measured by RT-PCR testing and clinical measures of improvement, including pulmonary status, and inflammatory marker changes.

### Results

Four of 6 pts treated to date were evaluable at the time of submission. All had multiple medical co-morbidities. Peripheral oxygen saturation (SpO2) ranged between 88-92% on up to 8 L of supplemental oxygen and all had evidence of multilobar pneumonia on chest radiography. Two pts had received no prior therapy for COVID-19. The other 2 pts received remdesivir and dexamethasone, with the 4^{th} pt also receiving convalescent plasma. In all 4 pts, all infusions were well tolerated. In 3 of 4 pts, oxygenation improved after the first infusion of CYNK-001 and radiographic improvement was noted. The 4^{th} pt developed progressive hypoxemia prior to the administration of the first dose of CYNK-001, requiring more than 30 L of supplemental oxygen delivered by facemask to support a SpO2>90%. All 3 doses of CYNK-001 were administered, but oxygen requirements increased. Twelve days after first CYNK-001 dose, the pt declined mechanical ventilation and died of respiratory failure. Attribution to CYNK-001 could not be ruled out. The remaining 3 pts were discharged with an average follow-up of 16 (9-32) days after first infusion.

### Conclusion

In the first study to measure the safety and potential efficacy of CYNK-001 infusions to treat pts with COVID-19 disease, infusions were generally well tolerated with one Grade 5 event of hypoxic respiratory failure. Early efficacy has been seen in 3 of 4 pts with improvement of oxygenation, inflammatory markers, and radiographic findings. Once Phase 1 is completed, the Phase 2 portion of the study will test this approach in a randomized fashion compared to best available therapy to confirm efficacy of this approach.

### CYNK-001-COVID-19: Initial Three Phase 1 Clinical Trial Participants Summary

As per the protocol NCT04365101, the first three participants enrolled in the Phase I portion of CYNK-001-COVID-19 were summarized and the safety data was review by the independent Data Monitoring Committee. A summary of these enrolled subjects, along with their clinical course, is shown in Table 9. All patients initially presented with symptoms consistent with COVID-19 disease and were found to be positive for SARS-CoV-2 in the nasopharynx as evidenced by a positive RT-PCR.

TABLE 9
Summary of Clinical Experience in the First Three Patients Enrolled

| Patient | 1 | 2 | 3 |
|---|---|---|---|
| Protocol Amendment | 3.1 | 3.1 | 4.0 |
| Age | 52 | 43 | 61 |
| Gender | M | M | F |
| Race/Ethnicity | White/Latino | Not reported/Latino | White/Not Latino |
| Co-Morbid Medical Conditions | Diabetes (current), Tachycardia | Hypertension (current), Type 2 | Hyperlipidemia, migraine, anxiety, |
| | (past) | Diabetes Mellitus (current), | depression, obstructive sleep |
| | | Gout (past) | apnea, nausea (all current) |
| Initial Room Air Sa02 (Screening) | 92% | 89% | 91% |
| Final Post treatment Room Air | 100% and 96% | 98% | 96% |
| Sa02 (Post-treatment on infusion | (Day 4 at 30 mins and 4 hrs post- | (Day 7 at 4 hrs post-infusion) | (Day 4 at 4 hrs post-infusion) |
| days) | infusion) | | |
| Prior COVID-19 Therapies | None | None | Remdesivir, Dexamethasone |
| Initial Chest Radiograph Findings | Diffuse pulmonary infiltrates | Diffuse pulmonary infiltrates | Pulmonary infiltrate (unspecified |
| (Screening) | (three lung fields) | (four lung fields) | location) |
| Post-treatment Chest Radiograph | Pulmonary infiltrates | Pulmonary infiltrates | Increased Pulmonary infiltrates |
| Findings (Pre-treatment on | (two lung fields) | (two lung fields) (Day 7) | (unspecified location) |
| infusion days) | (Day 4) | Clear (Day 15) | (Day 7) |
| Clinical Impression/Safety | Improved. Discharged home. | Improved. Discharged home. No | Did not receive the final (Day 7) |
| | Did not return for final (Day 7) dose. | suspected TEAEs. | dose due to adverse event. SAE |
| | Patient withdrew. No suspected TEAEs. | | orthostatic hypotension resulting |
| | | | in prolonged hospitalization. |

| | |
|---|---|
| Safety Conclusion: | There were no treatment-emergent adverse events that were assessed as DLTs nor suspected unexpected serious |
| | adverse events (SUSARs) to date. There were no incidents of cytokine release syndrome, graft-versus-host disease, |
| | neurotoxicity, or infusion-site reactions that were suspected to be related to CYNK-001 in all three subjects. |

The first subject (ID #3070001) is a 52 year old Male, White, Hispanic or Latino with a past medical history of tachycardia and diabetes mellitus. He presented to the University of California-Irvine hospital on Sep. 2, 2020 with symptoms of low-grade fever (37.7° C.), cough, dyspnea, myalgia, headache, fatigue, dizziness, and loss of smell. COVID-19 was confirmed with SARS-CoV-2 detected by rRT-PCR from a nasopharyngeal swab on Sep. 1, 2020. The patient was enrolled in the CYNK-001 study on Sep. 3, 2020 and received two CYNK-001 infusions. At presentation, the subject had a recorded oxygen saturation (SpO2) on room air of 92%, and chest radiographs (CXR) revealed diffuse interstitial infiltrates in three lung fields. Day 1 CYNK-001 infusion was administered on Sep. 3, 2020 (150 million cells). Day 4 infusion was administered on Sep. 8, 2020 (600 million cells). Day 7 infusion was not administered as the patient withdrew from the study. Both study infusions were well tolerated, with an episode of loose stools following the first infusion that was though by local study clinicians to be unrelated to the study drug. Immediately following the Day 4 infusion, the patient's SpO2 had improved to 100%, CXR revealed a resolution of infiltrate in one lung field, and all inflammatory markers (C-reactive protein (CRP), Interleukin-6 (IL-6), D-Dimer, and Ferritin) improved. The clinical impression was one of improvement from admission to discharge.

The second subject (ID #3070002) is a is 43 year old Male, Hispanic or Latino with a medical history of diabetes mellitus, gout, hypertension, loose stool, poor appetite, and nasal congestion all current at baseline. He presented to the University of California-Irvine hospital on Sep. 14, 2020 with symptoms of cough, dyspnea, myalgia, arthralgia, headache, fatigue, and diarrhea. At baseline, a recorded SpO2 of 89% on room air and CXR showed diffuse infiltrates in four lung fields. The patient was enrolled in the CYNK-001 study on Sep. 15, 2020 and received three CYNK-001 infusions. Day 1 CYNK-001 infusion was administered on September 15^{th} (150 million cells). Day 4 infusion was administered on September 18^{th} (600 million cells). Day 7 infusion was administered on September 21^{st} (600 million cells). Two events were considered unrelated to the study drug but were noted by study clinicians: worsening headache and bilateral lymph node pain. Following the Day 7 infusion, the patient's SpO2 had improved to 98%, CXR revealed a resolution of infiltrate in two lung fields, and all inflammatory markers improved. The clinical impression was one of improvement from admission to discharge.

The third subject (ID3020001) is a 61 year old Female, White, Not Hispanic or Latino with a medical history of hyperlipidemia, migraine, anxiety, depression, obstructive sleep apnea, and nausea. Upon presentation, she had a SpO2 of 91% on room air and a CXR with diffuse pulmonary infiltrates. She was hospitalized at Atlantic Health, Morristown site since Oct. 7, 2020 and treated with Remdesivir from October 8-12^{th}, and Dexamethasone from October 8-17. Patient was screened for the study on October 12^{th}, and at that time was requiring 6 L supplemental oxygen by nasal cannula. The patient was enrolled in the CYNK-001 study on October 13^{th} and received two CYNK-001 infusions. Day 1 CYNK-001 infusion was administered on October 13^{th} (150 million cells). Day 4 infusion was administered on October 16^{th} (600 million cells). Following the Day 4 infusion, a serious adverse event (SAE) of Grade 3 orthostatic hypotension (that prolonged the subject's hospitalization) was reported. Because hypotension of grade 3 severity was considered an expected event (noted in previous PNK-007 study), this reported SAE was assessed as not a Suspected Unexpected Serious Adverse Reaction (SUSAR), The Day 7 infusion was not administered due to the SAE. The subject's SpO2 improved to 96% following the Day 4 infusion, but the CXR was noted to have progressive disease. Her CRP and D-dimer both improved from enrollment to Day 4, and there were small increases in IL-6 and Ferritin levels.

In the first three subjects, there were no treatment-emergent adverse events (TEAEs) that were assessed to be Dose-Limiting Toxicities (DLTs) nor Suspected Unexpected Serious Adverse Reactions (SUSARs). The safety of CYNK-001 was deemed favorable by the independent Data Monitoring Committee based on the evaluation of the first three subjects in this study.

### 7.8 Example 8: Human Placental Hematopoietic Stem Cell Derived Natural Killer Cells (CYNK-001) Mediate Protection Against Influenza A Viral Infection

Background: Influenza A virus (IAV) infections are associated with a high healthcare burden around the world and there is an urgent need to develop more effective therapies. Natural killer (NK) cells provide the first line of innate defense against IAV by killing infected epithelial cells, by producing antiviral cytokines and affecting adaptive immunity. Preclinical studies have demonstrated that NK cells play a pivotal role in reducing IAV-induced pulmonary infection; however, little is known about the therapeutic potential of adoptively transferred NK cells for IAV infections. Celularity Inc. is developing human placental hematopoietic stem cell-derived allogeneic, off-the-shelf NK cell therapy (CYNK-001) for the treatment of viral infections, including coronavirus disease of 2019. Here, we report the evaluation of antiviral activities of CYNK-001 against IAV infection.

Methods: In vitro antiviral activities of CYNK-001 were evaluated using human alveolar epithelial cell line A549, infected with IAV strain A/PR/8/34 (H1N1) at variable multiplicity of infection (MOI). The expression of ligands for NK cell receptors was analyzed on infected A549 cells using Fc-coupled recombinant proteins. CYNK-001 was added to A549 cells 16 hours post infection. CYNK-001 degranulation was measured after 4 hours of coculture, and CYNK-001 cytotoxicity against IAV-infected A549 was measured real-time using impedance-based xCELLigence platform. In vivo antiviral and immunomodulatory activities of CYNK-001 were assessed in A/PR/8/34 (H1N1)-induced severe acute lung injury mouse model. Mice were intranasally infected with 2500 PFU IAV. PBS or 1×10⁷ CYNK-001 cells were intravenously administered twice at 1 and 3 days post infection (dpi). At 6 dpi, lungs were collected for the evaluation of viral load by qPCR, lung injury and immune cell profiling by histology. Bronchoalveolar lavage fluid (BALF) was collected at 6 dpi for cytokine analysis by multiplex assays, total protein concentration by ELISA and immune cell profiling by flow cytometry.

Results: In vitro, IAV infection corresponded with dose-dependent expression of ligands to NK cell-activating receptors, including NKp44, NKp46 and NKG2D. CYNK-001 cells exhibited increased IFNγ, TNFα and GM-CSF production, and elevated level of degranulation upon coculture with IAV-infected A549 cells. Cytokines in culture supernatant and CD107a expression in CYNK-001 cells were upregulated in a virus dose-dependent manner. Consistent with this finding, CYNK-001 cytotoxicity against IAV-infected A549 cells increased from 35% at 0 MOI to 50%, 60% and 75% at 0.001, 0.01 and 0.1 MOI, respectively. These data indicate that CYNK-001 cells recognize virally infected cells, resulting in specific cytotoxic elimination of the source of infection. In vivo, treatment of IAV-infected mice with CYNK-001 reduced weight loss and increased their likelihood of survival. PBS control group developed a severe disease and 37.5% mortality was observed as early as day 4. In the group treated with CYNK-001, disease onset was delayed by 2 days. qPCR analysis of viral RNA showed that CYNK-001-treated mice had lower viral load in the lung than vehicle-treated mice, demonstrating antiviral function of CYNK-001 in vivo. CYNK-001-treated mice had reduced lung injury as assessed by lower total protein concentration in BALF. Moreover, CYNK-001 reduced BALF murine cytokines and chemokines, including IFNγ (p<0.001), IL-6, TNFα, MCP-1 (p<0.05), CXCL2 and CXCL9. Lastly, immunohistochemical analysis of the lung showed that CYNK-001-treated mice had an altered immune response to IAV with higher number of CD68⁺ macrophages and CD8⁺ T cells at 6 dpi.

Conclusions: Our in vitro and in vivo data show the promising antiviral activities of CYNK-001 against IAV infection. In a severe IAV infection mouse model, CYNK-001 treatment demonstrates lower mortality rate, lower weight loss, lower lung viral load and reduced lung injury along with reduced inflammation. These results support our hypothesis that the adoptive transfer of CYNK-001 could reduce the burden of viral infection through the elimination of infected epithelial cells, coordinate a more effective immune response, and result in a clinical benefit in patients with severe viral infection.

### 7.9 Example 9: In Vitro Antiviral Effects of CYNK-001

Celularity is developing CYNK-001, previously designated as PNK-007, for the treatment of coronavirus disease 2019 (COVID-19). CYNK-001 is an allogeneic, culture-expanded natural killer (NK) cell population derived from human placental hematopoietic stem cells. CYNK-001 is formulated for intravenous (IV) administration and is currently being studied in three ongoing clinical trials: Phase 1 study under IND 016792 in patients who have relapsed and/or refractory AML, Phase 1/2 study under IND 017030 for multiple myeloma (MM), Phase I study under IND 019486 for glioblastoma multiforme (GBM).

CYNK-001 consists of culture-expanded NK cells which are harvested and washed in Plasma-Lyte A, then packaged at 30×10⁶ cells/mL in a total volume of 20-mL of cryopreservation solution containing 10% (w/v) HSA, 5.5% (w/v) Dextran 40, 0.21% NaCl (w/v), 32% (v/v) Plasma-Lyte A, and 5% (v/v) dimethyl sulfoxide (DMSO). It is filled into the container closure, frozen using a controlled rate freezer, and cryopreserved. When required for administration by a site, CYNK-001 is shipped in vapor phase liquid nitrogen (LN2) to the designated clinical site where it is processed for dose preparation in a standardized manner just prior to IV or intratumoral administration.

CYNK-001 is well characterized with respect to key cellular attributes: identity, morphology, immunophenotype, and functionality. The identity that defines the majority (≥85%) of CYNK-001 cells is CD56⁺ and CD3⁻, as measured by flow cytometry. CYNK-001 cells morphologically appear as large granular lymphocytes, and they are roughly spherical in shape with an average cell diameter of 9.5±0.1 μm. CYNK-001 contains very low to non-detectable levels of CD3⁺ T cells (≤1.0%) or CD19⁺ B cells (≤1.0%), as measured by flow cytometry.

CYNK-001 demonstrates a range of biological activities expected of NK cells, including expression of perforin and granzyme B cytotoxic granules, cytolytic activity against hematological tumor cells lines and GBM solid tumor cell lines, and secretion of immunomodulatory cytokines such as IFN-γ, TNF-α and GM-CSF in the presence of tumors cell lines. These cells express the nominal NK surface phenotype CD3⁻, CD56⁺, CD19⁻ and additionally express activating receptors including NKG2D⁺, NKp46⁺, NKp30⁺ and DNAM-1⁺ (CELU-RES-2019-001, CELU-RES-2019-002, CELU-RES-2019-003).

Severe acute respiratory syndrome coronavirus 2 (SARS-CoV-2) is a highly transmissible and pathogenic coronavirus that emerged in late 2019 and has caused a pandemic of acute respiratory disease, named ‘coronavirus disease 2019’ (COVID-19), which threatens human health and public safety.

Severe acute respiratory syndrome coronavirus 2 (SARS-CoV-2) emerged in Wuhan City, Hubei Province, China in late 2019 (Chen, 2020; Huang, 2020). The highly transmissible and pathogenic virus fast spread around the globe causing a pandemic of acute respiratory disease, also known as coronavirus disease 2019 (COVID-19). The major cause of death is acute respiratory distress syndrome (ARDS) (PMID: 33024307). As of Jan. 21, 2021, there had been 95,612,831 confirmed cases and 2,066,176 deaths globally (World Health Organization). A lack of specific antiviral drugs for SARS-CoV-2 or effective anti-inflammatory treatment for ARDS has resulted in extreme burden for the global health care system and the economy. There is an urgent need for novel medical interventions and clinically effective solutions.

NK cells are innate immune cells with an important role in early host response against various pathogens. Multiple NK cell receptors are involved in the recognition of infected cells, including NKG2D, DNAM-1 and the natural cytotoxicity receptors NKp30, NKp44 and NKp46, which bind common stress ligands or pathogen-associated molecules (Cook, 2014). NK cells kill their target cells by cytotoxic molecules perforin and granzymes, and via death receptor-mediated apoptosis (Loh, 2005). In addition to their cytotoxic functions, NK cells are important for priming adaptive immunity by the secretion of various chemokines and cytokines, including IFN-g (Lanier, 2008).

Studies in humans and mice have established that there is robust activation of NK cells during viral infection, regardless of the virus class (Ivanova, 2014), and that the depletion of or a defect in NK cells aggravates viral pathogenesis (Littwitz, 2013; Bukowski, 1983; Gazit, 2006; Nogusa, 2008; Stein-Streilein, 1986). The important role of NK cells in virus control is illustrated by the diverse mechanisms human viruses, exemplified by CMV, have evolved to evade the NK cell recognition pathways (Lanier, 2008). In murine and human CMV infection, NK cell-mediated anti-viral activity is dependent on IFN-g secretion and perforin-dependent lysis of infected cells (Loh, 2005; Wu, 2015). HIV-1 infection in pregnancy is inhibited by decidual NK cells (Quillay, 2016) and hepatitis C virus infection is controlled by NK cells in the liver (Guidotti, 2006). NK cells have a major role in the early control of lung infections with pathogenic organisms. Timely NK cell-mediated cytotoxicity and IFN-g production limit diverse respiratory bacterial, fungal and viral infections (Ivanova, 2014).

NK cells sense the environment using a broad repertoire of surface receptors that can differentiate between normal and malignant cells (cancerous or infected) by binding to stress ligands and viral antigens. In particular, the stress ligand-induced NKG2D-MICA/B pathway has been shown to be important for NK cell activation and recognition of infected cells in multiple viral infections, including coronaviruses (Walsh, 2008; Lanier, 2008). Various viral glycoproteins expressed by enveloped viruses, including coronaviruses (Zeng, 2008), are specifically recognized by the natural cytotoxicity receptors NKp30, NKp44, and NKp46 (Cook, 2014). NK cell cytolytic activity against Influenza virus is triggered by the recognition of viral haemagglutinin by NKp46 receptor, but also induced by antibody-dependent cell-mediated cytotoxicity (ADCC) (Mandelboim, 2001). In infected tissue microenvironment, NK cell activation leads to increase activating receptor expression and their cytotoxic responses are strongly potentiated by type I IFNs produced by dendritic cells and infected epithelial cells, also enabling subsequent priming and T cell activation and memory (Lanier, 2008).

It was shown that coronavirus infection stimulates the recruitment of NK cells to control infection. Research following the SARS-CoV outbreak revealed that SARS-CoV infection in a mouse model resulted in acute expression of CCL5, CXCL10, and CCL3 chemokines in lung epithelial cells (Law, 2007). In a separate study, NK cells migrated to coronavirus-infected organs in a CXCL10 dependent manner and was associated with reduced coronavirus titers. Anti-viral activity accompanied NK cell homing to the tissue and IFN-g secretion (Trifdo, 2004).

A study of NK cells from peripheral blood of patients with SARS coronavirus (SARS-CoV) was evaluated for the number of NK cells, as it was previously noted that patients with lower NK cells in the HIV population were susceptible to retrovirus resistance. It was noted that patients with SARS coronavirus had significantly lower counts of NK cells in their peripheral blood compared to patients with mycoplasma pneumonia and healthy adults. It was unclear as to why the number was lower. It was hypothesized that either the NK cells had died as a direct attack from the virus or the NK cells were redistributed to targeted organs, such as the lungs (National Research Project of SARS, 2004). Hematological abnormalities such as thrombocytopenia and lymphopenia were common in both SARS-CoV and MERS-CoV patients. Thrombocytopenia and lymphopenia may be predictive of fatal outcome in MERS-CoV patients (Yin, 2018). Based on these observations, it is hypothesized that adoptive NK cell therapy may provide the antiviral activities in those with SARS-CoV-2 infection.

CYNK-001 are human placental hematopoietic stem cell-derived NK cells that express the dominant NK cell marker CD56 and lack lineage markers such as CD3, CD14 and CD19 (FIG. 8). CYNK-001 cells express the NK cell activating receptors NKG2D, DNAM1, NKp30, NKp46, and NKp44 that recognize stressed and virus-infected cells (Walsh, 2008; Lanier, 2008; Zeng, 2008; Cook, 2014) (FIG. 7, FIG. 8 and Table 1).

Regarding the homing to infected tissues, Celularity has shown that CYNK-001 cells have immediate biodistribution in the lungs following intravenous injection in preclinical models (IND 016792, CELU-2018-003; CELU-2019-001). It has been shown that CXCR3 expression on NK cells is involved in NK cell trafficking to the lung in Influenza virus infection (Carlin, 2018; Scharenberg, 2019). CXCR3 is also involved in CXCL10-directed NK cell homing to coronavirus infected tissues (Trifilo, 2004). Single cell RNA sequencing (scRNAseq) demonstrated that CYNK-001 cells highly express CXCR3 transcript (FIG. 13). The data suggest that CYNK cells have the potential to be efficacious and retained in the lungs given the heightened local biodistribution and chemoattraction to CXCL10.

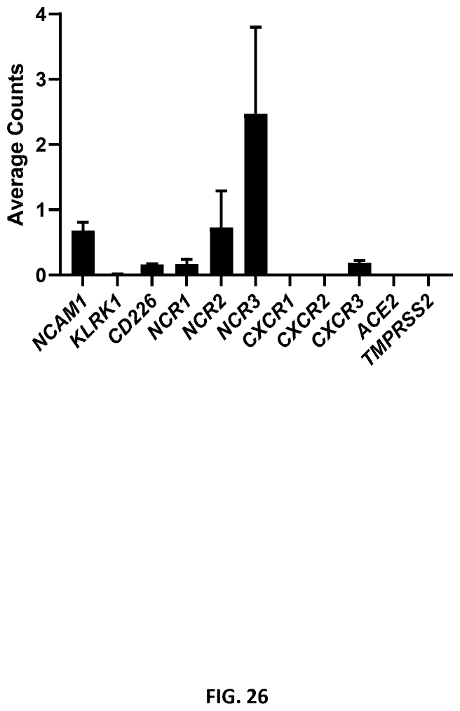

NK cells can become infected with viral pathogens, therefore, either contributing to virus dissemination or resulting in decreased innate immune responses (Mao, 2009). SARS-CoV-2 uses angiotensin-converting enzyme 2 (ACE2) and the cellular protease TMPRSS2 for entry into target cells (Hoffmann, 2020). scRNAseq data demonstrated that CYNK-001 do not express the transcript of either of the SARS-CoV-2 entry proteins, strongly suggesting that CYNK-001 do not get infected by SARS-CoV-2 (FIG. 26).

To investigate the antiviral effect of CYNK-001 cells, we used an in vitro virus infection model on the human lung alveolar basal epithelial cell line A549 cell line. A549 cells are infected with influenza A virus strain A/PR8/8/34 (PR8) and the expression of NK cell activating markers analyzed after 24 h hours. In addition, CYNK-001 cells are added to infected A549 cells to analyze the prototypic NK cell effector functions: degranulation, cytokine production and secretion, and cytotoxicity against virus-infected cells.

Infection of A549 cells with PR8 virus resulted in dose-dependent expression of the viral nucleoprotein (NP) in infected cells (FIG. 15). To analyze whether virus infection induces the expression of NK cell-activating stress ligands on A549 cells, we used recombinant Fc-coupled NK cell receptors to analyze their binding to virus-infected cells (FIG. 15). Of the three receptors, NKp46, NKp44 and NKG2D that are highly expressed on CYNK-001 cells, all demonstrated binding to infected cells, whereas NK cell receptor binding was in direct correlation with virus dose used for A549 infection. NKp44-Fc demonstrated highest binding to infected cells, indicating that PR8 infection induces significant cell-surface expression of NKp44 ligands on A549 cells. We also used antibodies that recognize well-known ligands for NKG2D receptor of the UL 16-binding protein (ULBP) and MHC class I-related chain (MIC) families. ULBP-2/5/6 and MICA/B-recognizing antibodies bound to virus-infected cells in a virus dose-dependent manner. Altogether, the data suggest that ligands that activate NK cells via the common NK cell activating receptors are induced by PR8 virus infection on A549 cells.

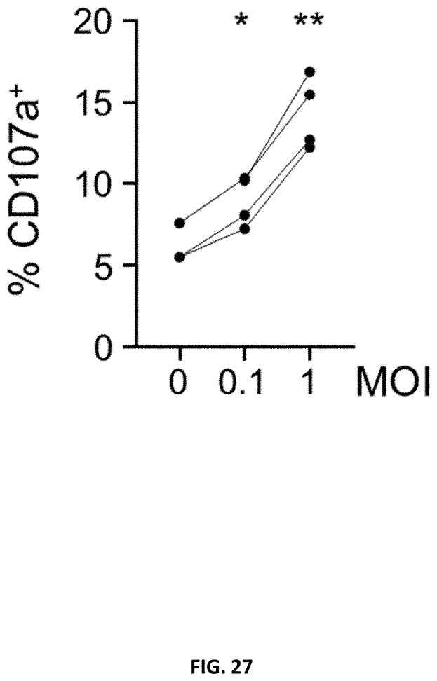

To investigate whether virus infection would activate NK cell effector functions in CYNK-001 cells cultured together with infected A549, we first measured CYNK-001 degranulation by CD107a expression. At steady state, CD107a is expressed in cytotoxic granule membranes and not detectable at high level on the cell membrane, however, upon degranulation is relocated to the cell surface. Co-culture of CYNK-001 with virus-infected A549 cells induced CD107a relocalization to the cell membrane in a virus dose-dependent manner (FIG. 27). The data shows that secretion of cytotoxic granules by CYNK-001 is increased upon contact with virus-induced cells.

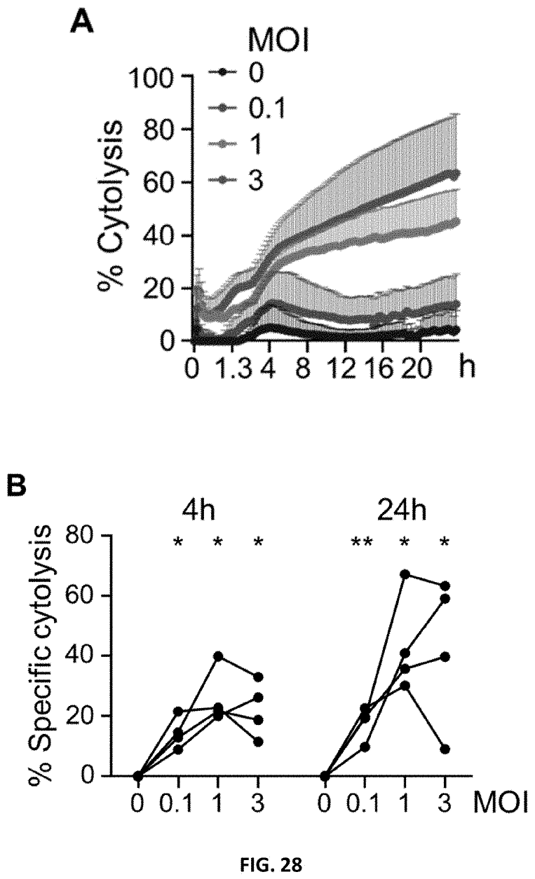

Next, we assessed the lytic functionality of CYNK-001 against virus-infected A549 cells. CYNK-001 cells were co-cultured with virus-infected A549 cells and the cytolytic capacity assessed by a real-time impedance-based assay. CYNK-001 cells lysed virus-infected A549 cells more efficiently compared to non-infected cells (FIGS. 28A-28B). Furthermore, CYNK-001-mediated cytolysis of virus-infected cells was dependent on the virus dose used for A549 infection. These data suggest that higher virus burden results in increased expression of NK cell activating receptor ligands and better recognition of the target cells, resulting in increased cytolysis.

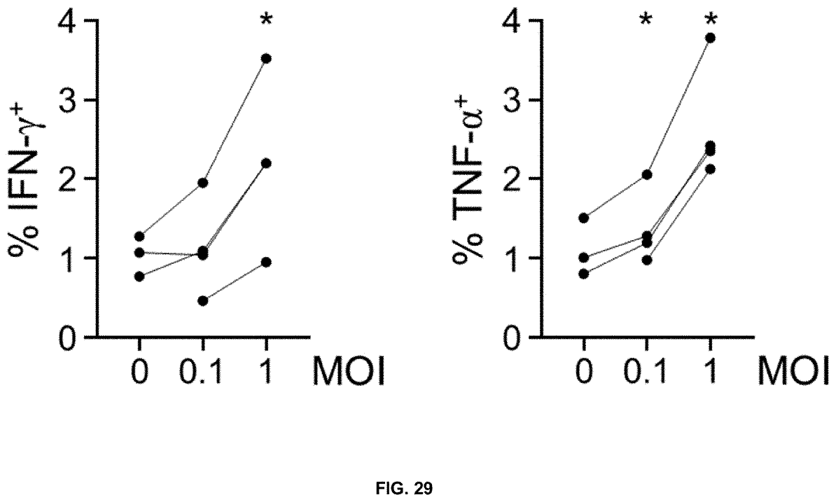

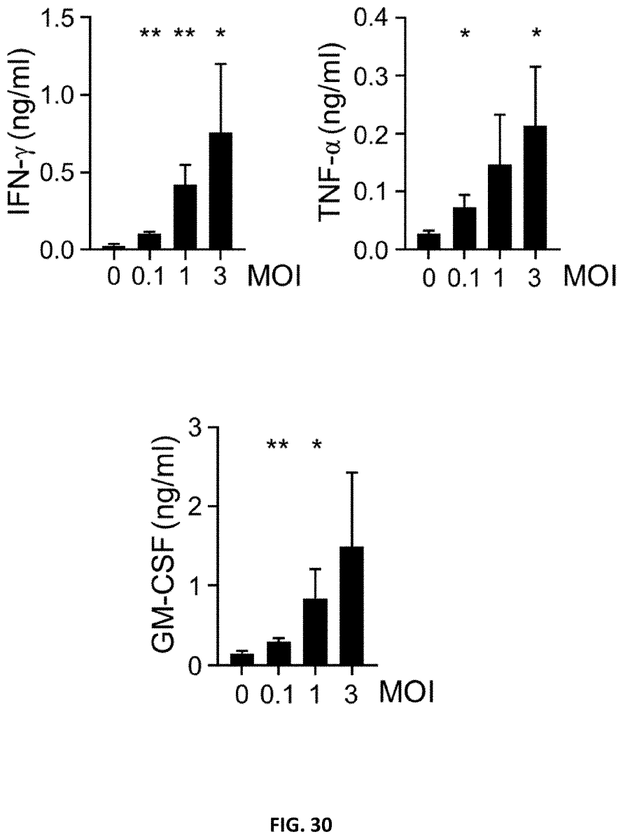

To analyze whether CYNK-001 cells secrete major inflammatory cytokines, such as interferon (IFN)-γ and tumor necrosis factor (TNF)-α that are important for viral or tumor clearance, we stained intracellular cytokines in CYNK-001 cells co-cultured with PR8 virus-infected A549 cells. A low but significant increase in IFN-γ and TNF-α production was detected in CYNK-001 cells that were exposed to virus-infected A549 cells when compared to exposure to non-infected A549 (FIG. 29). Increasing amount of secreted IFN-γ and TNF-α were detected in the supernatants of CYNK-001 co-cultures with virus-infected A549 cells (FIG. 30). We also detected increased secretion of granulocyte-macrophage colony-stimulating factor (GM-CSF) from CYNK-001 cells that were exposed to infected A549 cells.

Altogether, our data demonstrate that virus infection induces the expression of NK cell-activating ligands in epithelial cells that are recognized by CYNK-001 cells, resulting in lysis of the infected cells and the production of pro-inflammatory cytokines by CYNK-001.

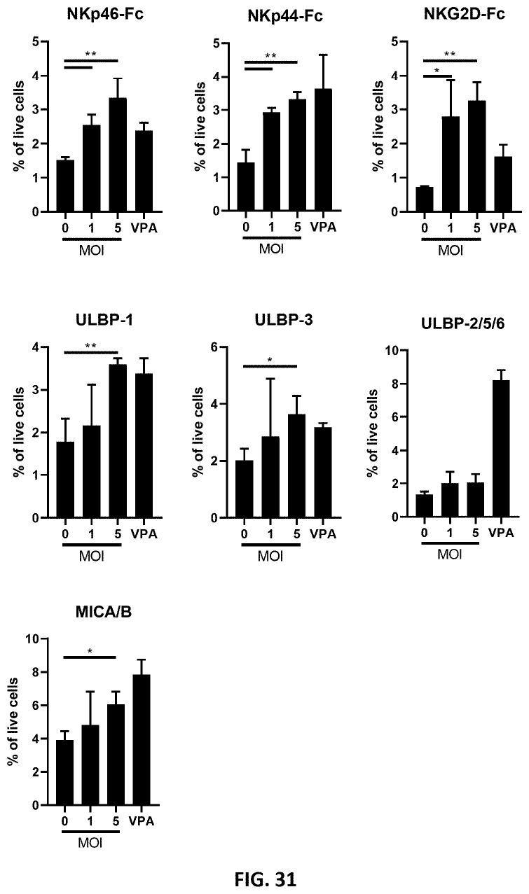

To understand whether CYNK-001 could recognize SARS-CoV-2-infected cells similarly to influenza virus-infected cells, we analyzed NK cell ligand expression on SARS-CoV-2-infected Calu-3 cells. Increased binding of recombinant NK cell receptors NKG2D, NKp44 and NKp46 was seen to SARS-CoV-2-infected cells (FIG. 31). Analysis of individual NKG2D ligands on SARS-CoV-2-infected Calu-3 cells demonstrated higher expression of ULBP-1, ULBP-3 and MICA/B. These data suggest that SARS-CoV-2 infection of epithelial cells induces cell stress and increased expression of ligands that can be recognized by NK cells, similarly to influenza virus.

### Materials and Methods

Placenta CD34⁺ Cell Isolation and CYNK-001 Culture: Placental CD34⁺ cells were acquired from healthy donors under fully-informed consent. With donor eligibility documentation, they were qualified using a series of tests including serology and bacteriology (Lifebank USA). Blood was isolated from healthy donor tissues and processed by red blood cell depletion using Hetastarch (Hospira). The resulting cells were then magnetically labeled using Direct CD34 Progenitor Cell Isolation Kit (Miltenyi Biotec). CD34⁺ cells were positively selected using AutoMACS Cell Separator following manufacture's protocol. Placenta CD34⁺ cells were then cryopreserved in CryoStor® CS10 (Biolife Solutions) and stored in liquid nitrogen tank before use.

For CYNK-001 culture, placental CD34⁺ cells were thawed and cultivated following a 3-stage process in the presence of cytokines including thrombopoietin, SCF, Flt3 ligand, IL-7, IL-15 and IL-2 (Thermo Fisher Scientific) for 35 days to generate CYNK-001 cells. Nucleofection of CRISPR reagents was performed at day 5 of culture. Cell count and passage were performed every 2-3 days and cell expansion was recorded. At the end of the culture, cell phenotype was evaluated by flow cytometry to confirm that the cells expressed typical NK receptors and cytolytic markers.

Single cell RNA sequencing: Frozen CYNK-001 cells (donor IDs:) were processed for capture on the 10× Genomics Chromium platform (10× Genomics, Pleasanton, Calif., USA) by Genewiz, LLC (South Plainfield, N.J., US). Custom single-cell RNA-seq library was prepared using the 10× Genomics® Chromium™ (10× Genomics, Pleasanton, Calif., USA). Sequencing was performed on Illumina HiSeq. Differential gene expression analysis was performed using 10× Genomics® Cell Ranger™ single-cell RNA-seq pipeline and data analyzed using Loupe Cell Browser.

Immunophenotypic characterization of CYNK-001: The phenotype of CYNK-001 cells was analyzed by multi-color flow cytometry. First, CYNK-001 cells were washed and dead cells were stained using the Live/Dead Fixable Aqua Stain (Thermo Fisher Scientific), followed by fluorochrome-conjugated antibodies diluted in staining buffer (10% fetal bovine serum (FBS) in phosphate buffered saline (PBS)) according to manufacturer's instructions. The following antibodies were used: CD56 (clone: NCAM16.2)—Pe-Cy7 (BD Biosciences), CD3 (SK7)—APC-H7 (BD Biosciences), CD14 (McpP9)—APC-H7 (BD Biosciences), CD19 (clone: HIB19)—APC-Cy7 (BD Biosciences), CD226 (DNAM-1) (clone: DX11)—PE (Miltenyi Biotec), CD314 (NKG2D) (clone: 1D11)—APC (Miltenyi Biotec), NKp46 (CD335) (clone: 9E2)—BV650 (BD Biosciences), CD336 (NKp44) (clone: p44-8)—BUV395 (BD Biosciences), CD337 (NKp30) (clone: p30-15)—BV421 (BD Biosciences). Stained cells were acquired on BD Fortessa X20 flow cytometer (BD Biosciences) and data was analyzed using FlowJo software.

Influenza Virus Infection of A549 cells: Human lung A549 cells (CCL-185, ATCC) were cultured in Dulbecco's modified MEM (DMEM) supplemented with 10% FBS, penicillin-streptomycin (Thermo Fisher Scientific). Approximately 75% confluent A549 cell cultures were washed twice with PBS and infected with influenza A virus strain A/PR8/8/34 (PR8)(ATCC) diluted in OptiMEM media (Gibco) at the indicated dose (multiplicity of infection—MOI) over 1 h at room temperature. Virus inoculum was removed and replaced with TPCK trypsin (MilliporeSigma)-containing DMEM media.

NK cell ligand analysis on A549 cells: NK cell ligand expression on PR8-infected A549 cells was analyzed 24 h post infection using flow cytometry. In brief, A549 cells were detached using TrypLE reagent (Gibco), washed with fully complemented media and dead cells were stained using the Live/Dead Fixable Aqua Stain (Thermo Fisher Scientific), followed by fluorochrome-conjugated antibodies ULBP2/5/6 (clone: 165903)—PE (R&D Systems) and MICA/B (clone: 6D4)—PE (BioLegend) or recombinant Fc-coupled NK cell receptors NKp46-Fc, NKp44-Fc and NKG2D-Fc (all from R&D Systems) diluted in staining buffer (10% fetal bovine serum (FBS) in PBS). Recombinant NK cell receptors were detected with a secondary anti-human IgG (clone: 97924)—PE (R&D Systems). Intracellular viral nucleoprotein (NP) was stained with anti-NP (clone: D67J)—FITC (Thermo Fisher Scientific) after cell permeabilization using the Fixation/Permeabilization Solution Kit (BD Biosciences) according to manufacturer's recommendations.

CYNK-001 Degranulation Assay and Cytokine Production: Cryopreserved CYNK-001 cells were recovered in IL-15-containing medium for two days, washed with PBS and resuspended in assay buffer (RPMI-1640 (Gibco) medium containing 10% FBS). 24 h post infection media was removed from PR8-infected A549 cells cultured in a 96-well plate and CYNK-001 were added to A549 cells at an effector-to-target ratio (E:T) of 1:1 in the presence of anti-CD107a (clone: H4A3)—BV786 (BD Pharmingen). Monensin at a final concentration of 10 μM was added after 1 h of co-culture. After a total of 5 h of co-culture, cells were collected, stained using the Live/Dead Fixable Aqua Stain (Thermo Fisher Scientific) and CD56 (clone: NCAM16.2)—Pe-Cy7 (BD Biosciences), CD3 (SK7)—APC-H7 (BD Biosciences), CD14 (McpP9)—APC-H7 (BD Biosciences), CD19 (clone: HIB19)—APC-Cy7 (BD Biosciences) diluted in staining buffer according to the manufacturer's instructions. Intracellular IFN-γ (clone: B27)—APC (BD Biosciences) and TNF-α (clone: MAb11)—BV421 (BD Biosciences) were stained after fixation and permeabilization using the Fixation/Permeabilization Solution Kit (BD Biosciences) according to manufacturer's recommendations. The expression of CD107a, IFN-γ and TNF-α was analyzed on live single cells negative for lineage markers (CD3, CD14, CD19) and expressing CD56 by flow cytometry.

Multiplex Assay: Cytokines in cell culture supernatants were measured using the Luminex FlexMAP3D platform (EMD Millipore) using a custom-made Milliplex 7-plex magnetic bead kit according to Manufacturer's instructions.

In Vitro Cytotoxicity Assay: Cytotoxicity against the A549 cell line was measured using the xCELLigence RTCA platform (ACEA Bioscience). A549 were cultured in 96-well electronic microtiter plates overnight, followed by infection with PR8 virus at the indicated MOI. 24 h post infection, CYNK-001 cells were added at an E:T ratio of 2.5:1. Cell index, indicating the impedance of electron flow caused by adherent cells, was recorded real-time over 24 h. Percentage of cytolysis was calculated using the formula provided by ACEA Bioscience: (Cell Index of no effector−Cell Index of effector)/Cell Index of no effector×100. Specific cytolysis was calculated by subtracting CYNK-001 cell cytolysis on non-infected cells (MOI 0) from cytolysis values on infected cells.

NK cell ligand analysis on SARS-CoV-2-infected Calu-3 cells: Human lung cell line Calu-3 (HTB-55, ATCC) were cultured in Eagle's modified MEM (EMEM) supplemented with 10% FBS and penicillin-streptomycin (Thermo Fisher Scientific). Calu-3 cells were infected upon confluency with SARS-CoV-2 at an MOI of 1 and 5. 24 h post infection Calu-3 cells were collected using TrypLE reagent (Gibco). The cells were washed with fully complemented media and dead cells were stained using the Live/Dead Fixable Aqua Stain (Thermo Fisher Scientific), followed by fluorochrome-conjugated antibodies ULBP2/5/6 (clone: 165903)—PE (R&D Systems), ULBP-3 (clone: 166510)—PE (R&D Systems), ULBP-1 (clone: 170818)—PE (R&D Systems) and MICA/B (clone: 6D4)—PE (BioLegend) or recombinant Fc-coupled NK cell receptors NKp46-Fc, NKp44-Fc and NKG2D-Fc (all from R&D Systems) diluted in staining buffer (10% fetal bovine serum (FBS) in PBS). Recombinant NK cell receptors were detected with a secondary anti-human IgG (clone: 97924)—PE (R&D Systems). Cells were fixed with 4% paraformaldehyde (PFA) before analysis on a flow cytometer.

### REFERENCES

Bukowski J F, Woda B A, Habu S, Okumura K, Welsh R M. Natural killer cell depletion enhances virus synthesis and virus-induced hepatitis in vivo. J Immunol. 1983; 131(3):1531-1538.
Carlin L E, Hemann E A, Zacharias Z R, Heusel J W, Legge K L. Natural Killer Cell Recruitment to the Lung During Influenza A Virus Infection Is Dependent on CXCR3, CCR5, and Virus Exposure Dose. Front Immunol, 2018; 9:781. Published 2018 Apr. 17. doi: 10.3389/fimmu.2018.00781
Chen N, Zhou M, Dong X, Qu J, Gong F, Han Y, et al. Epidemiological and clinical characteristics of 99 cases of 2019 novel coronavirus pneumonia in Wuhan, China: a descriptive study. Lancet, 2020 Feb. 15; 395(10223):507-513.
Cook, K. D., S. N. Waggoner, and J. K. Whitmire, NK cells and their ability to modulate T cells during virus infections. Crit Rev Immunol, 2014. 34(5): p. 359-88.
Gazit R, Gruda R, Elboim M, et al. Lethal influenza infection in the absence of the natural killer cell receptor gene Ncr1. Nat Immunol, 2006; 7(5):517-523. doi: 10.1038/ni 1322
Guidotti L G, Chisari F V. Immunobiology and pathogenesis of viral hepatitis. Annu Rev Pathol. 2006; 1:23-61.
Hoffmann M, Kleine-Weber H, Kruger N, Muller M, Drosten C, Pohlmann S. The novel coronavirus 2019 (2019-nCoV) uses the SARS-coronavirus receptor ACE2 and the cellular protease TMPRSS2 for entry into target cells. bioRxiv preprint doi: https://doi.org/10.1101/2020.01.31.929042.
Huang C, Wang Y, Li X, Ren L, Zhao J, Hu Y, et al. Clinical features of patients infected with 2019 novel coronavirus in Wuhan, China. Lancet, 2020 Feb. 15; 395(10223):497-506.
Ivanova D, Krempels R, Ryfe J, Weitzman K, Stephenson D, Gigley J P. NK cells in mucosal defense against infection. Biomed Res Int, 2014; 2014:413982.
Landau G, Greenbaum E, Zakay-Rones Z, Porgador A, Mandelboim O. Lethal influenza infection in the absence of the natural killer cell receptor gene Ncr1. Nat Immunol, 2006 May; 7(5):517-23.
Lanier, L. L., Evolutionary struggles between NK cells and viruses. Nat Rev Immunol, 2008. 8(4): p. 259-68.
Law A H, Lee D C, Cheung B K, Yim H C, Lau A S. Role for nonstructural protein 1 of severe acute respiratory syndrome coronavirus in chemokine dysregulation. J Virol, 2007 January; 81(1): 416-22.
Littwitz E, Francois S, Dittmer U, Gibbert K. Distinct roles of NK cells in viral immunity during different phases of acute Friend retrovirus infection. Retrovirology, 2013 Nov. 1; 10:127. doi: 10.1186/1742-4690-10-127. PubMed PMID: 24182203; PubMed Central PMCID: PMC3826539.
Loh J, Chu D T, O'Guin A K, Yokoyama W M, Virgin H W 4th. Natural killer cells utilize both perforin and gamma interferon to regulate murine cytomegalovirus infection in the spleen and liver. J Virol, 2005 January; 79(1):661-7.
Mandelboim O, Lieberman N, Lev M, Paul L, Arnon T I, Bushkin Y, et al. Recognition of haemagglutinins on virus-infected cells by NKp46 activates lysis by human NK cells. Nature. 2001 Feb. 22; 409(6823): 1055-60.
Mao H, Tu W, Qin G, et al. Influenza virus directly infects human natural killer cells and induces cell apoptosis. J Virol, 2009; 83(18):9215-9222. doi:10.1128/JVI.00805-09
Nogusa S, Ritz B W, Kassim S H, Jennings S R, Gardner E M. Characterization of age-related changes in natural killer cells during primary influenza infection in mice. Mech Ageing Dev. 2008; 129(4):223-230. doi:10.1016/j.mad.2008.01.003
Quillay H, El Costa H, Duriez M, Marlin R, Cannou C, Madec Y, et al. NK cells control HIV-1 infection of macrophages through soluble factors and cellular contacts in the human decidua. Retrovirology, 2016 Jun. 6; 13(1):39.
Scharenberg M, Vangeti S, Kekäläinen E, et al. Influenza A Virus Infection Induces Hyperresponsiveness in Human Lung Tissue-Resident and Peripheral Blood NK Cells. Front Immunol, 2019; 10:1116. Published 2019 May 17. doi:10.3389/fimmu.2019.01116
Stein-Streilein J, Guffee J. In vivo treatment of mice and hamsters with antibodies to asialo GM1 increases morbidity and mortality to pulmonary influenza infection. J Immunol. 1986 Feb. 15; 136(4): 1435-41.
Trifdo M J, Montalto-Morrison C, Stiles L N, Hurst K R, Hardison J L, Manning J E, et al. CXC chemokine ligand 10 controls viral infection in the central nervous system: evidence for a role in innate immune response through recruitment and activation of natural killer cells. J Virol, 2004 January; 78(2):585-94.
Walsh K B, Lodoen M B, Edwards R_{A}, Lanier L L, Lane T E. Evidence for differential roles for NKG2D receptor signaling in innate host defense against coronavirus-induced neurological and liver disease. J Virol, 2008; 82(6):3021-3030. doi:10.1128/JVI.02032-07
Wang C, Horby P W, Hayden F G, Gao G F. A novel coronavirus outbreak of global health concern. Lancet, 2020 Feb. 15; 395(10223):470-473.
Wrapp D, Wang N, Corbett K S, Goldsmith J A, Hsieh C L, Abiona O, et al. Cryo-EM structure of the 2019-nCoV spike in the prefusion conformation. Science, 2020 Feb. 19. pii: eabb2507. doi: 10.1126/science.abb2507. [Epub ahead of print] PubMedPMID: 32075877.
Wu Z, Sinzger C, Reichel J J, Just M, Mertens T. Natural killer cells can inhibit the transmission of human cytomegalovirus in cell culture by using mechanisms from innate and adaptive immune responses. J Virol, 2015 March; 89(5):2906-17. doi: 10.1128/JVI.03489-14.
Yin Y, Wunderink R G. MERS, SARS and other coronaviruses as causes of pneumonia. Respirology, 2018 February; 23(2): 130-137.
Zeng Q, Langereis M A, van Vliet A L, Huizinga E G, de Groot R J. Structure of coronavirus hemagglutinin-esterase offers insight into corona and influenza virus evolution. Proc Natl Acad Sci USA, 2008 Jul. 1; 105(26):9065-9.

All references cited herein are incorporated herein by reference in their entirety and for all purposes to the same extent as if each individual publication, patent or patent application was specifically and individually indicated to be incorporated by reference in its entirety for all purposes. The citation of any publication is for its disclosure prior to the filing date and should not be construed as an admission that the present invention is not entitled to antedate such publication by virtue of prior invention.

What is claimed is:

1. A method of treating a viral infection in a subject, comprising administering to the subject an amount of a composition comprising a plurality of placenta derived natural killer cells, effective to treat the viral infection in the subject.

2. The method of claim 1, wherein said administration is intravenous.

3. The method of claim 1, wherein said administration is by bronchiolar lavage or whole lung lavage.

4. The method of any of claims 1-3, wherein said natural killer cells have been cryopreserved prior to said administering.

5. The method of any one of claims 1-4, wherein said subject is administered about 1×10⁴, 3×10⁴, 1×10⁵, 3×10⁵, 1×10⁶, 3×10⁶, 1×10⁷, 3×10⁷, 1×10⁸, or 3×10⁸ natural killer cells per kilogram of the subject.

6. The method of any one of claims 1-5, wherein the treatment comprises administration of more than one dose of the cell population comprising human placenta-derived natural killer cells.

7. The method of claim 6, wherein the treatment comprises administration of two, three, four, or more doses of the cell population comprising human placenta-derived natural killer cells.

8. The method of any one of claims 1-7, wherein the subject is a mammal.

9. The method of any one of claims 1-8, wherein the subject is a human.

10. The method of any one of claims 1-9, wherein the treating further comprises administering to the subject an effective amount of an additional anti-viral treatment.

11. The method of any of claims 1-10, wherein said composition comprises a population of cells that comprise at least 20% CD56+CD3− natural killer cells.

12. The method of any of claims 1-10, wherein said composition comprises a population of cells that comprise at least 40% CD56+CD3− natural killer cells.

13. The method of any of claims 1-10, wherein said composition comprises a population of cells that comprise at least 60% CD56+CD3− natural killer cells.

14. The method of any of claims 1-10, wherein said composition comprises a population of cells that comprise at least 80% CD56+CD3− natural killer cells.

15. The method of any of claims 1-14, wherein said placenta derived natural killer cells are human placenta derived natural killer cells.

16. The method of any of claims 1-15, wherein said placenta derived natural killer cells are hematopoietic stem cell-derived natural killer cells.

17. The method of any of claims 1-15, wherein said placenta derived natural killer cells are CD34+ hematopoietic stem cell-derived natural killer cells.

18. The method of any of claims 1-17, wherein said placenta derived natural killer cells are characterized by expression of one or more markers selected from the group consisting of FGFBP2, GZMH, CCL3L3, GZMM, CXCR4, ZEB2, KLF2, LITAF, RORA, LYAR, CNOT1, IFNG, DUSP2, ATG2A, CD7, PMAIP1, PPP2R5C, NR4A2, ZFP36L2, PIK3R1, KLRF1, SNHG9, MT2A, RGS2, CHD1, DUSP1, EML4, ZFP36, ZC3H12A, DNAJB6, SBDS, IRF1, TSC22D3, TSPYL2, PNRC1, ISCA1, JUNB, WHAMM, RICTOR, TNFAIP3, EPC1, MVD, CLK1, ARL4C, REL, KMT2E, YPEL5, AMD1, BTG2, and IDS which is lower than expression of said markers in peripheral blood natural killer cells and/or expression of one or more markers selected from the group consisting of NDFIP2, LINC00996, MAL, CCL1, MB, SPINK2, C15orf48, CAMK1, KLRC1, TNFSF10, TNFRSF18, IL32, CAPG, AC092580.4, S100A11, TNFRSF4, ENO1, FCER1G, CCND2, KRT81, MRPS6, ANXA2, PTGER2, GLO1, HAVCR2, PYCARD, LAT2, SLC16A3, COTL1, PKM, TALDO1, CD96, NCR3, KRT86, STMN1, LTB, ARPC1B, ARPC5, FKBP1A, TIMP1, GZMK, CD59, PGK1, RGS10, EVL, RAC2, LGALS1, ITGB7, TUBB, PGAM1, PRF1, GZMB, IL2RB, KLRC2, and KLRB1 which is higher than expression of said markers in peripheral blood natural killer cells.

19. The method of any of claims 1-17, wherein said placenta derived natural killer cells are characterized by expression of one or more markers selected from the group consisting of FGFBP2, GZMH, CCL3L3, GZMM, CXCR4, ZEB2, KLF2, LITAF, RORA, LYAR, CNOT1, IFNG, DUSP2, ATG2A, CD7, PMAIP1, PPP2R5C, NR4A2, ZFP36L2, PIK3R1, KLRF1, SNHG9, MT2A, RGS2, CHD1, DUSP1, EML4, ZFP36, ZC3H12A, DNAJB6, SBDS, IRF1, TSC22D3, TSPYL2, PNRC1, ISCA1, JUNB, WHAMM, RICTOR, TNFAIP3, EPC1, MVD, CLK1, ARL4C, REL, KMT2E, YPEL5, AMD1, BTG2, and IDS which is lower than expression of said markers in peripheral blood natural killer cells.

20. The method of any of claims 1-17, wherein said placenta derived natural killer cells are characterized by expression of 2, 3, 4, 5, 6, 7, 8, 9, 10, or more markers selected from the group consisting of FGFBP2, GZMH, CCL3L3, GZMM, CXCR4, ZEB2, KLF2, LITAF, RORA, LYAR, CNOT1, IFNG, DUSP2, ATG2A, CD7, PMAIP1, PPP2R5C, NR4A2, ZFP36L2, PIK3R1, KLRF1, SNHG9, MT2A, RGS2, CHD1, DUSP1, EML4, ZFP36, ZC3H12A, DNAJB6, SBDS, IRF1, TSC22D3, TSPYL2, PNRC1, ISCA1, JUNB, WHAMM, RICTOR, TNFAIP3, EPC1, MVD, CLK1, ARL4C, REL, KMT2E, YPEL5, AMD1, BTG2, and IDS is lower than expression of said markers in peripheral blood natural killer cells.

21. The method of any of claims 1-17, wherein said placenta derived natural killer cells are characterized by expression of one or more markers selected from the group consisting of NDFIP2, LINC00996, MAL, CCL1, MB, SPINK2, C15orf48, CAMK1, KLRC1, TNFSF10, TNFRSF18, IL32, CAPG, AC092580.4, S100A11, TNFRSF4, ENO1, FCER1G, CCND2, KRT81, MRPS6, ANXA2, PTGER2, GLO1, HAVCR2, PYCARD, LAT2, SLC16A3, COTL1, PKM, TALDO1, CD96, NCR3, KRT86, STMN1, LTB, ARPC1B, ARPC5, FKBP1A, TIMP1, GZMK, CD59, PGK1, RGS10, EVL, RAC2, LGALS1, ITGB7, TUBB, PGAM1, PRF1, GZMB, IL2RB, KLRC2, and KLRB1 which is higher than expression of said markers in peripheral blood natural killer cells.

22. The method of any of claims 1-17, wherein said placenta derived natural killer cells are characterized by expression of 2, 3, 4, 5, 6, 7, 8, 9, 10, or more markers selected from the group consisting of NDFIP2, LINC00996, MAL, CCL1, MB, SPINK2, C15orf48, CAMK1, KLRC1, TNFSF10, TNFRSF18, IL32, CAPG, AC092580.4, S100A11, TNFRSF4, ENO1, FCER1G, CCND2, KRT81, MRPS6, ANXA2, PTGER2, GLO1, HAVCR2, PYCARD, LAT2, SLC16A3, COTL1, PKM, TALDO1, CD96, NCR3, KRT86, STMN1, LTB, ARPC1B, ARPC5, FKBP1A, TEMPI, GZMK, CD59, PGK1, RGS10, EVL, RAC2, LGALS1, ITGB7, TUBB, PGAM1, PRF1, GZMB, IL2RB, KLRC2, and KLRB1 is higher than expression of said markers in peripheral blood natural killer cells.

23. The method of any one of claims 1-22, wherein said human placenta derived natural killer cells are CYNK cells.

24. The method of any one of claims 1-22, wherein said viral infection is a coronavirus infection.

25. The method of claim 24, wherein said coronavirus infection is selected from the group consisting of human coronavirus 229E (HCoV-229E), human coronavirus OC43 (HCoV-OC43), SARS-CoV, human coronavirus NL63 (HCoV-NL63, New Haven coronavirus), human coronavirus HKU1, middle east respiratory syndrome coronavirus (MERS-CoV, novel coronavirus 2012, HCoV-EMC), and novel coronavirus 2019-nCoV (SARS-CoV-2).

26. The method of claim 24, wherein said coronavirus infection is SARS-CoV-2.

27. The method of any one of claims 1-26, wherein the treatment comprises an improvement in score as measured by the Ordinal Scale for Clinical Improvement (OSCI).

28. The method of any one of claims 1-26, wherein the treatment comprises a reduction in the time to improvement in score as measured by the Ordinal Scale for Clinical Improvement (OSCI).

29. The method of any one of claims 1-26, wherein the treatment comprises an improvement in stratus by OSCI.

30. The method of any one of claims 1-26, wherein the treatment comprises an improvement in time to and/or rate of clinical improvement by NEWS2 Score.

31. The method of any one of claims 1-26, wherein the treatment comprises medical discharge or a reduced time to medical discharge.

32. The method of any one of claims 1-26, wherein the treatment comprises reduced hospital utilization.

33. The method of any one of claims 1-26, wherein the treatment comprises reduced mortality.

34. The method of any one of claims 1-26, wherein the treatment comprises clearance of the virus or reduced time to clearance of the virus.

35. The method of any one of claims 1-26, wherein the treatment comprises improved time to and/or rate of pulmonary clearance.

36. The method of any one of claims 1-26, wherein the treatment comprises reduced duration of hospitalization.

37. The method of any one of claims 1-26, wherein the treatment comprises an increase in supplemental oxygen-free days, a reduced need for supplemental oxygen, or a reduced time to cessation of supplemental oxygen.

38. The method of any one of claims 1-26, wherein the treatment comprises a reduction in the requirement for ventilation.

39. The method of any one of claims 1-26, wherein the treatment comprises an improvement in SOFA score.

40. The method of any one of claims 1-26, wherein the treatment comprises an improvement in radiologic evaluation score.

41. The method of any one of claims 1-26, wherein the treatment comprises an improvement in cytokine and/or chemokine assessment, preferably wherein the improvement in cytokine and/or chemokine assessment comprises a reduction in one or more inflammatory markers.

42. The method of any one of claims 1-26, wherein the treatment comprises reduced or eliminated viral detection by RT-PCR.

43. The method of any one of claims 1-42, wherein the treatment comprises two or more doses of natural killer cells.

44. The method of any one of claims 1-42, wherein the treatment comprises a first dose and one or more subsequent doses of natural killer cells.

45. The method of any one of claims 1-42, wherein the treatment comprises a first dose of between about 50×10⁶ natural killer cells to about and about 600×10⁶ natural killer cells.

46. The method of any one of claims 1-42, wherein the treatment comprises a first dose and one or more subsequent doses of natural killer cells, wherein the one or more subsequent doses comprise about 150×10⁶ natural killer cells to about and about 2400×10⁶ natural killer cells.

47. The method of any one of claims 1-42, wherein the treatment comprises a first dose and one or more subsequent doses of natural killer cells, wherein the one or more subsequent doses of natural killer cells are administered from about one to about five days after the previous doses, preferably wherein the one or more subsequent doses of natural killer cells are administered about three days after the previous dose.

48. The method of any one of claims 1-42, wherein the treatment comprises an initial dose of 150×10⁶ cells on Day 1 followed by 600×10⁶ cells IV Days 4 and 7 or an initial dose of 150×10⁶ cells on Day 1 followed by 600×10⁶ cells IV Day 7.

49. A natural killer cell characterized by expression of one or more markers selected from the group consisting of FGFBP2, GZMH, CCL3L3, GZMM, CXCR4, ZEB2, KLF2, LITAF, RORA, LYAR, CNOT1, IFNG, DUSP2, ATG2A, CD7, PMAIP1, PPP2R5C, NR4A2, ZFP36L2, PIK3R1, KLRF1, SNHG9, MT2A, RGS2, CHD1, DUSP1, EML4, ZFP36, ZC3H12A, DNAJB6, SBDS, IRF1, TSC22D3, TSPYL2, PNRC1, ISCA1, JUNB, WHAMM, RICTOR, TNFAIP3, EPC1, MVD, CLK1, ARL4C, REL, KMT2E, YPEL5, AMD1, BTG2, and IDS which is lower than expression of said markers in peripheral blood natural killer cells and/or expression of one or more markers selected from the group consisting of NDFIP2, LINC00996, MAL, CCL1, MB, SPINK2, C15orf48, CAMK1, KLRC1, TNFSF10, TNFRSF18, IL32, CAPG, AC092580.4, S100A11, TNFRSF4, ENO1, FCER1G, CCND2, KRT81, MRPS6, ANXA2, PTGER2, GLO1, HAVCR2, PYCARD, LAT2, SLC16A3, COTL1, PKM, TALDO1, CD96, NCR3, KRT86, STMN1, LTB, ARPC1B, ARPC5, FKBP1A, TIMP1, GZMK, CD59, PGK1, RGS10, EVL, RAC2, LGALS1, ITGB7, TUBB, PGAM1, PRF1, GZMB, IL2RB, KLRC2, and KLRB1 which is higher than expression of said markers in peripheral blood natural killer cells for use in treating a viral infection.

50. The natural killer of claim 49, characterized by expression of one or more markers selected from the group consisting of FGFBP2, GZMH, CCL3L3, GZMM, CXCR4, ZEB2, KLF2, LITAF, RORA, LYAR, CNOT1, IFNG, DUSP2, ATG2A, CD7, PMAIP1, PPP2R5C, NR4A2, ZFP36L2, PIK3R1, KLRF1, SNHG9, MT2A, RGS2, CHD1, DUSP1, EML4, ZFP36, ZC3H12A, DNAJB6, SBDS, IRF1, TSC22D3, TSPYL2, PNRC1, ISCA1, JUNB, WHAMM, RICTOR, TNFAIP3, EPC1, MVD, CLK1, ARL4C, REL, KMT2E, YPEL5, AMD1, BTG2, and IDS which is lower than expression of said markers in peripheral blood natural killer cells.

51. The natural killer cell of claim 49 or claim 50, wherein expression of 2, 3, 4, 5, 6, 7, 8, 9, 10, or more markers selected from the group consisting of FGFBP2, GZMH, CCL3L3, GZMM, CXCR4, ZEB2, KLF2, LITAF, RORA, LYAR, CNOT1, IFNG, DUSP2, ATG2A, CD7, PMAIP1, PPP2R5C, NR4A2, ZFP36L2, PIK3R1, KLRF1, SNHG9, MT2A, RGS2, CHD1, DUSP1, EML4, ZFP36, ZC3H12A, DNAJB6, SBDS, IRF1, TSC22D3, TSPYL2, PNRC1, ISCA1, JUNB, WHAMM, RICTOR, TNFAIP3, EPC1, MVD, CLK1, ARL4C, REL, KMT2E, YPEL5, AMD1, BTG2, and IDS is lower than expression of said markers in peripheral blood natural killer cells.

52. The natural killer cell of claim 49, characterized by expression of one or more markers selected from the group consisting of NDFIP2, LINC00996, MAL, CCL1, MB, SPINK2, C15orf48, CAMK1, KLRC1, TNFSF10, TNFRSF18, IL32, CAPG, AC092580.4, S100A11, TNFRSF4, ENO1, FCER1G, CCND2, KRT81, MRPS6, ANXA2, PTGER2, GLO1, HAVCR2, PYCARD, LAT2, SLC16A3, COTL1, PKM, TALDO1, CD96, NCR3, KRT86, STMN1, LTB, ARPC1B, ARPC5, FKBP1A, TIMP1, GZMK, CD59, PGK1, RGS10, EVL, RAC2, LGALS1, ITGB7, TUBB, PGAM1, PRF1, GZMB, IL2RB, KLRC2, and KLRB1 which is higher than expression of said markers in peripheral blood natural killer cells.

53. The natural killer cell of any one of claims 49-52, wherein expression of 2, 3, 4, 5, 6, 7, 8, 9, 10, or more markers selected from the group consisting of NDFIP2, LINC00996, MAL, CCL1, MB, SPINK2, C15orf48, CAMK1, KLRC1, TNFSF10, TNFRSF18, IL32, CAPG, AC092580.4, S100A11, TNFRSF4, ENO1, FCER1G, CCND2, KRT81, MRPS6, ANXA2, PTGER2, GLO1, HAVCR2, PYCARD, LAT2, SLC16A3, COTL1, PKM, TALDO1, CD96, NCR3, KRT86, STMN1, LTB, ARPC1B, ARPC5, FKBP1A, TIMP1, GZMK, CD59, PGK1, RGS10, EVL, RAC2, LGALS1, ITGB7, TUBB, PGAM1, PRF1, GZMB, IL2RB, KLRC2, and KLRB1 is higher than expression of said markers in peripheral blood natural killer cells.

54. The natural killer cell of any one of claims 49-53, wherein said viral infection is a coronavirus infection.

55. The natural killer cell of claim 54, wherein said coronavirus infection is selected from the group consisting of human coronavirus 229E (HCoV-229E), human coronavirus OC43 (HCoV-OC43), SARS-CoV, human coronavirus NL63 (HCoV-NL63, New Haven coronavirus), human coronavirus HKU1, middle east respiratory syndrome coronavirus (MERS-CoV, novel coronavirus 2012, HCoV-EMC), and novel coronavirus 2019-nCoV (SARS-CoV2).

56. The natural killer cell of claim 54, wherein said coronavirus infection is novel coronavirus SARS-CoV-2).

57. The natural killer cell of any one of claims 49-56, wherein the treatment comprises an improvement in score as measured by the Ordinal Scale for Clinical Improvement (OSCI).

58. The natural killer cell of any one of claims 49-56, wherein the treatment comprises a reduction in the time to improvement in score as measured by the Ordinal Scale for Clinical Improvement (OSCI).

59. The natural killer cell of any one of claims 49-56, wherein the treatment comprises an improvement in stratus by OSCI.

60. The natural killer cell of any one of claims 49-56, wherein the treatment comprises an improvement in time to and/or rate of clinical improvement by NEWS2 Score.

61. The natural killer cell of any one of claims 49-56, wherein the treatment comprises medical discharge or a reduced time to medical discharge.

62. The natural killer cell of any one of claims 49-56, wherein the treatment comprises reduced hospital utilization.

63. The natural killer cell of any one of claims 49-56, wherein the treatment comprises reduced mortality.

64. The natural killer cell of any one of claims 49-56, wherein the treatment comprises clearance of the virus or reduced time to clearance of the virus.

65. The natural killer cell of any one of claims 49-56, wherein the treatment comprises improved time to and/or rate of pulmonary clearance.

66. The natural killer cell of any one of claims 49-56, wherein the treatment comprises reduced duration of hospitalization.

67. The natural killer cell of any one of claims 49-56, wherein the treatment comprises an increase in supplemental oxygen-free days, a reduced need for supplemental oxygen, or a reduced time to cessation of supplemental oxygen.

68. The natural killer cell of any one of claims 49-56, wherein the treatment comprises a reduction in the requirement for ventilation.

69. The natural killer cell of any one of claims 49-56, wherein the treatment comprises an improvement in SOFA score.

70. The natural killer cell of any one of claims 49-56, wherein the treatment comprises an improvement in radiologic evaluation score.

71. The natural killer cell of any one of claims 49-56, wherein the treatment comprises an improvement in cytokine and/or chemokine assessment, preferably wherein the improvement in cytokine and/or chemokine assessment comprises a reduction in one or more inflammatory markers.

72. The natural killer cell of any one of claims 49-56, wherein the treatment comprises reduced or eliminated viral detection by RT-PCR.

73. The natural killer cell of any one of claims 49-72, wherein the treatment comprises two or more doses of natural killer cells.

74. The natural killer cell of any one of claims 49-72, wherein the treatment comprises a first dose and one or more subsequent doses of natural killer cells.

75. The natural killer cell of any one of claims 49-72, wherein the treatment comprises a first dose of between about 50×10⁶ natural killer cells to about and about 600×10⁶ natural killer cells.

76. The natural killer cell of any one of claims 49-72, wherein the treatment comprises a first dose and one or more subsequent doses of natural killer cells, wherein the one or more subsequent doses comprise about 150×10⁶ natural killer cells to about and about 2400×10⁶ natural killer cells.

77. The natural killer cell of any one of claims 49-72, wherein the treatment comprises a first dose and one or more subsequent doses of natural killer cells, wherein the one or more subsequent doses of natural killer cells are administered from about one to about five days after the previous doses, preferably wherein the one or more subsequent doses of natural killer cells are administered about three days after the previous dose.

78. The natural killer cell of any one of claims 49-72, wherein the treatment comprises an initial dose of 150×10⁶ cells on Day 1 followed by 600×10⁶ cells IV Days 4 and 7 or an initial dose of 150×10⁶ cells on Day 1 followed by 600×10⁶ cells IV Day 7.
